(19) United States
(12) Patent Application Publication
(10) Pub. No.: US 2023/0407311 A1
(43) Pub. Date: Dec. 21, 2023

(54) NUCLEIC ACIDS CONTAINING ABASIC NUCLEOTIDES

(71) Applicant: E-Therapeutics PLC, London (GB)

(72) Inventors: Ahmad Ali MORTAZAVI, London (GB); Viviana MANNELLA, London (GB); Muthusamy JAYARAMAN, London (GB)

(21) Appl. No.: 18/106,166

(22) Filed: Feb. 6, 2023

Related U.S. Application Data
(63) Continuation of application No. PCT/EP2022/052070, filed on Jan. 28, 2022.
(60) Provisional application No. 63/271,684, filed on Oct. 25, 2021.
(60) Provisional application No. 63/262,316, filed on Oct. 8, 2021.
(60) Provisional application No. 63/143,805, filed on Jan. 30, 2021.

(51) Int. Cl. C12N 15/113

(52) U.S. Cl. CPC C12N 15/1137; C12Y 101/03015; C12N 2310/14; C12N 2310/351; C12N 2310/315; C12N 2310/332; C12N 2310/344; C12N 2310/317

(57) ABSTRACT

The present invention relates to nucleic acid molecules for use in the treatment or prevention of disease.

### CROSS REFERENCE TO RELATED APPLICATIONS

This application is Continuation of International Application No. PCT/EP2022/052070, filed internationally on Jan. 28, 2022, which claims priority to U.S. Provisional Application No. 63/143,805, filed Jan. 30, 2021, U.S. Provisional Application No. 63/262,316, filed on Oct. 8, 2021, and U.S. Provisional Application No. 63/271,684, filed on Oct. 25, 2021, the contents of each of which are incorporated herein by reference in their entireties.

### REFERENCE TO AN ELECTRONIC SEQUENCE LISTING

The contents of the electronic sequence listing (228792001501substituteseglist.xml; Size: 223,050 bytes; and Date of Creation: May 15, 2023) is herein incorporated by reference in its entirety.

### FIELD

The present invention provides novel oligonucleotide compounds, which are nucleic acid compounds, suitable for therapeutic use. Additionally, the present invention provides methods of making these compounds, as well as methods of using such compounds for the treatment of various diseases and conditions.

### BACKGROUND OF THE INVENTION

Oligonucleotide compounds have important therapeutic applications in medicine. Oligonucleotides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleotides that prevent the formation of proteins by gene-silencing.

A number of modified siRNA compounds in particular have been developed in the last two decades for diagnostic and therapeutic purposes, including siRNA/RNAi therapeutic agents for the treatment of various diseases including central-nervous-system diseases, inflammatory diseases, metabolic disorders, oncology, infectious diseases, and ocular diseases.

The present invention relates to such oligonucleotide compounds, which are nucleic acid compounds, for use in the treatment and/or prevention of disease.

### STATEMENTS OF INVENTION

A nucleic acid, optionally an RNA, for inhibiting expression of a target gene in a cell, comprising at least one duplex region that comprises at least a portion of a first strand and at least a portion of a second strand that is at least partially complementary to the first strand, wherein said first strand is at least partially complementary to at least a portion of RNA transcribed from said target gene to be inhibited, wherein the second strand comprises one or more abasic nucleotides in a terminal region of the second strand, and wherein said abasic nucleotide(s) is/are connected to an adjacent nucleotide through a reversed internucleotide linkage.

A conjugate for inhibiting expression of a target gene in a cell, said conjugate comprising a nucleic acid portion and one or more ligand moieties, said nucleic acid portion comprising a nucleic acid as disclosed herein.

A pharmaceutical composition comprising a nucleic acid as disclosed herein or a conjugate as disclosed herein and a physiologically acceptable excipient.

### FIGURES

FIG. 1 shows analysis of hsC5 mRNA expression levels in a total of 45 human-derived cancer cell lysates and lysates of primary human hepatocytes (PHHs). mRNA expression levels are shown in relative light units [RLUs].

FIG. 2 shows analysis of hsHAO1 mRNA expression levels in a total of 45 human-derived cancer cell lysates and lysates of primary human hepatocytes (PHHs). mRNA expression levels are shown in relative light units [RLUs].

FIG. 3 shows analysis of hsTTR mRNA expression levels in a total of 45 human-derived cancer cell lysates and lysates of primary human hepatocytes (PHHs). mRNA expression levels are shown in relative light units [RLUs].

FIGS. 4A-4B shows the results from the dose-response analysis of hsTTR targeting GalNAc-siRNAs in HepG2 cells in Example 1.

FIGS. 5A-5B shows the results from the dose-response analysis of hsC5 targeting GalNAc-siRNAs in HepG2 cells in Example 1.

FIG. 6 shows the analysis of hsTTR (top), hsC5 (middle) and hsHAO1 (bottom) mRNA expression levels in all three batches of primary human hepatocytes BHuf16087 (left), CHF2101 (middle) and CyHuf19009 (right) each after 0 h, 24 h, 48 h and 72 h in culture. mRNA expression levels are shown in relative light units [RLUs].

FIG. 7 shows the analysis of hsGAPDH (top) and hsAHSA1 (bottom) mRNA expression levels in all three batches of primary human hepatocytes BHuf16087 (left), CHF2101 (middle) and CyHuf19009 (right) each after 0 h, 24 h, 48 h and 72 h in culture. mRNA expression levels are shown in relative light units [RLUs].

FIGS. 8A-8B shows the results from the dose-response analysis of hsHAO1 targeting GalNAc-siRNAs in PHHs in Example 1.

FIGS. 9A-9B shows the results from the dose-response analysis of hsC5 targeting GalNAc-siRNAs in PHHs in Example 1.

FIGS. 10A-10B shows the results from the dose-response analysis of hsTTR targeting GalNAc-siRNAs in PHHs in Example 1.

FIGS. 11-A-11B shows the results from the dose-response analysis of hsTTR targeting GalNAc-siRNAs in HepG2 cells in Example 3.

FIGS. 12-A-12B shows the results from the dose-response analysis of hsC5 targeting GalNAc-siRNAs in HepG2 cells in Example 3.

FIGS. 13-A-13B shows the results from the dose-response analysis of hsHAO1 targeting GalNAc-siRNAs in PHHs in Example 3.

FIGS. 14-A-14B shows the results from the dose-response analysis of hsC5 targeting GalNAc-siRNAs in PHHs in Example 3.

FIGS. 15-A-15B shows the results from the dose-response analysis of hsTTR targeting GalNAc-siRNAs in PHHs in Example 3.

FIG. 16. Single dose mouse pharmacology of ETX005. HAO1 mRNA expression is shown relative to the saline control group. Each point represents the mean and standard deviation of 3 mice.

FIG. 17. Single dose mouse pharmacology of ETX005. Serum glycolate concentration is shown. Each point represents the mean and standard deviation of 3 mice, except for baseline glycolate concentration (day 0) which was derived from a group of 5 mice.

FIG. 18. Single dose mouse pharmacology of ETX006. HAO1 mRNA expression is shown relative to the saline control group. Each point represents the mean and standard deviation of 3 mice.

FIG. 19. Single dose mouse pharmacology of ETX006. Serum glycolate concentration is shown. Each point represents the mean and standard deviation of 3 mice, except for baseline glycolate concentration (day 0) which was derived from a group of 5 mice.

FIG. 20. Single dose mouse pharmacology of ETX014. C5 mRNA expression is shown relative to the saline control group. Each point represents the mean and standard deviation of 3 mice.

FIG. 21. Single dose mouse pharmacology of ETX0014. Serum C5 concentration is shown relative to the saline control group. Each point represents the mean and standard deviation of 3 mice.

FIG. 22. Single dose mouse pharmacology of ETX015. C5 mRNA expression is shown relative to the saline control group. Each point represents the mean and standard deviation of 3 mice.

FIG. 23. Single dose mouse pharmacology of ETX0015. Serum C5 concentration is shown relative to the saline control group. Each point represents the mean and standard deviation of 3 mice.

FIG. 24. Single dose NHP pharmacology of ETX023. Serum TTR concentration is shown relative to day 1 of the study. Each point represents the mean and standard deviation of 3 animals.

FIG. 25. Single dose NHP pharmacology of ETX024. Serum TTR concentration is shown relative to day 1 of the study. Each point represents the mean and standard deviation of 3 animals.

FIG. 26. Single dose NHP pharmacology of ETX019. Serum TTR concentration is shown relative to day 1 of the study and also pre-dose. Each point represents the mean and standard deviation of 3 animals. Time points up to 84 days are shown.

FIG. 27. Single dose NHP pharmacology of ETX020. Serum TTR concentration is shown relative to day 1 of the study and also pre-dose. Each point represents the mean and standard deviation of 3 animals. Time points up to 84 days are shown.

FIG. 28A. Single dose NHP pharmacology of ETX023. Serum TTR concentration is shown relative to day 1 of the study and also pre-dose. Each point represents the mean and standard deviation of 3 animals. Time points up to 84 days are shown.

FIG. 28B. Sustained suppression of TTR gene expression in the liver after a single 1 mg/kg dose of ETX023. TTR mRNA is shown relative to baseline levels measured pre-dose. Each point represents the mean and standard deviation of 3 animals. Time points up to 84 days are shown.

FIG. 28C. Body weight of animals dosed with a single 1 mg/kg dose of ETX023. Each point represents the mean and standard deviation of 3 animals. Time points up to 84 days are shown.

FIG. 28D. ALT concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.) Time points up to 84 days are shown.

FIG. 28E. AST concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86. Time points up to 84 days are shown.

FIG. 29A. Single dose NHP pharmacology of ETX024. Serum TTR concentration is shown relative to day 1 of the study and also pre-dose. Each point represents the mean and standard deviation of 3 animals. Time points up to 84 days are shown.

FIG. 29B. Sustained suppression of TTR gene expression in the liver after a single 1 mg/kg dose of ETX024. TTR mRNA is shown relative to baseline levels measured pre-dose. Each point represents the mean and standard deviation of 3 animals. Time points up to 84 days are shown.

FIG. 29C. Body weight of animals dosed with a single 1 mg/kg dose of ETX024. Each point represents the mean and standard deviation of 3 animals. Time points up to 84 days are shown.

FIG. 29D. ALT concentration in serum from animals treated with a single 1 mg/kg dose of ETX024. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.) Time points up to 84 days are shown.

FIG. 29E. AST concentration in serum from animals treated with a single 1 mg/kg dose of ETX024. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86. Time points up to 84 days are shown.

FIG. 30. Linker and ligand portion of ETX005, 014, 023.

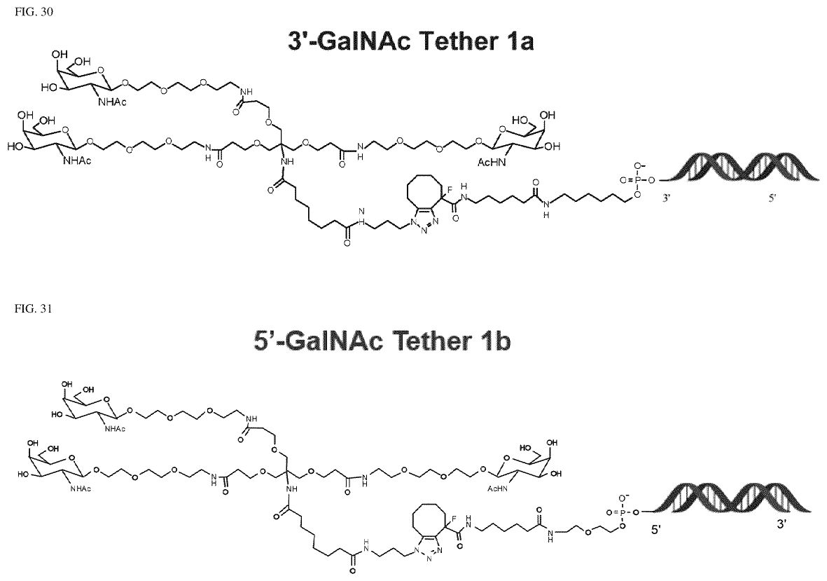

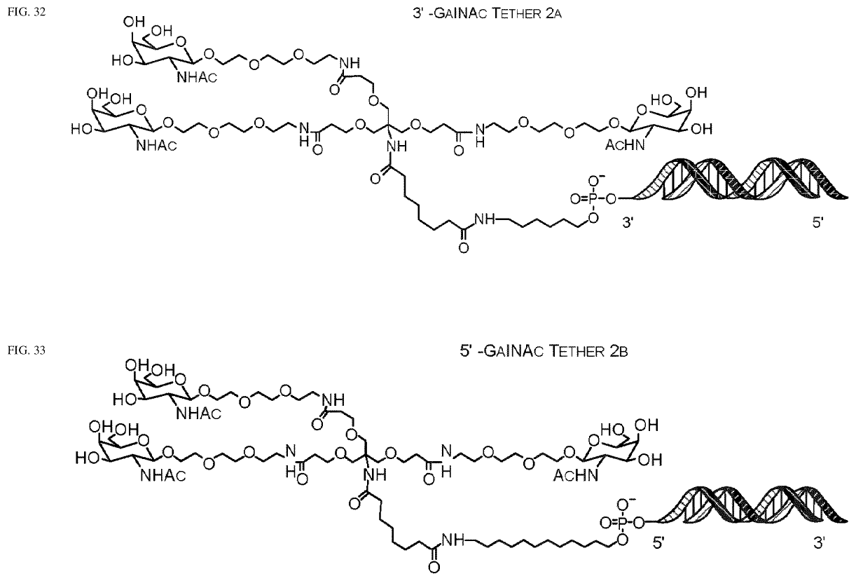

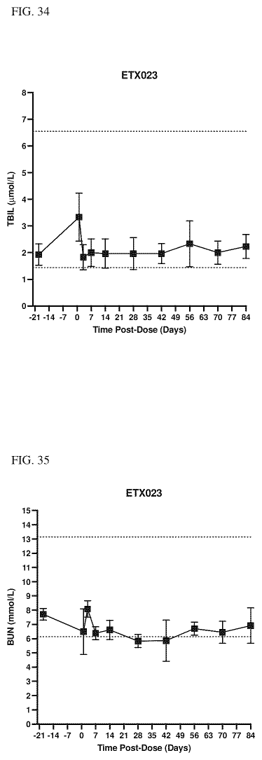

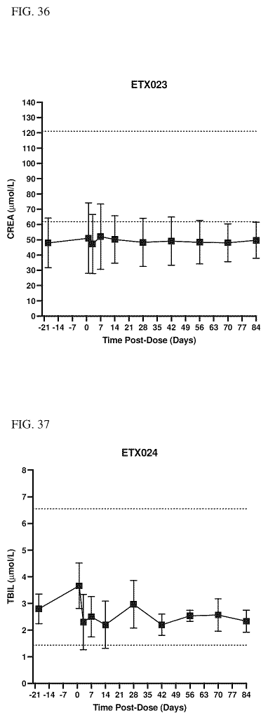

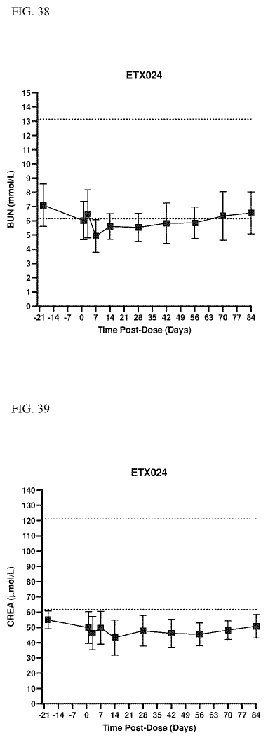

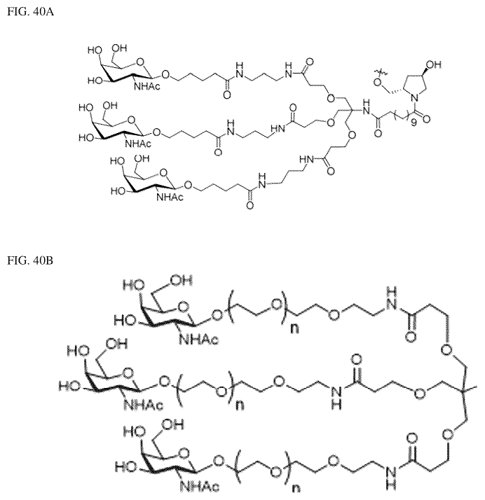

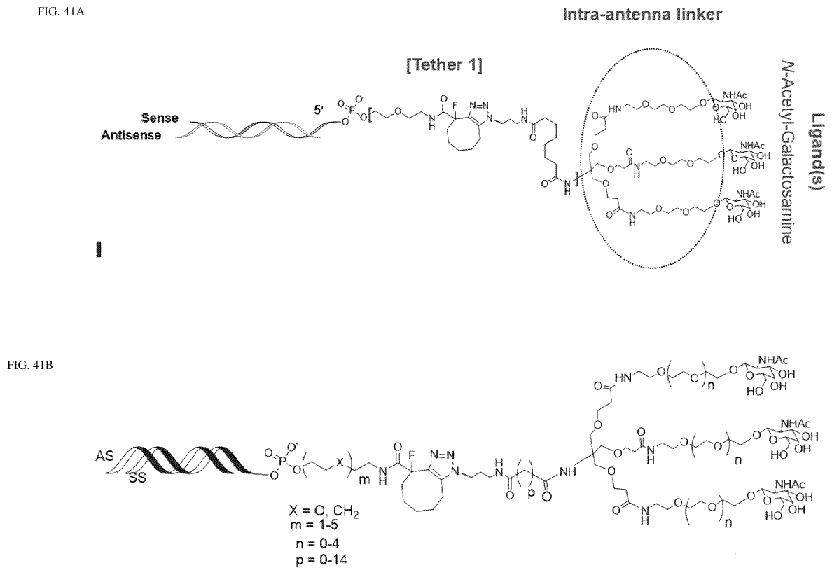

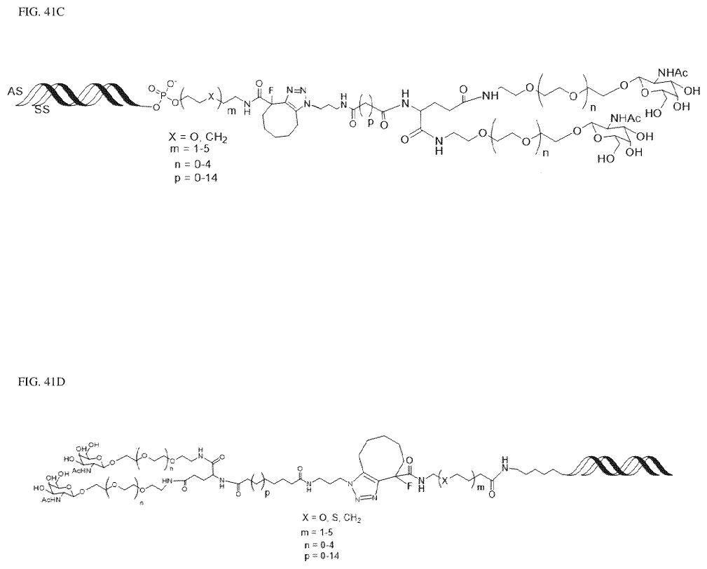

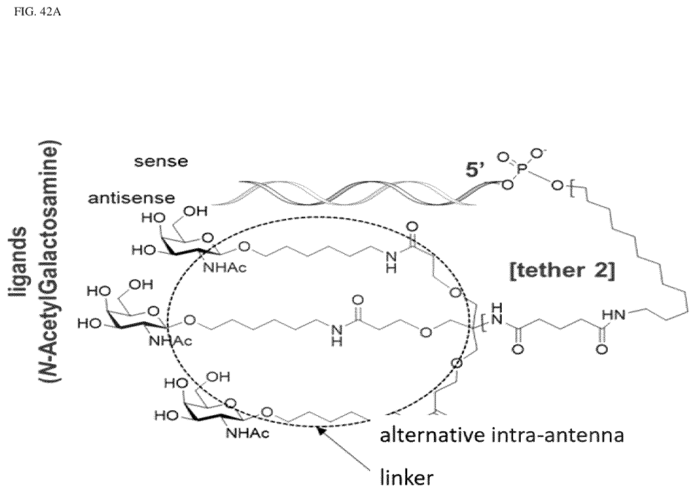

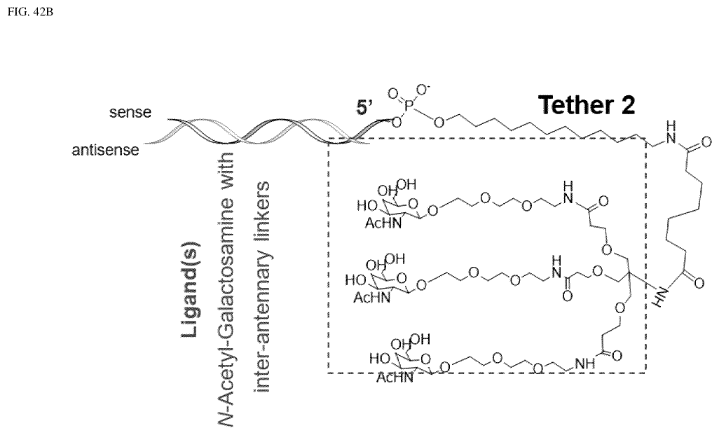

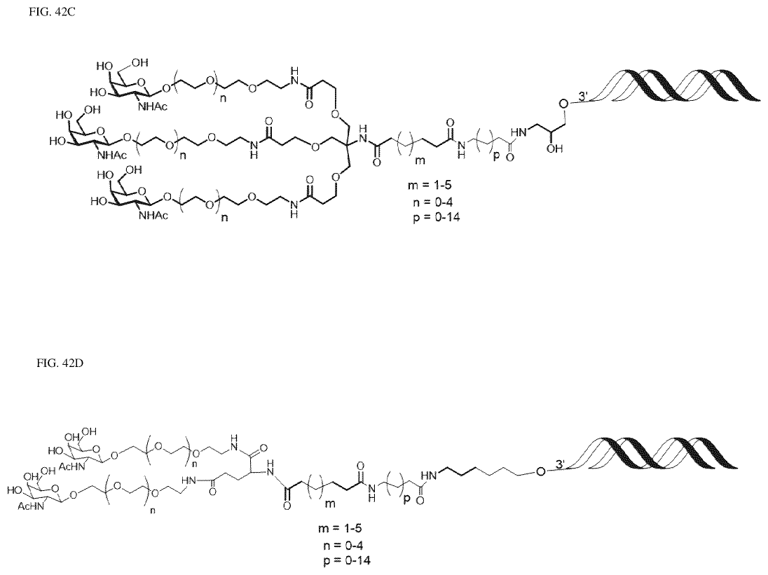

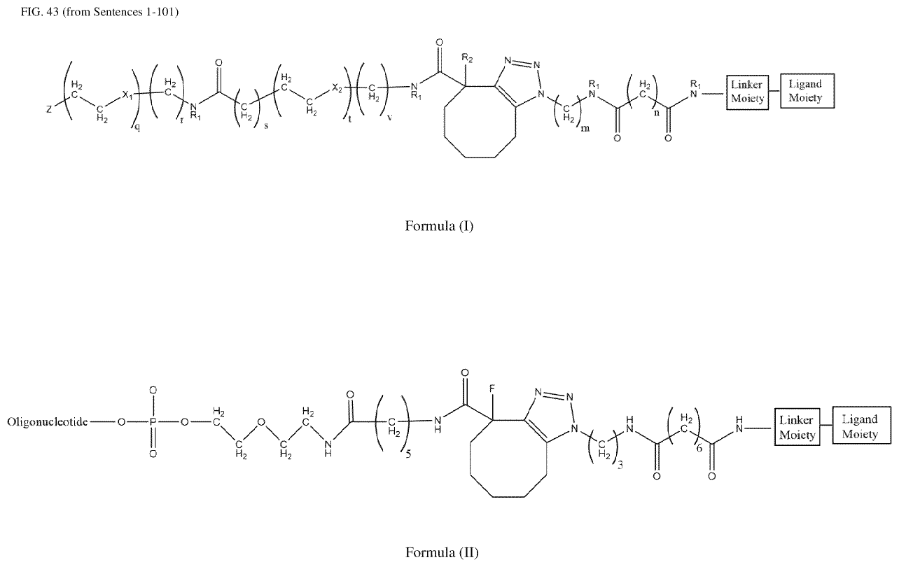

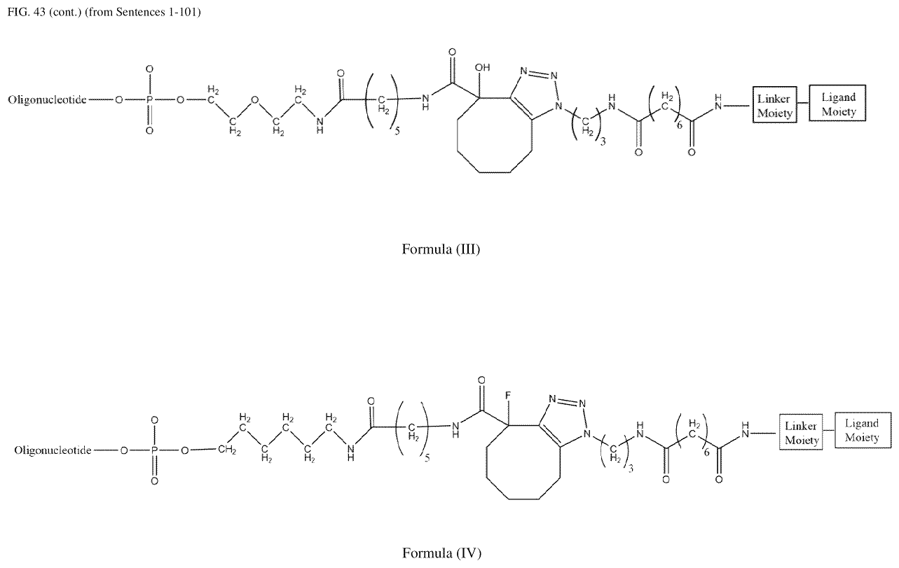

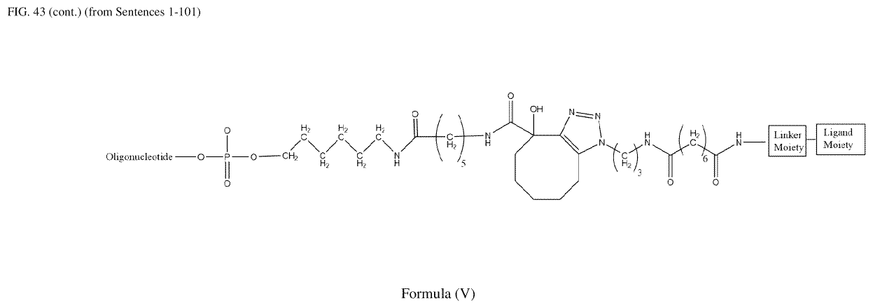

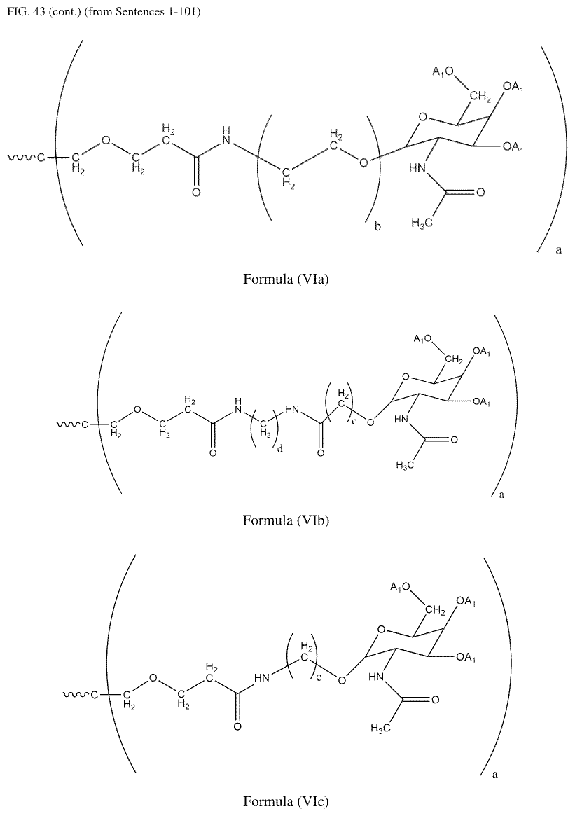

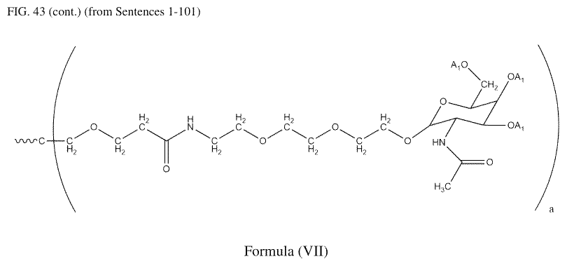

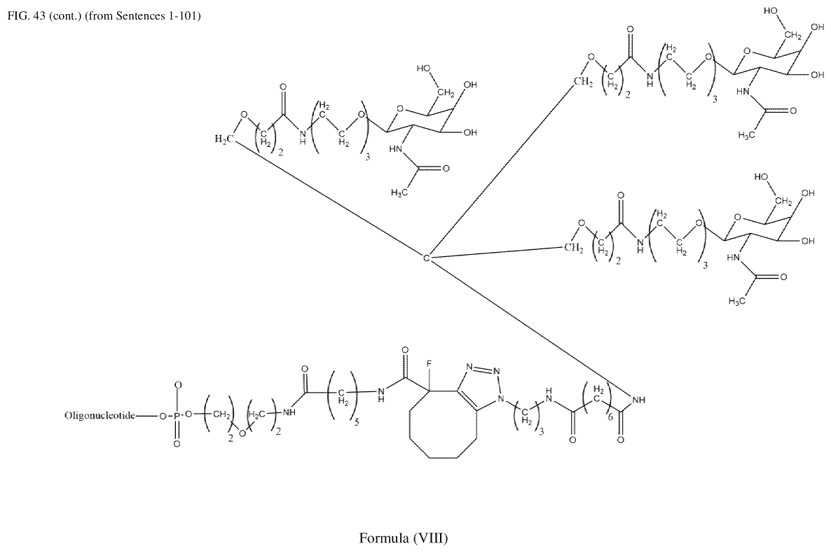

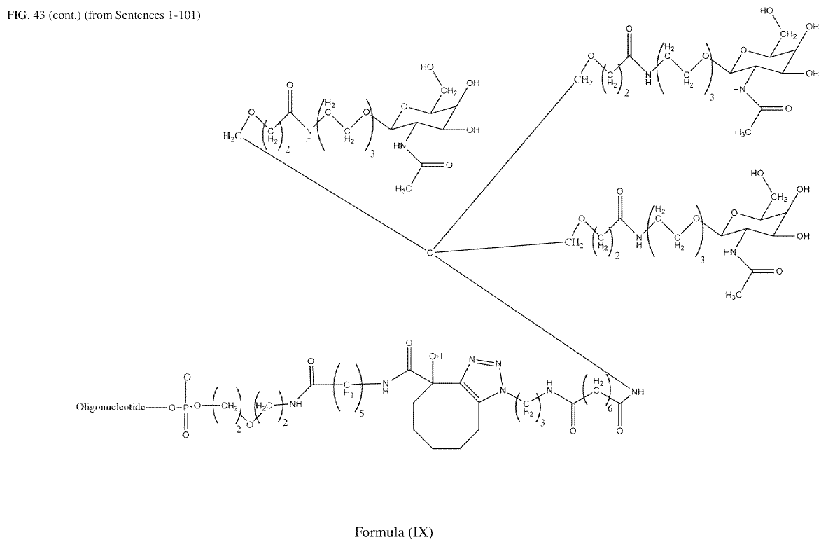

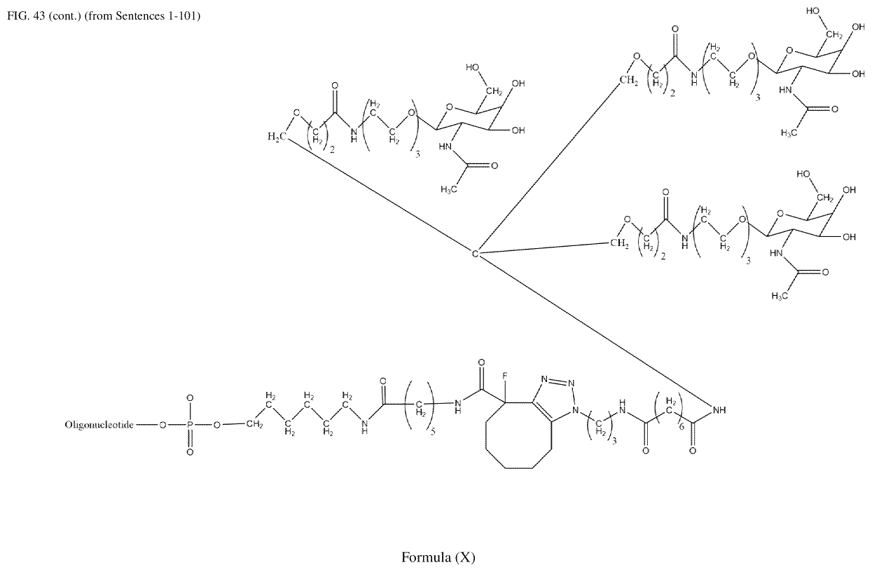

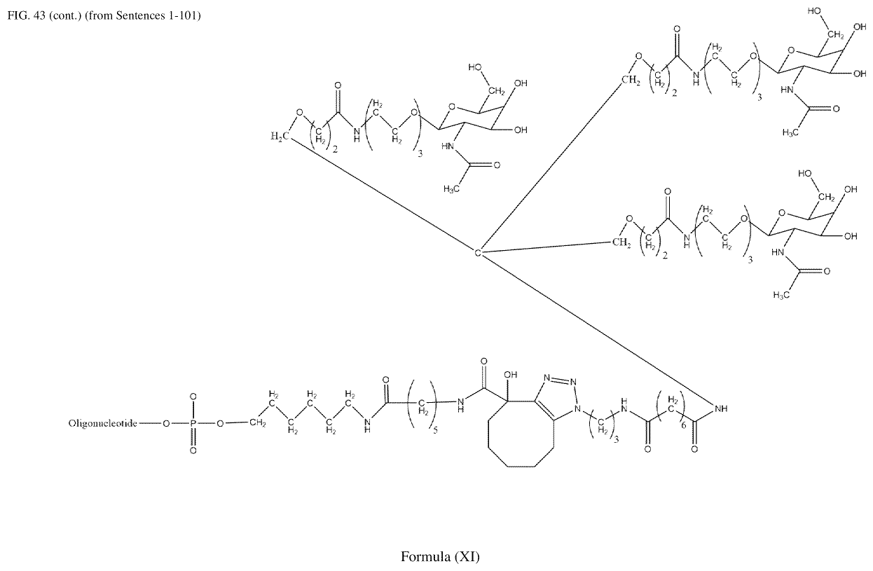

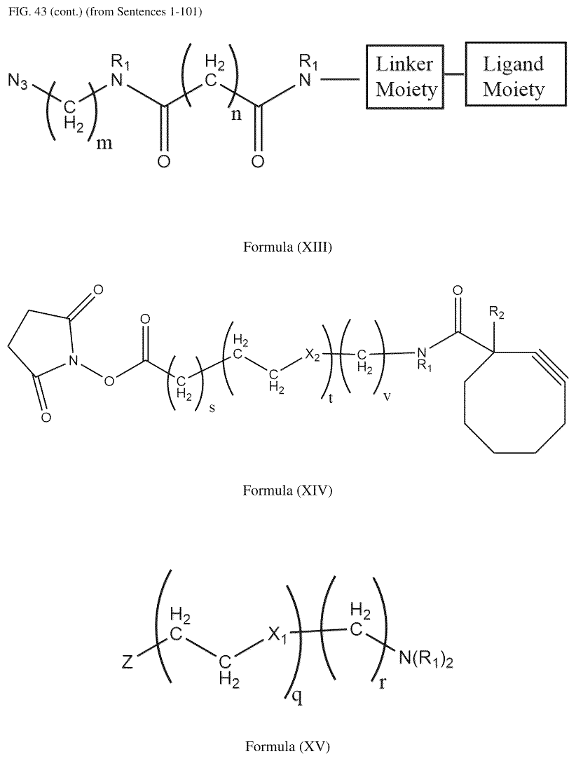

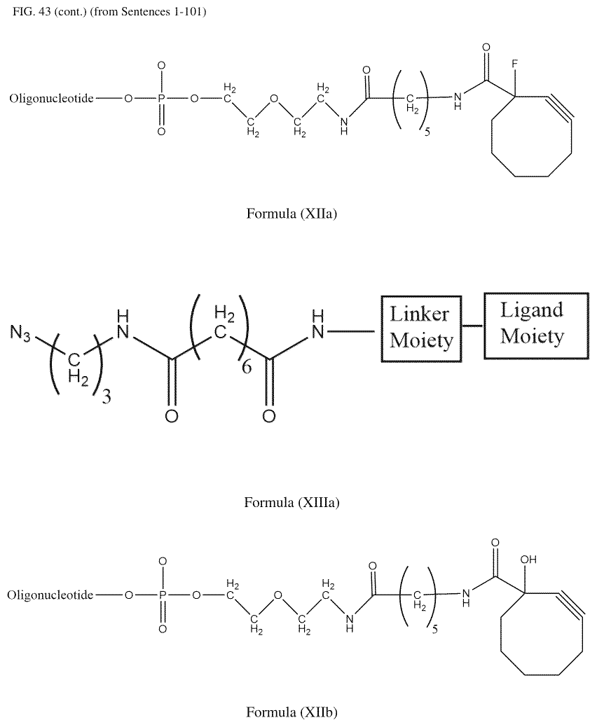

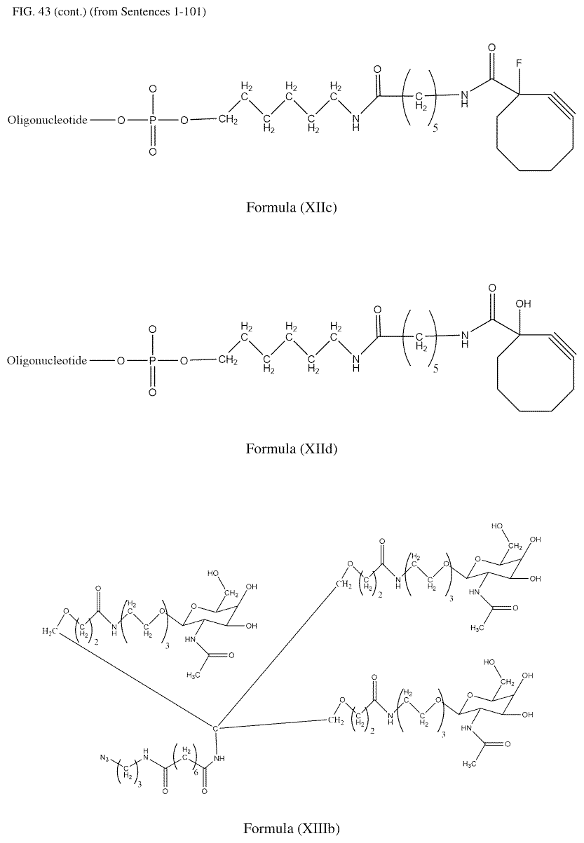

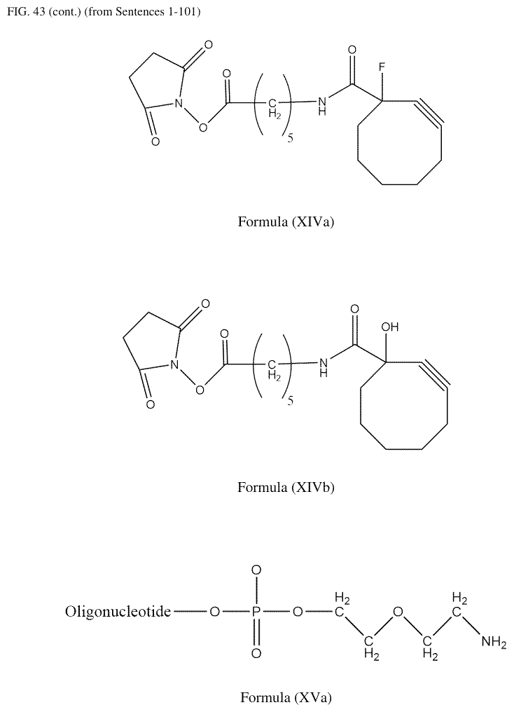

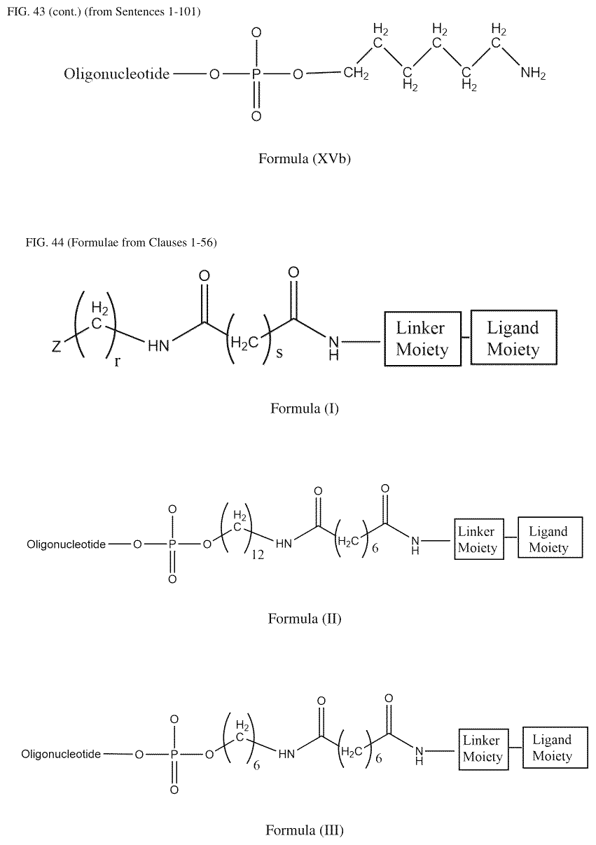

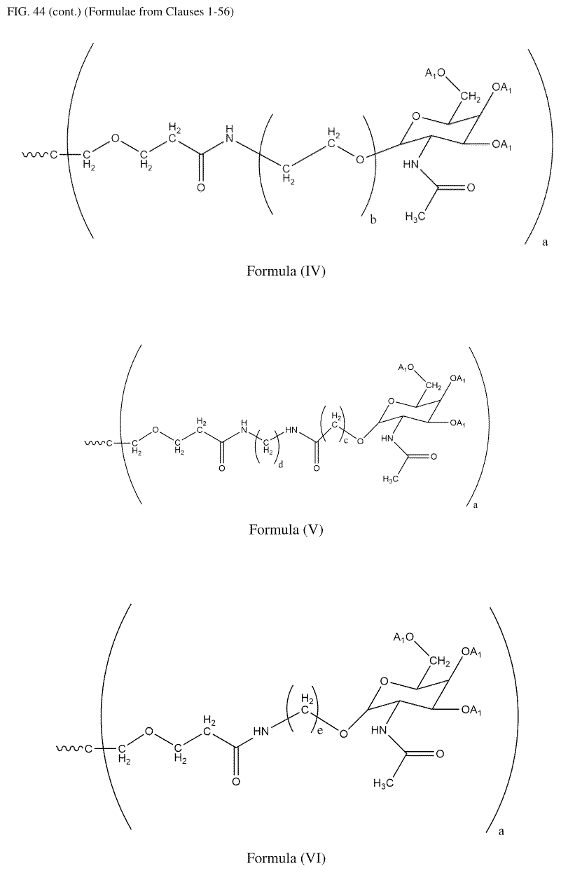

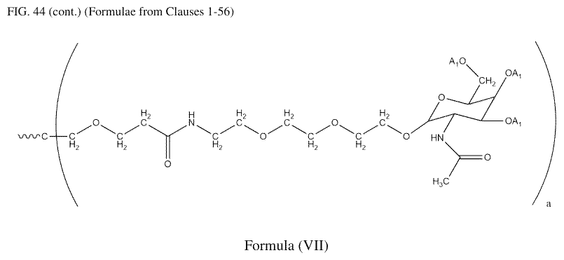

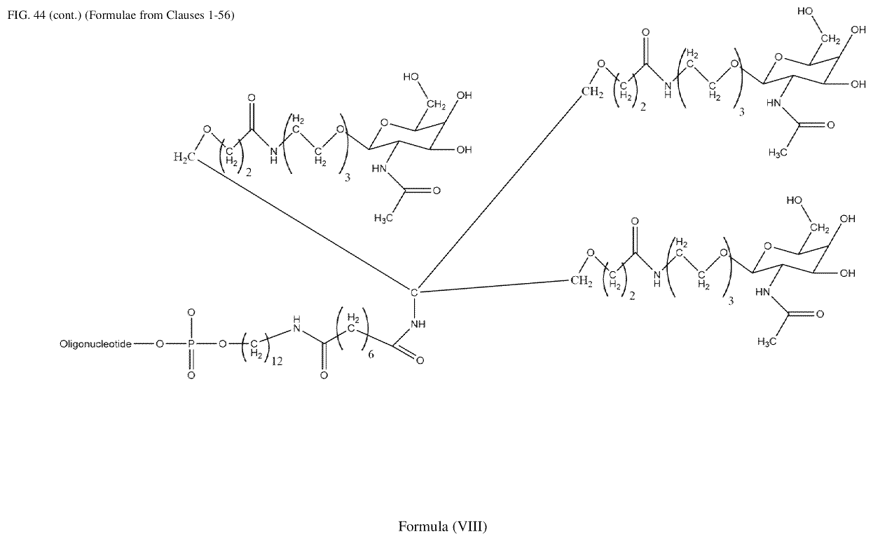

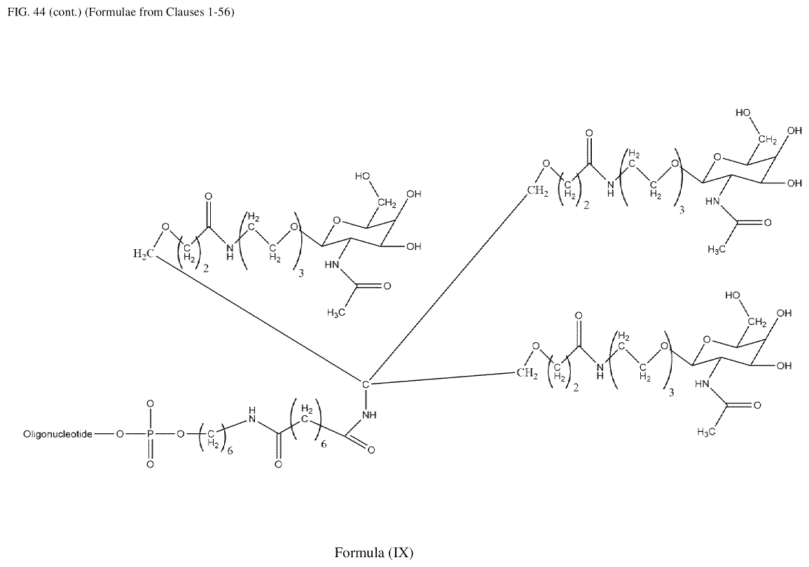

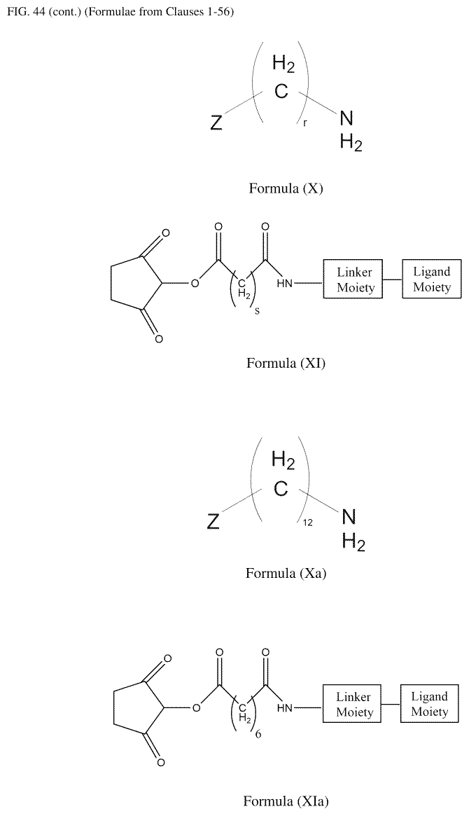

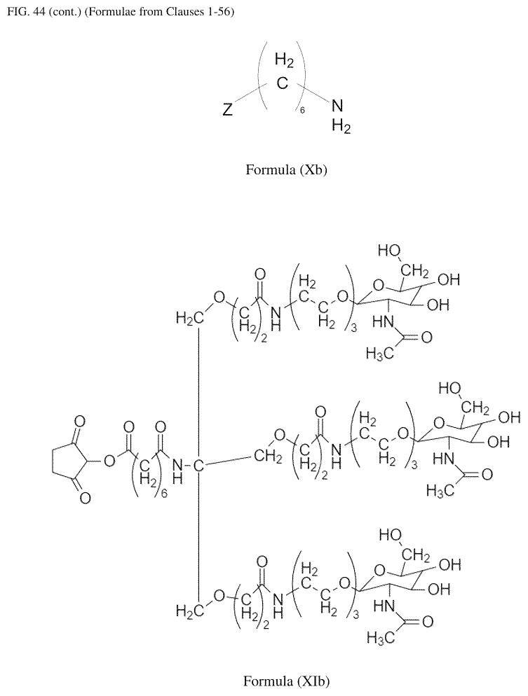

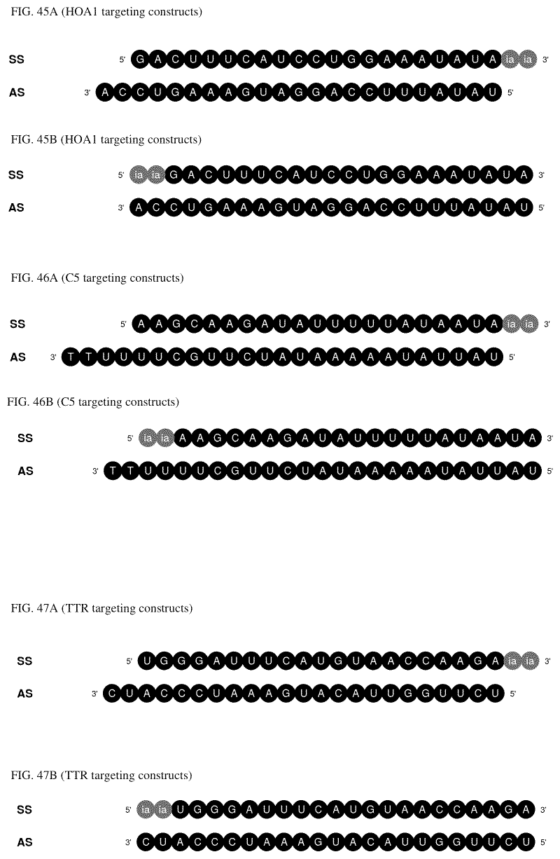

It should also be understood that where appropriate while ETX005 as a product includes molecules based on the linker and ligand portions as specifically depicted in FIG. 30 attached to an oligonucleotide moiety as also depicted herein, this ETX005 product may alternatively further comprise, or consist essentially of, molecules wherein the linker and ligand portions are essentially as depicted in FIG. 30 attached to an oligonucleotide moiety but having the F substituent as shown in FIG. 30 on the cyclo-octyl ring replaced by a substituent occurring as a result of hydrolytic displacement, such as an OH substituent. In this way, (a) ETX005 can consist essentially of molecules having linker and ligand portions specifically as depicted in FIG. 30, with a F substituent on the cyclo-octyl ring; or (b) ETX005 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX005 can comprise a mixture of molecules as defined in (a) and/or (b).

It should also be understood that where appropriate while ETX014 as a product includes molecules based on the linker and ligand portions as specifically depicted in FIG. 30 attached to an oligonucleotide moiety as also depicted herein, this ETX014 product may alternatively further comprise, or consist essentially of, molecules wherein the linker and ligand portions are essentially as depicted in FIG. 30 attached to an oligonucleotide moiety but having the F substituent as shown in FIG. 30 on the cyclo-octyl ring replaced by a substituent occurring as a result of hydrolytic displacement, such as an OH substituent. In this way, (a) ETX014 can consist essentially of molecules having linker and ligand portions specifically as depicted in FIG. 30, with a F substituent on the cyclo-octyl ring; or (b) ETX014 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX014 can comprise a mixture of molecules as defined in (a) and/or (b).

It should also be understood that where appropriate while ETX023 as a product includes molecules based on the linker and ligand portions as specifically depicted in FIG. 30 attached to an oligonucleotide moiety as also depicted herein, this ETX023 product may alternatively further comprise, or consist essentially of, molecules wherein the linker and ligand portions are essentially as depicted in FIG. 30 attached to an oligonucleotide moiety but having the F substituent as shown in FIG. 30 on the cyclo-octyl ring replaced by a substituent occurring as a result of hydrolytic displacement, such as an OH substituent. In this way, (a) ETX023 can consist essentially of molecules having linker and ligand portions specifically as depicted in FIG. 30, with a F substituent on the cyclo-octyl ring; or (b) ETX023 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX023 can comprise a mixture of molecules as defined in (a) and/or (b).

FIG. 31. Linker and ligand portion of ETX001, 010 and 019.

It should also be understood that where appropriate while ETX001 as a product includes molecules based on the linker and ligand portions as specifically depicted in FIG. 31 attached to an oligonucleotide moiety as also depicted herein, this ETX001 product may alternatively further comprise, or consist essentially of, molecules wherein the linker and ligand portions are essentially as depicted in FIG. 31 attached to an oligonucleotide moiety but having the F substituent as shown in FIG. 31 on the cyclo-octyl ring replaced by a substituent occurring as a result of hydrolytic displacement, such as an OH substituent. In this way, (a) ETX001 can consist essentially of molecules having linker and ligand portions specifically as depicted in FIG. 31, with a F substituent on the cyclo-octyl ring; or (b) ETX001 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX001 can comprise a mixture of molecules as defined in (a) and/or (b).

It should also be understood that where appropriate while ETX010 as a product includes molecules based on the linker and ligand portions as specifically depicted in FIG. 31 attached to an oligonucleotide moiety as also depicted herein, this ETX010 product may alternatively further comprise, or consist essentially of, molecules wherein the linker and ligand portions are essentially as depicted in FIG. 31 attached to an oligonucleotide moiety but having the F substituent as shown in FIG. 31 on the cyclo-octyl ring replaced by a substituent occurring as a result of hydrolytic displacement, such as an OH substituent. In this way, (a) ETX010 can consist essentially of molecules having linker and ligand portions specifically as depicted in FIG. 31, with a F substituent on the cyclo-octyl ring; or (b) ETX010 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX010 can comprise a mixture of molecules as defined in (a) and/or (b).

It should also be understood that where appropriate while ETX019 as a product includes molecules based on the linker and ligand portions as specifically depicted in FIG. 31 attached to an oligonucleotide moiety as also depicted herein, this ETX019 product may alternatively further comprise, or consist essentially of, molecules wherein the linker and ligand portions are essentially as depicted in FIG. 31 attached to an oligonucleotide moiety but having the F substituent as shown in FIG. 31 on the cyclo-octyl ring replaced by a substituent occurring as a result of hydrolytic displacement, such as an OH substituent. In this way, (a) ETX019 can consist essentially of molecules having linker and ligand portions specifically as depicted in FIG. 31, with a F substituent on the cyclo-octyl ring; or (b) ETX019 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX019 can comprise a mixture of molecules as defined in (a) and/or (b).

FIG. 32. Linker and ligand portion of ETX006, 015 and 024.

FIG. 33. Linker and ligand portion of ETX002, 011 and 020.

FIG. 34. Total bilirubin concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.).

FIG. 35. Blood urea nitrogen (BUN) concentration from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.).

FIG. 36. Creatinine (CREA) concentration from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.).

FIG. 37. Total bilirubin concentration in serum from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.).

FIG. 38. Blood urea nitrogen (BUN) concentration from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.).

FIG. 39. Creatinine (CREA) concentration from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.).

FIG. 40A depicts a tri-antennary GalNAc (N-acetylgalactosamine) unit. FIG. 40B depicts an alternative tri-antennary GalNAc according to one embodiment of the invention, showing variance in linking groups. FIG. 41A depicts tri-antennary GalNAc-conjugated siRNA according to the invention, showing variance in the linking groups. FIG. 41B depicts a genera of tri-antennary GalNAc-conjugated siRNAs according to one embodiment of the invention. FIG. 41C depicts a genera of bi-antennary GalNAc-conjugated siRNAs according to one embodiment of the invention, showing variance in the linking groups. FIG. 41D depicts a genera of bi-antennary GalNAc-conjugated siRNAs according to another embodiment of the invention, showing variance in the linking groups. FIG. 42A depicts another embodiment of the tri-antennary GalNAc-conjugated siRNA according to one embodiment of the invention. FIG. 42B depicts a variant shown in FIG. 27A, having an alternative branching GalNAc conjugate. FIG. 42C depicts a genera of tri-antennary GalNAc-conjugated siRNAs according to one embodiment of the invention, showing variance in the linking groups. FIG. 42D depicts a genera of bi-antennary GalNAc-conjugated siRNAs according to one embodiment the invention, showing variance in the linking groups.

FIG. 43 shows the detail of the formulae described in Sentences 1-101 disclosed herein.

FIG. 44 shows the detail of formulae described in Clauses 1-56 disclosed herein

FIG. 45A shows the underlying nucleotide sequences for the sense (SS) and antisense (AS) strands of constructs ETX001, ETX002 as described herein. For both ETX001 and ETX002 a galnac linker is attached to the 5′ end region of the sense strand in use (not depicted in FIG. 45A). For ETX001 the galnac linker is attached and as shown in FIG. 31. For ETX002 the galnac linker is attached and as shown in FIG. 33.

iaia as shown at the 3′ end region of the sense strand in FIG. 45A represents (i) two abasic nucleotides provided as the penultimate and terminal nucleotides at the 3′ end region of the sense strand, (ii) wherein a 3′-3′ reversed linkage is provided between the antepenultimate nucleotide (namely A at position 21 of the sense strand, wherein position 1 is the terminal 5′ nucleotide of the sense strand, namely terminal G at the 5′end region of the sense strand) and the adjacent penultimate abasic residue of the sense strand, and (iii) the linkage between the terminal and penultimate abasic nucleotides is 5′-3′ when reading towards the 3′ end region comprising the terminal and penultimate abasic nucleotides.

For the sense strand of FIG. 45A, when reading from position 1 of the sense strand (which is the terminal 5′ nucleotide of the sense strand, namely terminal G at the 5′end region of the sense strand), then: (i) the nucleotides at positions 1 to 6, 8, and 12 to 21 have sugars that are 2′ O-methyl modified, (ii) the nucleotides at positions 7, and 9 to 11 have sugars that are 2′ F modified, (iii) the abasic nucleotides have sugars that have H at positions 1 and 2.

For the antisense strand of FIG. 45A, when reading from position 1 of the antisense strand (which is the terminal 5′ nucleotide of the antisense strand, namely terminal U at the 5′end region of the antisense strand), then: (i) the nucleotides at positions 1, 3 to 5, 7, 10 to 13, 15, 17 to 23 have sugars that are 2′ O-methyl modified, (ii) the nucleotides at positions 2, 6, 8, 9, 14, 16 have sugars that are 2′ F modified.

FIG. 45B shows the underlying nucleotide sequences for the sense (SS) and antisense (AS) strands of constructs ETX005, ETX006 as described herein. For both ETX005 and ETX006 a galnac linker is attached to the 3′ end region of the sense strand in use (not depicted in FIG. 45B). For ETX005 the galnac linker is attached and as shown in FIG. 30. For ETX006 the galnac linker is attached and as shown in FIG. 32.

iaia as shown at the 5′ end region of the sense strand in FIG. 45B represents (i) two abasic nucleotides provided as the penultimate and terminal nucleotides at the 5′ end region of the sense strand, (ii) wherein a 5′-5′ reversed linkage is provided between the antepenultimate nucleotide (namely G at position 1 of the sense strand, not including the iaia motif at the 5′ end region of the sense strand in the nucleotide position numbering on the sense strand) and the adjacent penultimate abasic residue of the sense strand, and (iii) the linkage between the terminal and penultimate abasic nucleotides is 3′-5′ when reading towards the 5′ end region comprising the terminal and penultimate abasic nucleotides.

For the sense strand of FIG. 45B, when reading from position 1 of the sense strand (which is the terminal 5′ nucleotide of the sense strand, namely terminal G at the 5′end region of the sense strand, not including the iaia motif at the 5′ end region of the sense strand in the nucleotide position numbering on the sense strand), then: (i) the nucleotides at positions 1 to 6, 8, and 12 to 21 have sugars that are 2′ O-methyl modified, (ii) the nucleotides at positions 7, and 9 to 11 have sugars that are 2′ F modified, (iii) the abasic nucleotides have sugars that have H at positions 1 and 2.

For the antisense strand of FIG. 45B, when reading from position 1 of the antisense strand (which is the terminal 5′ nucleotide of the antisense strand, namely terminal U at the 5′end region of the antisense strand), then: (i) the nucleotides at positions 1, 3 to 5, 7, 10 to 13, 15, 17 to 23 have sugars that are 2′ O-methyl modified, (ii) the nucleotides at positions 2, 6, 8, 9, 14, 16 have sugars that are 2′ F modified.

FIG. 46A shows the underlying nucleotide sequences for the sense (SS) and antisense (AS) strands of constructs ETX010, ETX011 as described herein. For both ETX010 and ETX011 a galnac linker is attached to the 5′ end region of the sense strand in use (not depicted in FIG. 46A). For ETX010 the galnac linker is attached and as shown in FIG. 31. For ETX011 the galnac linker is attached and as shown in FIG. 33.

iaia as shown at the 3′ end region of the sense strand in FIG. 46A represents (i) two abasic nucleotides provided as the penultimate and terminal nucleotides at the 3′ end region of the sense strand, (ii) wherein a 3′-3′ reversed linkage is provided between the antepenultimate nucleotide (namely A at position 21 of the sense strand, wherein position 1 is the terminal 5′ nucleotide of the sense strand, namely terminal A at the 5′end region of the sense strand) and the adjacent penultimate abasic residue of the sense strand, and (iii) the linkage between the terminal and penultimate abasic nucleotides is 5′-3′ when reading towards the 3′ end region comprising the terminal and penultimate abasic nucleotides.

For the sense strand of FIG. 46A, when reading from position 1 of the sense strand (which is the terminal 5′ nucleotide of the sense strand, namely terminal A at the 5′end region of the sense strand), then: (i) the nucleotides at positions 1, 2, 4, 6, 8, 12, 14, 15, 17, 19 to 21 have sugars that are 2′ O-methyl modified, (ii) the nucleotides at positions 3, 5, 7, 9 to 11, 13, 16, 18 have sugars that are 2′ F modified, (iii) the abasic nucleotides have sugars that have H at positions 1 and 2.

For the antisense strand of FIG. 46A, when reading from position 1 of the antisense strand (which is the terminal 5′ nucleotide of the antisense strand, namely terminal U at the 5′end region of the antisense strand), then: (i) the nucleotides at positions 1, 4, 6, 7, 9, 11 to 13, 15, 17, 19 to 23 have sugars that are 2′ O-methyl modified, (ii) the nucleotides at positions 2, 3, 5, 8, 10, 14, 16, 18 have sugars that are 2′ F modified, (iii) the penultimate and terminal T nucleotides at positions 24, 25 at the 3′ end region of the antisense strand have sugars that have H at position 2.

FIG. 46B shows the underlying nucleotide sequences for the sense (SS) and antisense (AS) strands of constructs ETX014, ETX015 as described herein. For both ETX014 and ETX015 a galnac linker is attached to the 3′ end region of the sense strand in use (not depicted in FIG. 46B). For ETX014 the galnac linker is attached and as shown in FIG. 30. For ETX015 the galnac linker is attached and as shown in FIG. 32.

iaia as shown at the 5′ end region of the sense strand in FIG. 46B represents (i) two abasic nucleotides provided as the penultimate and terminal nucleotides at the 5′ end region of the sense strand, (ii) wherein a 5′-5′ reversed linkage is provided between the antepenultimate nucleotide (namely A at position 1 of the sense strand, not including the iaia motif at the 5′ end region of the sense strand in the nucleotide position numbering on the sense strand) and the adjacent penultimate abasic residue of the sense strand, and (iii) the linkage between the terminal and penultimate abasic nucleotides is 3′-5′ when reading towards the 5′ end region comprising the terminal and penultimate abasic nucleotides.

For the sense strand of FIG. 46B, when reading from position 1 of the sense strand (which is the terminal 5′ nucleotide of the sense strand, namely terminal A at the 5′end region of the sense strand, not including the iaia motif at the 5′ end region of the sense strand in the nucleotide position numbering on the sense strand), then: (i) the nucleotides at positions 1, 2, 4, 6, 8, 12, 14, 15, 17, 19 to 21 have sugars that are 2′ O-methyl modified, (ii) the nucleotides at positions 3, 5, 7, 9 to 11, 13, 16, 18 have sugars that are 2′ F modified, (iii) the abasic nucleotides have sugars that have H at positions 1 and 2.

For the antisense strand of FIG. 46B, when reading from position 1 of the antisense strand (which is the terminal 5′ nucleotide of the antisense strand, namely terminal U at the 5′end region of the antisense strand), then: (i) the nucleotides at positions 1, 4, 6, 7, 9, 11 to 13, 15, 17, 19 to 23 have sugars that are 2′ O-methyl modified, (ii) the nucleotides at positions 2, 3, 5, 8, 10, 14, 16, 18 have sugars that are 2′ F modified, (iii) the penultimate and terminal T nucleotides at positions 24, 25 at the 3′ end region of the antisense strand have sugars that have H at position 2.

FIG. 47A shows the underlying nucleotide sequences for the sense (SS) and antisense (AS) strands of constructs ETX019, ETX020 as described herein. For both ETX019 and ETX020 a galnac linker is attached to the 5′ end region of the sense strand in use (not depicted in FIG. 47A). For ETX019 the galnac linker is attached and as shown in FIG. 31. For ETX020 the galnac linker is attached and as shown in FIG. 33.

iaia as shown at the 3′ end region of the sense strand in FIG. 47A represents (i) two abasic nucleotides provided as the penultimate and terminal nucleotides at the 3′ end region of the sense strand, (ii) wherein a 3′-3′ reversed linkage is provided between the antepenultimate nucleotide (namely A at position 21 of the sense strand, wherein position 1 is the terminal 5′ nucleotide of the sense strand, namely terminal U at the 5′end region of the sense strand) and the adjacent penultimate abasic residue of the sense strand, and (iii) the linkage between the terminal and penultimate abasic nucleotides is 5′-3′ when reading towards the 3′ end region comprising the terminal and penultimate abasic nucleotides.

For the sense strand of FIG. 47A, when reading from position 1 of the sense strand (which is the terminal 5′ nucleotide of the sense strand, namely terminal U at the 5′end region of the sense strand), then: (i) the nucleotides at positions 1 to 6, 8, and 12 to 21 have sugars that are 2′ O-methyl modified, (ii) the nucleotides at positions 7, and 9 to 11 have sugars that are 2′ F modified, (iii) the abasic nucleotides have sugars that have H at positions 1 and 2.

For the antisense strand of FIG. 47A, when reading from position 1 of the antisense strand (which is the terminal 5′ nucleotide of the antisense strand, namely terminal U at the 5′end region of the antisense strand), then: (i) the nucleotides at positions 1, 3 to 5, 7, 8, 10 to 13, 15, 17 to 23 have sugars that are 2′ O-methyl modified, (ii) the nucleotides at positions 2, 6, 9, 14, 16 have sugars that are 2′ F modified.

FIG. 47B shows the underlying nucleotide sequences for the sense (SS) and antisense (AS) strands of constructs ETX023, ETX024 as described herein. For both ETX023 and ETX024 a galnac linker is attached to the 3′ end region of the sense strand in use (not depicted in FIG. 47B). For ETX023 the galnac linker is attached and as shown in FIG. 30. For ETX024 the galnac linker is attached and as shown in FIG. 32.

iaia as shown at the 5′ end region of the sense strand in FIG. 47B represents (i) two abasic nucleotides provided as the penultimate and terminal nucleotides at the 5′ end region of the sense strand, (ii) wherein a 5′-5′ reversed linkage is provided between the antepenultimate nucleotide (namely U at position 1 of the sense strand, not including the iaia motif at the 5′ end region of the sense strand in the nucleotide position numbering on the sense strand) and the adjacent penultimate abasic residue of the sense strand, and (iii) the linkage between the terminal and penultimate abasic nucleotides is 3′-5′ when reading towards the 5′ end region comprising the terminal and penultimate abasic nucleotides.

For the sense strand of FIG. 47B, when reading from position 1 of the sense strand (which is the terminal 5′ nucleotide of the sense strand, namely terminal U at the 5′end region of the sense strand, not including the iaia motif at the 5′ end region of the sense strand in the nucleotide position numbering on the sense strand), then: (i) the nucleotides at positions 1 to 6, 8, and 12 to 21 have sugars that are 2′ O-methyl modified, (ii) the nucleotides at positions 7, and 9 to 11 have sugars that are 2′ F modified, (iii) the abasic nucleotides have sugars that have H at positions 1 and 2.

For the antisense strand of FIG. 47B, when reading from position 1 of the antisense strand (which is the terminal 5′ nucleotide of the antisense strand, namely terminal U at the 5′end region of the antisense strand), then: (i) the nucleotides at positions 1, 3 to 5, 7, 8, 10 to 13, 15, 17 to 23 have sugars that are 2′ O-methyl modified, (ii) the nucleotides at positions 2, 6, 9, 14, 16 have sugars that are 2′ F modified.

### SUMMARY OF THE INVENTION

The present invention provides nucleic acids such as inhibitory RNA molecules (which may be referred to as iRNA), and compositions containing the same which can affect expression of a target gene. The gene may be within a cell, e.g. a cell within a subject, such as a human. The nucleic acids can be used to prevent and/or treat medical conditions associated with the expression of a target gene. Inhibitory RNA (iRNA) is the preferred nucleic acid herein.

The invention provides, in a first aspect, a nucleic acid, optionally an RNA, for inhibiting expression of a target gene in a cell, comprising at least one duplex region that comprises at least a portion of a first strand and at least a portion of a second strand that is at least partially complementary to the first strand, wherein said first strand is at least partially complementary to at least a portion of RNA transcribed from said target gene to be inhibited, wherein the second strand comprises one or more abasic nucleotides in a terminal region of the second strand, and wherein said abasic nucleotide(s) is/are connected to an adjacent nucleotide through a reversed internucleotide linkage.

The invention in particular includes double stranded RNA molecules (dsRNA) which includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a gene of interest. The other strand (the sense strand) includes a region that is complementary to the antisense strand, such that the two strands hybridize and form a duplex structure when combined under suitable conditions. Complementary sequences of a dsRNA can also be self-complementary regions of a single nucleic acid molecule.

### Definitions

The “first strand”, also called the antisense strand or guide strand herein and which can be used interchangeably herein, refers to the nucleic acid strand, e.g. the strand of an iRNA, e.g. a dsRNA, which includes a region that is substantially complementary to a target sequence, e.g. to an mRNA. As used herein, the term “region of complementarity” refers to the region on the antisense strand that is substantially complementary to a sequence, for example a target sequence. Where the region of complementarity is not fully complementary to the target sequence, the mismatches can be in the internal or terminal regions of the molecule. In some embodiments, a double stranded nucleic acid e.g. RNAi agent of the invention includes a nucleotide mismatch in the antisense strand.

In the context of molecule comprising a nucleic acid provided with a ligand moiety, optionally also with a linker moiety, the nucleic acid of the invention may be referred to as an oligonucleotide moiety.

In some embodiments, a double stranded nucleic acid e.g. RNAi agent of the invention includes a nucleotide mismatch in the sense strand. In some embodiments, the nucleotide mismatch is, for example, within 5, 4, 3, 2, or 1 nucleotides from the 3′-end of the nucleic acid e.g. iRNA.

In another embodiment, the nucleotide mismatch is, for example, in the 3′-terminal nucleotide of the nucleic acid e.g. iRNA.

The “second strand” (also called the sense strand or passenger strand herein, and which can be used interchangeably herein), refers to the strand of a nucleic acid e.g. iRNA that includes a region that is substantially complementary to a region of the antisense strand as that term is defined herein.

A “target sequence” (which may also be called a target RNA or a target mRNA) refers to a contiguous portion of the nucleotide sequence of an mRNA molecule formed during the transcription of a gene, including mRNA that is a product of RNA processing of a primary transcription product.

The target sequence may be from about 10-35 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

The term “ribonucleotide” or “nucleotide” can also refer to a modified nucleotide, as further detailed below.

A nucleic acid can be a DNA or an RNA, and can comprise modified nucleotides. RNA is a preferred nucleic acid.

The terms “iRNA”, “RNAi agent,” and “iRNA agent,” “RNA interference agent” as used interchangeably herein, refer to an agent that contains RNA, and which mediates the targeted cleavage of an RNA transcript via an RNA-induced silencing complex (RISC) pathway. iRNA directs the sequence-specific degradation of mRNA through RNA interference (RNAi).

A double stranded RNA is referred to herein as a “double stranded RNAi agent,” “double stranded RNA (dsRNA) molecule,” “dsRNA agent,” or “dsRNA”, which refers to a complex of ribonucleic acid molecules, having a duplex structure comprising two anti-parallel and substantially complementary nucleic acid strands, referred to as having “sense” and “antisense” orientations with respect to a target RNA.

The majority of nucleotides of each strand of the nucleic acid, e.g. a dsRNA molecule, are preferably ribonucleotides, but in that case each or both strands can also include one or more non-ribonucleotides, e.g., a deoxyribonucleotide or a modified nucleotide. In addition, as used in this specification, an “iRNA” may include ribonucleotides with chemical modifications.

The term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. Any such modifications, as used in a siRNA type molecule, are encompassed by “iRNA” or “RNAi agent” for the purposes of this specification and claims.

The duplex region of a nucleic acid of the invention e.g. a dsRNA may range from about 9 to 40 base pairs in length such as 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length.

The two strands forming the duplex structure may be different portions of one larger molecule, or they may be separate molecules e.g. RNA molecules.

The term “nucleotide overhang” refers to at least one unpaired nucleotide that extends from the duplex structure of a double stranded nucleic acid. A ds nucleic acid can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand.

In certain embodiments, the antisense strand has a 1-10 nucleotide, e.g., 0-3, 1-3, 2-4, 2-5, 4-10, 5-10, 1, 2, 3, 4, 5, 6, 7, 8, 9, or 10 nucleotide, overhang at the 3′-end or the 5′-end.

“Blunt” or “blunt end” means that there are no unpaired nucleotides at that end of the double stranded nucleic acid, i.e., no nucleotide overhang. The nucleic acids of the invention include those with no nucleotide overhang at one end or with no nucleotide overhangs at either end.

Unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press).

Complementary sequences within nucleic acid e.g. a dsRNA, as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more mismatched base pairs, such as 2, 4, or 5 mismatched base pairs, but preferably not more than 5, while retaining the ability to hybridize under the conditions most relevant to their ultimate application, e.g., inhibition of gene expression via a RISC pathway. Overhangs shall not be regarded as mismatches with regard to the determination of complementarity. For example, a nucleic acid e.g. dsRNA comprising one oligonucleotide 17 nucleotides in length and another oligonucleotide 19 nucleotides in length, wherein the longer oligonucleotide comprises a sequence of 17 nucleotides that is fully complementary to the shorter oligonucleotide, can yet be referred to as “fully complementary”.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a nucleic acid eg dsRNA, or between the antisense strand of a double stranded nucleic acid e.g. RNAi agent and a target sequence.

As used herein, a nucleic acid or polynucleotide that is “substantially complementary” to at least part of a messenger RNA (mRNA) refers to a polynucleotide that is substantially complementary to a contiguous portion of the mRNA of interest (e.g., an mRNA encoding a gene). For example, a polynucleotide is complementary to at least a part of an mRNA of a gene of interest if the sequence is substantially complementary to a non-interrupted portion of an mRNA encoding that gene.

Accordingly, in some preferred embodiments, the sense strand polynucleotides and the antisense polynucleotides disclosed herein are fully complementary to the target gene sequence.

In other embodiments, the antisense polynucleotides disclosed herein are substantially complementary to a target RNA sequence and comprise a contiguous nucleotide sequence which is at least about 80% complementary over its entire length to the equivalent region of the target RNA sequence, such as at least about 85%, 86%, 87%, 88%, 89%, about 90%, 91%, 92%, 93%, 94%, 95%, 96%, 97%, 98%, or 99% complementary or 100% complementary.

In some embodiments, a nucleic acid e.g. an iRNA of the invention includes a sense strand that is substantially complementary to an antisense polynucleotide which, in turn, is complementary to a target gene sequence and comprises a contiguous nucleotide sequence which is at least about 80% complementary over its entire length to the equivalent region of the nucleotide sequence of the antisense strand, such as about 85%, 86%, 87%, 88%, 89%, 90%, 91%, 92%, 93%, 94%, 95%, 96%, 97%, 98%, or 99% complementary, or 100% complementary.

In some embodiments, a nucleic acid e.g. an iRNA of the invention includes an antisense strand that is substantially complementary to the target sequence and comprises a contiguous nucleotide sequence which is at least 80% complementary over its entire length to the target sequence such as about 85%, 86%, 87%, 88%, 89%, 90%, 91%, 92%, 93%, 94%, 95%, 96%, 97%, 98%, or 99% complementary, or 100% complementary.

As used herein, a “subject” is an animal, such as a mammal, including a primate (such as a human, a non-human primate, e.g., a monkey, and a chimpanzee), or a non-primate or a bird that expresses the target gene, either endogenously or heterologously, when the target gene sequence has sufficient complementarity to the nucleic acid e.g. iRNA agent to promote target knockdown. In certain preferred embodiments, the subject is a human.

The terms “treating” or “treatment” refer to a beneficial or desired result including, but not limited to, alleviation or amelioration of one or more symptoms associated with gene expression. “Treatment” can also mean prolonging survival as compared to expected survival in the absence of treatment. Treatment can include prevention of development of co-morbidities, e.g., reduced liver damage in a subject with a hepatic infection.

“Therapeutically effective amount,” as used herein, is intended to include the amount of a nucleic acid e.g. an iRNA that, when administered to a patient for treating a subject having disease, is sufficient to effect treatment of the disease (e.g., by diminishing, ameliorating or maintaining the existing disease or one or more symptoms of disease or its related comorbidities).

The phrase “pharmaceutically acceptable” is employed herein to refer to compounds, materials, compositions, or dosage forms which are suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated.

Where a value or range of values of a parameter are recited, it is intended that values and ranges intermediate to the recited values are also intended to be part of this invention.

The articles “a” and “an” are used herein to refer to one or to more than one (i.e. to at least one) of the grammatical object of the article.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

The terminal region of a strand is the last 5 nucleotides from the 5′ or the 3′ end.

Various embodiments of the invention can be combined as determined appropriate by one of skill in the art.

### Abasic Nucleotides

There are 1, e.g. 2, e.g. 3, e.g. 4 or more abasic nucleotides present in the nucleic acid. Abasic nucleotides are modified nucleotides because they lack the base normally seen at position 1 of the sugar moiety. Typically, there will be a hydrogen at position 1 of the sugar moiety of the abasic nucleotides present in a nucleic acid according to the present invention.

The abasic nucleotides are in the terminal region of the second strand, preferably located within the terminal 5 nucleotides of the end of the strand. The terminal region may be the terminal 5 nucleotides, which includes abasic nucleotides.

The second strand may comprise, as preferred features (which are all specifically contemplated in combination unless mutually exclusive):
2, or more than 2, abasic nucleotides in a terminal region of the second strand; and/or
2, or more than 2, abasic nucleotides in either the 5′ or 3′ terminal region of the second strand; and/or
2, or more than 2, abasic nucleotides in either the 5′ or 3′ terminal region of the second strand, wherein the abasic nucleotides are present in an overhang as herein described; and/or
2, or more than 2, consecutive abasic nucleotides in a terminal region of the second strand, wherein preferably one such abasic nucleotide is a terminal nucleotide; and/or
2, or more than 2, consecutive abasic nucleotides in either the 5′ or 3′ terminal region of the second strand, wherein preferably one such abasic nucleotide is a terminal nucleotide in either the 5′ or 3′ terminal region of the second strand; and/or
a reversed internucleotide linkage connects at least one abasic nucleotide to an adjacent basic nucleotide in a terminal region of the second strand; and/or
a reversed internucleotide linkage connects at least one abasic nucleotide to an adjacent basic nucleotide in either the 5′ or 3′ terminal region of the second strand; and/or
an abasic nucleotide as the penultimate nucleotide which is connected via the reversed linkage to the nucleotide which is not the terminal nucleotide (called the antepenultimate nucleotide herein); and/or
abasic nucleotides as the 2 terminal nucleotides connected via a 5′-3′ linkage when reading the strand in the direction towards the terminus comprising the terminal nucleotides;
abasic nucleotides as the 2 terminal nucleotides connected via a 3′-5′ linkage when reading the strand in the direction towards the terminus comprising the terminal nucleotides;
abasic nucleotides as the terminal 2 positions, wherein the penultimate nucleotide is connected via the reversed linkage to the antepenultimate nucleotide, and wherein the reversed linkage is a 5-5′ reversed linkage or a 3′-3′ reversed linkage;
abasic nucleotides as the terminal 2 positions, wherein the penultimate nucleotide is connected via the reversed linkage to the antepenultimate nucleotide, and wherein either
(1) the reversed linkage is a 5-5′ reversed linkage and the linkage between the terminal and penultimate abasic nucleotides is 3′5′ when reading towards the terminus comprising the terminal and penultimate abasic nucleotides; or
(2) the reversed linkage is a 3-3′ reversed linkage and the linkage between the terminal and penultimate abasic nucleotides is 5′3′ when reading towards the terminus comprising the terminal and penultimate abasic nucleotides.

Preferably there is an abasic nucleotide at the terminus of the second strand.

Preferably there are 2 or at least 2 abasic nucleotides in the terminal region of the second strand, preferably at the terminal and penultimate positions.

Preferably 2 or more abasic nucleotides are consecutive, for example all abasic nucleotides may be consecutive. For example, the terminal 1 or terminal 2 or terminal 3 or terminal 4 nucleotides may be abasic nucleotides.

An abasic nucleotide may also be linked to an adjacent nucleotide through a 5′-3′ phosphodiester linkage or reversed linkage unless there is only 1 abasic nucleotide at the terminus, in which case it will have a reversed linkage to the adjacent nucleotide.

A reversed linkage (which may also be referred to as an inverted linkage, which is also seen in the art), comprises either a 5′-5′, a 3′3′, a 3′-2′ or a 2′-3′ phosphodiester linkage between the adjacent sugar moieties of the nucleotides.

Abasic nucleotides which are not terminal will have 2 phosphodiester bonds, one with each adjacent nucleotide, and these may be a reversed linkage or may be a 5′-3 phosphodiester bond or may be one of each.

A preferred embodiment comprises 2 abasic nucleotides at the terminal and penultimate positions of the second strand, and wherein the reversed internucleotide linkage is located between the penultimate (abasic) nucleotide and the antepenultimate nucleotide.

Preferably there are 2 abasic nucleotides at the terminal and penultimate positions of the second strand and the penultimate nucleotide is linked to the antepenultimate nucleotide through a reversed internucleotide linkage and is linked to the terminal nucleotide through a 5′-3′ or 3′-5′ phosphodiester linkage (reading in the direction of the terminus of the molecule).

Different preferred features are as follows:

The reversed internucleotide linkage is a 3′3 reversed linkage. The reversed internucleotide linkage is at a terminal region which is distal to the 5′ terminal phosphate of the second strand.

The reversed internucleotide linkage is a 5′5 reversed linkage. The reversed internucleotide linkage is at a terminal region which is distal to the 3′ terminal hydroxide of the second strand.

Examples of the structures are as follows (where the specific RNA nucleotides shown are not limiting and could be any RNA nucleotide):
A A 3′-3′ reversed bond (and also showing the 5′-3 direction of the last phosphodiester bond between the two abasic molecules reading towards the terminus of the molecule)

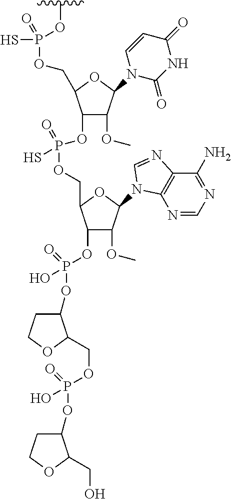 [SMILES: *OP(=O)(S)OCC1O[C@@H](n2ccc(=O)[nH]c2=O)C(OC)C1OP(=O)(S)OCC1O[C@@H](n2cnc3c(N)ncnc32)C(OC)C1OP(=O)(O)OC1CCOC1COP(=O)(O)OC1CCOC1CO]

B Illustrating a 5′-5′ reversed bond (and also showing the 3′-5′ direction of the last phosphodiester bond between the two abasic molecules reading towards the terminus of the molecule)

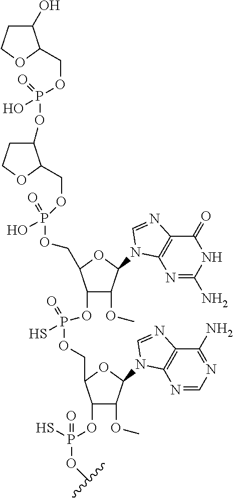 [SMILES: *OP(=O)(S)OC1C(COP(=O)(S)OC2C(COP(=O)(O)OCC3OCCC3OP(=O)(O)OCC3OCCC3O)O[C@@H](n3cnc4c(=O)[nH]c(N)nc43)C2OC)O[C@@H](n2cnc3c(N)ncnc32)C1OC]

The abasic nucleotide or abasic nucleotides present in the nucleic acid are provided in the presence of a reversed internucleotide linkage or linkages, namely a 5′-5′ or a 3′-3′ reversed internucleotide linkage. A reversed linkage occurs as a result of a change of orientation of an adjacent nucleotide sugar, such that the sugar will have a 3′-5′ orientation as opposed to the conventional 5′-3′ orientation (with reference to the numbering of ring atoms on the nucleotide sugars). The abasic nucleotide or nucleotides as present in the nucleic acids of the invention preferably include such inverted nucleotide sugars.

In the case of a terminal nucleotide having an inverted orientation, then this will result in an “inverted” end configuration for the overall nucleic acid. Whilst certain structures drawn and referenced herein are represented using conventional 5′-3′ direction (with reference to the numbering of ring atoms on the nucleotide sugars), it will be appreciated that the presence of a terminal nucleotide having a change of orientation and a proximal 3′-3′ reversed linkage, will result in a nucleic acid having an overall 5′-5′ end structure (i.e. the conventional 3′ end nucleotide becomes a 5′ end nucleotide). Alternatively, it will be appreciated that the presence of a terminal nucleotide having a change of orientation and a proximal 5′-5′ reversed linkage will result in a nucleic acid with an overall 3′-3′ end structure.

The proximal 3′-3′ or 5′-5′ reversed linkage as herein described, may comprise the reversed linkage being directly adjacent/attached to a terminal nucleotide having an inverted orientation, such as a single terminal nucleotide having an inverted orientation. Alternatively, the proximal 3′-3′ or 5′-5′ reversed linkage as herein described, may comprise the reversed linkage being adjacent 2, or more than 2, nucleotides having an inverted orientation, such as 2, or more than 2, terminal region nucleotides having an inverted orientation, such as the terminal and penultimate nucleotides. In this way, the reversed linkage may be attached to a penultimate nucleotide having an inverted orientation. While a skilled addressee will appreciate that inverted orientations as described above can result in nucleic acid molecules having overall 3′-3′ or 5′-5′ end structures as described herein, it will also be appreciated that with the presence of one or more additional reversed linkages and/or nucleotides having an inverted orientation, then the overall nucleic acid may have 3′-5′ end structures corresponding to the conventionally positioned 5′/3′ ends.

In one aspect the nucleic acid may have a 3′-3′ reversed linkage, and the terminal sugar moiety may comprise a 5′ OH rather than a 5′ phosphate group at the 5′ position of that terminal sugar.

A skilled person would therefore clearly understand that 5′-5′, 3′-3′ and 3′-5′ (reading in the direction of that terminus) end variants of the more conventional 5′-3′ structures (with reference to the numbering of ring atoms on the end nucleotide sugars) drawn herein are included in the scope of the disclosure, where a reversed linkage or linkages is/are present.

In the situation of eg a reversed internucleotide linkage and/or one or more nucleotides having an inverted orientation creating an inverted end, and where the relative position of a linkage (eg to a linker) or the location of an internal feature (such as a modified nucleotide) is defined relative to the 5′ or 3′ end of the nucleic acid, then the 5′ or 3′ end is the conventional 5′ or 3′ end which would have existed had a reversed linkage not been in place, and wherein the conventional 5′ or 3′ end is determined by consideration of the directionality of the majority of the internal nucleotide linkages and/or nucleotide orientation within the nucleic acid. It is possible to tell from these internal bonds and/or nucleotide orientation which ends of the nucleic acid would constitute the conventional 5′ and 3′ ends (with reference to the numbering of ring atoms on the end nucleotide sugars) of the molecule absent the reversed linkage.

For example, in the structure shown below there are abasic residues in the first 2 positions located at the “5′” end. Where the terminal nucleotide has an inverted orientation then the “5′” end indicated in the diagram below, which is the conventional 5′ end, can in fact comprise a 3′ OH in view of the inverted nucleotide at the terminal position. Nevertheless the majority of the molecule will comprise conventional internucleotide linkages that run from the 3′ OH of the sugar to the 5′ phosphate of the next sugar, when reading in the standard 5′ [PO4] to 3′ [OH] direction of a nucleic acid molecule (with reference to the numbering of ring atoms on the nucleotide sugars), which can be used to determine the conventional 5′ and 3′ ends that would be found absent the inverted end configuration.

A 5′ A-A-Me-Me-Me-Me-Me-Me-F-Me-F-F-F-Me-Me-Me-Me-
Me-Me-Me-Me-Me-Me 3′

The reversed bond is preferably located at the end of the nucleic acid eg RNA which is distal to a ligand moiety, such as a GalNAc containing portion, of the molecule.

GalNAc-siRNA constructs with a 5′-GalNAc on the sense strand can have a reversed linkage on the opposite end of the sense strand.

GalNAc-siRNA constructs with a 3′-GalNAc on the sense strand can have a reversed linkage on the opposite end of the sense strand. Nucleic Acid Lengths

In one aspect the i) the first strand of the nucleic acid has a length in the range of 15 to 30 nucleotides, preferably 19 to 25 nucleotides, more preferably 23 or 25; and/or ii) the second strand of the nucleic acid has a length in the range of 15 to 30 nucleotides, preferably 19 to 25 nucleotides, more preferably 23.

Generally, the duplex structure of the nucleic acid e.g. an iRNA is about 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity of an antisense sequence to a target sequence and/or the region of complementarity of an antisense sequence to a sense sequence is about 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In certain preferred embodiments, the region of complementarity of an antisense sequence to a target sequence and/or the region of complementarity of an antisense sequence to a sense sequence is at least 17 nucleotides in length. For example, the region of complementarity between the antisense strand and the target is 19 to 21 nucleotides in length, for example, the region of complementarity is 21 nucleotides in length.

In preferred embodiments, each strand is no more than 30 nucleotides in length.

A nucleic acid e.g. a dsRNA as described herein can further include one or more single-stranded nucleotide overhangs e.g., 1-4, 2-4, 1-3, 2-3, 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a nucleic acid e.g. a dsRNA.

In certain preferred embodiments, at least one strand comprises a 3′ overhang of at least 1 nucleotide, e.g., at least one strand comprises a 3′ overhang of at least 2 nucleotides. The overhang is suitably on the antisense/guide strand and/or the sense/passenger strand.

### Nucleic Acid Modifications

In certain embodiments, the nucleic acid e.g. an RNA of the invention e.g., a dsRNA, does not comprise further modifications (beyond the required abasic modifications), e.g., chemical modifications or conjugations known in the art and described herein.

In other preferred embodiments, the nucleic acid e.g. RNA of the invention, e.g., a dsRNA, is further chemically modified (beyond the abasic modifications) to enhance stability or other beneficial characteristics.

In certain embodiments of the invention, substantially all of the nucleotides are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

Modifications include, for example, end modifications, e.g., 5′-end modifications (phosphorylation, conjugation, inverted linkages) or 3′-end modifications (conjugation, DNA nucleotides within an RNA, or RNA nucleotides within a DNA, inverted linkages, etc.); base modifications, e.g., replacement with stabilizing bases, destabilizing bases, or bases that base pair with an expanded repertoire of partners, conjugated bases; sugar modifications (e.g., at the 2′-position or 4′-position) or replacement of the sugar; or backbone modifications, including modification or replacement of the phosphodiester linkages.

Specific examples of nucleic acids such as iRNA compounds useful in the embodiments described herein include, but are not limited to RNAs containing modified backbones or no natural internucleoside linkages. Nucleic acids such as RNAs having modified backbones include, among others, those that do not have a phosphorus atom in the backbone. For the purposes of this specification, and as sometimes referenced in the art, modified nucleic acids e.g. RNAs that do not have a phosphorus atom in their internucleoside backbone can also be considered to be oligonucleosides. In some embodiments, a modified nucleic acid e.g. an iRNA will have a phosphorus atom in its internucleoside backbone.

Modified nucleic acid e.g. RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 5′-3′ or 5′-2′. Various salts, mixed salts and free acid forms are also included.

Modified nucleic acids e.g. RNAs can also contain one or more substituted sugar moieties. The nucleic acids e.g. iRNAs, e.g., dsRNAs, featured herein can include one of the following at the 2′-position: OH; F; 0-, S-, or N-alkyl; 0-, S-, or N-alkenyl; 0-, S- or N-alkynyl; or O-alkyl-O-alkyl, wherein the alkyl, alkenyl and alkynyl can be substituted or unsubstituted. 2′ O-methyl and 2′-F are preferred modifications.

In certain preferred embodiments, the nucleic acid comprises at least one modified nucleotide.

The nucleic acid of the invention may comprise one or more modified nucleotides on the first strand and/or the second strand.

In some embodiments, substantially all of the nucleotides of the sense strand and all of the nucleotides of the antisense strand comprise a modification.

In some embodiments, all of the nucleotides of the sense strand and substantially all of the nucleotides of the antisense strand comprise a modification.

In some embodiments, all of the nucleotides of the sense strand and all of the nucleotides of the antisense strand comprise a modification.

In one embodiment, at least one of the modified nucleotides is selected from the group consisting of a deoxy-nucleotide, a 3′-terminal deoxy-thymine (dT) nucleotide, a 2′-O-methyl modified nucleotide (also called herein 2′-Me, where Me is a methoxy), a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, and a nucleotide comprising a 5′-phosphate mimic. In another embodiment, the modified nucleotides comprise a short sequence of 3′-terminal deoxy-thymine nucleotides (dT).

Modifications on the nucleotides may preferably be selected from the group including, but not limited to, LNA, HNA, CeNA, 2-methoxyethyl, 2-O-alkyl, 2-O-allyl, 2′-C-allyl, 2′-fluoro, 2′-deoxy, 2′-hydroxyl, and combinations thereof. In another embodiment, the modifications on the nucleotides are 2-O-methyl (“2-Me”) or 2′-fluoro modifications.

One preferred modification is a modification at the 2′—OH group of the ribose sugar, optionally selected from 2′-Me or 2′-F modifications.

Preferred nucleic acid comprise one or more nucleotides on the first strand and/or the second strand which are modified, to form modified nucleotides, as follows:

A nucleic acid wherein the modification is a modification at the 2′—OH group of the ribose sugar, optionally selected from 2′-Me or 2′-F modifications.

A nucleic acid wherein the first strand comprises a 2′-F at any of position 14, position 2, position 6, or any combination thereof, counting from position 1 of said first strand.

A nucleic acid wherein the second strand comprises a 2′-F modification at position 7 and/or 9, and/or 11, and/or 13, counting from position 1 of said second strand.

A nucleic acid wherein the second strand comprises a 2′-F modification at position 7 and 9 and 11 counting from position 1 of said second strand.

A nucleic acid wherein the first and second strand each comprise 2′-Me and 2′-F modifications.

A nucleic acid wherein the nucleic acid comprises at least one thermally destabilizing modification, suitably at one or more of positions 1 to 9 of the first strand, and/or at one or more of positions on the second strand aligned with positions 1 to 9 of the first strand, wherein the destabilizing modification is selected from a modified unlocked nucleic acid (IMUNA) and a glycol nucleic acid (GNA), preferably a glycol nucleic acid. The nucleic acid may be a double stranded molecule, preferably double stranded RNA, which has a melting temperature in the range of about 40 to 80° C. The nucleic acid may comprise at least one thermally destabilizing modification at position 7 of the first strand.

A nucleic acid wherein the nucleic acid comprises 3 or more 2′-F modifications at positions 7 to 13 of the second strand, such as 4, 5, 6 or 7 2′-F modifications at positions 7 to 13 of the second strand, counting from position 1 of said second strand.

A nucleic acid wherein said second strand comprises at least 3, such as 4, 5 or 6, 2′-Me modifications at positions 1 to 6 of the second strand, counting from position 1 of said second strand.

A nucleic acid wherein said first strand comprises at least 5 2′-Me consecutive modifications at the 3′ terminal region, preferably including the terminal nucleotide at the 3′ terminal region, or at least within 1 or 2 nucleotides from the terminal nucleotide at the 3′ terminal region.

A nucleic acid wherein said first strand comprises 7 2′-Me consecutive modifications at the 3′ terminal region, preferably including the terminal nucleotide at the 3′ terminal region.

Preferred modification patterns include:
A nucleic acid wherein the second strand includes the following modification pattern:

N^{A}—(N)₃₋₅—N^{B}
wherein (above and below) N represents a nucleotide with a first modification;
N^{A} represents a nucleotide with a second modification different to the first modification of N;
N^{B} represents a nucleotide with a third modification different to the first modification of N, but either the same or different to the second modification of N^{A}; and
wherein said pattern has a 5′ to 3′ directionality along the second strand.

A nucleic acid wherein the second strand includes the following modification pattern:

N^{A}—(N)₃—N^{B}.

A nucleic acid wherein the second strand includes the following modification pattern:

N^{A}—(N)₅—N^{B}.

A nucleic acid wherein the second strand includes the following modification pattern:

Me-(F)₃-Me.

A nucleic acid wherein the second strand includes the following modification pattern:

Me-(F)₅-Me.

A nucleic acid wherein the second strand includes the following modification pattern:

N^{C}—N^{A}—(N)₃₋₅—N^{B}—N^{D}

wherein N^{C} and N^{D}, which may be the same or different, respectively denote a plurality of 5′ and 3′ terminal region chemically modified nucleotides, wherein at least N^{C} comprises at least two differently modified nucleotides.

A nucleic acid wherein N^{D} comprises at least two differently modified nucleotides, or a plurality of nucleotides each having the same modification, preferably 2′-Me consecutive modifications.

A nucleic acid wherein the second strand includes the following modification pattern:

5′ A-A-Me-Me-Me-Me-Me-Me-F-Me-F-F-F-Me-Me-Me-Me-
Me-Me-Me-Me-Me-Me 3′

where A represents an abasic modification.

A nucleic acid wherein the second strand includes the following modification pattern:

5′ A-A-Me-Me-F-Me-F-Me-F-Me-F-F-F-Me-F-Me-Me-F-
Me-F-Me-Me-Me 3′

where A represents an abasic modification.

A nucleic acid wherein the second strand includes the following modification pattern:

5′ Me-Me-Me-Me-Me-Me-F-Me-F-F-F-Me-Me-Me-Me-Me-
Me-Me-Me-Me-Me-A-A 3′

where A represents an abasic modification.

A nucleic acid wherein the second strand includes the following modification pattern:

5′ Me-Me-F-Me-F-Me-F-Me-F-F-F-Me-F-Me-Me-F-Me-F-
Me-Me-Me-A-A 3′

where A represents an abasic modification.

A nucleic acid wherein the first strand includes the following modification pattern:

M^{A}-(M)₃₋₅-M^{B}
wherein M represents a nucleotide with a first modification and wherein typically (M)₃_₅ are substantially aligned with (N)₃_₅ in said second strand;
M^{A} represents a nucleotide with a second modification different to the first modification of M;
M^{B} represents a nucleotide with a third modification different to the first modification of M, but either the same or different to the second modification of M^{A}.

A nucleic acid, wherein the first strand includes the following modification pattern:

M^{A}-(M)₃-M^{B}.

A nucleic acid, wherein the first strand includes the following modification pattern:

M^{A}-(M)₄-M^{B}.

A nucleic acid, wherein the first strand includes the following modification pattern:

M^{A}-(M)₅-M^{B}.

A nucleic acid wherein the first strand includes the following modification pattern:

F-(Me)₃-F.

A nucleic acid wherein the first strand includes the following modification pattern:

F-(Me)₄-F.

A nucleic acid, wherein the first strand includes the following modification pattern:

F-(Me)₅-F.

A nucleic acid, wherein the first strand includes the following modification pattern:

M^{C}-M^{A}-(M)₃₋₅-M^{B}-M^{D}
wherein M^{C} and M^{D}, which may be the same or different, respectively denote a plurality of 5′ and 3′ terminal region chemically modified nucleotides each comprising at least two differently modified nucleotides.

A nucleic acid wherein the first strand includes the following modification pattern:

3′ Me-Me-Me-Me-Me-Me-Me-F-Me-F-Me-Me-Me-Me-F-F-
Me-F-Me-Me-Me-F-Me 5′.

A nucleic acid wherein the first strand includes the following modification pattern:

3′ H-H-Me-Me-Me-Me-Me-F-Me-F-Me-F-Me-Me-Me-F-Me-
F-Me-Me-F-Me-F-F-Me 5′.

A nucleic acid wherein the first strand includes the following modification pattern:

3′ Me-Me-Me-Me-Me-Me-Me-F-Me-F-Me-Me-Me-Me-F-Me-
Me-F-Me-Me-Me-F-Me 5′.

Position 1 of the first or the second strand is the nucleotide which is the closest to the end of the nucleic acid (ignoring any abasic nucleotides) and that is joined to an adjacent nucleotide (at Position 2) via a 3′ to 5′ internal bond, with reference to the bonds between the sugar moieties of the backbone, and reading in a direction away from that end of the molecule.

It can therefore be seen that “position 1 of the sense strand” is the 5′ most nucleotide (not including abasic nucleotides) at the conventional 5′ end of the sense strand. Typically, the nucleotide at this position 1 of the sense strand will be equivalent to the 5′ nucleotide of the selected target nucleic acid sequence, and more generally the sense strand will have equivalent nucleotides to those of the target nucleic acid sequence starting from this position 1 of the sense strand, whilst also allowing for acceptable mismatches between the sequences.

As used herein, “position 1 of the antisense strand” is the 5′ most nucleotide (not including abasic nucleotides) at the conventional 5′ end of the antisense strand. As hereinbefore described, there will be a region of complementarity between the sense and antisense strands, and in this way the antisense strand will also have a region of complementarity to the target nucleic acid sequence as referred to above.

In certain embodiments, the nucleic acid e.g. RNAi agent further comprises at least one phosphorothioate or methylphosphonate internucleotide linkage. For example the phosphorothioate or methylphosphonate internucleotide linkage can be at the 3′-terminus or in the terminal region of one strand, i.e., the sense strand or the antisense strand; or at the ends of both strands, the sense strand and the antisense strand.

In certain embodiments, the phosphorothioate or methylphosphonate internucleotide linkage is at the 5′terminus or in the terminal region of one strand, i.e., the sense strand or the antisense strand; or at the ends of both strands, the sense strand and the antisense strand.

In certain embodiments, a phosphorothioate or a methylphosphonate internucleotide linkage is at both the 5′- and 3′-terminus or in the terminal region of one strand, i.e., the sense strand or the antisense strand; or at the ends of both strands, the sense strand and the antisense strand.

Any nucleic acid may comprise one or more phosphorothioate (PS) modifications within the nucleic acid, such as at least two PS internucleotide bonds at the ends of a strand.

At least one of the oligoribonucleotide strands preferably comprises at least two consecutive phosphorothioate modifications in the last 3 nucleotides of the oligonucleotide.

The invention therefore also relates to: A nucleic acid disclosed herein which comprises phosphorothioate internucleotide linkages respectively between at least two or three consecutive positions, such as in a 5′ and/or 3′ terminal region and/or near terminal region of the second strand, whereby said near terminal region is preferably adjacent said terminal region wherein said one or more abasic nucleotides of said second strand is/are located.

A nucleic acid disclosed herein which comprises phosphorothioate internucleotide linkages respectively between at least two or three consecutive positions in a 5′ and/or 3′ terminal region of the first strand, whereby preferably the terminal position at the 5′ and/or 3′ terminal region of said first strand is attached to its adjacent position by a phosphorothioate internucleotide linkage.

The nucleic acid strand may be an RNA comprising a phosphorothioate internucleotide linkage between the three nucleotides contiguous with 2 terminally located abasic nucleotides.

A preferred nucleic acid is a double stranded RNA comprising 2 adjacent abasic nucleotides at the 5′ terminus of the second strand and a ligand moiety comprising one or more GalNAc ligand moieties at the opposite 3′ end of the second strand. Further preferred, the same nucleic acid may also comprise a phosphorothioate bond between nucleotides at positions 3-4 and 4-5 of the second strand, reading from the position 1 of the second strand. Further preferred, the same nucleic acid may also comprise a 2′ F modification at positions 7, 9 and 11 of the second strand.

### Conjugation

Another modification of the nucleic acid e.g. RNA e.g. an iRNA of the invention involves linking the nucleic acid e.g. the iRNA to one or more ligand moieties e.g. to enhance the activity, cellular distribution, or cellular uptake of the nucleic acid e.g. iRNA e.g., into a cell.

In some embodiments, the ligand moiety described can be attached to a nucleic acid e.g. an iRNA oligonucleotide, via a linker that can be cleavable or non-cleavable. The term “linker” or “linking group” means an organic moiety that connects two parts of a compound, e.g., covalently attaches two parts of a compound.

The ligand can be attached to the 3′ or 5′ end of the sense strand.

The ligand is preferably conjugated to 3′ end of the sense strand of the nucleic acid e.g. an RNAi agent.

The invention therefore relates in a further aspect to a conjugate for inhibiting expression of a target gene in a cell, said conjugate comprising a nucleic acid portion and one or more ligand moieties, said nucleic acid portion comprising a nucleic acid as disclosed herein.

In one aspect the second strand of the nucleic acid is conjugated directly or indirectly (e.g. via a linker) to the one or more ligand moiety(s), wherein said ligand moiety is typically present at a terminal region of the second strand, preferably at the 3′ terminal region thereof.

In certain embodiments, the ligand moiety comprises a GalNAc or GalNAc derivative attached to the nucleic acid eg dsRNA through a linker.

Therefore the invention relates to a conjugate wherein the ligand moiety comprises
i) one or more GalNAc ligands; and/or
ii) one or more GalNAc ligand derivatives; and/or
iii) one or more GalNAc ligands conjugated to said nucleic acid through a linker.

Said GalNAc ligand may be conjugated directly or indirectly to the 5′ or 3′ terminal region of the second strand of the nucleic acid, preferably at the 3′ terminal region thereof.

GalNAc ligands are well known in the art and described in, inter alia, EP3775207A1.

### Vector And Cell

In one aspect, the invention provides a cell containing a nucleic acid, such as inhibitory RNA [RNAi] as described herein.

In one aspect, the invention provides a cell comprising a vector as described herein.

### Pharmaceutically Acceptable Compositions

In one aspect, the invention provides a pharmaceutical composition for inhibiting expression of a target gene, the composition comprising a nucleic acid as disclosed herein.

The pharmaceutically acceptable composition may comprise an excipient and or carrier.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or poly anhydrides; (22) bulking agents, such as polypeptides and amino acids (23) serum component, such as serum albumin, HDL and LDL; and (22) other non-toxic compatible substances employed in pharmaceutical formulations.

Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable excipients include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

In one embodiment, the nucleic acid or composition is administered in an unbuffered solution. In certain embodiments, the unbuffered solution is saline or water. In other embodiments, the nucleic acid e.g. RNAi agent is administered in a buffered solution. In such embodiments, the buffer solution can comprise acetate, citrate, prolamine, carbonate, or phosphate, or any combination thereof. For example, the buffer solution can be phosphate buffered saline (PBS).

### Dosages

The pharmaceutical compositions of the invention may be administered in dosages sufficient to inhibit expression of a gene. In general, a suitable dose of a nucleic acid e.g. an iRNA of the invention will be in the range of about 0.001 to about 200.0 milligrams per kilogram body weight of the recipient per day, generally in the range of about 1 to 50 mg per kilogram body weight per day. Typically, a suitable dose of a nucleic acid e.g. an iRNA of the invention will be in the range of about 0.1 mg/kg to about 5.0 mg/kg, e.g., about 0.3 mg/kg and about 3.0 mg/kg.

A repeat-dose regimen may include administration of a therapeutic amount of a nucleic acid e.g. iRNA on a regular basis, such as every other day or once a year. In certain embodiments, the nucleic acid e.g. iRNA is administered about once per month to about once per quarter (i.e., about once every three months).

In various embodiments, the nucleic acid e.g. RNAi agent is administered at a dose of about 0.01 mg/kg to about 10 mg/kg or about 0.5 mg/kg to about 50 mg/kg. In some embodiments, the nucleic acid e.g. RNAi agent is administered at a dose of about 10 mg/kg to about 30 mg/kg. In certain embodiments, the nucleic acid e.g. RNAi agent is administered at a dose selected from about 0.5 mg/kg 1 mg/kg, 1.5 mg/kg, 3 mg/kg, 5 mg/kg, 10 mg/kg, and 30 mg/kg. In certain embodiments, the nucleic acid e.g. RNAi agent is administered about once per week, once per month, once every other two months, or once a quarter (i.e., once every three months) at a dose of about 0.1 mg/kg to about 5.0 mg/kg. In certain embodiments, the nucleic acid e.g. RNAi agent is administered to the subject once a week. In certain embodiments, the nucleic acid e.g. RNAi agent is administered to the subject once a month. In certain embodiments, the nucleic acid e.g. RNAi agent is administered once per quarter (i.e., every three months).

After an initial treatment regimen, the treatments can be administered on a less frequent basis. For example, after administration weekly or biweekly for three months, administration can be repeated once per month, for six months, or a year; or longer.

The pharmaceutical composition can be administered once daily, or administered as two, three, or more sub-doses at appropriate intervals throughout the day or even using continuous infusion or delivery through a controlled release formulation. In that case, the nucleic acid e.g. iRNA contained in each sub-dose must be correspondingly smaller in order to achieve the total daily dosage. The dosage unit can also be compounded for delivery over several days, e.g., using a conventional sustained release formulation which provides sustained release of the nucleic acid e.g. iRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bimonthly. In certain embodiments, the iRNA is administered about once per month to about once per quarter (i.e., about once every three months), or even every 6 months or 12 months.

Estimates of effective dosages and in vivo half-lives for the individual nucleic acid e.g. iRNAs encompassed by the invention can be made using conventional methodologies or on the basis of in vivo testing using an appropriate animal model, as known in the art.

The pharmaceutical compositions of the present invention can be administered in a number of ways depending upon whether local or systemic treatment is desired and upon the area to be treated. Administration can be topical {e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal, or intramuscular injection or infusion; subdermal, e.g., via an implanted device; or intracranial, e.g., by intraparenchymal, intrathecal or intraventricular administration. In certain preferred embodiments, the compositions are administered by intravenous infusion or injection. In certain embodiments, the compositions are administered by subcutaneous injection.

In one embodiment, the nucleic acid e.g. RNAi agent is administered to the subject subcutaneously.

The nucleic acid e.g. iRNA can be delivered in a manner to target a particular tissue {e.g. in particular liver cells).

### Methods for Inhibiting Gene Expression

The present invention also provides methods of inhibiting expression of a gene in a cell. The methods include contacting a cell with an nucleic acid of the invention e.g. RNAi agent, such as double stranded RNAi agent, in an amount effective to inhibit expression of the gene in the cell, thereby inhibiting expression of the gene in the cell.

Contacting of a cell with the nucleic acid e.g. an iRNA, such as a double stranded RNAi agent, may be done in vitro or in vivo. Contacting a cell in vivo with nucleic acid e.g. iRNA includes contacting a cell or group of cells within a subject, e.g., a human subject, with the nucleic acid e.g. iRNA. Combinations of in vitro and in vivo methods of contacting a cell are also possible. Contacting a cell may be direct or indirect, as discussed above. Furthermore, contacting a cell may be accomplished via a targeting ligand moiety, including any ligand moiety described herein or known in the art. In preferred embodiments, the targeting ligand moiety is a carbohydrate moiety, e.g. a GalNAc3 ligand, or any other ligand moiety that directs the RNAi agent to a site of interest.

The term “inhibiting,” as used herein, is used interchangeably with “reducing,” “silencing,” “downregulating”, “suppressing”, and other similar terms, and includes any level of inhibition.

In some embodiments of the methods of the invention, expression of a gene is inhibited by at least 30%, 35%, 40%, 45%, 50%, 55%, 60%, 65%, 70%, 75%, 80%, 85%, 90%, or 95%, or to below the level of detection of the assay. In certain embodiments, the methods include a clinically relevant inhibition of expression of a target gene e.g. as demonstrated by a clinically relevant outcome after treatment of a subject with an agent to reduce the expression of the gene

Inhibition of the expression of a gene may be manifested by a reduction of the amount of mRNA of the target gene of interest in comparison to a suitable control.

In other embodiments, inhibition of the expression of a gene may be assessed in terms of a reduction of a parameter that is functionally linked to gene expression, e.g, protein expression or signalling pathways.

Methods of Treating or Preventing Diseases Associated with Gene Expression

The present invention also provides methods of using nucleic acid e.g. an iRNA of the invention or a composition containing nucleic acid e.g. an iRNA of the invention to reduce or inhibit gene expression in a cell. The methods include contacting the cell with a nucleic acid e.g. dsRNA of the invention and maintaining the cell for a time sufficient to obtain degradation of the mRNA transcript of a gene, thereby inhibiting expression of the gene in the cell. Reduction in gene expression can be assessed by any methods known in the art.

In the methods of the invention the cell may be contacted in vitro or in vivo, i.e., the cell may be within a subject.

A cell suitable for treatment using the methods of the invention may be any cell that expresses a gene of interest associated with disease.

The in vivo methods of the invention may include administering to a subject a composition containing a nucleic acid of the invention e.g. an iRNA, where the nucleic acid e.g. iRNA includes a nucleotide sequence that is complementary to at least a part of an RNA transcript of the gene of the mammal to be treated.

The present invention further provides methods of treatment of a subject in need thereof. The treatment methods of the invention include administering a nucleic acid such as an iRNA of the invention to a subject, e.g., a subject that would benefit from a reduction or inhibition of the expression of a gene, in a therapeutically effective amount e.g. a nucleic acid such as an iRNA targeting a gene or a pharmaceutical composition comprising the nucleic acid targeting a gene.

An nucleic acid e.g. iRNA of the invention may be administered as a “free” nucleic acid or “free iRNA, administered in the absence of a pharmaceutical composition. The naked nucleic acid may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, a nucleic acid e.g. iRNA of the invention may be administered as a pharmaceutical composition, such as a dsRNA liposomal formulation.

In one embodiment, the method includes administering a composition featured herein such that expression of the target gene is decreased, such as for about 1, 2, 3, 4, 5, 6, 7, 8, 12, 16, 18, 24 hours, 28, 32, or about 36 hours. In one embodiment, expression of the target gene is decreased for an extended duration, e.g., at least about two, three, four days or more, e.g., about one week, two weeks, three weeks, or four weeks or longer, e.g., about 1 month, 2 months, or 3 months.

Subjects can be administered a therapeutic amount of nucleic acid e.g. iRNA, such as about 0.01 mg/kg to about 200 mg/kg.

The nucleic acid e.g. iRNA can be administered by intravenous infusion over a period of time, on a regular basis. In certain embodiments, after an initial treatment regimen, the treatments can be administered on a less frequent basis. Administration of the iRNA can reduce gene product levels of a target gene, e.g., in a cell or tissue of the patient by at least about 20%, 25%, 30%, 35%, 40%, 45%, 50%, 55%, 60%, 65%, 70%, 75%, 80%, 85%, 90%, or 95%, or below the level of detection of the assay method used. In certain embodiments, administration results in clinical stabilization or preferably clinically relevant reduction of at least one sign or symptom of a gene-associated disorder.

Alternatively, the nucleic acid e.g. iRNA can be administered subcutaneously, i.e., by subcutaneous injection. One or more injections may be used to deliver the desired daily dose of nucleic acid e.g. iRNA to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of nucleic acid on a regular basis, such as every other day or to once a year. In certain embodiments, the nucleic acid is administered about once per month to about once per quarter (i.e., about once every three months).

In one aspect the present invention may be applied in the compounds, processes, compositions or uses of the following Sentences numbered 1-101 (wherein reference to any Formula in the Sentences 1-101 refers only to those Formulas that are defined within Sentences 1-101. These formulae are reproduced in FIG. 43) 1. A compound comprising the following structure:

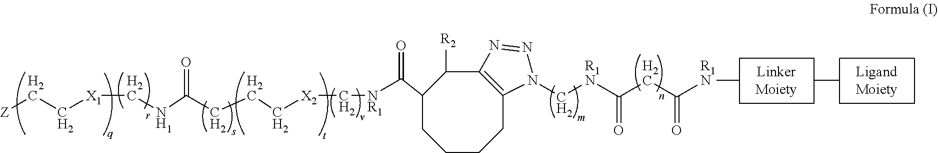

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleotide moiety.
2. A compound according to Sentence 1, wherein R1 is hydrogen at each occurrence.
3. A compound according to Sentence 1, wherein R1 is methyl.
4. A compound according to Sentence 1, wherein R1 is ethyl.
5. A compound according to any of Sentences 1 to 4, wherein R2 is hydroxy.
6. A compound according to any of Sentences 1 to 4, wherein R2 is halo.
7. A compound according to Sentence 6, wherein R2 is fluoro.
8. A compound according to Sentence 6, wherein R2 is chloro.
9. A compound according to Sentence 6, wherein R2 is bromo.
10. A compound according to Sentence 6, wherein R2 is iodo.
11. A compound according to Sentence 6, wherein R₂ is nitro.
12. A compound according to any of Sentences 1 to 11, wherein X1 is methylene.
13. A compound according to any of Sentences 1 to 11, wherein X1 is oxygen.
14. A compound according to any of Sentences 1 to 11, wherein X1 is sulfur.
15. A compound according to any of Sentences 1 to 14, wherein X2 is methylene.
16. A compound according to any of Sentences 1 to 15, wherein X2 is oxygen.
17. A compound according to any of Sentences 1 to 16, wherein X₂ is sulfur.
18. A compound according to any of Sentences 1 to 17, wherein m=3.
19. A compound according to any of Sentences 1 to 18, wherein n=6.
20. A compound according to Sentences 13 and 15, wherein X₁ is oxygen and X₂ is methylene, and preferably wherein:
q=1,
r=2,
s=1,
t=1,
v=1.
21. A compound according to Sentences 12 and 15, wherein both X₁ and X₂ are methylene, and preferably wherein:
q=1,
r=3,
s=1,
t=1,
v=1.
22. A compound according to any of Sentences 1 to 21, wherein Z is:

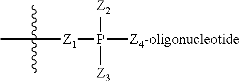

wherein:
Z1, Z2, Z3, Z4 are independently at each occurrence oxygen or sulfur; and
one the bonds between P and Z2, and P and Z3 is a single bond and the other bond is a double bond.
23. A compound according to Sentence 22, wherein said oligonucleotide is an RNA compound capable of modulating, preferably inhibiting, expression of a target gene.
24. A compound according to Sentence 23, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
25. A compound according to Sentence 24, wherein the RNA compound is attached at the 5′ end of its second strand to the adjacent phosphate.
26. A compound according to Sentence 24, wherein the RNA compound is attached at the 3′ end of its second strand to the adjacent phosphate.
27. A compound of Formula (II):

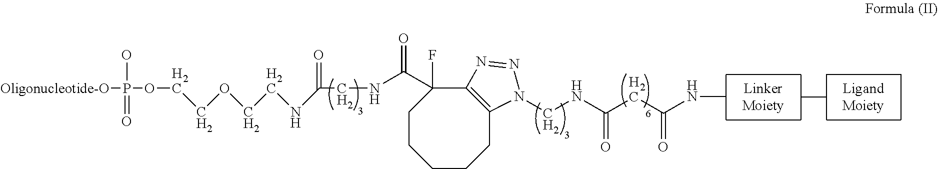

28. A compound of Formula (III):

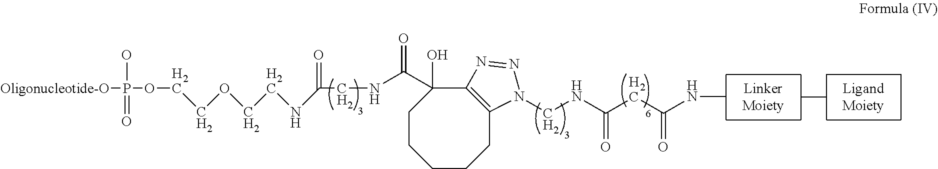

29. A compound according to Sentence 27 or 28, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
30. A composition comprising a compound of Formula (II) as defined in Sentence 27, and a compound of Formula (III) as defined in Sentence 28, optionally dependent on Sentence 29.
31. A composition according to Sentence 30, wherein said compound of Formula (III) as defined in Sentence 28 is present in an amount in the range of 10 to 15% by weight of said composition.
32. A compound of Formula (IV):

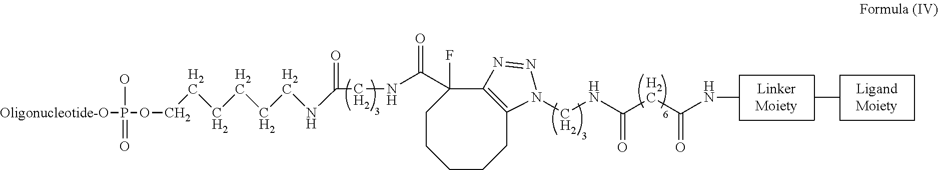

33. A compound of Formula (V):

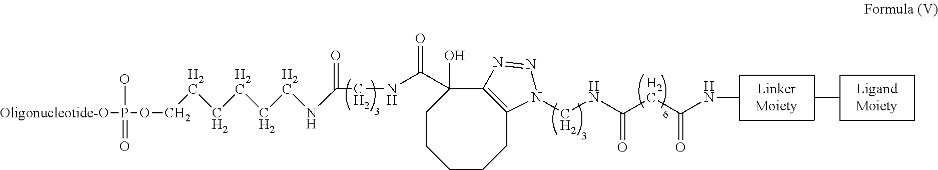

34. A compound according to Sentence 32 or 33, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
35. A composition comprising a compound of Formula (IV) as defined in Sentence 32, and a compound of Formula (V) as defined in Sentence 33, optionally dependent on Sentence 34.
36. A composition according to Sentence 35, wherein said compound of Formula (V) as defined in Sentence 33 is present in an amount in the range of 10 to 15% by weight of said composition.
37. A compound as defined in any of Sentences 1 to 29, or 32 to 34, wherein the oligonucleotide comprises an RNA duplex which further comprises one or more riboses modified at the 2′ position, preferably a plurality of riboses modified at the 2′ position.
38. A compound according to Sentence 37, wherein the modifications are chosen from 2′-O-methyl, 2′-deoxy-fluoro, and 2′-deoxy.
39. A compound according to any of Sentences 1 to 29, or 32 to 34, or 37 to 38, wherein the oligonucleotide further comprises one or more degradation protective moieties at one or more ends.
40. A compound according to Sentence 39, wherein said one or more degradation protective moieties are not present at the end of the oligonucleotide strand that carries the ligand moieties, and/or wherein said one or more degradation protective moieties is selected from phosphorothioate internucleotide linkages, phosphorodithioate internucleotide linkages and inverted abasic nucleotides, wherein said inverted abasic nucleotides are present at the distal end of the strand that carries the ligand moieties.
41. A compound according to any of Sentences 1 to 29, or 32 to 34, or 37 to 40, wherein said ligand moiety as depicted in Formula (I) in Sentence 1 comprises one or more ligands.
42. A compound according to Sentence 41, wherein said ligand moiety as depicted in Formula (I) in Sentence 1 comprises one or more carbohydrate ligands.
43. A compound according to Sentence 42, wherein said one or more carbohydrates can be a monosaccharide, disaccharide, trisaccharide, tetrasaccharide, oligosaccharide or polysaccharide.
44. A compound according to Sentence 43, wherein said one or more carbohydrates comprise one or more galactose moieties, one or more lactose moieties, one or more N-AcetylGalactosamine moieties, and/or one or more mannose moieties.
45. A compound according to Sentence 44, wherein said one or more carbohydrates comprise one or more N-Acetyl-Galactosamine moieties.
46. A compound according to Sentence 45, which comprises two or three N-AcetylGalactosamine moieties.
47. A compound according to any of Sentences 41 to 46, wherein said one or more ligands are attached in a linear configuration, or in a branched configuration.
48. A compound according to Sentence 47, wherein said one or more ligands are attached as a biantennary or triantennary branched configuration.
49. A compound according to Sentences 46 to 48, wherein said moiety:

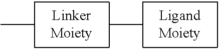

as depicted in Formula (I) in Sentence 1 is any of Formulae (VIa), (VIb) or (VIc), preferably Formula (VIa):

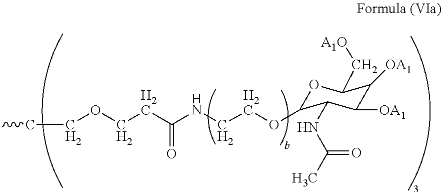

wherein:
A₁ is hydrogen, or a suitable hydroxy protecting group;
a is an integer of 2 or 3; and
b is an integer of 2 to 5; or

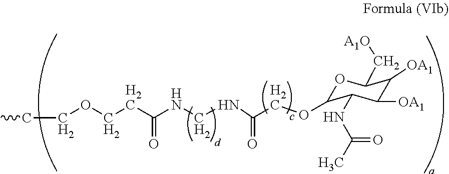

wherein:
A₁ is hydrogen, or a suitable hydroxy protecting group;
a is an integer of 2 or 3; and
c and d are independently integers of 1 to 6; or

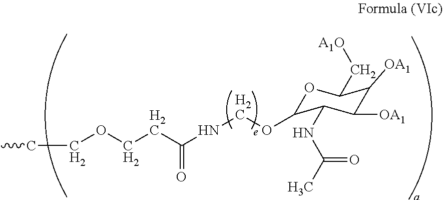

wherein:
A₁ is hydrogen, or a suitable hydroxy protecting group;
a is an integer of 2 or 3; and
e is an integer of 2 to 10.
50. A compound according to Sentences 46 to 48, wherein said moiety:

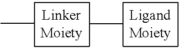

as depicted in Formula (I) in Sentence 1 is Formula (VII):

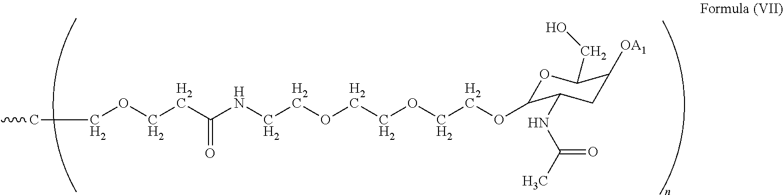

wherein:
A1 is hydrogen;
a is an integer of 2 or 3.
51. A compound according to Sentence 49 or 50, wherein a=2.
52. A compound according to Sentence 49 or 50, wherein a=3.
53. A compound according to Sentence 49, wherein b=3.
54. A compound of Formula (VIII):

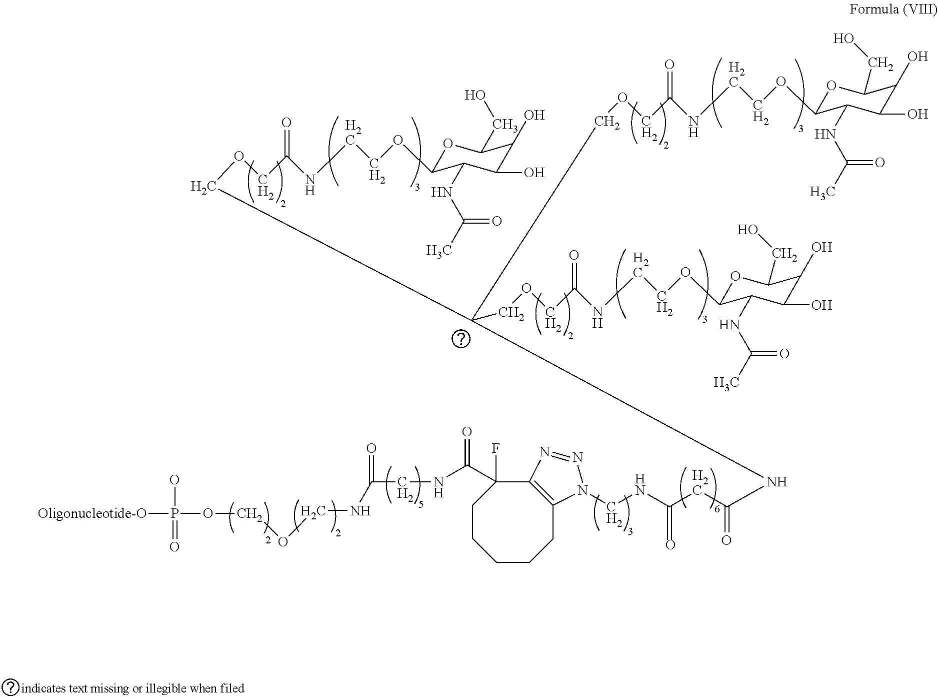

55. A compound of Formula (IX):

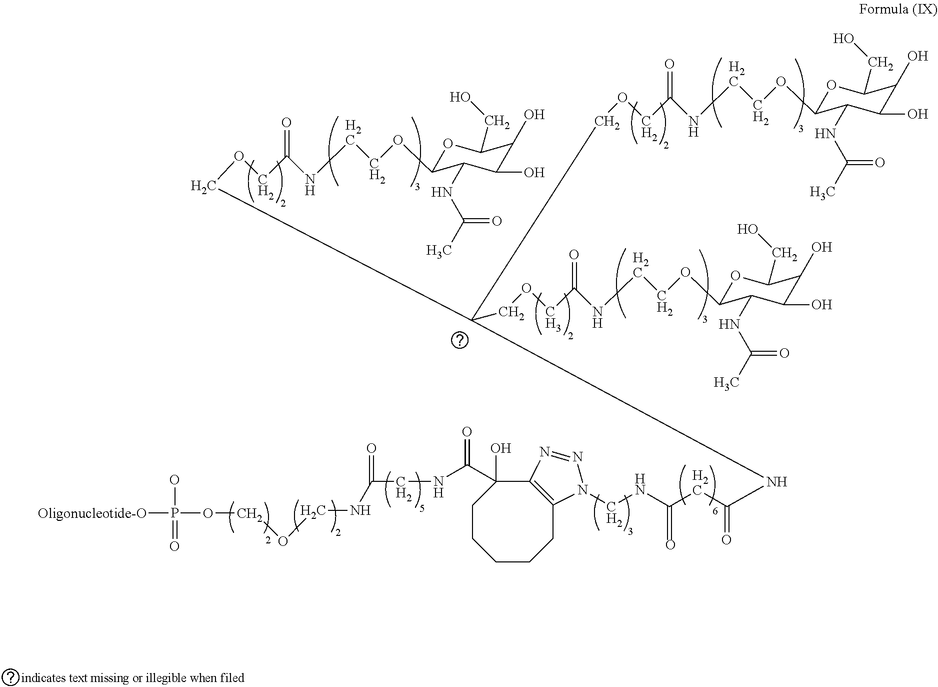

56. A compound according to Sentence 58 or 55, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
57. A composition comprising a compound of Formula (VIII) as defined in Sentence 54, and a compound of Formula (IX) as defined in Sentence 55, optionally dependent on Sentence 56.
58. A composition according to Sentence 57, wherein said compound of Formula (IX) as defined in Sentence 55 is present in an amount in the range of 10 to 15% by weight of said composition.
59. A compound of Formula (X):

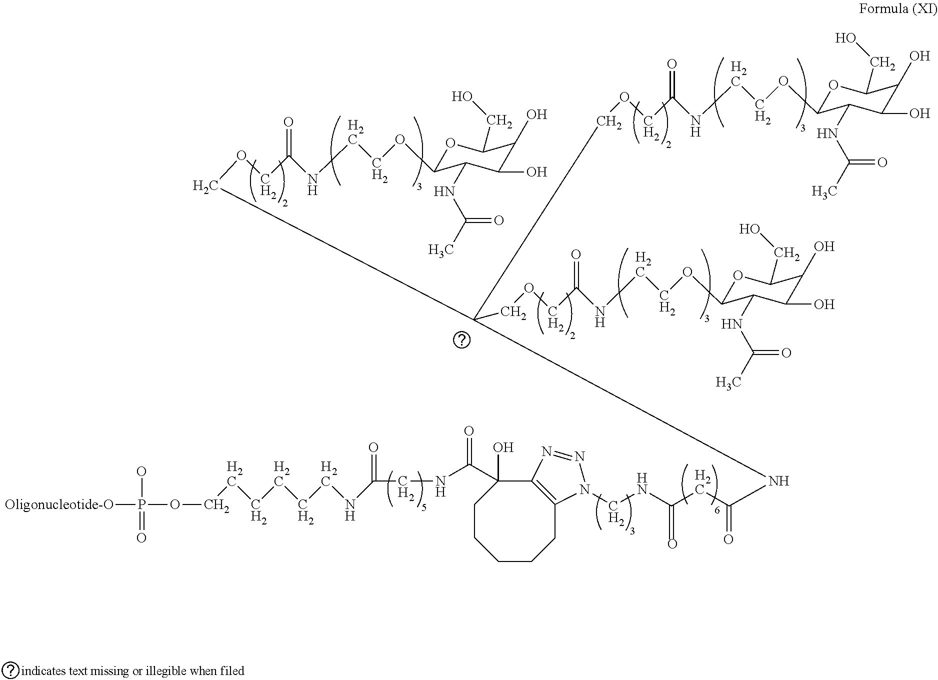

60. A compound of Formula (XI):

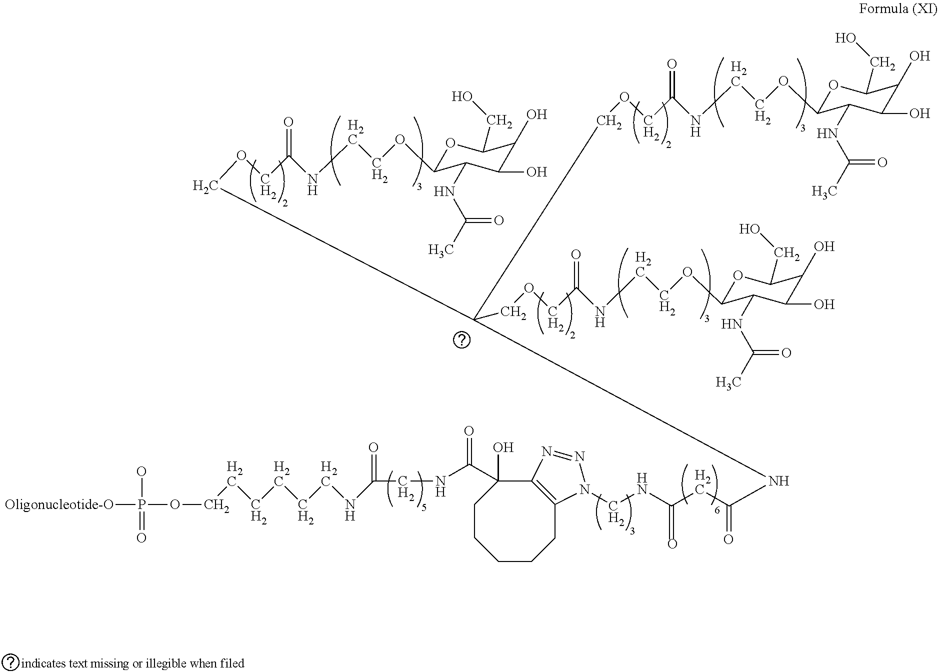

61. A compound according to Sentence 59 or 60, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
62. A composition comprising a compound of Formula (X) as defined in Sentence 59, and a compound of Formula (XI) as defined in Sentence 60, optionally dependent on Sentence 61.
63. A composition according to Sentence 62, wherein said compound of Formula (XI) as defined in Sentence 60 is present in an amount in the range of 10 to 15% by weight of said composition.
64. A compound as defined in any of Sentences 54 to 63, wherein the oligonucleotide comprises an RNA duplex which further comprises one or more riboses modified at the 2′ position, preferably a plurality of riboses modified at the 2′ position.
65. A compound according to Sentence 64, wherein the modifications are chosen from 2′-O-methyl, 2′-deoxy-fluoro, and 2′-deoxy.
66. A compound according to any of Sentences 54 to 65, wherein the oligonucleotide further comprises one or more degradation protective moieties at one or more ends.
67. A compound according to Sentence 66, wherein said one or more degradation protective moieties are not present at the end of the oligonucleotide strand that carries the ligand moieties, and/or wherein said one or more degradation protective moieties is selected from phosphorothioate internucleotide linkages, phosphorodithioate internucleotide linkages and inverted abasic nucleotides, wherein said inverted abasic nucleotides are present at the distal end of the strand that carries the ligand moieties, as shown in any of Formulae (VIII), (IX), (X) or (XI) in any of Sentences 54, 55, 59 or 60.
68. A process of preparing a compound according to any of Sentences 1 to 29, 32 to 34, 37 to 56, 59 to 61, and 64 to 67, and/or a composition according to any of Sentences 30, 31, 35, 36, 57, 58, 62, 63, which comprises reacting compounds of Formulae (XII) and (XIII):

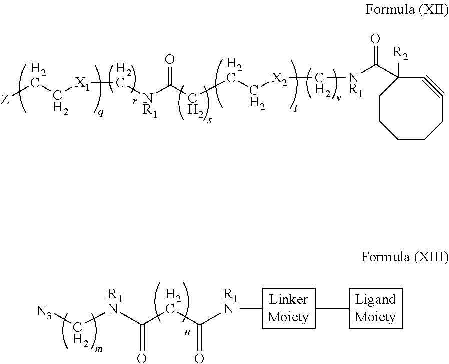

herein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleotide moiety;
and where appropriate carrying out deprotection of the ligand and/or annealing of a second strand for the oligonucleotide moiety.
69. A process according to Sentence 68, wherein a compound of Formula (XII) is prepared by reacting compounds of Formulae (XIV) and (XV):

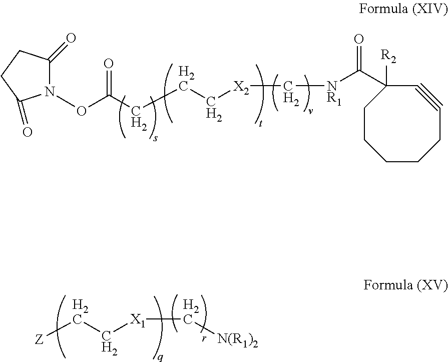

R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleotide moiety.
70. A process according to Sentence 68, to prepare a compound according to any of Sentences 20, 25, 27, 29, 54, 56, and/or a composition according to any of Sentences 30, 31, 57, 58, wherein:
compound of Formula (XII) is Formula (XIIa):

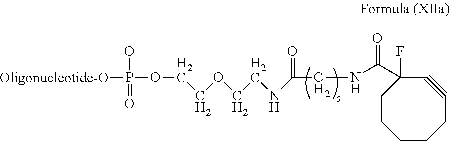

and compound of Formula (XIII) is Formula (XIIIa):

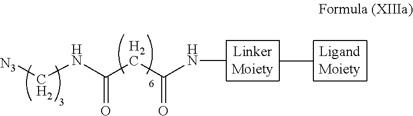

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
71. A process according to Sentence 68, to prepare a compound according to any of Sentences 20, 25, 28, 29, 55, 56, and/or a composition according to any of Sentences 30, 31, 57, 58, wherein:
compound of Formula (XII) is Formula (XIIb):

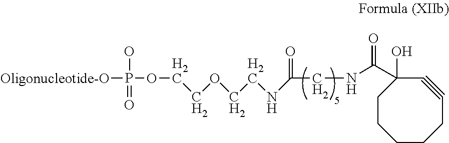

and compound of Formula (XIII) is Formula (XIIIa):

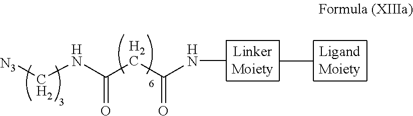

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
72. A process according to Sentence 68, to prepare a compound according to any of Sentences 21, 26, 32, 34, 59, 61, and/or a composition according to any of Sentences 35, 36, 62, 63, wherein:
compound of Formula (XII) is Formula (XIIc):

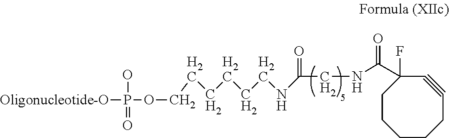

and compound of Formula (XIII) is Formula (XIIIa):

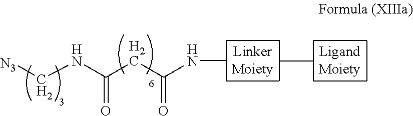

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
73. A process according to Sentence 68, to prepare a compound according to any of Sentences 21, 26, 33, 34, 60, 61, and/or a composition according to any of Sentences 35, 36, 62, 63, wherein:
compound of Formula (XII) is Formula (XIId):

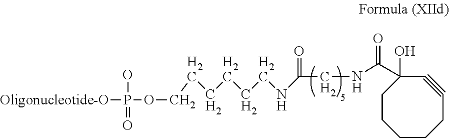

and compound of Formula (XIII) is Formula (XIIIa):

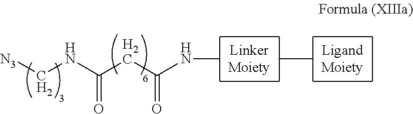

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
74. A process according to any of Sentences 70 to 73, wherein:
compound of Formula (XIIIa) is Formula (XIIIb):

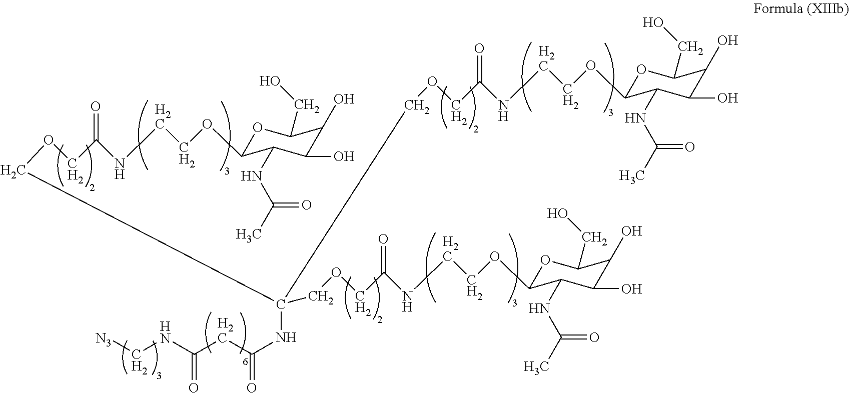

75. A process according to Sentences 69, as dependent on Sentences 70 to 73, wherein:
compound of Formula (XIV) is either Formula (XIVa) or Formula (XIIIVb):

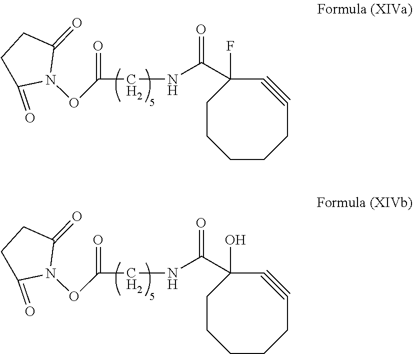

and compound of Formula (XV) is either Formula (XVa) or Formula (XIVb):

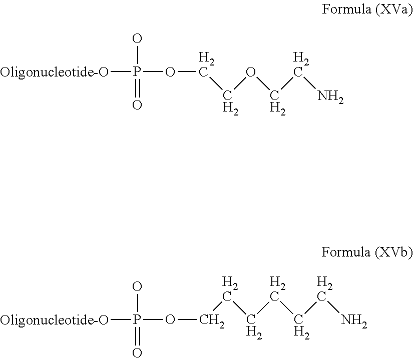

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein (i) said RNA duplex is attached at the 5′ end of its second strand to the adjacent phosphate in Formula (XVa), or (ii) said RNA duplex is attached at the 3′ end of its second strand to the adjacent phosphate in Formula (XVb).
76. A compound of Formula (XII):

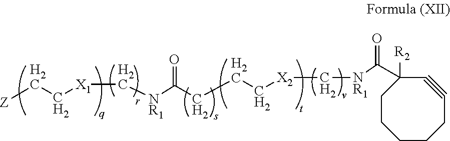

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleotide moiety.
77. A compound of Formula (XIIa):

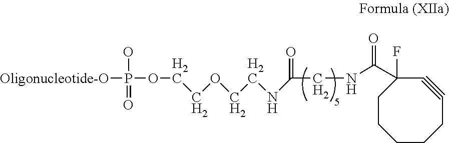

78. A compound of Formula (XIIb):

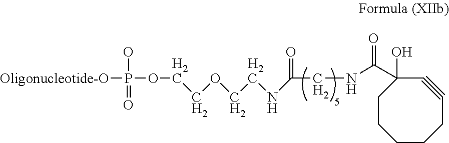

79. A compound of Formula (XIIc):

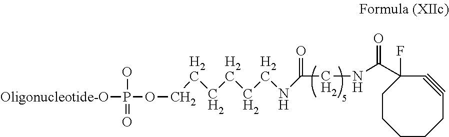

80. compound of Formula (XIId):

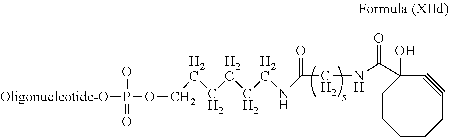

81. A compound of Formula (XIII):

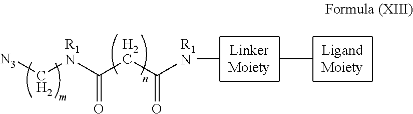

wherein:
R₁ at each occurrence is independently selected from the group consisting of hydrogen, methyl and ethyl;
m is an integer of from 1 to 6;
n is an integer of from 1 to 10.
82. A compound of Formula (XIIIa):

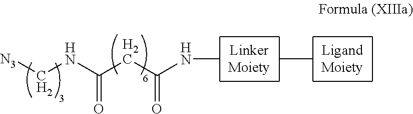

83. A compound of Formula (XIIIb):

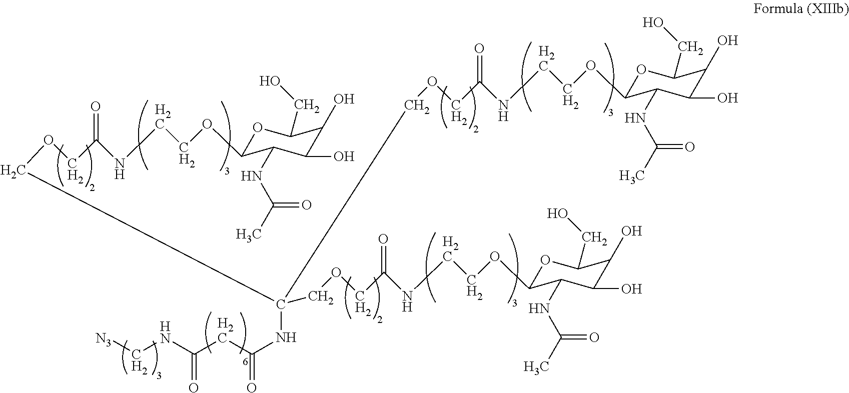

84. A compound of Formula (XIV)

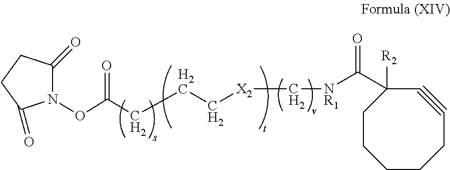

wherein:
R₁ is selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₂ is selected from the group consisting of methylene, oxygen and sulfur;
s, t, v are independently integers from 0 to 4, with the proviso that s, t and v cannot all be 0 at the same time.
85. A compound of Formula (XIVa):

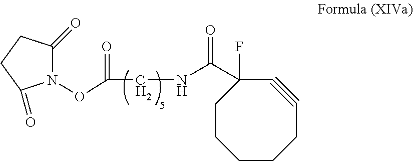

86. A compound of Formula (XIVb):

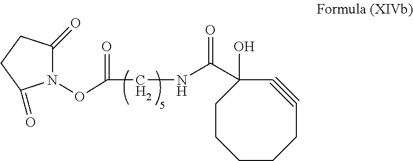

87. A compound of Formula (XV):

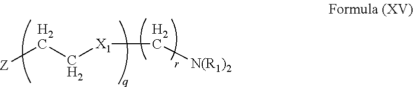

wherein:
R₁ at each occurrence is independently selected from the group consisting of hydrogen, methyl and ethyl;
X₁ is selected from the group consisting of methylene, oxygen and sulfur;
q and r are independently integers from 0 to 4, with the proviso that q and r cannot both be 0 at the same time;
Z is an oligonucleotide moiety.
88. A compound of Formula (XVa):

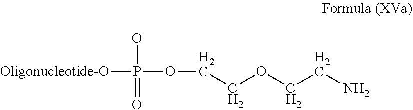

89. A compound of Formula (XVb):

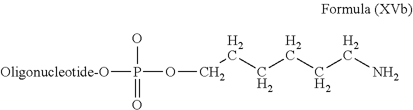

90. Use of a compound according to any of Sentences 76, 81 to 84, 87, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63.
91. Use of a compound according to Sentence 85, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═F.
92. Use of a compound according to Sentence 86, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R₂═OH.
93. Use of a compound according to Sentence 77, for the preparation of a compound according to any of Sentences 20, 25, 27, 29, 54, 56, and/or a composition according to any of Sentences 30, 31, 57, 58.
94. Use of a compound according to Sentence 78, for the preparation of a compound according to any of Sentences 20, 25, 28, 29, 55, 56, and/or a composition according to any of Sentences 30, 31, 57, 58.
95. Use of a compound according to Sentence 79, for the preparation of a compound according to any of Sentences 21, 26, 32, 34, 59, 61, and/or a composition according to any of Sentences 35, 36, 62, 63.
96. Use of a compound according to Sentence 80, for the preparation of a compound according to any of Sentences 21, 26, 33, 34, 60, 61, and/or a composition according to any of Sentences 35, 36, 62, 63.
97. Use of a compound according to Sentence 88, for the preparation of a compound according to any of Sentences 20, 25, 27 to 29, 54 to 56, and/or a composition according to any of Sentences 30, 31, 57, 58.
98. Use of a compound according to Sentence 89, for the preparation of a compound according to any of Sentences 21, 26, 32 to 34, 59 to 61, and/or a composition according to any of Sentences 35, 36, 62, 63.
99. A compound or composition obtained, or obtainable by a process according to any of Sentences 68 to 75.
100. A pharmaceutical composition comprising of a compound according to any of Sentences 1 to 29, 32 to 34, 37 to 56, 59 to 61, and 64 to 67, and/or a composition according to any of Sentences 30, 31, 35, 36, 57, 58, 62 and 63, together with a pharmaceutically acceptable carrier, diluent or excipient.
101. A compound according to any of Sentences 1 to 29, 32 to 34, 37 to 56, 59 to 61, and 64 to 67, and/or a composition according to any of Sentences 30, 31, 35, 36, 57, 58, 62 and 63, for use in therapy.

In another aspect the present invention may be applied in the compounds, processes, compositions or uses of the following Clauses numbered 1-56 (wherein reference to any Formula in the Clauses refers only to those Formulas that are defined within Clause 1-56. These formulae are reproduced in FIG. 44).
1. A compound comprising the following structure:

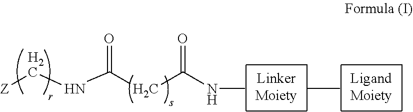

wherein:
r and s are independently an integer selected from 1 to 16; and
Z is an oligonucleotide moiety.
2. A compound according to Clause 1, wherein s is an integer selected from 4 to 12.
3. A compound according to Clause 2, wherein s is 6.
4. A compound according to any of Clauses 1 to 3, wherein r is an integer selected from 4 to 14.
5. A compound according to Clause 4, wherein r is 6.
6. A compound according to Clause 4, wherein r is 12.
7. A compound according to Clause 5, which is dependent on Clause 3.
8. A compound according to Clause 6, which is dependent on Clause 3.
9. A compound according to any of Clauses 1 to 8, wherein Z is:

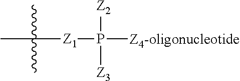

wherein:
Z₁, Z₂, Z₃, Z₄ are independently at each occurrence oxygen or sulfur; and
one the bonds between P and Z₂, and P and Z₃ is a single bond and the other bond is a double bond.
10. A compound according to any of Clauses 1 to 9, wherein said oligonucleotide is an RNA compound capable of modulating, preferably inhibiting, expression of a target gene.
11. A compound according to any of Clause 10, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
12. A compound according to Clause 11, preferably also dependent on Clauses 3 and 6, wherein the RNA compound is attached at the 5′ end of its second strand to the adjacent phosphate.
13. A compound according to Clause 11, preferably also dependent on Clauses 3 and 5, wherein the RNA compound is attached at the 3′ end of its second strand to the adjacent phosphate.
14. A compound of Formula (II), preferably dependent on Clause 12:

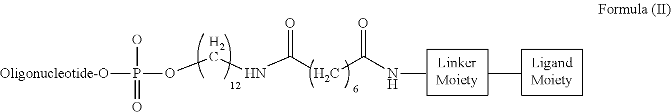

15. A compound of Formula (III), preferably dependent on Clause 13:

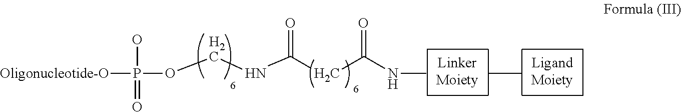

16. A compound as defined in any of Clauses 1 to 15, wherein the oligonucleotide comprises an RNA duplex which further comprises one or more riboses modified at the 2′ position, preferably a plurality of riboses modified at the 2′ position.
17. A compound according to Clause 16, wherein the modifications are chosen from 2′-O-methyl, 2′-deoxy-fluoro, and 2′-deoxy.
18. A compound according to any of Clauses 1 to 17, wherein the oligonucleotide further comprises one or more degradation protective moieties at one or more ends.
19. A compound according to Clause 18, wherein said one or more degradation protective moieties are not present at the end of the oligonucleotide strand that carries the linker/ligand moieties, and/or wherein said one or more degradation protective moieties is selected from phosphorothioate internucleotide linkages, phosphorodithioate internucleotide linkages and inverted abasic nucleotides, wherein said inverted abasic nucleotides are present at the distal end of the same strand to the end that carries the linker/ligand moieties.
20. A compound according to any of Clauses 1 to 19, wherein said ligand moiety as depicted in Formula (I) in Clause 1 comprises one or more ligands.
21. A compound according to Clause 20, wherein said ligand moiety as depicted in Formula (I) in Clause 1 comprises one or more carbohydrate ligands.
22. A compound according to Clause 21, wherein said one or more carbohydrates can be a monosaccharide, disaccharide, trisaccharide, tetrasaccharide, oligosaccharide or polysaccharide.
23. A compound according to Clause 22, wherein said one or more carbohydrates comprise one or more galactose moieties, one or more lactose moieties, one or more N-AcetylGalactosamine moieties, and/or one or more mannose moieties.
24. A compound according to Clause 23, wherein said one or more carbohydrates comprise one or more N-Acetyl-Galactosamine moieties.
25. A compound according to Clause 24, which comprises two or three N-AcetylGalactosamine moieties.
26. A compound according to any of the preceding Clauses, wherein said one or more ligands are attached in a linear configuration, or in a branched configuration.
27. A compound according to Clause 26, wherein said one or more ligands are attached as a biantennary or triantennary branched configuration.
28. A compound according to Clauses 20 to 27, wherein said moiety:

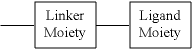

as depicted in Formula (I) in Clause 1 is any of Formulae (IV), (V) or (VI), preferably Formula (IV):

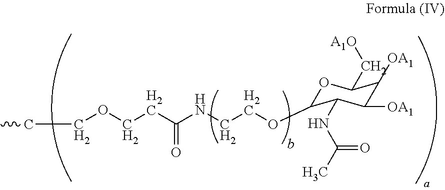

wherein:
A₁ is hydrogen, or a suitable hydroxy protecting group;
a is an integer of 2 or 3; and
b is an integer of 2 to 5; or

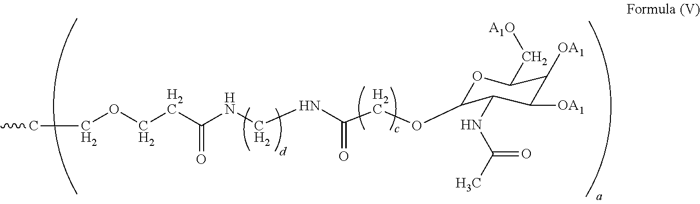

wherein:
A₁ is hydrogen, or a suitable hydroxy protecting group;
a is an integer of 2 or 3; and
c and d are independently integers of 1 to 6; or

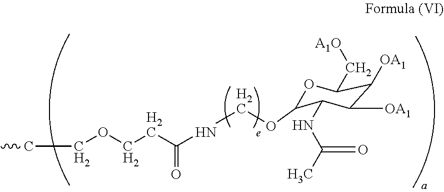

wherein:
A₁ is hydrogen, or a suitable hydroxy protecting group;
a is an integer of 2 or 3; and
e is an integer of 2 to 10.
29. A compound according to any of Clauses 1 to 28, wherein said moiety:

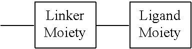

as depicted in Formula (I) in Clause 1 is Formula (VII):

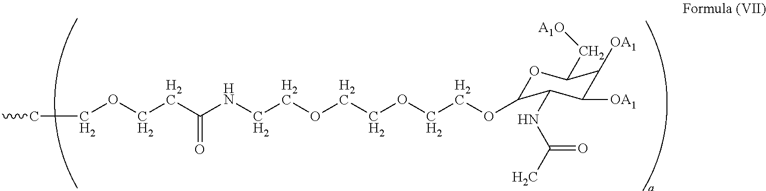

wherein:
A₁ is hydrogen;
a is an integer of 2 or 3.
30. A compound according to Clause 28 or 29, wherein a=2.
31. A compound according to Clause 28 or 29, wherein a=3.
32. A compound according to Clause 28, wherein b=3.
33. A compound of Formula (VIII):

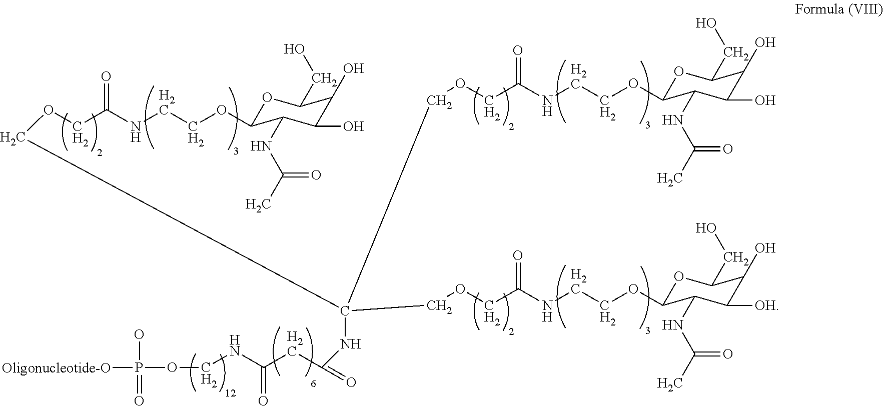

34. A compound of Formula (I):

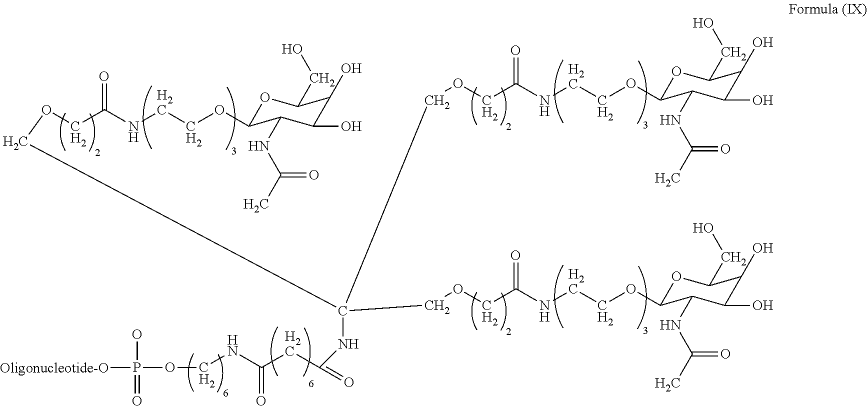

35. A compound according to Clause 33 or 34, wherein the oligonucleotide comprises an RNA duplex which further comprises one or more riboses modified at the 2′ position, preferably a plurality of riboses modified at the 2′ position.
36. A compound according to Clause 35, wherein the modifications are chosen from 2′-O-methyl, 2′-deoxy-fluoro, and 2′-deoxy.
37. A compound according to any of Clauses 33 to 36, wherein the oligonucleotide further comprises one or more degradation protective moieties at one or more ends.
38. A compound according to Clause 37, wherein said one or more degradation protective moieties are not present at the end of the oligonucleotide strand that carries the linker/ligand moieties, and/or wherein said one or more degradation protective moieties is selected from phosphorothioate internucleotide linkages, phosphorodithioate internucleotide linkages and inverted abasic nucleotides, wherein said inverted abasic nucleotides are present at the distal end of the same strand to the end that carries the linker/ligand moieties.
39. A compound according to Clause 33, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
40. A compound according to Clause 34, wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
41. A process of preparing a compound according to any of Clauses 1 to 40, which comprises reacting compounds of Formulae (X) and (XI):

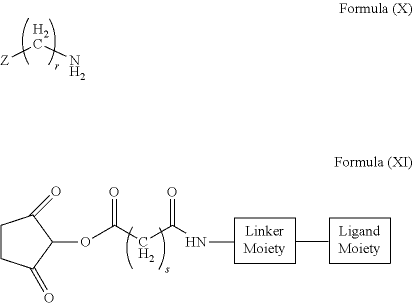

wherein:
r and s are independently an integer selected from 1 to 16; and
Z is an oligonucleotide moiety; and where appropriate carrying out deprotection of the ligand and/or annealing of a second strand for the oligonucleotide.
42. A process according to Clause 41, to prepare a compound according to any of Clauses 6, 8 to 14, 16 to 33, and 35 to 40, wherein:
compound of Formula (X) is Formula (Xa):

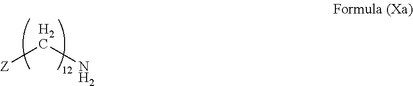

and compound of Formula (XI) is Formula (XIa):

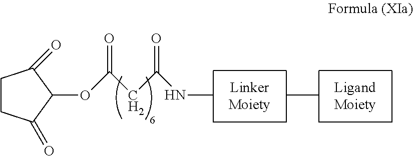

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
43. A process according to Clause 41, to prepare a compound according to any of Clauses 5, 7, 9 to 13, 15 to 32, and 34 to 40, wherein:
compound of Formula (X) is Formula (Xb):

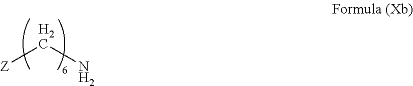

and compound of Formula (XI) is Formula (XIa):

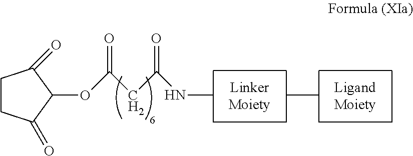

wherein the oligonucleotide comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
44. A process according to Clauses 42 or 43, wherein:
compound of Formula (XIa) is Formula (XIb):

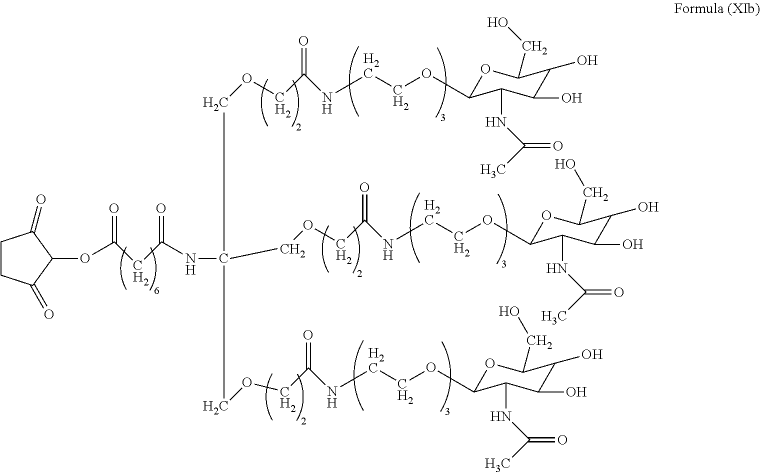

45. A compound of Formula (X):

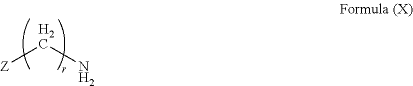

wherein:
r is independently an integer selected from 1 to 16; and
Z is an oligonucleotide moiety.
46. A compound of Formula (Xa):

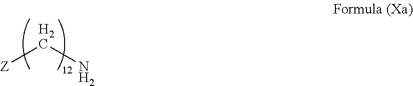

47. A compound of Formula (Xb):

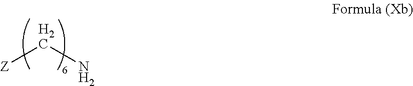

48. A compound of Formula (XI):

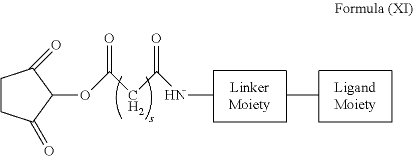

wherein:
s is independently an integer selected from 1 to 16; and
Z is an oligonucleotide moiety.
49. A compound of Formula (XIa):

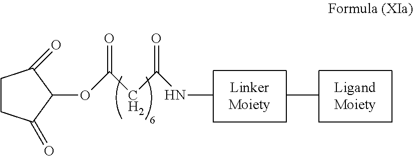

50. A compound of Formula (XIb):

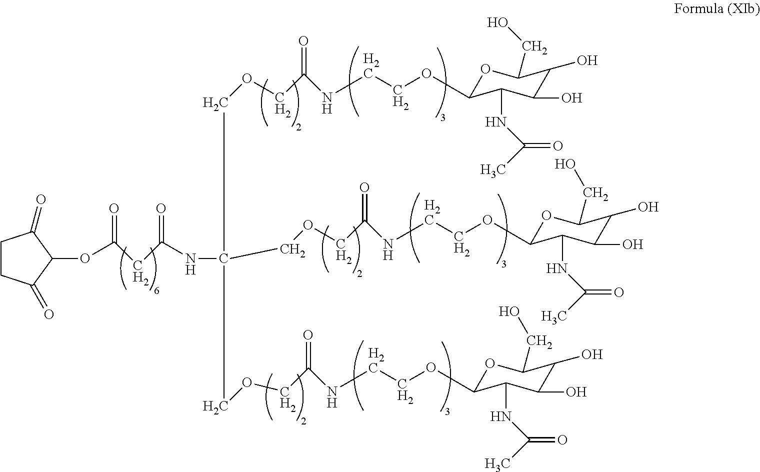

51. Use of a compound according to any of Clauses 45 and 48 to 50, for the preparation of a compound according to any of Clauses 1 to 40.
52. Use of a compound according to Clause 46, for the preparation of a compound according to any of Clauses 6, 8 to 14, 16 to 33, and 35 to 40.
53. Use of a compound according to Clause 47, for the preparation of a compound according to any of Clauses 5, 7, 9 to 13, 15 to 32, and 34 to 40.
54. A compound or composition obtained, or obtainable by a process according to any of Clauses 41 to 44.
55. A pharmaceutical composition comprising of a compound according to any of Clauses 1 to 40, together with a pharmaceutically acceptable carrier, diluent or excipient.
56. A compound according to any of Clauses 1 to 40, for use in therapy.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended Clauses.

The following constructs are used in the examples

TABLE 1

| | | | Antisense |
|---|---|---|---|
| Target | ID | Sense Sequence 5′→3′ | Sequence 5′→3′ |
| hsHAO1 | ETX005 | (invabasic)(invabasic) | usAfsuauUfuCfCfag |
| | | gsascuuuCfaUfCfCfuggaaauasusa | gaUfgAfaagucscsa |
| | | (NHC6)(MFCO)(ET-GalNAc-T1N3) | |
| hsHAO1 | ETX001 | (ET-GalNAc-T1N3)(MFCO)(NH-DEG) | usAfsuauUfuCfCfag |
| | | gacuuuCfaUfCfCfuggaaauasusa | gaUfgAfaagucscsa |
| | | (invabasic)(invabasic) | |
| hsC5 | ETX014 | (invabasic)(invabasic) | usAfsUfuAfuaAfaAf |
| | | asasGfcAfaGfaUfAfUfuUfuuAfuAfaua | auaUfcUfuGfcuusus |
| | | (NHC6)(MFCO)(ET-GalNAc-T1N3) | udTdT |
| hsC5 | ETX010 | (ET-GalNAc-T1N3)(MFCO)(NH-DEG) | usAfsUfuAfuaAfaAf |
| | | aaGfcAfaGfaUfAfUfuUfuuAfuAfasusa | auaUfcUfuGfcuusus |
| | | (invabasic)(invabasic) | udTdT |
| hsTTR | ETX019 | (ET-GalNAc-T1N3)(MFCO)(NH-DEG) | usCfsuugGfuuAfcau |
| | | ugggauUfuCfAfUfguaaccaasgsa | gAfaAfucccasusc |
| | | (invabasic)(invabasic) | |
| hsTTR | ETX023 | (invabasic)(invabasic) | usCfsuugGfuuAfcau |
| | | usgsggauUfuCfAfUfguaaccaaga | gAfaAfucccasusc |
| | | (NHC6)(MFCO)(ET-GalNAc-T1N3) | |

In Table 1 the components in brackets having the following nomenclature (ET-GalNAc-T1N3), (MFCO), and (NH-DEG) are descriptors of elements of the linkers, and the complete corresponding linker structures are shown in FIG. 30 and FIG. 31 herein. This correspondence of abbreviation to actual linker structure similarly applies to all other references of the above abbreviations herein.

Reference to (invabasic)(invabasic) refers to nucleotides in an overall polynucleotide which are the terminal 2 nucleotides which have sugar moieties that are (i) abasic, and (ii) in an inverted configuration, whereby the bond between the penultimate nucleotide and the antepenultimate nucleotide has a reversed linkage, namely either a 5-5 or a 3-3 linkage. Again, this similarly applies to all other references to (invabasic)(invabasic) herein.

TABLE 1A

| | | | Linker plus ligand |
|---|---|---|---|
| Target | ID | Short Descriptor | SiRNA as Table 1 |
| hsHAO1 | ETX005 | 3′-GalNAc T1a inverted abasic | Linker + ligand as FIG. 30 |
| hsHAO1 | ETX001 | 5′-GalNAc T1b inverted abasic | Linker + ligand as [symbol] FIG. 31 |
| hsC5 | ETX014 | 3′-GalNAc T1a inverted abasic | Linker + ligand as [symbol] FIG. 30 |
| hsC5 | ETX010 | 5′-GalNAc T1b inverted abasic | Linker + ligand as [symbol] FIG. 31 |
| hsTTR | ETX019 | 5′-GalNAc T1b inverted abasic | Linker + ligand as [symbol] FIG. 31 |
| hsTTR | ETX023 | 3′-GalNAc T1a inverted abasic | Linker + ligand as [symbol] FIG. 30 |

It should also be understood as already explained herein with reference to FIG. 30/FIG. 31, that where appropriate for the linker portions as shown in FIG. 30/FIG. 31 which can be present in any of products ETX001, ETX005, ETX010, ETX014, ETX019, ETX023 according to the present invention, that while these products can include molecules based on the linker and ligand portions as specifically depicted in FIG. 30/FIG. 31 attached to an oligonucleotide moiety as also depicted herein, these products may alternatively further comprise, or consist essentially of, molecules wherein the linker and ligand portions are essentially as depicted in FIG. 30/FIG. 31 attached to an oligonucleotide moiety but having the F substituent as shown in FIG. 30/FIG. 31 on the cyclo-octyl ring replaced by a substituent occurring as a result of hydrolytic displacement, such as an OH substituent. In this way, (a) these products can consist essentially of molecules having linker and ligand portions specifically as depicted in FIG. 30/FIG. 31, with a F substituent on the cyclo-octyl ring; or (b) these products can consist essentially of molecules having linker and ligand portions essentially as depicted in FIG. 30/FIG. 31 but having the F substituent as shown in FIG. 30/FIG. 31 on the cyclo-octyl ring replaced by a substituent occurring as a result of hydrolytic displacement, such as an OH substituent, or (c) these products can comprise a mixture of molecules as defined in (a) or (b).

The following control constructs are also used in the examples:

TABLE 2

| | | Sense | Antisense |
|---|---|---|---|
| Target | ID | Sequence 5′→3′ | Sequence 5′→3′ |
| F-Luc | XD- | cuuAcGcuGAGuAc | UCGAAGuACUcAGC |
| | 00914 | uucGAdTsdT | GuAAGdTsdT |
| hsFVII | XD- | AGAuAuGcAcAcAc | UCCGUGUGUGUGcA |
| | 03999 | AcGGAdTsdT | uAUCUdTsdT |
| hsAHSA1 | XD- | uscsUfcGfuGfgC | UfsUfsuCfaUfuA |
| | 15421 | fcUfuAfaUfgAfa | faGfgCfcAfcGfa |
| | | Af(invdT) | Gfasusu |

### Abbreviations

AHSA1 Activator of heat shock protein ATPase1
ASGR1 Asialoglycoprotein Receptor 1
ASO Antisense oligonucleotide
bDNA branched DNA
bp base-pair
C5 complement C5
conc. concentration
ctrl. control
CV coefficient of variation
dG, dC, dA, dT DNA residues
F Fluoro
FCS fetal calf serum
GalNAc N-Acetylgalactosamine
GAPDH Glyceraldehyde 3-phosphate dehydrogenase
G, C, A, U RNA residues
g, c, a, u 2′-O-Methyl modified residues
Gf, Cf, Af, Uf 2′-Fluoro modified residues
h hour
HAO1 Hydroxyacid Oxidase 1
HPLC High performance liquid chromatography
Hs Homo sapiens
IC50 concentration of an inhibitor where the response is reduced by 50%
ID identifier
KD knockdown
LF2000 Lipofectamine2000
M molar
Mf Macaca fascicularis
min minute
MV mean value
n.a. or N/A not applicable
NEAA non-essential amino acid
nt nucleotide
QC Quality control
QG2.0 QuantiGene 2.0
RLU relative light unit
RNAi RNA interference
RT room temperature
s Phosphorothioate backbone modification
SAR structure-activity relationship
SD standard deviation
siRNA small interfering RNA
TTR Transthyretin

### Example 1

### Summary

GalNAc-siRNAs targeting either hsHAO1, hsC5 or hsTTR mRNA were synthesized and QC-ed. The entire set of siRNAs (except siRNAs targeting HAO1) was first studied in a dose-response setup in HepG2 cells by transfection using RNAiMAX, followed by a dose-response analysis in a gymnotic free uptake setup in primary human hepatocytes.

Direct incubation of primary human hepatocytes with GalNAc-siRNAs targeting hsHAO1, hsC5 or hsTTR mRNA resulted in dose-dependent on-target mRNA silencing to varying degrees.

Aim of Study

The aim of this set of experiments was to analyze the in vitro activity of different GalNAc-ligands in the context of siRNAs targeting three different on-targets, namely hsHAO1, hsC5 or hsTTR mRNA.

Work packages of this study included (i) assay development to design, synthesize and test bDNA probe sets specific for each and every individual on-target of interest, (ii) to identify a cell line suitable for subsequent screening experiments, (iii) dose-response analysis of potentially all siRNAs (by transfection) in one or more human cancer cell lines, and (iv) dose-response analysis of siRNAs in primary human hepatocytes in a gymnotic, free uptake setting. In both settings, IC50 values and maximal inhibition values should be calculated followed by ranking of the siRNA study set according to their potency.

Material and Methods

### Oligonucleotide Synthesis

Standard solid-phase synthesis methods were used to chemically synthesize siRNAs of interest (see Table 1) as well as controls (see Table 2).

### Cell Culture and In-Vitro Transfection Experiments

Cell culture, transfection and QuantiGene2.0 branched DNA assay are described below, and siRNA sequences are listed in Tables 1 and 2. HepG2 cells were supplied by American Tissue Culture Collection (ATCC) (HB-8065, Lot #: 63176294) and cultured in ATCC-formulated Eagle's Minimum Essential Medium supplemented to contain 10% fetal calf serum (FCS). Primary human hepatocytes (PHHs) were sourced from Primacyt (Schwerin, Germany) (Lot #: CyHuf19009HEc). Cells are derived from a malignant glioblastoma tumor by explant technique. All cells used in this study were cultured at 37° C. in an atmosphere with 5% CO₂ in a humidified incubator.

For transfection of HepG2 cells with hsC5 or hsTTR targeting siRNAs (and controls), cells were seeded at a density of 20.000 cells/well in regular 96-well tissue culture plates. Transfection of cells with siRNAs was carried out using the commercially available transfection reagent RNAiMAX (Invitrogen/Life Technologies) according to the manufacturer's instructions. 10 point dose-response experiments of 20 candidates (11×hsC5, 9×hsTTR) were done in HepG2 cells with final siRNA concentrations of 24, 6, 1.5, 0.4, 0.1, 0.03, 0.008, 0.002, 0.0005 and 0.0001 nM, respectively.

Dose response analysis in PHHs was done by direct incubation of cells in a gymnotic, free uptake setting starting with 1.5 μM highest final siRNA concentration, followed by 500 nM and from there on going serially down in twofold dilution steps.

Control wells were transfected into HepG2 cells or directly incubated with primary human hepatocytes at the highest test siRNA concentrations studied on the corresponding plate. All control siRNAs included in the different project phases next to mock treatment of cells are summarized and listed in Table 2. For each siRNA and control, at least four wells were transfected/directly incubated in parallel, and individual data points were collected from each well.

After 24 h of incubation with siRNA post-transfection, media was removed and HepG2 cells were lysed in Lysis Mixture (1 volume of lysis buffer plus 2 volumes of nuclease-free water) and then incubated at 53° C. for at least 45 minutes. In the case of PHHs, plating media was removed 5 h post treatment of cells followed by addition of 50 μl of complete maintenance medium per well. Media was exchanged in that way every 24 h up to a total incubation period of 72 h. At either 4 h or 72 h time point, cell culture supernatant was removed followed by addition of 200 μl of Lysis Mixture supplemented with 1:1000 v/v of Proteinase K.

The branched DNA (bDNA) assay was performed according to manufacturer's instructions. Luminescence was read using a 1420 Luminescence Counter (WALLAC VICTOR Light, Perkin Elmer, Rodgau-Jügesheim, Germany) following 30 minutes incubation in the presence of substrate in the dark. For each well, the on-target mRNA levels were normalized to the hsGAPDH mRNA levels. The activity of any siRNA was expressed as percent on-target mRNA concentration (normalized to hsGAPDH mRNA) in treated cells, relative to the mean on-target mRNA concentration (normalized to hsGAPDH mRNA) across control wells.

### Assay Development

QuantiGene2.0 branched DNA (bDNA) probe sets were designed and synthesised specific for Homo sapiens GAPDH, AHSA1, hsHAO1, hsC5 and hsTTR. bDNA probe sets were initially tested by bDNA analysis according to manufacturer's instructions, with evaluation of levels of mRNAs of interest in two different lysate amounts, namely 10 μl and 50 μl, of the following human and monkey cancer cell lines next to primary human hepatocytes: SJSA-1, TF1, NCI-H1650, Y-79, Kasumi-1, EAhy926, Caki-1, Colo205, RPTEC, A253, HeLaS3, Hep3B, BxPC3, DU145, THP-1, NCI-H460, IGR37, LS174T, Be(2)-C, SW 1573, NCI-H358, TC71, 22Rv1, BT474, HeLa, KBwt, Panc-1, U87MG, A172, C42, HepG2, LNCaP, PC3, SupT11, A549, HCT116, HuH7, MCF7, SH-SY5Y, HUVEC, C33A, HEK293, HT29, MOLM 13 and SK-MEL-2. Wells containing only bDNA probe set without the addition of cell lysate were used to monitor technical background and noise signal.

Results

### Identification of Suitable Cell Types for Screening of GalNAc-siRNas

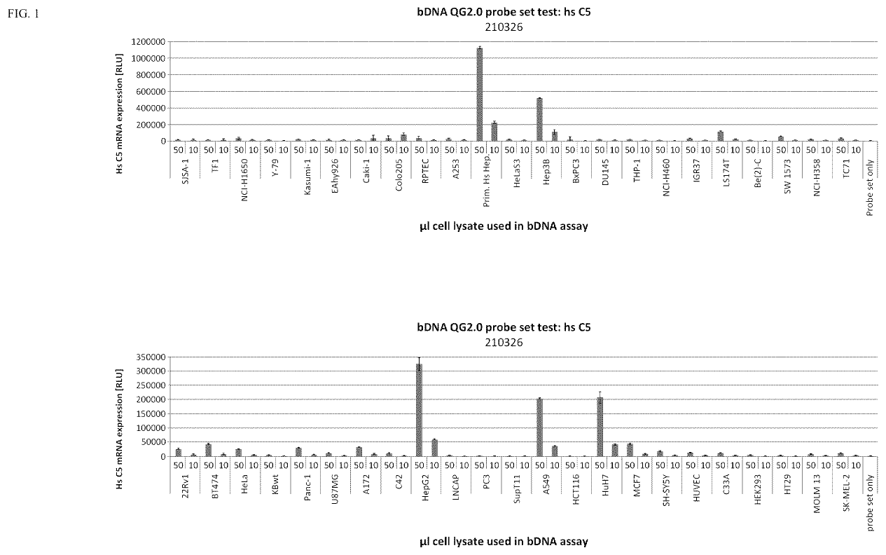

FIGS. 1-3 show mRNA expression data for the three on-targets of interest, namely hsC5, hsHAO1 and hsTTR, in lysates of a diverse set of human cancer cell lines plus primary human hepatocytes. Cell numbers per lysate volume are identical with each cell line tested, this is necessary to allow comparisons of expression levels amongst different cell types. FIG. 1 shows hsC5 mRNA expression data for all cell types tested.

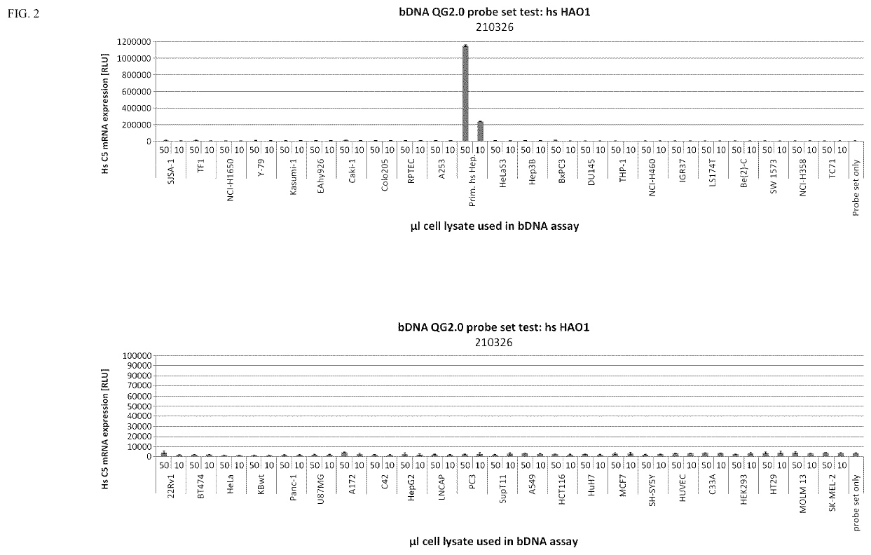

The identical type of cells were also screened for expression of hsHAO1 mRNA, results are shown in bar diagrams as part of FIG. 2.

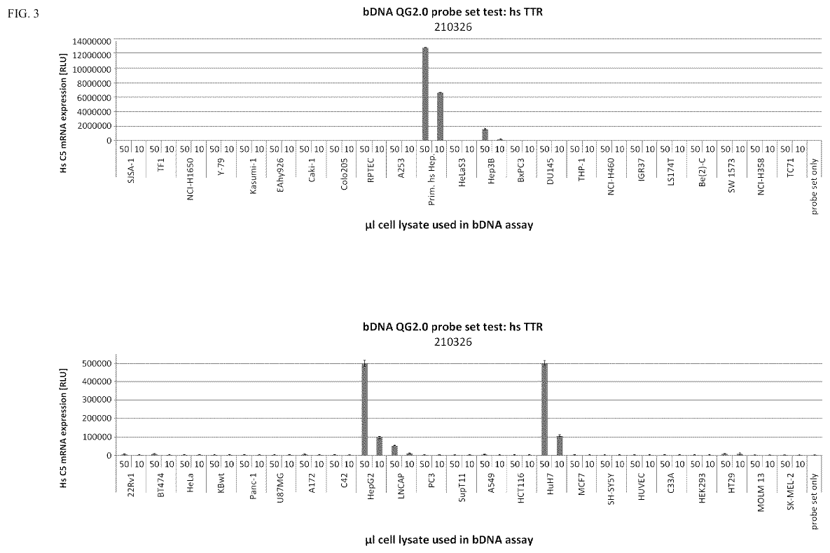

Lastly, suitable cell types were identified which would allow for screening of GalNAc-siRNAs targeting hsTTR, respective data are part of FIG. 3.

In summary, mRNA expression levels for all three on-targets of interest are high enough in primary human hepatocytes (PHHs). Further, HepG2 cells could be used to screen GalNAc-siRNAs targeting hsC5 and hsTTR mRNAs, in contrast, no cancer cell line could be identified which would be suitable to test siRNAs specific for hsHAO1 mRNA.

Dose-Response Analysis of hsTTR Targeting GalNAc-siRNAs in HepG2 Cells

Following transfection optimization, HepG2 cells were transfected with the entire set of hsTTR targeting GalNAc-siRNAs (see Table 1) in a dose-response setup using RNAiMAX. The highest final siRNA test concentration was 24 nM, going down in nine fourfold dilution steps. The experiment ended at 4 h and 24 h post transfection of HepG2 cells. Table 3 lists activity data for all hsTTR targeting GalNAC-siRNAs studied.

TABLE 3
Target, incubation time, external ID, IC20/IC50/IC80 values and
maximal inhibition of hsTTR targeting siRNAs in HepG2 cells.
The listing is ordered according to external ID, with 4 h of
incubation listed on top and 24 h of incubation on the bottom.

| | Incubation | External | IC20 | IC50 | IC80 | Max. Inhib. |
|---|---|---|---|---|---|---|
| Target | [h] | ID | [nM] | [nM] | [nM] | [%] |
| hsTTR | 4 | ETX019 | 0.206 | 3.769 | #N/A | 58.4 |
| hsTTR | 4 | ETX023 | 1.338 | #N/A | #N/A | 45.9 |
| hsTTR | 24 | ETX019 | 0.002 | 0.016 | 0.143 | 96.0 |
| hsTTR | 24 | ETX023 | 0.005 | 0.019 | 0.081 | 96.2 |

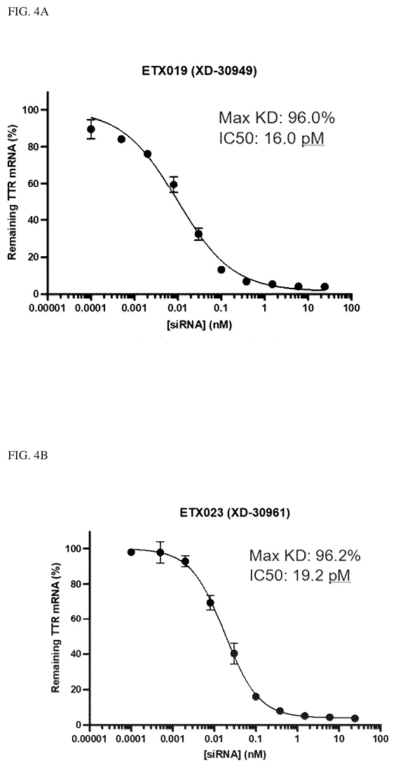

Results for the 24 h incubation are also shown in FIGS. 4A-4B.

In general, transfection of HepG2 cells with hsTTR targeting siRNAs results in on-target mRNA silencing spanning in general the entire activity range from 0% silencing to maximal inhibition. Data generated 24 h post transfection are more robust with lower standard variations, as compared to data generated only 4 h post transfection. Further, the extent of on-target knockdown generally increases over time from 4 h up to 24 h of incubation. hsTTR GalNAc-siRNAs have been identified that silence the on-target mRNA>95% with IC50 values in the low double-digit pM range.

Dose-Response Analysis of hsC5 Targeting GalNAc-siRNAs in HepG2 Cells

The second target of interest, hsC5 mRNA, was tested in an identical dose-response setup (with minimally different final siRNA test concentrations, however) by transfection of HepG2 cells using RNAiMAX with GalNAc-siRNAs sharing identical linger/position/GalNAc-ligand variations as with hsTTR siRNAs, but sequences specific for the on-target hsC5 mRNA.

TABLE 4
Target, incubation time, external ID, IC20/IC50/IC80 values and
maximal inhibition of hsC5 targeting siRNAs in HepG2 cells.
The listing is ordered according to external ID, with 4 h of
incubation listed on top and 24 h of incubation on the bottom.

| | Incubation | External | IC20 | IC50 | IC80 | Max. Inhib. |
|---|---|---|---|---|---|---|
| Target | [h] | ID | [nM] | [nM] | [nM] | [%] |
| C5 | 4 | ETX010 | 0.125 | 0.445 | #N/A | 71.1 |
| C5 | 4 | ETX014 | 0.595 | 2.554 | #N/A | 52.7 |
| C5 | 24 | ETX010 | 0.003 | 0.011 | 0.064 | 88.3 |
| C5 | 24 | ETX014 | 0.003 | 0.014 | 0.130 | 88.3 |

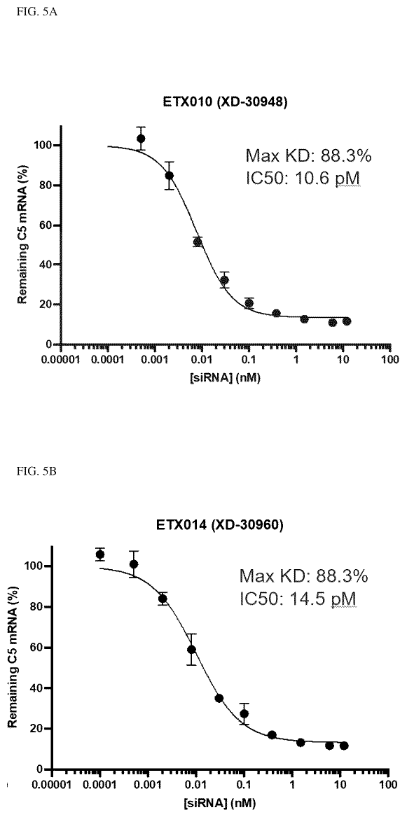

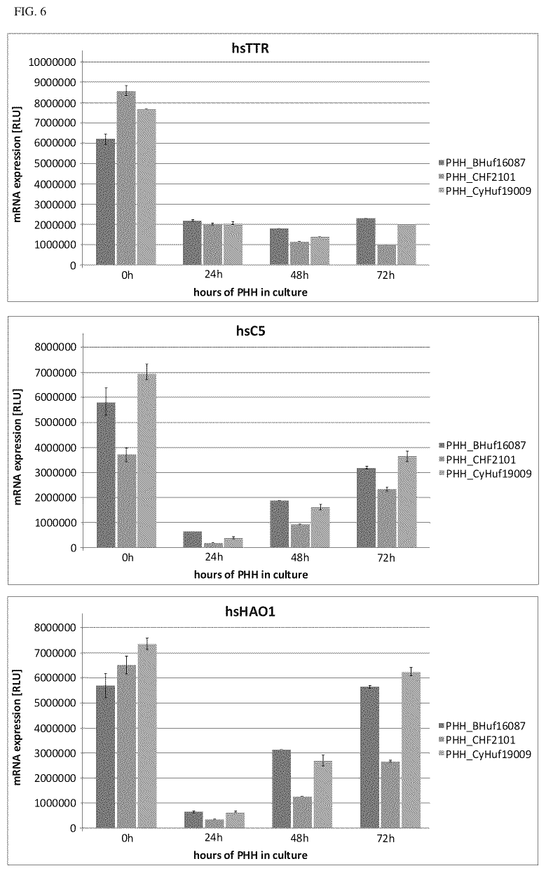

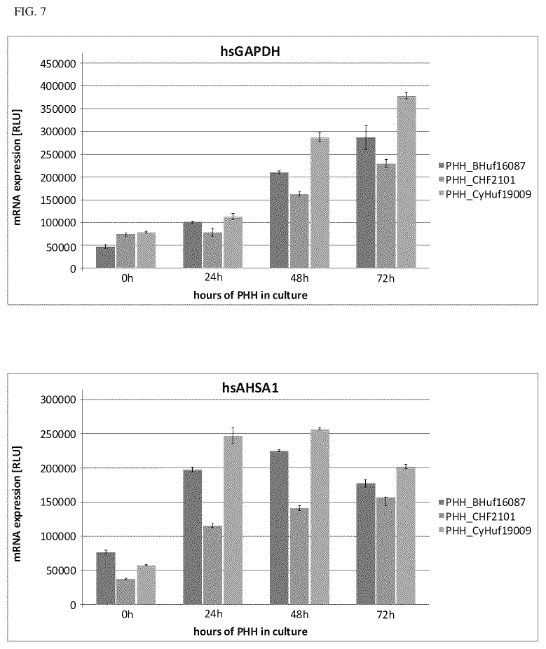

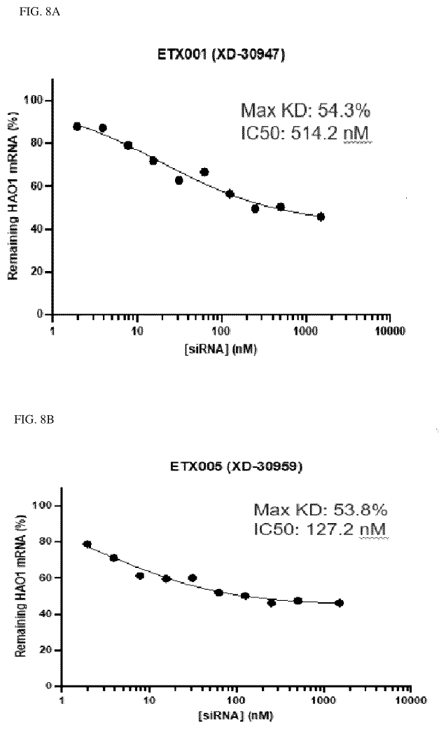

Results for the 24 h incubation are also shown in FIGS. 5A-5B.

There is dose-dependent on-target hsC5 mRNA silencing upon transfection of HepG2 cells with the GalNAc-siRNA set specific for hsC5. Some knockdown can already be detected at 4 h post-transfection of cells, an even higher on-target silencing is observed after a longer incubation period, namely 24 h. hsC5 GalNAc-siRNAs have been identified that silence the on-target mRNA almost 90% with IC50 values in the low single-digit pM range.

### Identification of a Primary Human Hepatocyte Batch Suitable for Testing of all GalNAc-siRNAs

The dose-response analysis of the two GalNAc-siRNA sets in human cancer cell line HepG2 should demonstrate (and ensure) that all new GalNAc-/linker/position/cap variants are indeed substrates for efficient binding to AGO2 and loading into RISC, and in addition, able to function in RNAi-mediated cleavage of target mRNA. However, in order to test whether the targeting GalNAc-ligand derivatives allow for efficient uptake into hepatocytes, dose-response analysis experiments should be done in primary human hepatocytes by gymnotic, free uptake setup. Hepatocytes do exclusively express the Asialoglycoprotein receptor (ASGR1) to high levels, and this receptor generally is used by the liver to remove target glycoproteins from circulation. It is common knowledge by now, that certain types of oligonucleotides, e.g. siRNAs or ASOs, conjugated to GalNAc-ligands are recognized by this high turnover receptor and efficiently taken up into the cytoplasm via clathrin-coated vesicles and trafficking to endosomal compartments. Endosomal escape is thought to be the rate-limiting step for oligonucleotide delivery.

An intermediate assay development experiment was done in which different batches of primary human hepatocytes were tested for their expression levels of relevant genes of interest, namely hsC5, hsTTR, hsHAO1, hsGAPDH and hsAHSA1. Primacyt (Schwerin, Germany) provided three vials of different primary human hepatocyte batches for testing, namely BHuf16087, CHF2101 and CyHuf19009. The cells were seeded on collagen-coated 96-well tissue culture plates, followed by incubation of cells for 0 h, 24 h, 48 h and 72 h before cell lysis and bDNA analysis to monitor mRNA levels of interest. FIG. 6 shows the absolute mRNA expression data for all three on-targets of interest—hsTTR, hsC5 and hsHAO1—in the primary human hepatocyte batches BHuf16087, CHF2101 and CyHuf19009. mRNA expression levels of hsGAPDH and hsAHSA1 are shown in FIG. 7.

In FIGS. 6 and 7 the left hand column of each data set triplet is BHuf16087, the middle column is CHF2101 and the right hand column is CyHuf19009.

Overall, the mRNA expression of all three on-targets of interest in the primary human hepatocyte batches BHuf16087 and CyHuf19009 are high enough after 72 h to continue with the bDNA assay. Due to the total amount of vials available for further experiments, we continued the experiments with the batch CyHuf19009.

Dose-Response Analysis of hsHAO1 Targeting GalNAc-siRNAs in PHHs

Following the identification of a suitable batch (CyHuf19009) of primary human hepatocytes (PHHs), a gymnotic, free uptake analysis was performed of hsHAO1 targeting GalNAc-siRNAs, listed in Table 1. The highest tested final siRNA concentration was 1.5 μM, followed by 500 nM, going down in eight two-fold serial dilution steps to the lowest final siRNA concentration of 1.95 nM. The experiments ended at 4 h and 72 h post direct incubation of PHH cells. Table 5 lists activity data for all hsHAO1 targeting GalNAc-siRNAs studied. All control siRNAs included in this experiment are summarized and listed in Table 2.

TABLE 5
Target, incubation time, external ID, IC20/IC50/IC80 values
and maximal inhibition of hsHAO1 targeting GalNAc-siRNAs
in primary human hepatocytes (PHHs). The listing is organized
according to external ID, with 4 h and 72 h incubation
listed on top and bottom, respectively.

| | Incubation | External | IC20 | IC50 | IC80 | Max. Inhib. |
|---|---|---|---|---|---|---|
| Target | [h] | ID | [nM] | [nM] | [nM] | [%] |
| hsHAO1 | 4 | ETX001 | #N/A | #N/A | #N/A | 3.5 |
| (hsGO1) | | | | | | |
| hsHAO1 | 4 | ETX005 | #N/A | #N/A | #N/A | 0.7 |
| (hsGO1) | | | | | | |
| hsHAO1 | 72 | ETX001 | 7.1 | 514.2 | #N/A | 54.3 |
| (hsGO1) | | | | | | |
| hsHAO1 | 72 | ETX005 | 1.5 | 127.2 | #N/A | 53.8 |
| (hsGO1) | | | | | | |

Results for the 72 h incubation are also shown in FIGS. 8A-8B.

Gymnotic, free uptake of GalNAc-siRNAs targeting hsHAO1 did not lead to significant on-target silencing within 4 h, however after 72 h incubation on-target silencing was visible in a range of 35.5 to 58.1% maximal inhibition.

Dose-Response Analysis of hsC5 Targeting GalNAc-siRNAs in PHHs

The second target of interest, hsC5 mRNA, was tested in an identical dose-response setup by gymnotic, free uptake in PHHs with GalNAc-siRNAs sharing identical linker/position/GalNAc-ligand variations as with hsTTR and hsHAO1 tested in the assays before, but sequences specific for the on-target hsC5 mRNA. Sequences for the GalNAc-siRNAs targeting hsC5 and all sequences and information about control siRNAs are listed in Table 1 and Table 2, respectively. The experiment ended after 4 h and 72 h direct incubation of PHHs. Table 6 lists activity data for all hsC5 targeting GalNAc-siRNAs studied.

TABLE 6
Target, incubation time, external ID, IC20/IC50/IC80 values and
maximal inhibition of hsC5 targeting GalNAc-siRNAs in PHHs.
The listing is organized according to external ID, with 4 h
and 72 h incubation listed on top and bottom, respectively.

| | Incubation | External | IC20 | IC50 | IC80 | Max. Inhib. |
|---|---|---|---|---|---|---|
| Target | [h] | ID | [nM] | [nM] | [nM] | [%] |
| C5 | 4 | ETX010 | #N/A | #N/A | #N/A | −1.3 |
| C5 | 4 | ETX014 | 51.8 | #N/A | #N/A | 23.7 |
| C5 | 72 | ETX010 | 4.3 | 72.1 | #N/A | 64.9 |
| C5 | 72 | ETX014 | 2.2 | 63.7 | #N/A | 65.6 |

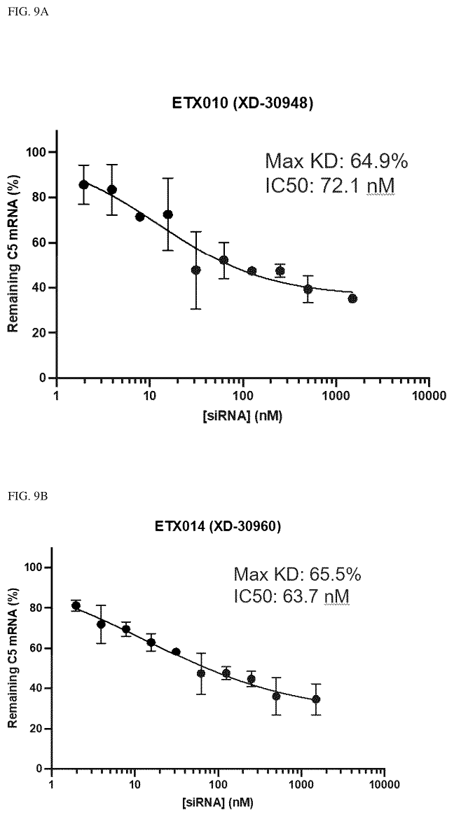

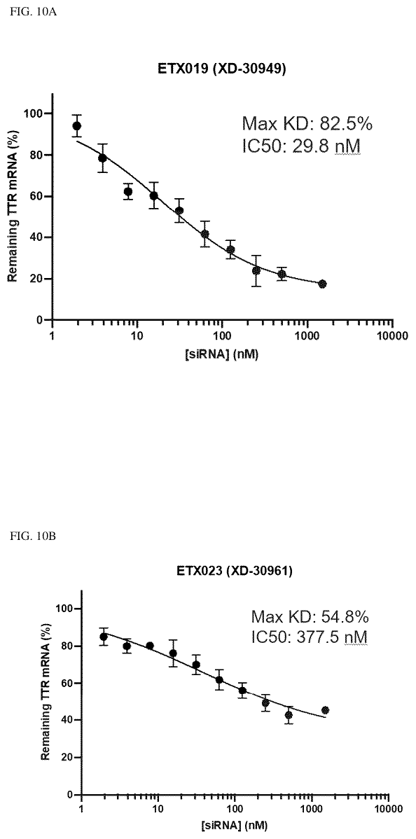

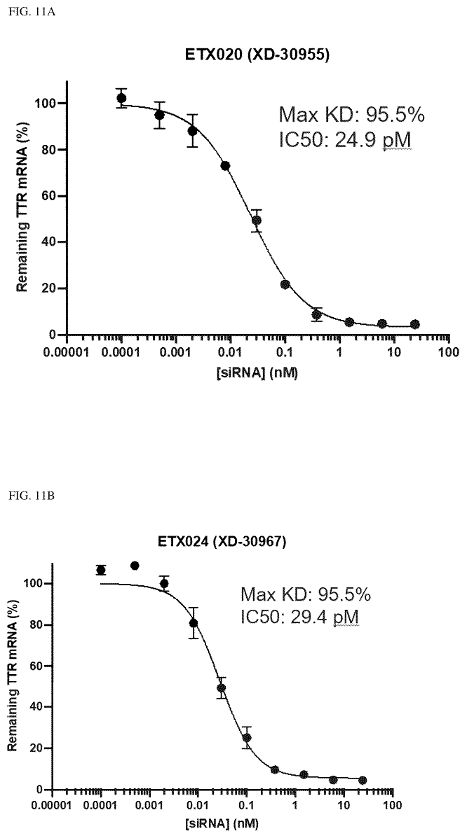

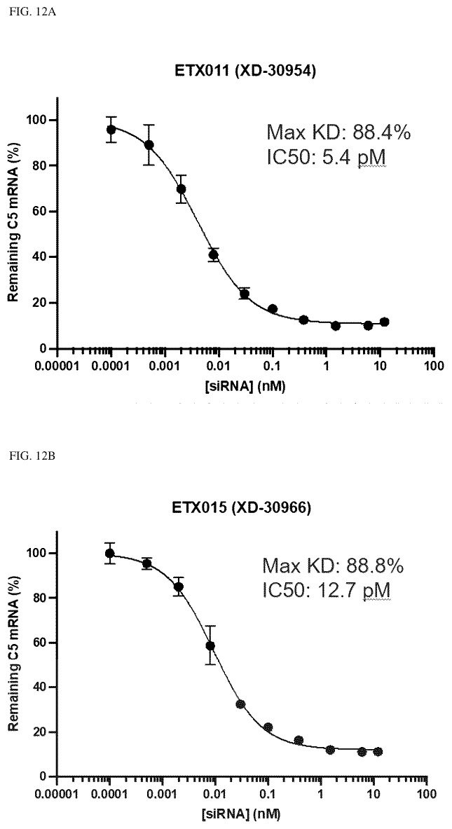

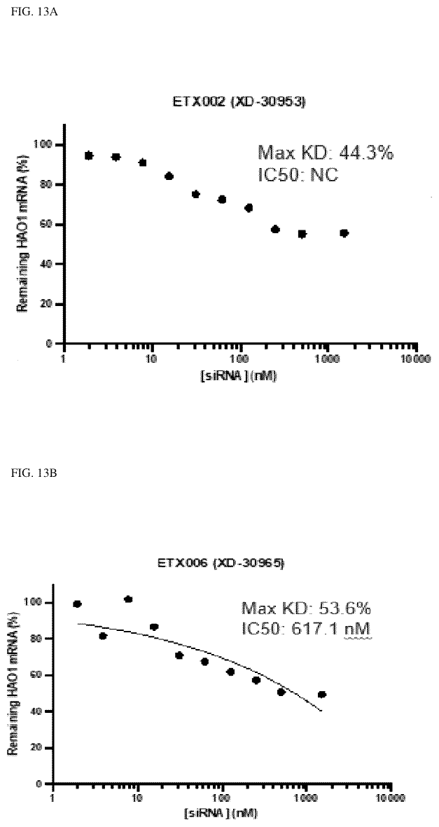

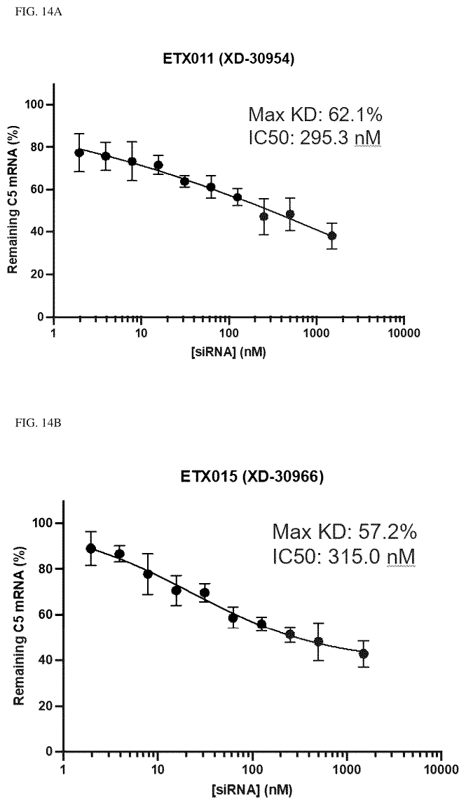

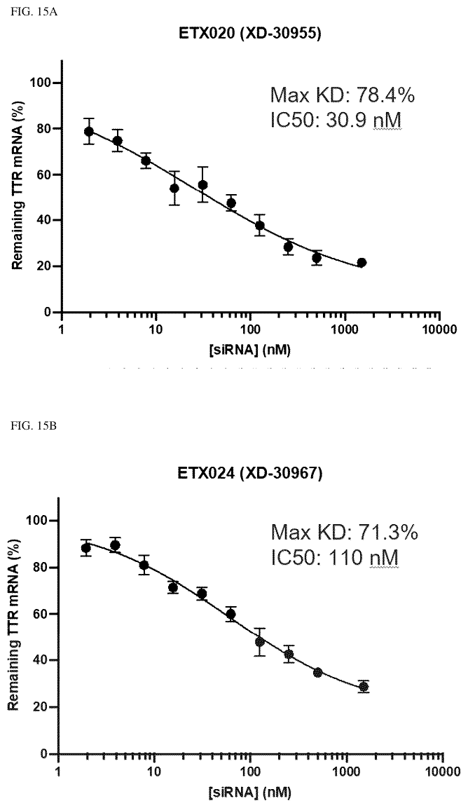

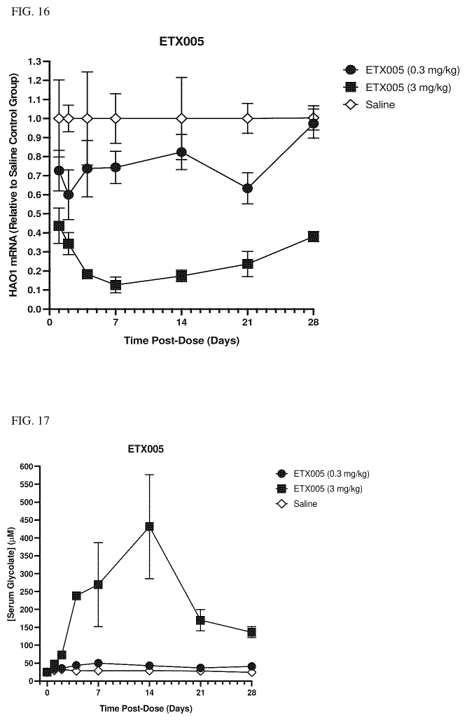

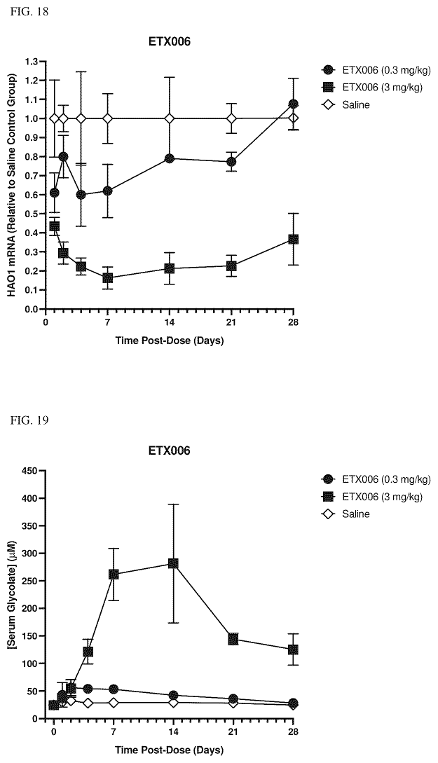

Results for the 72 h incubation are also shown in FIGS. 9A-9B.

No significant on-target silencing of GalNAc-siRNAs is visible after 4 h incubation. Data generated after an incubation period of 72 h showed a more robust on-target silencing of up to 65.5% maximal inhibition.

Dose-Response Analysis of hsTTR Targeting GalNAc-siRNAs in PHHs

The last target of interest, hsTTR mRNA, was again tested in a gymnotic, free uptake in PHHs in an identical dose-response setup as for the targets hsHAO1 and hsC5, with the only difference being that specific siRNA sequences for the on-target hsTTR mRNA was used (see Table 1).

The experiment ended after 72 h of direct incubation of PHHs. Table 7 lists activity data for all hsTTR targeting GalNAc-siRNAs studied.

TABLE 7
Target, incubation time, external ID, IC20/IC50/IC80
values and maximal inhibition of hsTTR targeting
GalNAc-siRNAs in primary human hepatocytes (PHHs).
The listing is organized according to external ID.

| | Incubation | External | IC20 | IC50 | IC80 | Max. Inhib. |
|---|---|---|---|---|---|---|
| Target | [h] | ID | [nM] | [nM] | [nM] | [%] |
| hsTTR | 72 | ETX019 | 3.9 | 29.8 | 1536.8 | 82.5 |
| hsTTR | 72 | ETX023 | 6.7 | 377.5 | #N/A | 54.8 |

Results are also shown in FIGS. 10A-10B.

Gymnotic, free uptake of GalNAc-siRNAs targeting hsTTR did lead to significant on-target silencing within 72 h, ranging between 46 to 82.5% maximal inhibition. hsTTR GalNAc-siRNAs were identified that silence the on-target mRNA with IC50 values in the low double-digit nM range.

Conclusions and Discussion

The scope of this study was to analyze the in vitro activity of GalNAc-ligands according to the present invention when used in the context of siRNAs targeting three different on-targets, namely hsHAO1, hsC5 and hsTTR mRNA. siRNA sets specific for each target were composed of siRNAs with different linker/cap/modification/GalNAc-ligand chemistries in the context of two different antisense strands each.

For all targets, GalNAc-siRNAs from Table 1 were identified that showed a high overall potency and low IC50 value.

### 1.1 Example 2

### Routes of Synthesis

i) Synthesis of the Conjugate Building Blocks TriGalNAc

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfar Silica 10, 25, 50 or 100 g columns (Uppsala, Sweden).

All moisture-sensitive reactions were carried out under anhydrous conditions using dry glassware, anhydrous solvents and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (¹H NMR) and 125 MHz (¹³C NMR). Chemical shifts are given in ppm referenced to the solvent residual peak (CDCl₃—¹H NMR: 6 at 7.26 ppm and ¹³C NMR δ at 77.2 ppm; DMSO-d₆—1H NMR: 6 at 2.50 ppm and ¹³C NMR δ at 39.5 ppm). Coupling constants are given in Hertz. Signal splitting patterns are described as singlet (s), doublet (d), triplet (t) or multiplet (m).

ii) Synthesis Route for the Conjugate Building Block TriGalNAc

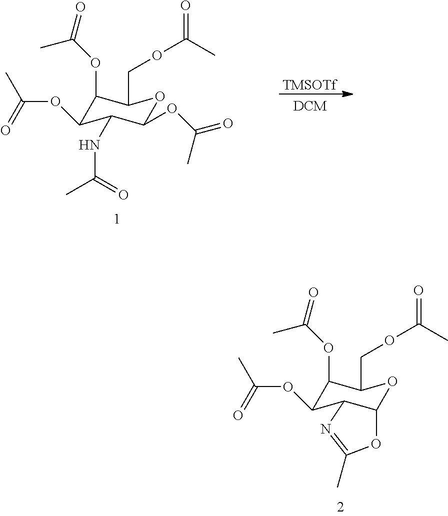 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃(100 mL) and water (100 mL). The organic layer was separated, dried over Na2SO4 and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

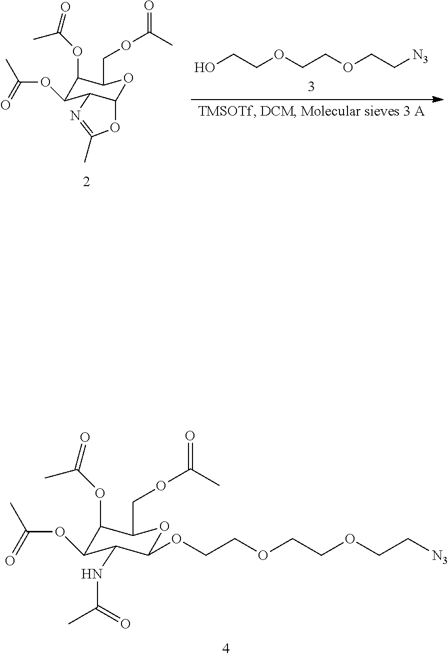 [SMILES: CC(=O)OCC1OC2OC(C)=NC2C(OC(C)=O)C1OC(C)=O ; [N-]=[N+]=NCCOCCOCCO ; CC(=O)NC1C(OCCOCCOCCN=[N+]=[N-])OC(COC(C)=O)C(OC(C)=O)C1OC(C)=O]

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) was added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6 (C), 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH₃).

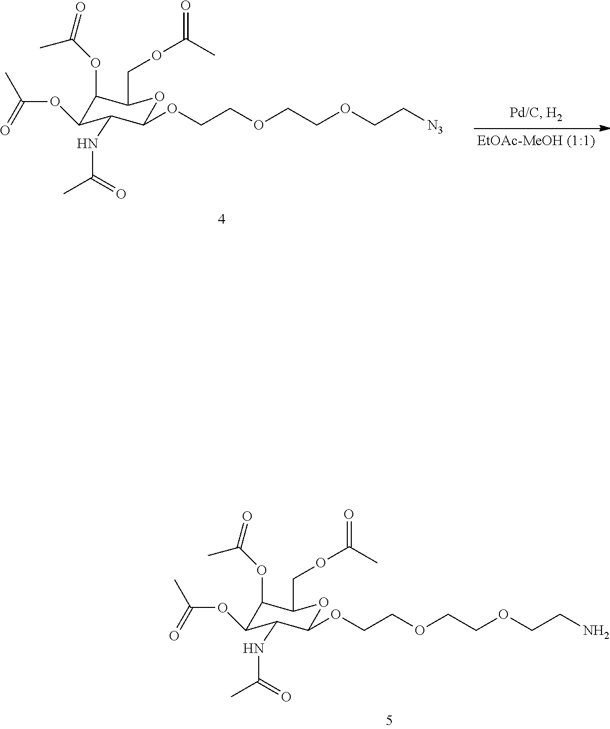 [SMILES: CC(=O)NC1C(OCCOCCOCCN=[N+]=[N-])OC(COC(C)=O)C(OC(C)=O)C1OC(C)=O ; CC(=O)NC1C(OCCOCCOCCN)OC(COC(C)=O)C(OC(C)=O)C1OC(C)=O]

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

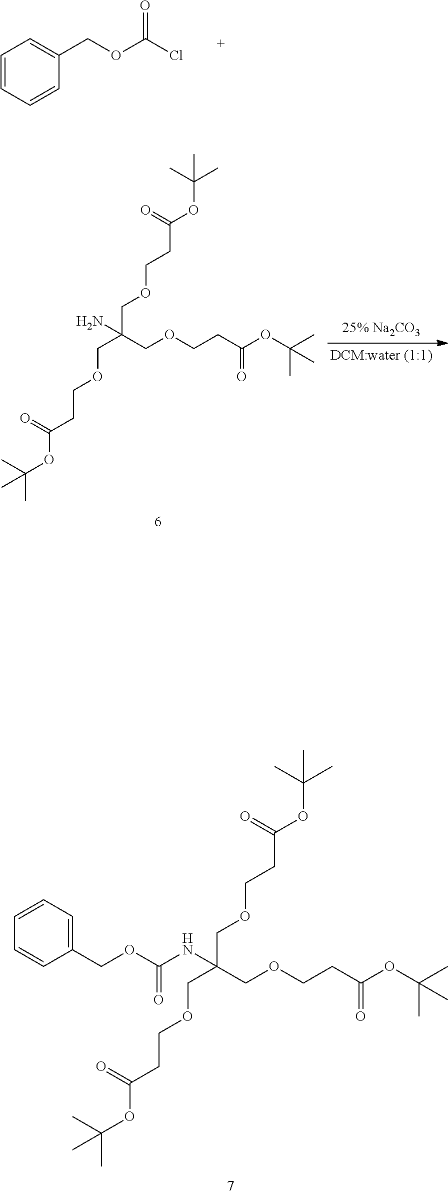 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH₂), 66.8 (3×CH₂), 64.9 (C), 58.7 (CH₂), 35.8 (3×CH₂), 27.7 (9×CH₃).

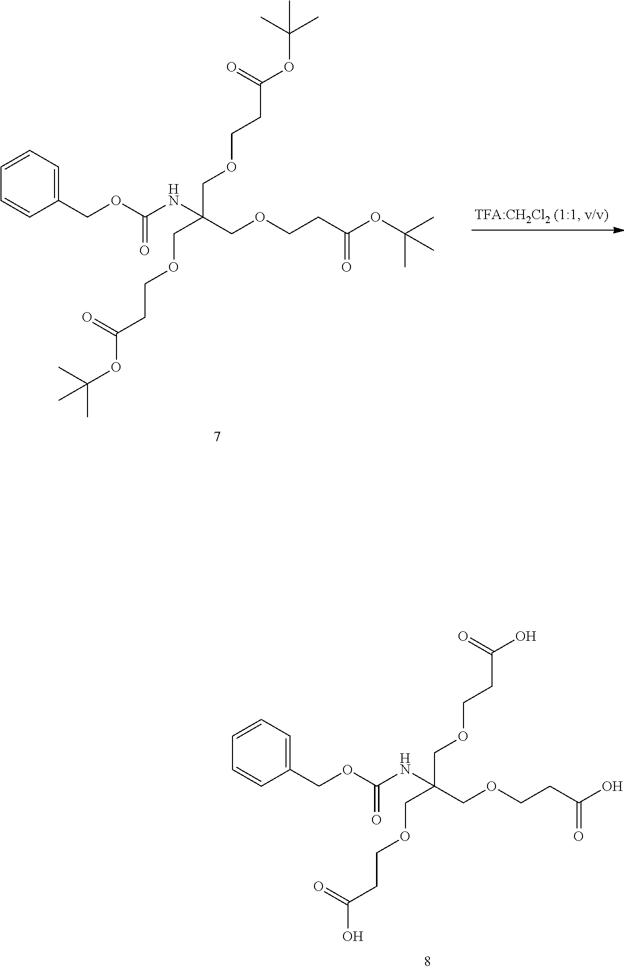 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

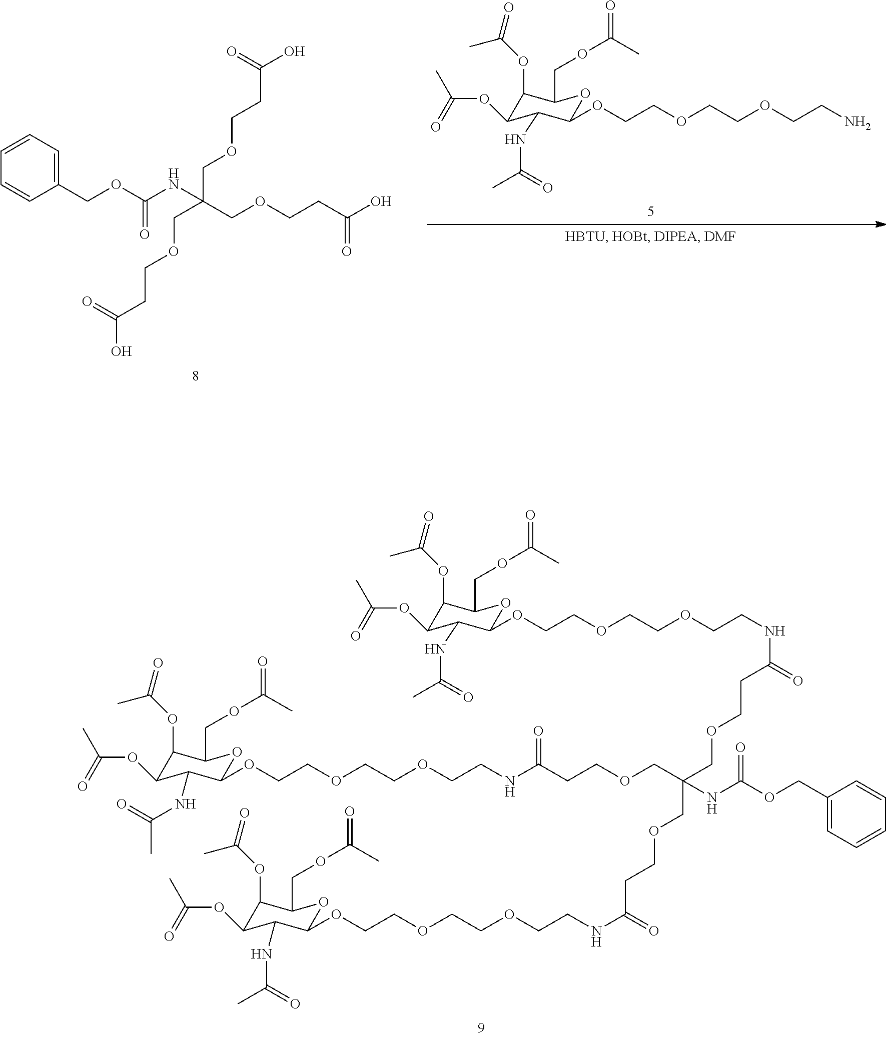 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uronium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH₂), 69.0 (CH₂), 68.2 (CH₂), 67.2 (CH₂), 66.7 (CH₂), 61.4 (CH₂), 22.6 (CH₂), 22.4 (3×CH₃), 20.7 (9×CH₃).

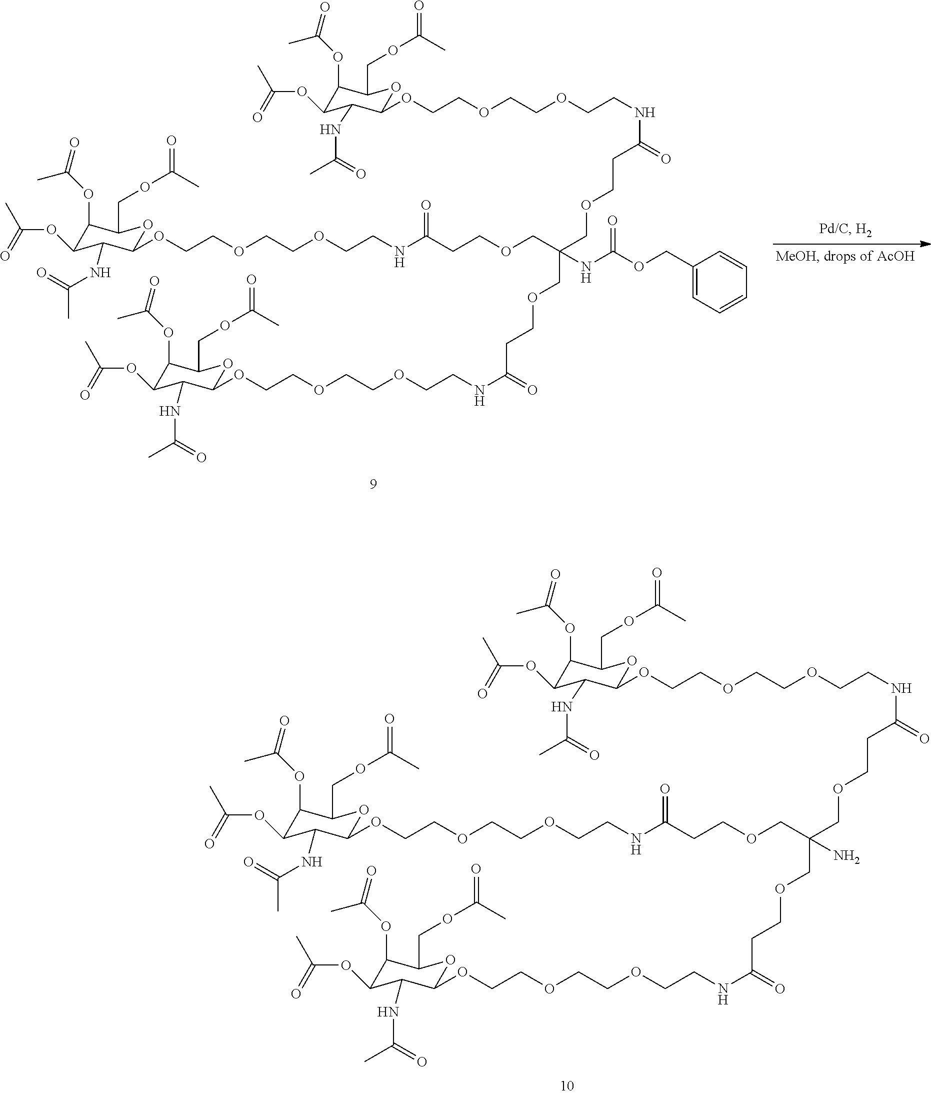 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

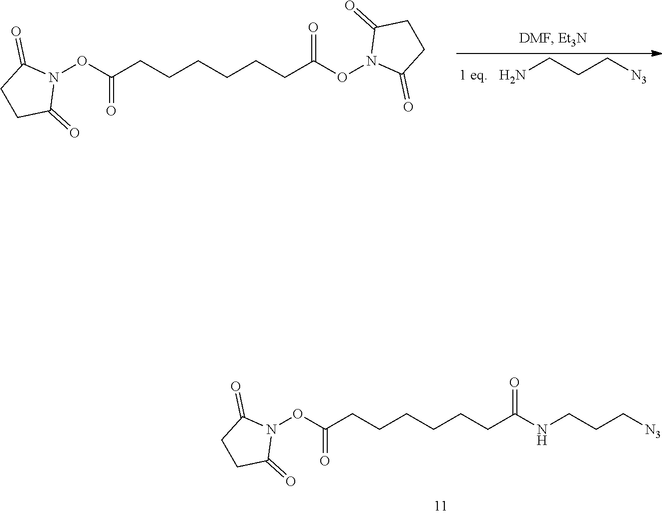 [SMILES: O=C(CCCCCCC(=O)ON1C(=O)CCC1=O)ON1C(=O)CCC1=O ; [N-]=[N+]=NCCCN ; [N-]=[N+]=NCCCNC(=O)CCCCCCC(=O)ON1C(=O)CCC1=O]

Preparation of compound 11: Commercially available suberic acid bis(N-hydroxysuccinimide ester) (3.67 g, 9.9 mmol, 1.0 eq.) was dissolved in DMF (5 mL) and triethylamine (1.2 mL) was added. To this solution was added dropwise a solution of 3-azido-1-propylamine (1.0 g, 9.9 mmol, 1.0 eq.) in DMF (5 mL). The reaction was stirred at room temperature for 3 h. The reaction mixture was diluted with EtOAc (100 mL) and washed with water (50 mL). The organic layer was separated, dried over Na₂SO₄ and the solvent was removed under reduced pressure. The crude material was purified by flash chromatography (gradient elution: 0-5% MeOH in DCM in 16 CV). The product was obtained as white solid (1.54 g, 43%, rf=0.71 (5% MeOH in DCM)). MS: calculated for C₁₅H₂₃N₅O₅, 353.4. Found 354.3.

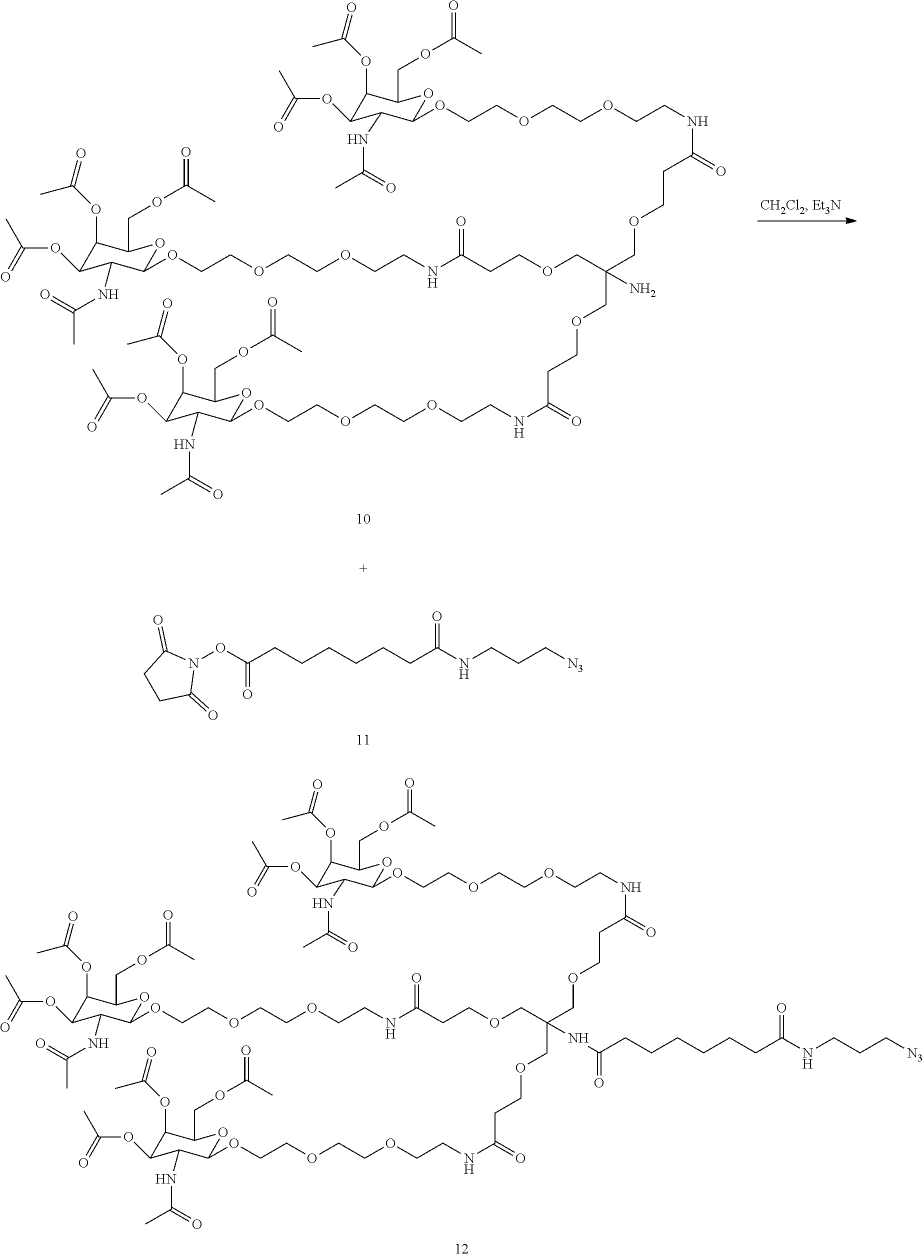 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; [N-]=[N+]=NCCCNC(=O)CCCCCCC(=O)ON1C(=O)CCC1=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)NCCCN=[N+]=[N-])OC(COC(C)=O)C(OC(C)=O)C1OC(C)=O]

Preparation of TriGalNAc (12): Triantennary GalNAc compound 10 (0.35 g, 0.24 mmol, 1.0 eq.) and compound 11 (0.11 g, 0.31 mmol, 1.5 eq.) were dissolved in DCM (5 mL) under argon and triethylamine (0.1 mL, 0.61 mmol, 3.0 eq.) was added. The reaction was stirred at room temperature overnight. The solvent was removed under reduced pressure, the residue was dissolved in EtOAc (100 mL) and washed with water (100 mL). The organic layer was separated and dried over Na₂SO₄. The solvent was evaporated and the resulting crude material was purified by flash chromatography (elution gradient: 0-10% MeOH in DCM in 20 CV) to afford the title compound as white fluffy solid (0.27 g, 67%, rf=0.5 (10% MeOH in DCM)). MS: calculated for C₈₄H₁₃₇N₁₁O₄₁, 1957.1. Found 1959.6.

Compound 12 was used for subsequent oligonucleotide conjugate preparations employing “click chemistry”.

iii) Oligonucleotide Synthesis

TABLE 8

| Single | | Purity |
|---|---|---|
| strand | | by RP |
| | | HPLC |
| ID | Sequence 5′-3′ | (%) |
| X91382 | (NH2-DEG)gacuuuCfaUfCfCfuggaaauasusa | 89.5 |
| | (invabasic)(invabasic) | |
| X91383 | (NH2-DEG)aaGfcAfaGfaUfAfUfuUfuuAfuAf | 91.6 |
| | asusa(invabasic)(invabasic) | |
| X91384 | (NH2-DEG)ugggauUfuCfAfUfguaaccaasgsa | 94.0 |
| | (invabasic)(invabasic) | |
| X91403 | (NH2C12)gacuuuCfaUfCfCfuggaaauasusa | 94.2 |
| | (invabasic)(invabasic) | |
| X91404 | (NH2C12)aaGfcAfaGfaUfAfUfuUfuuAfuAfa | 96.5 |
| | susa(invabasic)(invabasic) | |
| X91405 | (NH2C12)ugggauUfuCfAfUfguaaccaasgsa | 91.3 |
| | (invabasic)(invabasic) | |
| X91415 | (invabasic)(invabasic)gsascuuuCfaUfC | 96.4 |
| | fCfuggaaauasusa(NH2C6) | |
| X91416 | (invabasic)(invabasic)asasGfcAfaGfaU | 77.4 |
| | fAfUfuUfuuAfuAfaua(NH2C6) | |
| X91417 | (invabasic)(invabasic)usgsggauUfuCfA | 96.7 |
| | fUfguaaccaaga(NH2C6) | |
| X91379 | gsascuuuCfaUfCfCfuggaaauaua(GalNAc) | 92.8 |
| X91380 | asasGfcAfaGfaUfAfUfuUfuuAfuAfaua | 95.7 |
| | (GalNAc) | |
| X91446 | usgsggauUfuCfAfUfguaaccaaga(GalNAc) | 92.1 |
| X38483 | usAfsuauUfuCfCfaggaUfgAfaagucscsa | 91.0 |
| X91381 | us AfsUfuAfuaAfaAfauaUfcUfuGfcuususu | 90.0 |
| | dTdT | |
| X38104 | usCfsuugGfuuAfcaugAfaAfucccasusc | 95.4 |

Af, Cf, Gf, Uf: 2′-F RNA nucleotides
a, c, g, u: 2′-O-Me RNA nucleotides
dT: DNA nucleotides
s: Phosphorothioate
invabasic: 1,2-dideoxyribose
NH2-DEG: Aminoethoxyethyl linker
NH2C12: Aminododecyl linker
NH2C6: Aminohexyl linker

Oligonucleotides were synthesized on solid phase according to the phosphoramidite approach. Depending on the scale either a Mermade 12 (BioAutomation Corporation) or an ÄKTA Oligopilot (GE Healthcare) was used.

Syntheses were performed on commercially available solid supports made of controlled pore glass either loaded with invabasic (CPG, 480 Å, with a loading of 86 μmol/g; LGC Biosearch cat. #BCG-1047-B) or 2′-F A (CPG, 520 Å, with a loading of 90 μmol/g; LGC Biosearch cat. #BCG-1039-B) or NH2C6 (CPG, 520 Å, with a loading of 85 μmol/g LGC Biosearch cat. #BCG-1397-B) or GalNAc (CPG, 500 Å, with a loading of 57 μmol/g; Primetech) or 2′-O-Methyl C (CPG, 500 Å, with a loading of 84 μmol/g LGC Biosearch cat. #BCG-10-B) or 2′-O-Methyl A (CPG, 497 Å, with a loading of 85 μmol/g, LGC Biosearch, Cat. #BCG-1029-B) or dT (CPG, 497 Å, with a loading of 87 μmol/g LGC Biosearch, cat. #BCG-1055-B).

2′-O-Me, 2′-F RNA phosphoramidites and ancillary reagents were purchased from SAFC Proligo (Hamburg, Germany).

2-O-Methyl phosphoramidites include: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-benzoyl-cytidine 2′-O-methyl-3′-[(2-cyanoethyl)-(N,N-diisopropyl)]-phosphoramidite, 5′-(4,4′-dimethoxytrityl)-N-dimethylformamidine-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

2′-F phosphoramidites include: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

In order to introduce the required amino linkers at the 5′-end of the oligonucleotides the 2-[2-(4-Monomethoxytrityl)aminoethoxy]ethyl-(2-cyanoethyl)-N,N-diisopropyl)-phosphoramidite (Glen Research Cat. #1905) and the 12-(trifluoroacetylamino)dodecyl-[(2-cyanoethyl)-(N,N-diisopropyl)]-phosphoramidite (ChemGenes Cat. #CLP-1575) were employed. The invabasic modification was introduced using 5-O-dimethoxytrityl-1,2-dideoxyribose-3-[(2-cyanoethyl)-(N,N-diisopropyl)]-phosphoramidite (ChemGenes Cat. #ANP-1422).

All building blocks were dissolved in anhydrous acetonitrile (100 mM (Mermade12) or 200 mM (ÄKTA Oligopilot)) containing molecular sieves (3 Å) except 2′-O-methyl-uridine phosphoramidite which was dissolved in 50% anhydrous DCM in anhydrous acetonitrile. Iodine (50 mM in pyridine/H₂O 9:1 v/v) was used as oxidizing reagent. 5-Ethyl thiotetrazole (ETT, 500 mM in acetonitrile) was used as activator solution.

Thiolation for introduction of phosphorthioate linkages was carried out using 100 mM xanthane hydride (TCI, Cat. #6846-35-1) in acetonitrile/pyridine 4:6 v/v.

Coupling times were 5.4 minutes except when stated otherwise. 5′ amino modifications were incorporated into the sequence employing a double coupling step with a coupling time of 11 minutes per each coupling (total coupling time 22 min). The oxidizer contact time was set to 1.2 min and thiolation time was 5.2 min.

Sequences were synthesized with removal of the final DMT group, with exception of the MMT group from the NH2DEG sequences.

At the end of the synthesis, the oligonucleotides were cleaved from the solid support using a 1:1 volume solution of 28-30% ammonium hydroxide (Sigma-Aldrich, Cat. #221228) and 40% aqueous methylamine (Sigma-Aldrich, Cat. #8220911000) for 16 hours at 6° C. The solid support was then filtered off, the filter was thoroughly washed with H₂O and the volume of the combined solution was reduced by evaporation under reduced pressure. The pH of the resulting solution was adjusted to pH 7 with 10% AcOH (Sigma-Aldrich, Cat. #A6283).

The crude materials were purified either by reversed phase (RP) HPLC or anion exchange (AEX) HPLC.

RP HPLC purification was performed using a XBridge C18 Prep 19×50 mm column (Waters) on an ÄKTA Pure instrument (GE Healthcare). Buffer A was 100 mM triethyl-ammonium acetate (TEAAc, Biosolve) pH 7 and buffer B contained 95% acetonitrile in buffer A. A flow rate of 10 mL/min and a temperature of 60° C. were employed. UV traces at 280 nm were recorded. A gradient of 0% B to 100% B within 120 column volumes was employed. Appropriate fractions were pooled and precipitated in the freezer with 3 M sodium acetate (NaOAc) (Sigma-Aldrich), pH 5.2 and 85% ethanol (VWR). Pellets were isolated by centrifugation, redissolved in water (50 mL), treated with 5 M NaCl (5 mL) and desalted by Size exclusion HPLC on an Äkta Pure instrument using a 50×165 mm ECO column (YMC, Dinslaken, Germany) filled with Sephadex G25-Fine resin (GE Healthcare).

AEX HPLC purification was performed using a TSK gel SuperQ-5PW 20×200 mm (BISCHOFF Chromatography) on an ÄKTA Pure instrument (GE Healthcare). Buffer A was 20 mM sodium phosphate (Sigma-Aldrich) pH 7.8 and buffer B was the same as buffer A with the addition of 1.4 M sodium bromide (Sigma-Aldrich). A flow rate of 10 mL/min and a temperature of 60° C. were employed. UV traces at 280 nm were recorded. A gradient of 10% B to 100% B within 27 column volumes was employed. Appropriate fractions were pooled and precipitated in the freezer with 3 M NaOAc, pH 5.2 and 85% ethanol. Pellets were isolated by centrifugation, redissolved in water (50 mL), treated with 5 M NaCl (5 mL) and desalted by size exclusion chromatography.

The MMT group was removed with 25% acetic acid in water. Once the reaction was complete the solution was neutralized and the samples were desalted by size exclusion chromatography.

Single strands were analyzed by analytical LC-MS on a 2.1×50 mm XBridge C18 column (Waters) on a Dionex Ultimate 3000 (Thermo Fisher Scientific) HPLC system combined either with a LCQ Deca XP-plus Q-ESI-TOF mass spectrometer (Thermo Finnigan) or with a Compact ESI-Qq-TOF mass spectrometer (Bruker Daltonics). Buffer A was 16.3 mM triethylamine, 100 mM 1,1,1,3,3,3-hexafluoro-2-propanol (HFIP) and 1% MeOH in H₂O and buffer B contained buffer A in 95% MeOH. A flow rate of 250 μL/min and a temperature of 60° C. were employed. UV traces at 260 and 280 nm were recorded. A gradient of 1-40% B within 0.5 min followed by 40 to 100% B within 13 min was employed. Methanol (LC-MS grade), water (LC-MS grade), 1,1,1,3,3,3-hexafluoro-2-propanol (puriss. p.a.) and triethylamine (puriss. p.a.) were purchased from Sigma-Aldrich.

iv) Monofluoro cyclooctyne (MFCO) Conjugation at 5′- or 3′-End

### 5′-End MFCO Conjugation

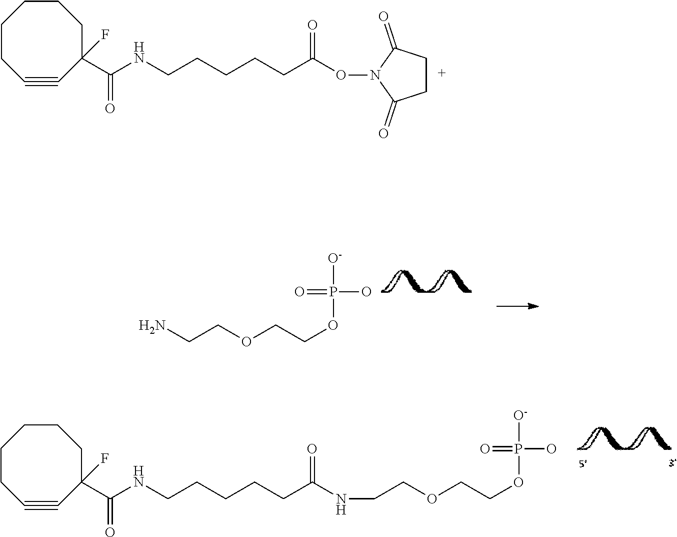 [SMILES: O=C(CCCCCNC(=O)C1(F)C#CCCCCC1)ON1C(=O)CCC1=O ; NCCOCCOP(=O)([O-])O ; O=C(CCCCCNC(=O)C1(F)C#CCCCCC1)NCCOCCOP(=O)([O-])O]

### 3-End MFCO Conjugation

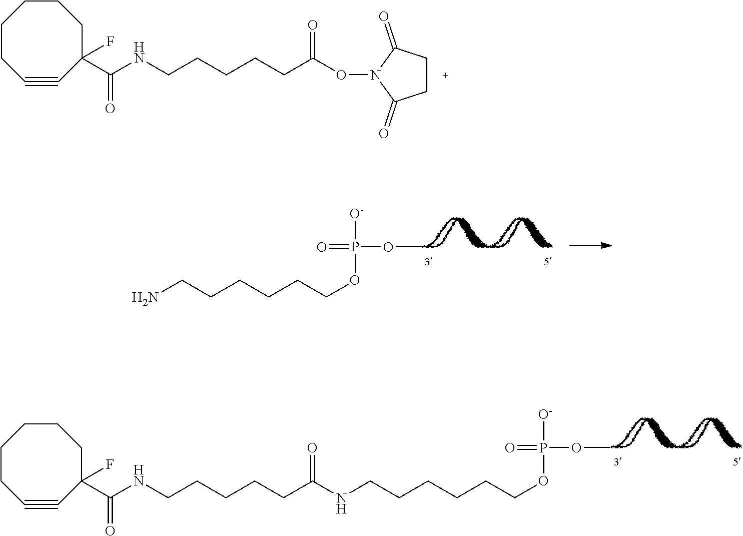 [SMILES: O=C(CCCCCNC(=O)C1(F)C#CCCCCC1)ON1C(=O)CCC1=O ; COP(=O)([O-])OCCCCCCN ; COP(=O)([O-])OCCCCCCNC(=O)CCCCCNC(=O)C1(F)C#CCCCCC1]

General conditions for MFCO conjugation: Amine-modified single strand was dissolved at 700 OD/mL in 50 mM carbonate/bicarbonate buffer pH 9.6/dimethyl sulfoxide (DMSO) 4:6 (v/v) and to this solution was added one molar equivalent of a 35 mM solution of MFCO-C6-NHS ester (Berry&Associates, Cat. #LK 4300) in DMF. The reaction was carried out at room temperature and after 1 h another molar equivalent of the MFCO solution was added. The reaction was allowed to proceed for an additional hour and was monitored by LC/MS. At least two molar equivalent excess of the MFCO NHS ester reagent relative to the amino modified oligonucleotide were needed to achieve quantitative consumption of the starting material. The reaction mixture was diluted 15-fold with water, filtered through a 1.2 μm filter from Sartorius and then purified by reserve phase (RP HPLC) on an Äkta Pure instrument (GE Healthcare).

Purification was performed using a XBridge C18 Prep 19×50 mm column from Waters. Buffer A was 100 mM TEAAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full length conjugated oligonucleotide were pooled, precipitated in the freezer with 3 M NaOAc, pH 5.2 and 85% ethanol and the collected pellet was dissolved in water. Samples were desalted by size exclusion chromatography and concentrated using a speed-vac concentrator to yield the conjugated oligonucleotide in an isolated yield of 40-80%.

TABLE 9

| Sense | | Purity |
|---|---|---|
| strand | | by RP |
| ID | Sense strand sequence 5′-3′ | HPLC (%) |
| X91388 | (MFCO)(NH-DEG)gacuuuCfaUfCfCfugg | 89.0 |
| | aaauasusa(invabasic)(invabasic) | |
| X91389 | (MFCO)(NH-DEG)aaGfcAfaGfaUfAfUfu | 91.0 |
| | UfuuAfuAfasusa(invabasic) | |
| | (invabasic) | |
| X91390 | (MFCO)(NH-DEG)ugggauUfuCfAfUfgua | 90.0 |
| | accaasgsa(invabasic)(invabasic) | |
| X91421 | (invabasic)(invabasic)gsascuuuCf | 94.0 |
| | aUfCfCfuggaaauasusa(NHC6)(MFCO) | |
| X91422 | (invabasic)(invabasic)asasGfcAfaG | 89.0 |
| | faUfAfUfuUfuuAfuAfaua(NHC6)(MFCO) | |
| X91423 | (invabasic)(invabasic)usgsggauUf | 89.0 |
| | uCfAfUfguaaccaaga(NHC6)(MFCO) | |

v) TriGalNAc (GalNAc-T1) Conjugation at 5′- or 3′-End

5′-GalNAc-T1 Conjugates

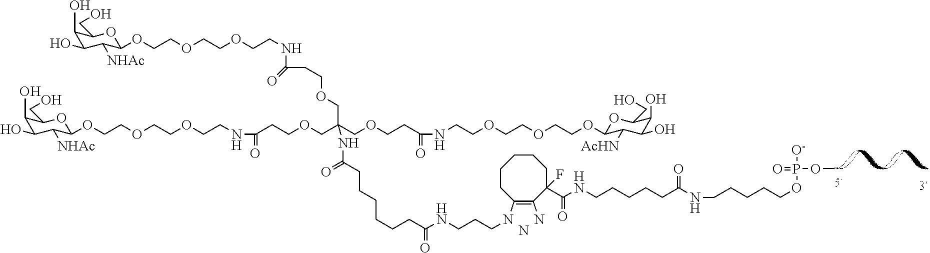

3′-GalNAc-T1 Conjugates

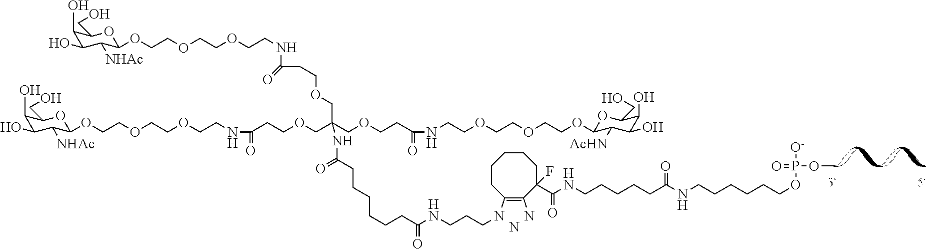

General procedure for TriGalNAc conjugation: MFCO-modified single strand was dissolved at 2000 OD/mL in water and to this solution was added one equivalent solution of compound 12 (10 mM) in DMF. The reaction was carried out at room temperature and after 3 h 0.7 molar equivalent of the compound 12 solution was added. The reaction was allowed to proceed overnight and completion was monitored by LCMS. The conjugate was diluted 15-fold in water, filtered through a 1.2 μm filter from Sartorius and then purified by RP HPLC on an Äkta Pure instrument (GE Healthcare).

RP HPLC purification was performed using a XBridge C18 Prep 19×50 mm column from Waters. Buffer A was 100 mM triethylammonium acetate pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleotide were pooled, precipitated in the freezer with 3 M NaOAc, pH 5.2 and 85% ethanol and the collected pellet was dissolved in water to give an oligonucleotide solution of about 1000 OD/mL. The O-acetates were removed by adding 20% aqueous ammonia. Quantitative removal of these protecting groups was verified by LC-MS.

The conjugates were desalted by size exclusion chromatography using Sephadex G25 Fine resin (GE Healthcare) on an Äkta Pure (GE Healthcare) instrument to yield the conjugated nucleotide in an isolated yield of 50-70%.

TABLE 10

| | | Purity |
|---|---|---|
| Sense | | by RP |
| strand | | HPLC |
| ID | Sense strand sequence 5′-3′ | (%) |
| X91394 | (GalNAc-T1)(MFCO)(NH-DEG)gacuuuC | 80.0 |
| | faUfCfCfuggaaauasusa(invabasic) | |
| | (invabasic) | |
| X91395 | (GalNAc-T1)(MFCO)(NH-DEG)aaGfcAf | 87.8 |
| | aGfaUfAfUfuUfuuAfuAfasusa | |
| | (invabasic)(invabasic) | |
| X91396 | (GalNAc-T1)(MFCO)(NH-DEG)ugggauU | 87.9 |
| | fuCfAfUfguaaccaasgsa(invabasic) | |
| | (invabasic) | |
| X91427 | (invabasic)(invabasic)gsascuuuCf | 88.0 |
| | aUfCfCfuggaaauasusa(NHC6)(MFCO) | |
| | (GalNAc-T1) | |
| X91428 | (invabasic)(invabasic)asasGfcAfa | 82.6 |
| | GfaUfAfUfuUfuuAfuAfaua(NHC6) | |
| | (MFCO)(GalNAc-T1) | |
| X91429 | (invabasic)(invabasic)usgsggauUf | 82.9 |
| | uCfAfUfguaaccaaga(NHC6)(MFCO) | |
| | (GalNAc-T1) | |

vi) Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

GalNAc conjugates prepared are compiled in the table below. These were directed against 3 different target genes. siRNA coding along with the corresponding single strands, sequence information as well as purity for the duplexes is captured.

TABLE 11

| | | | | Duplex |
|---|---|---|---|---|
| | | | | Purity |
| | Duplex | SSRN | | by HPLC |
| Target | ID | ID | ssRNA-Sequence 5′-3′ | (%) |
| GO | ETX001 | X91394 | (GalNAc-T1)(MFCO)(NHDEG) | 96.8 |
| | | | gacuuuCfaUfCfCfuggaaauasusa | |
| | | | (invabasic)(invabasic) | |
| | | X38483 | usAfsuauUfuCfCfaggaUfgAfaag | |
| | | | ucscsa | |
| | ETX005 | X91427 | (invabasic)(invabasic)gsasc | 92.8 |
| | | | uuuCfaUfCfCfuggaaauasusa | |
| | | | (NHC6)(MFCO)(GalNAc-T1) | |
| | | X38483 | usAfsuauUfuCfCfaggaUfgAfaag | |
| | | | ucscsa | |
| C5 | ETX010 | X91395 | (GalNAc-T1)(MFCO)(NH-DEG) | 96.4 |
| | | | aaGfcAfaGfaUfAfUfuUfuuAfuAf | |
| | | | asusa(invabasic)(invabasic) | |
| | | X91381 | usAfsUfuAfuaAfaAfauaUfcUfuG | |
| | | | fcuususudTdT | |
| | ETX014 | X91428 | (invabasic)(invabasic)asasG | 97.2 |
| | | | fcAfaGfaUfAfUfuUfuuAfuAfaua | |
| | | | (NHC6)(MFCO)(GalNAc-T1) | |
| | | X91381 | usAfsUfuAfuaAfaAfauaUfcUfuG | |
| | | | fcuususudTdT | |
| TTR | ETX019 | X91396 | (GalNAc-T1)(MFCO)(NH-DEG) | 97.2 |
| | | | ugggauUfuCfAfUfguaaccaasgsa | |
| | | | (invabasic)(invabasic) | |
| | | X38104 | usCfsuugGfuuAfcaugAfaAfuccc | |
| | | | asusc | |
| | ETX023 | X91429 | (invabasic)(invabasic)usgsg | 96.3 |
| | | | gauUfuCfAfUfguaaccaaga | |
| | | | (NHC6)(MFCO)(GalNAc-T1) | |
| | | X38104 | usCfsuugGfuuAfcaugAfaAfuccc | |
| | | | asusc | |

The following schemes further set out the routes of synthesis:

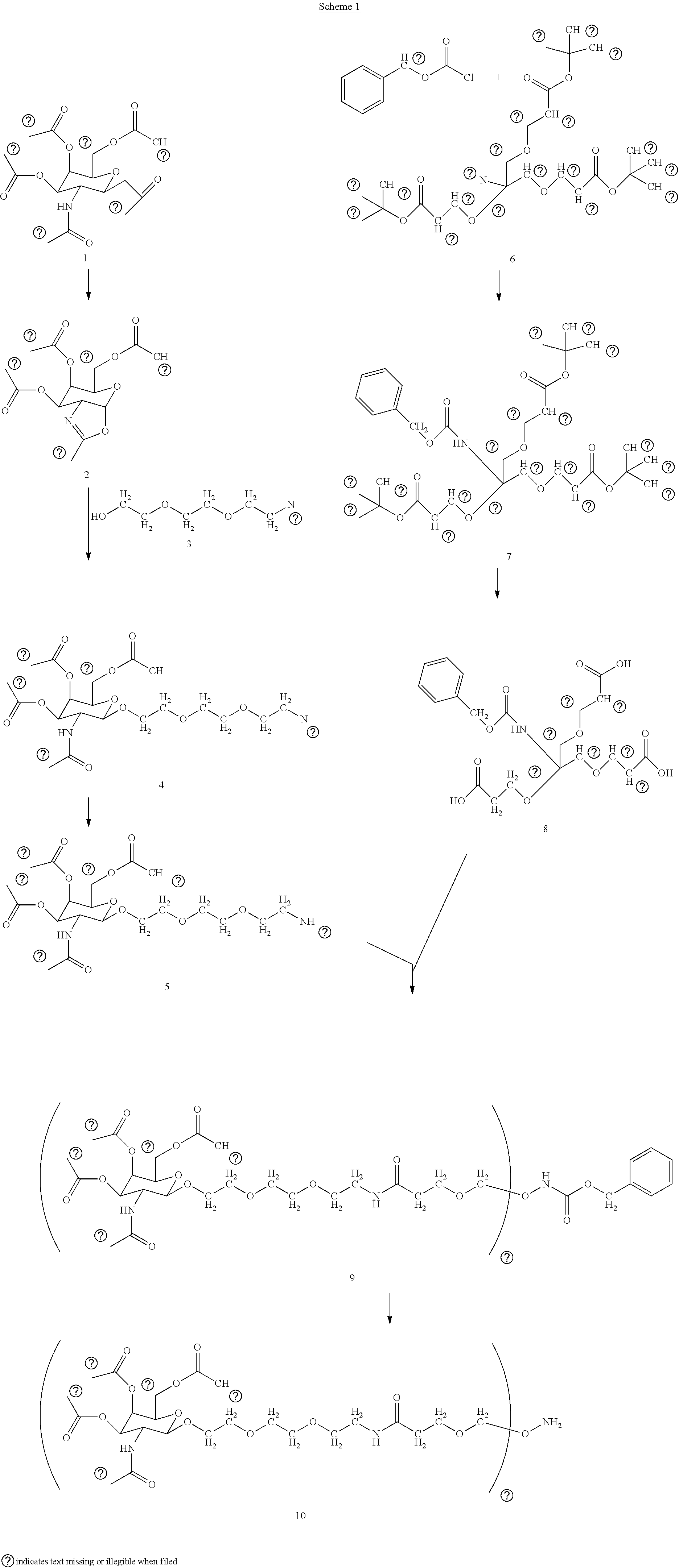

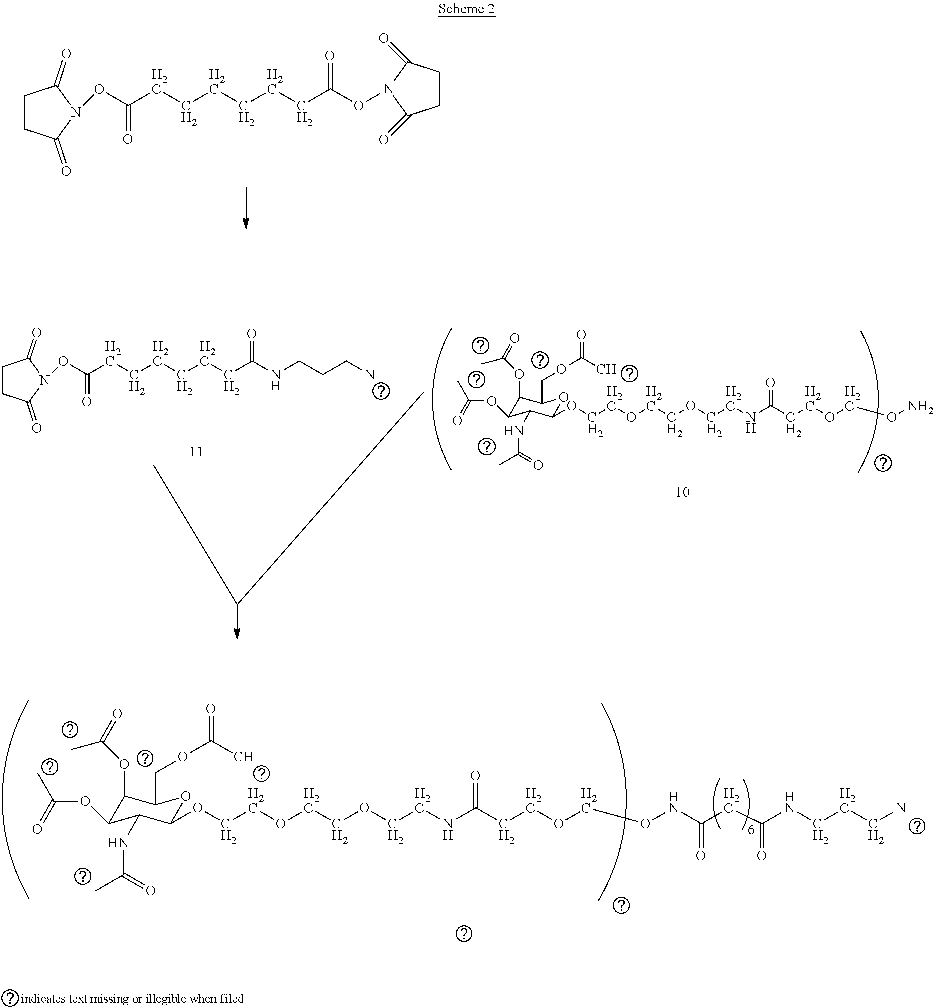

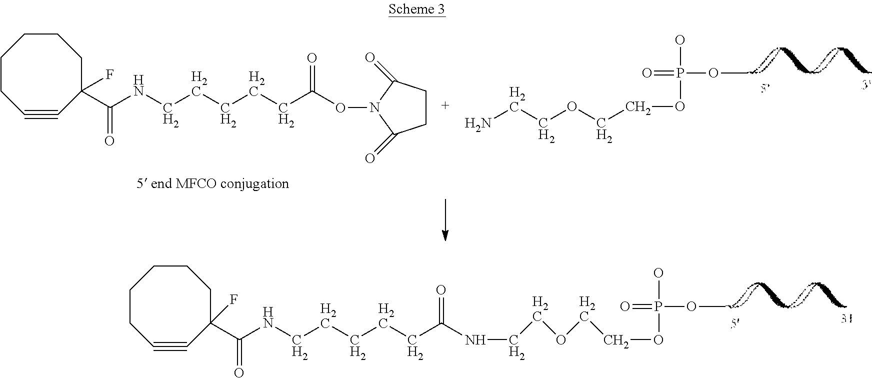 [SMILES: O=C(CCCCCNC(=O)C1(F)C#CCCCCC1)ON1C(=O)CCC1=O ; COP(=O)(O)OCCOCCN ; COP(=O)(O)OCCOCCNC(=O)CCCCCNC(=O)C1(F)C#CCCCCC1]

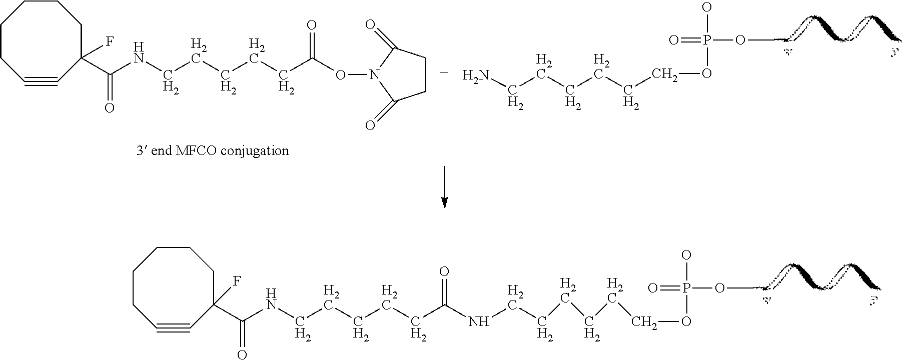 [SMILES: O=C(CCCCCNC(=O)C1(F)C#CCCCCC1)ON1C(=O)CCC1=O ; COP(=O)(O)OCCCCCCN ; COP(=O)(O)OCCCCCCNC(=O)CCCCCNC(=O)C1(F)C#CCCCCC1]

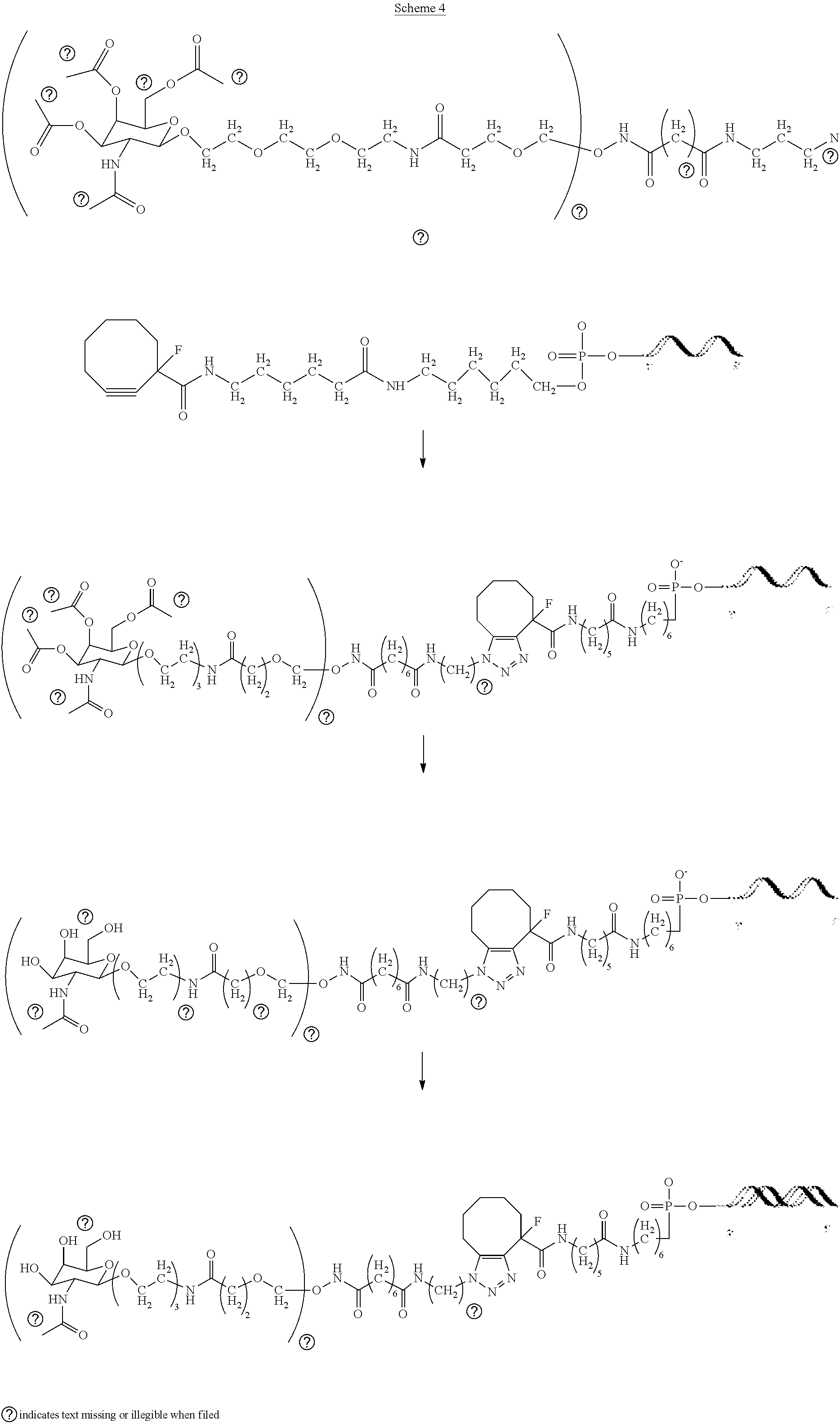

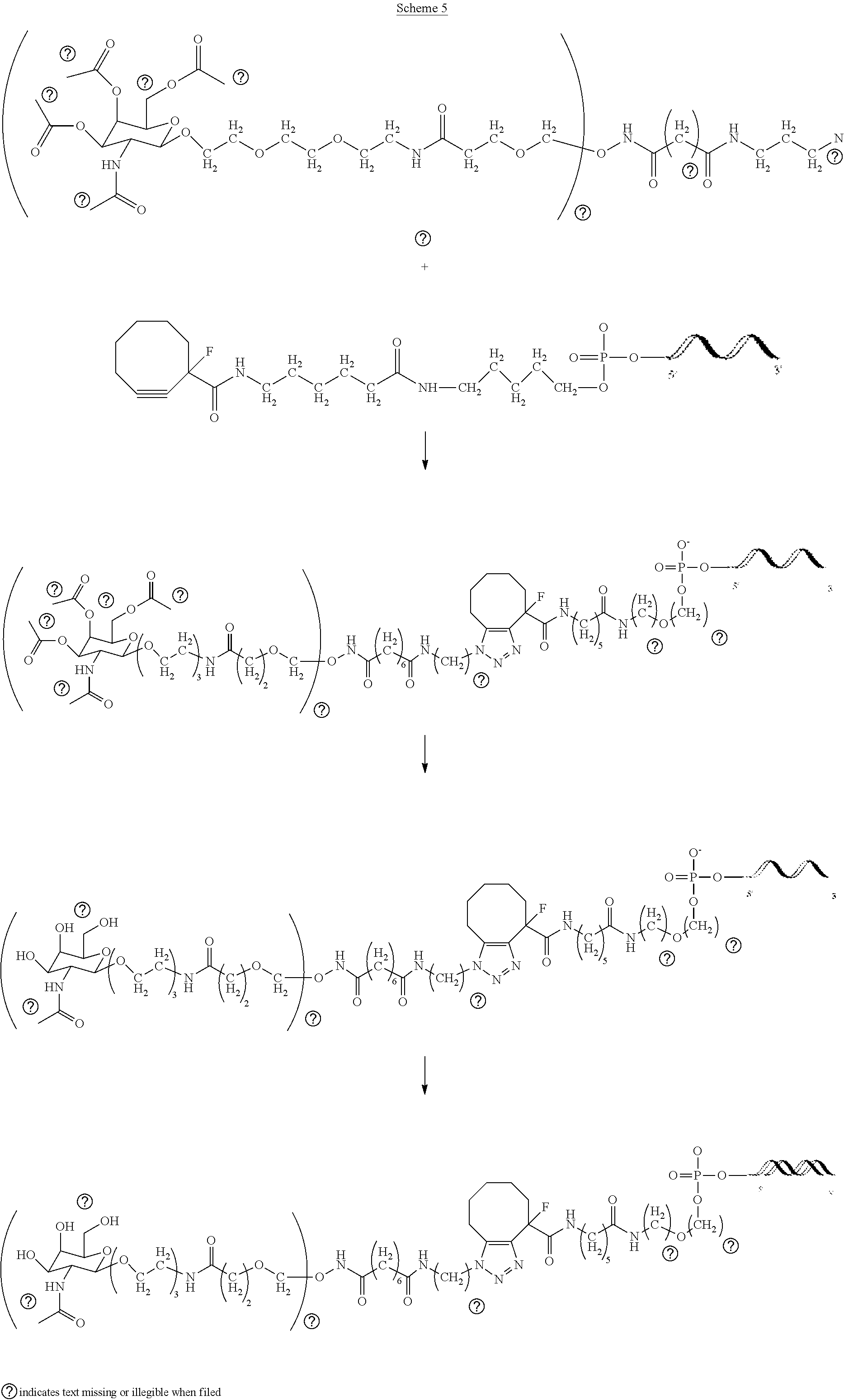

### Example 3

The following constructs are used in examples 3 and 4:

TABLE 12

| | | | Antisense |
|---|---|---|---|
| Target | ID | Sense Sequence 5′→3′ | Sequence 5′→3′ |
| hsHAO1 | ETX006 | (invabasic)(invabasic)gsascuuuCfaUfCfC | usAfsuauUfuCfCfaggaU |
| | | fuggaaauasusa(NHC6)(ET-GalNAc-T2CO) | fgAfaagucscsa |
| hsHAO1 | ETX002 | (ET-GalNAc-T2CO)(NH2C12)gacuuuCfaUfCfC | usAfsuauUfuCfCfaggaU |
| | | fuggaaauasusa(invabasic)(invabasic) | fgAfaagucscsa |
| hsC5 | ETX011 | (ET-GalNAc-T2CO)(NH2C12)aaGfcAfaGfaUfA | usAfsUfuAfuaAfaAfaua |
| | | fUfuUfuuAfuAfasusa(invabasic)(invabasic) | UfcUfuGfcuususudTdT |
| hsC5 | ETX015 | (invabasic)(invabasic)asasGfcAfaGfaUfA | usAfsUfuAfuaAfaAfaua |
| | | fUfuUfuuAfuAfaua(NHC6)(ET-GalNAc-T2CO) | UfcUfuGfcuususudTdT |
| hsTTR | ETX020 | (ET-GalNAc-T2CO)(NH2C12)ugggauUfuCfAfU | usCfsuugGfuuAfcaugAf |
| | | fguaaccaasgsa(invabasic)(invabasic) | aAfucccasusc |
| hsTTR | ETX024 | (invabasic)(invabasic)usgsggauUfuCfAfU | usCfsuugGfuuAfcaugAf |
| | | fguaaccaaga(NHC6)(ET-GalNAc-T2CO) | aAfucccasusc |

In Table 12 the components in brackets having the following nomenclature (NHC6), (NH2C12) and (ET-GalNAc-T2CO) are descriptors of elements of the linkers, and the complete corresponding linker structures are shown in FIG. 32 and FIG. 33 herein. This correspondence of abbreviation to actual linker structure similarly applies to all other references of the above abbreviations herein.

TABLE 12a

| | | | Linker plus ligand |
|---|---|---|---|
| Target | ID | Short Descriptor | SiRNA as Table 12 |
| hsHAO1 | ETX006 | 3′-GalNAc T2a | Linker + ligand as |
| | | inverted abasic | FIG. 32 |
| hsHAO1 | ETX002 | 5′-GalNAc T2b | Linker + ligand as |
| | | inverted abasic | FIG. 33 |
| hsC5 | ETX011 | 5′-GalNAc T2b | Linker + ligand as |
| | | inverted abasic | FIG. 33 |
| hsC5 | ETX015 | 3′-GalNAc T2a | Linker + ligand as |
| | | inverted abasic | FIG. 32 |
| hsTTR | ETX020 | 5′-GalNAc T2b | Linker + ligand as |
| | | inverted abasic | FIG. 33 |
| hsTTR | ETX024 | 3′-GalNAc T2a | Linker + ligand as |
| | | inverted abasic | FIG. 32 |

The following control constructs are also used in the examples:

TABLE 13

| Target | ID | Sense Sequence 5′→3′ | Antisense Sequence 5′→3′ |
|---|---|---|---|
| F-Luc | XD- | cuuAcGcuGAGuAcuucGAdTsdT | UCGAAGuACUcAGCGuAAGdTsdT |
| | 00914 | | |
| hsFVII | XD- | AGAuAuGcAcAcAcAcGGAdTsdT | UCCGUGUGUGUGcAuAUCUdTsdT |
| | 03999 | | |
| hsAHSA1 | XD- | uscsUfcGfuGfgCfcUfuAfaUf | UfsUfsuCfaUfuAfaGfgCfcAf |
| | 15421 | gAfaAf(invdT) | cGfaGfasusu |

### Abbreviations

AHSA1 Activator of heat shock protein ATPase1
ASGR1 Asialoglycoprotein Receptor 1
ASO Antisense oligonucleotide
bDNA branched DNA
bp base-pair
C5 complement C5
conc. concentration
ctrl. control
CV coefficient of variation
dG, dC, dA, dT DNA residues
F Fluoro
FCS fetal calf serum
GalNAc N-Acetylgalactosamine
GAPDH Glyceraldehyde 3-phosphate dehydrogenase
G, C, A, U RNA residues
g, c, a, u 2′-O-Methyl modified residues
Gf, Cf, Af, Uf 2′-Fluoro modified residues
h hour
HAO1 Hydroxyacid Oxidase 1
HPLC High performance liquid chromatography
Hs Homo sapiens
IC50 concentration of an inhibitor where the response is reduced by 50%
ID identifier
KD knockdown
LF2000 Lipofectamine2000
M molar
Mf Macaca fascicularis
min minute
MV mean value
n.a. or N/A not applicable
NEAA non-essential amino acid
nt nucleotide
QC Quality control
QG2.0 QuantiGene 2.0
RLU relative light unit
RNAi RNA interference
RT room temperature
s Phosphorothioate backbone modification
SAR structure-activity relationship
SD standard deviation
siRNA small interfering RNA
TTR Transthyretin

### Example 3

### Summary

GalNAc-siRNAs targeting either hsHAO1, hsC5 or hsTTR mRNA were synthesized and QC-ed. The entire set of siRNAs (except siRNAs targeting HAO1) was first studied in a dose-response setup in HepG2 cells by transfection using RNAiMAX, followed by a dose-response analysis in a gymnotic free uptake setup in primary human hepatocytes.

Direct incubation of primary human hepatocytes with GalNAc-siRNAs targeting hsHAO1, hsC5 or hsTTR mRNA resulted in dose-dependent on-target mRNA silencing to varying degrees.

Aim of Study

The aim of this set of experiments was to analyze the in vitro activity of different GalNAc-ligands in the context of siRNAs targeting three different on-targets, namely hsHAO1, hsC5 or hsTTR mRNA.

Work packages of this study included (i) assay development to design, synthesize and test bDNA probe sets specific for each and every individual on-target of interest, (ii) to identify a cell line suitable for subsequent screening experiments, (iii) dose-response analysis of potentially all siRNAs (by transfection) in one or more human cancer cell lines, and (iv) dose-response analysis of siRNAs in primary human hepatocytes in a gymnotic, free uptake setting. In both settings, IC50 values and maximal inhibition values should be calculated followed by ranking of the siRNA study set according to their potency.

Material and Methods

### Oligonucleotide Synthesis

Standard solid-phase synthesis methods were used to chemically synthesize siRNAs of interest (see Table 12) as well as controls (see Table 13).

### Cell Culture and In-Vitro Transfection Experiments

Cell culture, transfection and QuantiGene2.0 branched DNA assay are described below, and siRNA sequences are listed in Tables 12 and 13. HepG2 cells were supplied by American Tissue Culture Collection (ATCC) (HB-8065, Lot #: 63176294) and cultured in ATCC-formulated Eagle's Minimum Essential Medium supplemented to contain 10% fetal calf serum (FCS). Primary human hepatocytes (PHHs) were sourced from Primacyt (Schwerin, Germany) (Lot #: CyHuf19009HEc). Cells are derived from a malignant glioblastoma tumor by explant technique. All cells used in this study were cultured at 37° C. in an atmosphere with 5% CO₂ in a humidified incubator.

For transfection of HepG2 cells with hsC5 or hsTTR targeting siRNAs (and controls), cells were seeded at a density of 20.000 cells/well in regular 96-well tissue culture plates. Transfection of cells with siRNAs was carried out using the commercially available transfection reagent RNAiMAX (Invitrogen/Life Technologies) according to the manufacturer's instructions. 10 point dose-response experiments of 20 candidates (11×hsC5, 9×hsTTR) were done in HepG2 cells with final siRNA concentrations of 24, 6, 1.5, 0.4, 0.1, 0.03, 0.008, 0.002, 0.0005 and 0.0001 nM, respectively.

Dose response analysis in PHHs was done by direct incubation of cells in a gymnotic, free uptake setting starting with 1.5 μM highest final siRNA concentration, followed by 500 nM and from there on going serially down in twofold dilution steps.

Control wells were transfected into HepG2 cells or directly incubated with primary human hepatocytes at the highest test siRNA concentrations studied on the corresponding plate. All control siRNAs included in the different project phases next to mock treatment of cells are summarized and listed in Table 13. For each siRNA and control, at least four wells were transfected/directly incubated in parallel, and individual data points were collected from each well.

After 24 h of incubation with siRNA post-transfection, media was removed and HepG2 cells were lysed in Lysis Mixture (1 volume of lysis buffer plus 2 volumes of nuclease-free water) and then incubated at 53° C. for at least 45 minutes. In the case of PHHs, plating media was removed 5 h post treatment of cells followed by addition of 50 μl of complete maintenance medium per well. Media was exchanged in that way every 24 h up to a total incubation period of 72 h. At either 4 h or 72 h time point, cell culture supernatant was removed followed by addition of 200 μl of Lysis Mixture supplemented with 1:1000 v/v of Proteinase K.

The branched DNA (bDNA) assay was performed according to manufacturer's instructions. Luminescence was read using a 1420 Luminescence Counter (WALLAC VICTOR Light, Perkin Elmer, Rodgau-Jügesheim, Germany) following 30 minutes incubation in the presence of substrate in the dark. For each well, the on-target mRNA levels were normalized to the hsGAPDH mRNA levels. The activity of any siRNA was expressed as percent on-target mRNA concentration (normalized to hsGAPDH mRNA) in treated cells, relative to the mean on-target mRNA concentration (normalized to hsGAPDH mRNA) across control wells.

### Assay Development

QuantiGene2.0 branched DNA (bDNA) probe sets were designed and synthesised specific for Homo sapiens GAPDH, AHSA1, hsHAO1, hsC5 and hsTTR. bDNA probe sets were initially tested by bDNA analysis according to manufacturer's instructions, with evaluation of levels of mRNAs of interest in two different lysate amounts, namely 10 μl and 50 μl, of the following human and monkey cancer cell lines next to primary human hepatocytes: SJSA-1, TF1, NCI-H₁₆₅₀, Y-79, Kasumi-1, EAhy926, Caki-1, Colo205, RPTEC, A253, HeLaS3, Hep3B, BxPC3, DU145, THP-1, NCI-H₄₆₀, IGR37, LS174T, Be(2)-C, SW 1573, NCI-H₃₅₈, TC71, 22Rv1, BT474, HeLa, KBwt, Panc-1, U87MG, A172, C42, HepG2, LNCaP, PC3, SupTI1, A549, HCT116, HuH7, MCF7, SH-SY5Y, HUVEC, C33A, HEK293, HT29, MOLM 13 and SK-MEL-2. Wells containing only bDNA probe set without the addition of cell lysate were used to monitor technical background and noise signal.

Results

### Identification of Suitable Cell Types for Screening of GalNAc-siRNAs

FIGS. 1-3 show mRNA expression data for the three on-targets of interest, namely hsC5, hsHAO1 and hsTTR, in lysates of a diverse set of human cancer cell lines plus primary human hepatocytes. Cell numbers per lysate volume are identical with each cell line tested, this is necessary to allow comparisons of expression levels amongst different cell types. FIG. 1 shows hsC5 mRNA expression data for all cell types tested.

The identical type of cells were also screened for expression of hsHAO1 mRNA, results are shown in bar diagrams as part of FIG. 2.

Lastly, suitable cell types were identified which would allow for screening of GalNAc-siRNAs targeting hsTTR, respective data are part of FIG. 3.

In summary, mRNA expression levels for all three on-targets of interest are high enough in primary human hepatocytes (PHHs). Further, HepG2 cells could be used to screen GalNAc-siRNAs targeting hsC5 and hsTTR mRNAs, in contrast, no cancer cell line could be identified which would be suitable to test siRNAs specific for hsHAO1 mRNA.

Dose-Response Analysis of hsTTR Targeting GalNAc-siRNAs in HepG2 Cells

Following transfection optimization, HepG2 cells were transfected with the entire set of hsTTR targeting GalNAc-siRNAs (see Table 12) in a dose-response setup using RNAiMAX. The highest final siRNA test concentration was 24 nM, going down in ninecells. Table 14 lists activity data for all hsTTR targeting GalNAC-siRNAs studied.

TABLE 14
Target, incubation time, external ID, IC20/IC50/IC80 values and
maximal inhibition of hsTTR targeting siRNAs in HepG2 cells.
The listing is ordered according to external ID, with 4 h of
incubation listed on top and 24 h of incubation on the bottom.

| | Incubation | External | IC20 | IC50 | IC80 | Max. Inhib. |
|---|---|---|---|---|---|---|
| Target | [h] | ID | [nM] | [nM] | [nM] | [%] |
| hsTTR | 4 | ETX020 | 1.953 | #N/A | #N/A | 37.9 |
| hsTTR | 4 | ETX024 | 1.952 | #N/A | #N/A | 48.2 |
| hsTTR | 24 | ETX020 | 0.005 | 0.025 | 0.133 | 95.5 |
| hsTTR | 24 | ETX024 | 0.008 | 0.029 | 0.134 | 95.5 |

Results for the 24 h incubation are also shown in FIGS. 11A-11B.

In general, transfection of HepG2 cells with hsTTR targeting siRNAs results in on-target mRNA silencing spanning in general the entire activity range from 0% silencing to maximal inhibition. Data generated 24 h post transfection are more robust with lower standard variations, as compared to data generated only 4 h post transfection. Further, the extent of on-target knockdown generally increases over time from 4 h up to 24 h of incubation. hsTTR GalNAc-siRNAs have been identified that silence the on-target mRNA>95% with IC50 values in the low double-digit pM range.

Dose-Response Analysis of hsC5 Targeting GalNAc-siRNAs in HepG2 Cells

The second target of interest, hsC5 mRNA, was tested in an identical dose-response setup (with minimally different final siRNA test concentrations, however) by transfection of HepG2 cells using RNAiMAX with GalNAc-siRNAs sharing identical linger/position/GalNAc-ligand variations as with hsTTR siRNAs, but sequences specific for the on-target hsC5 mRNA.

TABLE 15
Target, incubation time, external ID, IC20/IC50/IC80 values and
maximal inhibition of hsC5 targeting siRNAs in HepG2 cells.
The listing is ordered according to external ID, with 4 h of
incubation listed on top and 24 h of incubation on the bottom.

| | Incubation | External | IC20 | IC50 | IC80 | Max. Inhib. |
|---|---|---|---|---|---|---|
| Target | [h] | ID | [nM] | [nM] | [nM] | [%] |
| C5 | 4 | ETX011 | 0.091 | 0.424 | #N/A | 74.6 |
| C5 | 4 | ETX015 | 0.407 | 0.578 | #N/A | 61.9 |
| C5 | 24 | ETX011 | 0.001 | 0.005 | 0.045 | 88.4 |
| C5 | 24 | ETX015 | 0.003 | 0.013 | 0.099 | 88.8 |

Results for the 24 h incubation are also shown in FIGS. 12A-12B.

There is dose-dependent on-target hsC5 mRNA silencing upon transfection of HepG2 cells with the GalNAc-siRNA set specific for hsC5. Some knockdown can already be detected at 4 h post-transfection of cells, an even higher on-target silencing is observed after a longer incubation period, namely 24 h. hsC5 GalNAc-siRNAs have been identified that silence the on-target mRNA almost 90% with IC50 values in the low single-digit pM range.

### Identification of a Primary Human Hepatocyte Batch Suitable for Testing of all GalNAc-siRNAs

The dose-response analysis of the two GalNAc-siRNA sets in human cancer cell line HepG2 should demonstrate (and ensure) that all new GalNAc-/linker/position/cap variants are indeed substrates for efficient binding to AGO2 and loading into RISC, and in addition, able to function in RNAi-mediated cleavage of target mRNA. However, in order to test whether the targeting GalNAc-ligand derivatives allow for efficient uptake into hepatocytes, dose-response analysis experiments should be done in primary human hepatocytes by gymnotic, free uptake setup. Hepatocytes do exclusively express the Asialoglycoprotein receptor (ASGR1) to high levels, and this receptor generally is used by the liver to remove target glycoproteins from circulation. It is common knowledge by now, that certain types of oligonucleotides, e.g. siRNAs or ASOs, conjugated to GalNAc-ligands are recognized by this high turnover receptor and efficiently taken up into the cytoplasm via clathrin-coated vesicles and trafficking to endosomal compartments. Endosomal escape is thought to be the rate-limiting step for oligonucleotide delivery.

An intermediate assay development experiment was done in which different batches of primary human hepatocytes were tested for their expression levels of relevant genes of interest, namely hsC5, hsTTR, hsHAO1, hsGAPDH and hsAHSA1. Primacyt (Schwerin, Germany) provided three vials of different primary human hepatocyte batches for testing, namely BHuf16087, CHF2101 and CyHuf19009. The cells were seeded on collagen-coated 96-well tissue culture plates, followed by incubation of cells for 0 h, 24 h, 48 h and 72 h before cell lysis and bDNA analysis to monitor mRNA levels of interest. FIG. 6 shows the absolute mRNA expression data for all three on-targets of interest—hsTTR, hsC5 and hsHAO1—in the primary human hepatocyte batches BHuf16087, CHF2101 and CyHuf19009. mRNA expression levels of hsGAPDH and hsAHSA1 are shown in FIG. 7.

Overall, the mRNA expression of all three on-targets of interest in the primary human hepatocyte batches BHuf16087 and CyHuf19009 are high enough after 72 h to continue with the bDNA assay. Due to the total amount of vials available for further experiments, we continued the experiments with the batch CyHuf19009.

Dose-Response Analysis of hsHAO1 Targeting GalNAc-siRNAs in PHHs

Following the identification of a suitable batch (CyHuf19009) of primary human hepatocytes (PHHs), a gymnotic, free uptake analysis was performed of hsHAO1 targeting GalNAc-siRNAs, listed in Table 12. The highest tested final siRNA concentration was 1.5 μM, followed by 500 nM, going down in eight two-fold serial dilution steps to the lowest final siRNA concentration of 1.95 nM. The experiments ended at 4 h and 72 h post direct incubation of PHH cells. Table 16 lists activity data for all hsHAO1 targeting GalNAc-siRNAs studied. All control siRNAs included in this experiment are summarized and listed in Table 13.

TABLE 16
Target, incubation time, external ID, IC20/IC50/IC80 values
and maximal inhibition of hsHAO1 targeting GalNAc-siRNAs
in primary human hepatocytes (PHHs). The listing is organized
according to external ID, with 4 h and 72 h incubation
listed on top and bottom, respectively.

| | Incubation | External | IC20 | IC50 | IC80 ] | Max. Inhib. |
|---|---|---|---|---|---|---|
| Target | [h] | ID | [nM] | [nM] | [nM | [%] |
| hsHAO1 | 4 | ETX002 | #N/A | #N/A | #N/A | 7.2 |
| (hsGO1) | | | | | | |
| hsHAO1 | 4 | ETX006 | #N/A | #N/A | #N/A | 0.5 |
| (hsGO1) | | | | | | |
| hsHAO1 | 72 | ETX002 | 23.9 | #N/A | #N/A | 44.3 |
| (hsGO1) | | | | | | |
| hsHAO1 | 72 | ETX006 | 27.5 | 617.1 | #N/A | 53.6 |
| (hsGO1) | | | | | | |

Results for the 72 h incubation are also shown in FIGS. 13A-13B.

Gymnotic, free uptake of GalNAc-siRNAs targeting hsHAO1 did not lead to significant on-target silencing within 4 h, however after 72 h incubation on-target silencing was visible in a range of 35.5 to 58.1% maximal inhibition.

Dose-Response Analysis of hsC5 Targeting GalNAc-siRNAs in PHHs

The second target of interest, hsC5 mRNA, was tested in an identical dose-response setup by gymnotic, free uptake in PHHs with GalNAc-siRNAs sharing identical linker/position/GalNAc-ligand variations as with hsTTR and hsHAO1 tested in the assays before, but sequences specific for the on-target hsC5 mRNA. Sequences for the GalNAc-siRNAs targeting hsC5 and all sequences and information about control siRNAs are listed in Table 12 and Table 13, respectively. The experiment ended after 4 h and 72 h direct incubation of PHHs. Table 17 lists activity data for all hsC5 targeting GalNAc-siRNAs studied.

TABLE 17
Target, incubation time, external ID, IC20/IC50/IC80 values and
maximal inhibition of hsC5 targeting GalNAc-siRNAs in PHHs.
The listing is organized according to external ID, with 4 h
and 72 h incubation listed on top and bottom, respectively.

| | Incubation | External | IC20 | IC50 | IC80 | Max. Inhib. |
|---|---|---|---|---|---|---|
| Target | [h] | ID | [nM] | [nM] | [nM] | [%] |
| C5 | 4 | ETX011 | #N/A | #N/A | #N/A | −2.9 |
| C5 | 4 | ETX015 | #N/A | #N/A | #N/A | 7.6 |
| C5 | 72 | ETX011 | 2.6 | 295.3 | #N/A | 62.1 |
| C5 | 72 | ETX015 | 7.2 | 315.0 | #N/A | 57.2 |

Results for the 72 h incubation are also shown in FIGS. 14A-14B.

No significant on-target silencing of GalNAc-siRNAs is visible after 4 h incubation. Data generated after an incubation period of 72 h showed a more robust on-target silencing of up to 65.5% maximal inhibition.

Dose-Response Analysis of hsTTR Targeting GalNAc-siRNAs in PHHs

The last target of interest, hsTTR mRNA, was again tested in a gymnotic, free uptake in PHHs in an identical dose-response setup as for the targets hsHAO1 and hsC5, with the only difference being that specific siRNA sequences for the on-target hsTTR mRNA was used (see Table 12).

The experiment ended after 72 h of direct incubation of PHHs. Table 18 lists activity data for all hsTTR targeting GalNAc-siRNAs studied.

TABLE 18
Target, incubation time, external ID, IC20/IC50/IC80
values and maximal inhibition of hsTTR targeting
GalNAc-siRNAs in primary human hepatocytes (PHHs).
The listing is organized according to external ID.

| | Incubation | External | IC20 | IC50 | IC80 | Max. Inhib. |
|---|---|---|---|---|---|---|
| Target | [h] | ID | [nM] | [nM] | [nM] | [%] |
| hsTTR | 72 | ETX020 | 2.2 | 31.0 | #N/A | 78.4 |
| hsTTR | 72 | ETX024 | 9.5 | 110.0 | #N/A | 71.3 |

Results are also shown in FIGS. 15A-15B.

Gymnotic, free uptake of GalNAc-siRNAs targeting hsTTR did lead to significant on-target silencing within 72 h, ranging between 46 to 82.5% maximal inhibition.

Conclusions and Discussion

The scope of this study was to analyze the in vitro activity of GalNAc-ligands according to the present invention when used in the context of siRNAs targeting three different on-targets, namely hsHAO1, hsC5 and hsTTR mRNA. siRNA sets specific for each target were composed of siRNAs with different linker/cap/modification/GalNAc-ligand chemistries in the context of two different antisense strands each.

For all targets, GalNAc-siRNAs from Table 12 were identified that showed a high overall potency and low IC50 value.

### Example 4

### Routes of Synthesis

vii) Synthesis of the Conjugate Building Blocks TriGalNAc

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfar Silica 10, 25, 50 or 100 g columns (Uppsala, Sweden).

All moisture-sensitive reactions were carried out under anhydrous conditions using dry glassware, anhydrous solvents and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (¹H NMR) and 125 MHz (¹³C NMR). Chemical shifts are given in ppm referenced to the solvent residual peak (CDCl₃—¹H NMR: 6 at 7.26 ppm and ¹³C NMR δ at 77.2 ppm; DMSO-d₆—¹H NMR: 6 at 2.50 ppm and ¹³C NMR δ at 39.5 ppm). Coupling constants are given in Hertz. Signal splitting patterns are described as singlet (s), doublet (d), triplet (t) or multiplet (m). viii) Synthesis route for the conjugate building block TriGalNAc

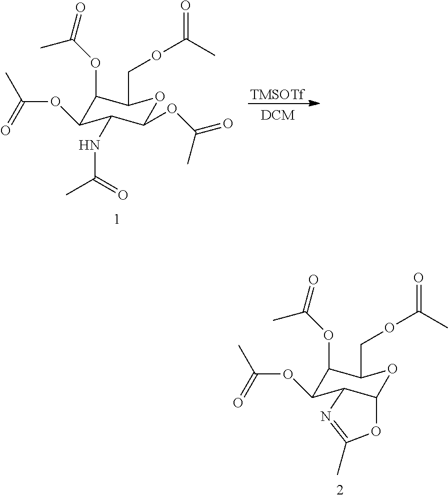

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃(100 mL) and water (100 mL). The organic layer was separated, dried over Na2SO4 and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

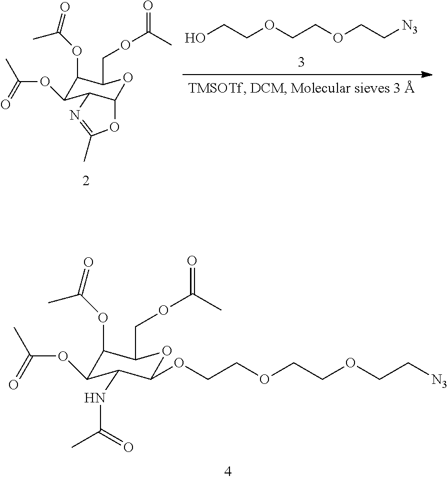

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) was added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na2SO4 and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6 (C), 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH₃).

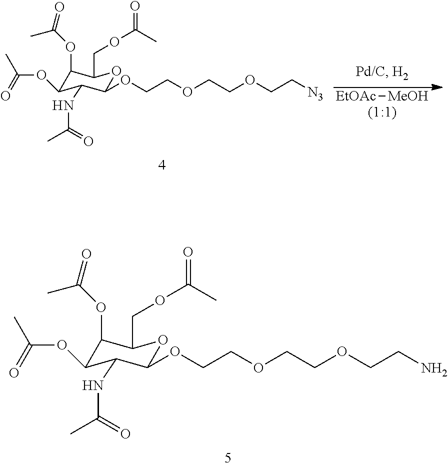

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

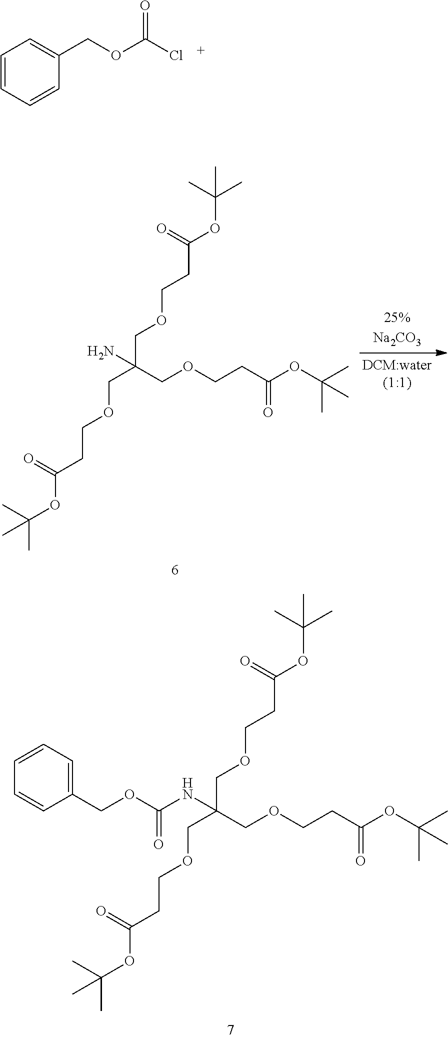 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na2SO4. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH₂), 66.8 (3×CH₂), 64.9 (C), 58.7 (CH₂), 35.8 (3×CH₂), 27.7 (9×CH₃).

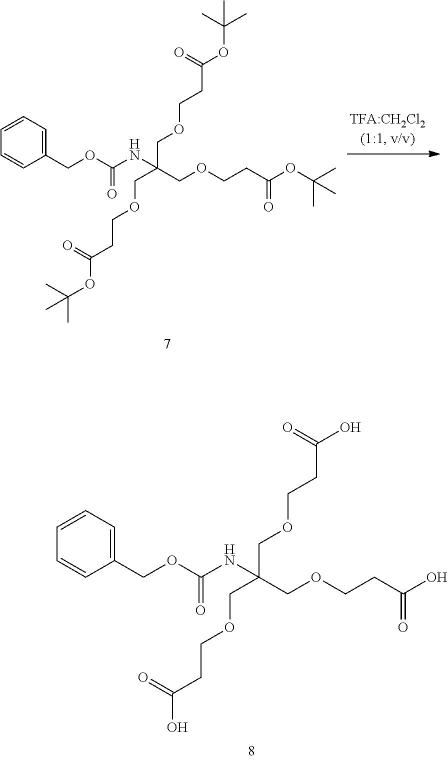 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

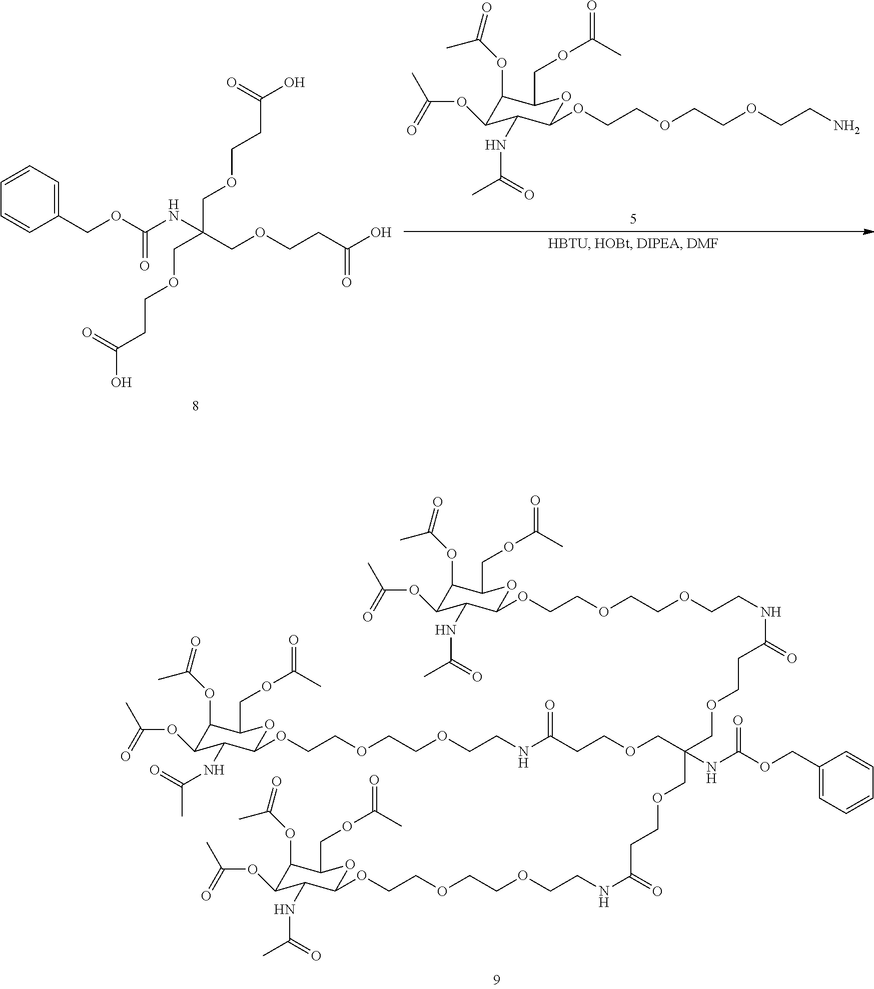 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uronium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (C

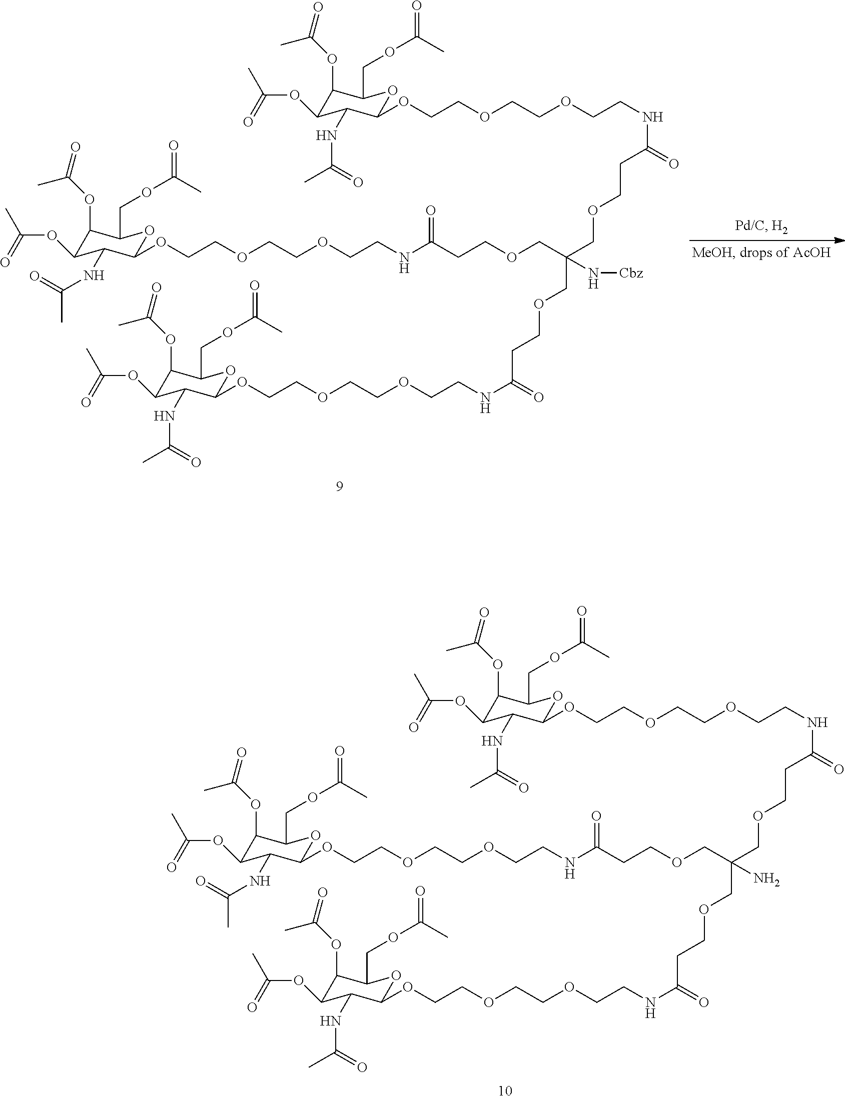 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

### H2), 22

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

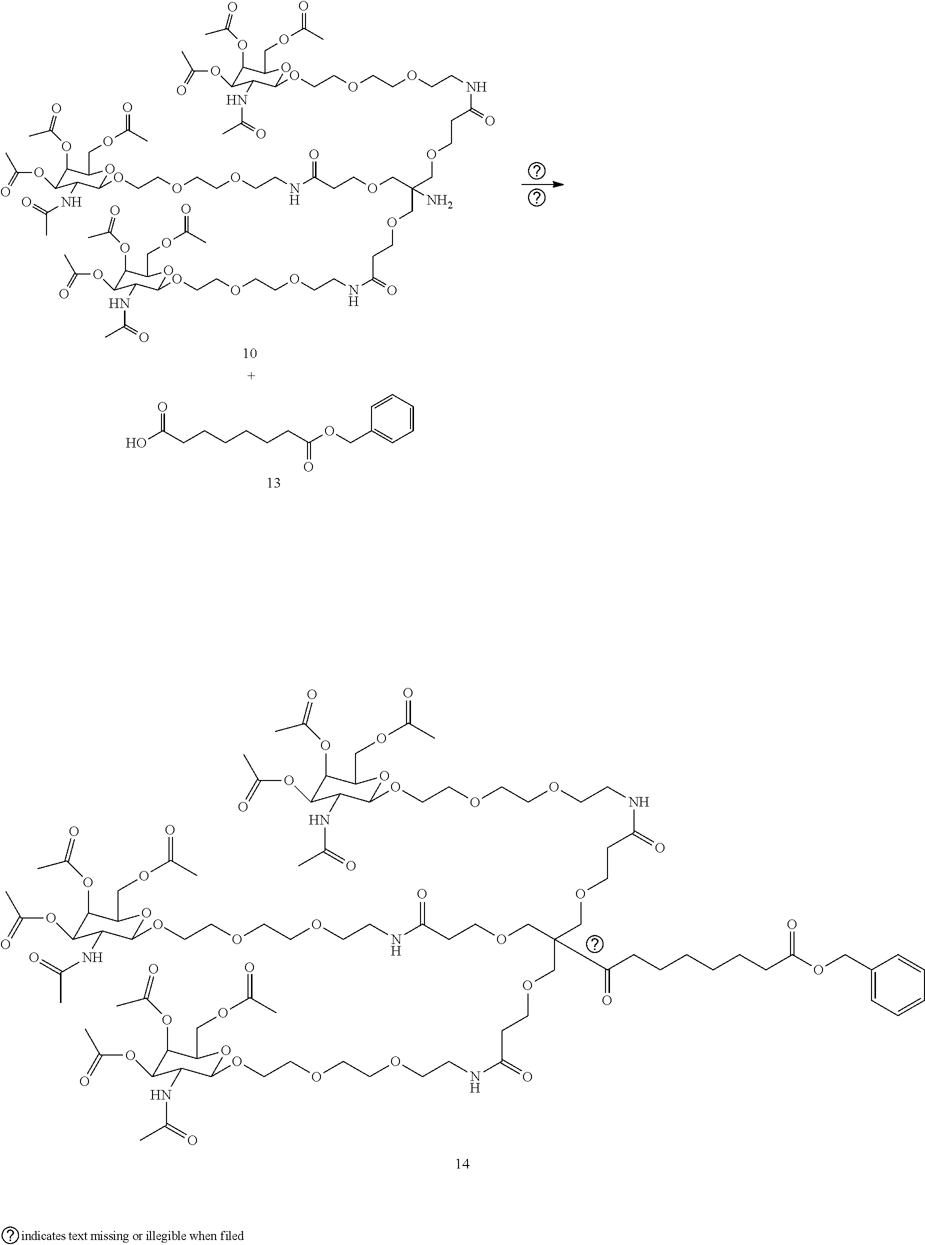 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; O=C(O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)CC(=O)CCCCCCC(=O)OCc2ccccc2)OC(COC(C)=O)C(OC(C)=O)C1OC(C)=O]

Preparation of compound 14: Triantennary GalNAc compound 10 (0.45 g, 0.26 mmol, 1.0 eq.), HBTU (0.19 g, 0.53 mmol, 2.0 eq.) and DIPEA (0.23 mL, 1.3 mmol, 5.0 eq.) were dissolved in DCM (10 mL) under argon. To this mixture, it was added dropwise a solution of compound 13 (0.14 g, 0.53 mmol, 2.0 eq.) in DCM (5 mL). The reaction was stirred at room temperature overnight. The solvent was removed and the residue was dissolved in EtOAc (50 mL), washed with water (50 mL) and dried over Na2SO4. The solvent was evaporated and the crude material was purified by flash chromatography (gradient elution: 0-5% MeOH in DCM in 20 CV). The product was obtained as white fluffy solid (0.25 g, 48%, rf=0.4 (10% MeOH in DCM)). MS: calculated for C88H137N7O42, 1965.1. Found 1965.6.

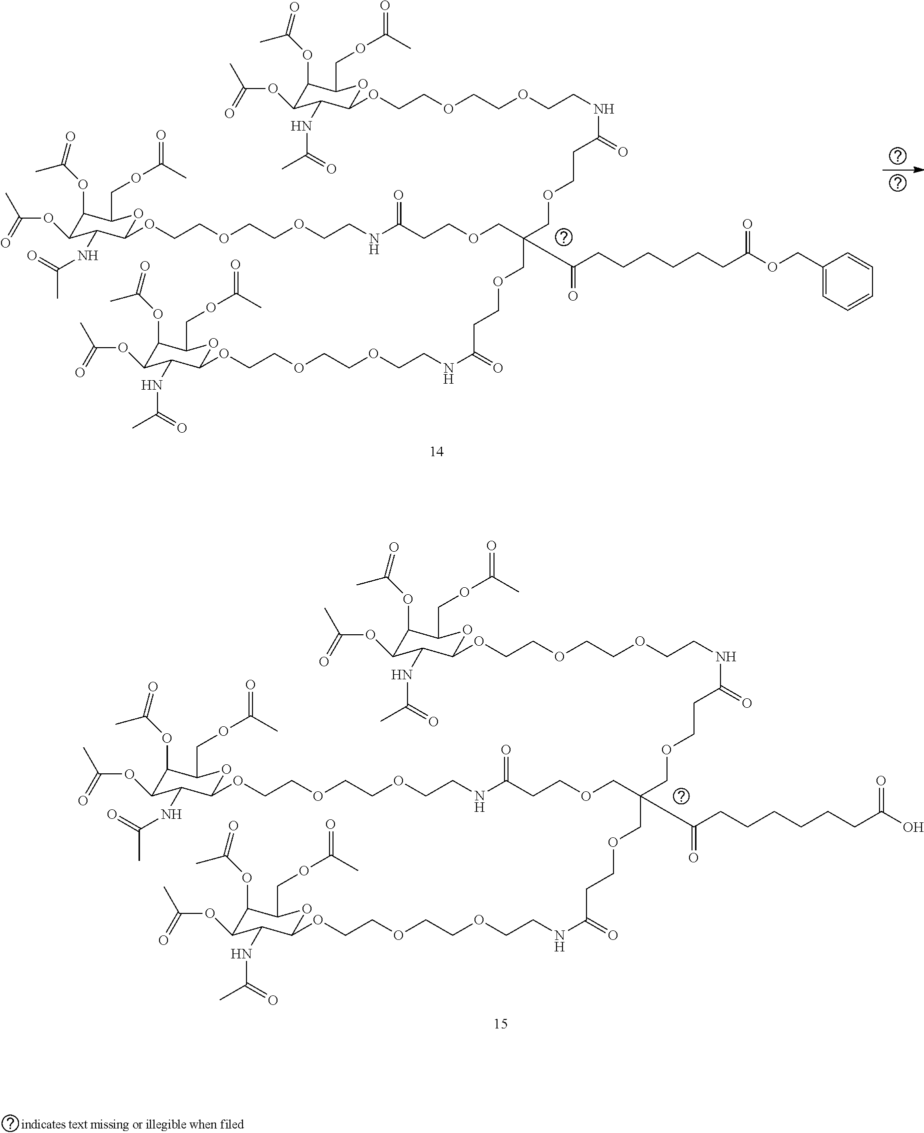 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)CC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)CC(=O)CCCCCCC(=O)O)OC(COC(C)=O)C(OC(C)=O)C1OC(C)=O]

Preparation of TriGalNAc (15): Triantennary GalNAc compound 14 (0.31 g, 0.15 mmol, 1.0 eq.) was dissolved in EtOAc (15 mL) and Pd/C (40 mg) was added. The reaction mixture was degassed by using vacuum/argon cycles (3×) and hydrogenated under balloon pressure overnight. The completion of the reaction was monitored by mass spectrometry and the resulting mixture was filtered through a thin pad of celite. The solvent was removed under reduced pressure and the resulting residue was dried under high vacuum over night. The residue was used for conjugations to oligonucleotides without further purification (0.28 g, quantitative yield). MS: calculated for C81H131N7O42, 1874.9. Found 1875.3.

ix) Oligonucleotide Synthesis

TABLE 19

| | | Purity |
|---|---|---|
| Single | | by RP |
| strand | | HPLC |
| ID | Sequence 5′-3′ | (%) |
| X91382 | (NH2-DEG)gacuuuCfaUfCfCfuggaaauasusa | 89.5 |
| | (invabasic)(invabasic) | |
| X91383 | (NH2-DEG)aaGfcAfaGfaUfAfUfuUfuuAfuAf | 91.6 |
| | asusa(invabasic)(invabasic) | |
| X91384 | (NH2-DEG)ugggauUfuCfAfUfguaaccaasgsa | 94.0 |
| | (invabasic)(invabasic) | |
| X91403 | (NH2C12)gacuuuCfaUfCfCfuggaaauasusa | 94.2 |
| | (invabasic)(invabasic) | |
| X91404 | (NH2C12)aaGfcAfaGfaUfAfUfuUfuuAfuAfa | 96.5 |
| | susa(invabasic)(invabasic) | |
| X91405 | (NH2C12)ugggauUfuCfAfUfguaaccaasgsa | 91.3 |
| | (invabasic)(invabasic) | |
| X91415 | (invabasic)(invabasic)gsascuuuCfaUfC | 96.4 |
| | fCfuggaaauasusa(NH2C6) | |
| X91416 | (invabasic)(invabasic)asasGfcAfaGfaU | 77.4 |
| | fAfUfuUfuuAfuAfaua(NH2C6) | |
| X91417 | (invabasic)(invabasic)usgsggauUfuCfA | 96.7 |
| | fUfguaaccaaga(NH2C6) | |
| X91379 | gsascuuuCfaUfCfCfuggaaauaua(GalNAc) | 92.8 |
| X91380 | asasGfcAfaGfaUfAfUfuUfuuAfuAfaua | 95.7 |
| | (GalNAc) | |
| X91446 | usgsggauUfuCfAfUfguaaccaaga(GalNAc) | 92.1 |
| X38483 | usAfsuauUfuCfCfaggaUfgAfaagucscsa | 91.0 |
| X91381 | usAfsUfuAfuaAfaAfauaUfcUfuGfcuususu | 90.0 |
| | dTdT | |
| X38104 | usCfsuugGfuuAfcaugAfaAfucccasusc | 95.4 |

Af, Cf, Gf, Uf. 2′-F RNA nucleotides
a, c, g, u: 2′-O-Me RNA nucleotides
dT: DNA nucleotides
s: Phosphorothioate
invabasic: 1,2-dideoxyribose
NH2-DEG: Aminoethoxyethyl linker
NH2C12: Aminododecyl linker
NH2C6: Aminohexyl linker

Oligonucleotides were synthesized on solid phase according to the phosphoramidite approach. Depending on the scale either a Mermade 12 (BioAutomation Corporation) or an ÄKTA Oligopilot (GE Healthcare) was used.

Syntheses were performed on commercially available solid supports made of controlled pore glass either loaded with invabasic (CPG, 480 Å, with a loading of 86 μmol/g; LGC Biosearch cat. #BCG-1047-B) or 2′-F A (CPG, 520 Å, with a loading of 90 μmol/g; LGC Biosearch cat. #BCG-1039-B) or NH2C6 (CPG, 520 Å, with a loading of 85 μmol/g LGC Biosearch cat. #BCG-1397-B) or GalNAc (CPG, 500 Å, with a loading of 57 μmol/g; Primetech) or 2′-O-Methyl C (CPG, 500 Å, with a loading of 84 μmol/g LGC Biosearch cat. #BCG-10-B) or 2′-O-Methyl A (CPG, 497 Å, with a loading of 85 μmol/g, LGC Biosearch, Cat. #BCG-1029-B) or dT (CPG, 497 Å, with a loading of 87 μmol/g LGC Biosearch, cat. #BCG-1055-B).

2′-O-Me, 2′-F RNA phosphoramidites and ancillary reagents were purchased from SAFC Proligo (Hamburg, Germany).

2′-O-Methyl phosphoramidites include: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-benzoyl-cytidine 2′-O-methyl-3′-[(2-cyanoethyl)-(N,N-diisopropyl)]-phosphoramidite, 5′-(4,4′-dimethoxytrityl)-N-dimethylformamidine-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

2′-F phosphoramidites include: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

In order to introduce the required amino linkers at the 5′-end of the oligonucleotides the 2-[2-(4-Monomethoxytrityl)aminoethoxy]ethyl-(2-cyanoethyl)-N,N-diisopropyl)-phosphoramidite (Glen Research Cat. #1905) and the 12-(trifluoroacetylamino)dodecyl-[(2-cyanoethyl)-(N,N-diisopropyl)]-phosphoramidite (ChemGenes Cat. #CLP-1575) were employed. The invabasic modification was introduced using 5-O-dimethoxytrityl-1,2-dideoxyribose-3-[(2-cyanoethyl)-(N,N-diisopropyl)]-phosphoramidite (ChemGenes Cat. #ANP-1422).

All building blocks were dissolved in anhydrous acetonitrile (100 mM (Mermade12) or 200 mM (ÄKTA Oligopilot)) containing molecular sieves (3 Å) except 2′-O-methyl-uridine phosphoramidite which was dissolved in 50% anhydrous DCM in anhydrous acetonitrile. Iodine (50 mM in pyridine/H₂O 9:1 v/v) was used as oxidizing reagent. 5-Ethyl thiotetrazole (ETT, 500 mM in acetonitrile) was used as activator solution. Thiolation for introduction of phosphorthioate linkages was carried out using 100 mM xanthane hydride (TCI, Cat. #6846-35-1) in acetonitrile/pyridine 4:6 v/v.

Coupling times were 5.4 minutes except when stated otherwise. 5′ amino modifications were incorporated into the sequence employing a double coupling step with a coupling time of 11 minutes per each coupling (total coupling time 22 min). The oxidizer contact time was set to 1.2 min and thiolation time was 5.2 min.

Sequences were synthesized with removal of the final DMT group, with exception of the MMT group from the NH2DEG sequences.

At the end of the synthesis, the oligonucleotides were cleaved from the solid support using a 1:1 volume solution of 28-30% ammonium hydroxide (Sigma-Aldrich, Cat. #221228) and 40% aqueous methylamine (Sigma-Aldrich, Cat. #8220911000) for 16 hours at 6° C. The solid support was then filtered off, the filter was thoroughly washed with H₂O and the volume of the combined solution was reduced by evaporation under reduced pressure. The pH of the resulting solution was adjusted to pH 7 with 10% AcOH (Sigma-Aldrich, Cat. #A6283).

The crude materials were purified either by reversed phase (RP) HPLC or anion exchange (AEX) HPLC.

RP HPLC purification was performed using a XBridge C18 Prep 19×50 mm column (Waters) on an ÄKTA Pure instrument (GE Healthcare). Buffer A was 100 mM triethyl-ammonium acetate (TEAAc, Biosolve) pH 7 and buffer B contained 95% acetonitrile in buffer A. A flow rate of 10 mL/min and a temperature of 60° C. were employed. UV traces at 280 nm were recorded. A gradient of 0% B to 100% B within 120 column volumes was employed. Appropriate fractions were pooled and precipitated in the freezer with 3 M sodium acetate (NaOAc) (Sigma-Aldrich), pH 5.2 and 85% ethanol (VWR). Pellets were isolated by centrifugation, redissolved in water (50 mL), treated with 5 M NaCl (5 mL) and desalted by Size exclusion HPLC on an Äkta Pure instrument using a 50×165 mm ECO column (YMC, Dinslaken, Germany) filled with Sephadex G25-Fine resin (GE Healthcare).

AEX HPLC purification was performed using a TSK gel SuperQ-5PW 20×200 mm (BISCHOFF Chromatography) on an ÄKTA Pure instrument (GE Healthcare). Buffer A was 20 mM sodium phosphate (Sigma-Aldrich) pH 7.8 and buffer B was the same as buffer A with the addition of 1.4 M sodium bromide (Sigma-Aldrich). A flow rate of 10 mL/min and a temperature of 60° C. were employed. UV traces at 280 nm were recorded. A gradient of 10% B to 100% B within 27 column volumes was employed. Appropriate fractions were pooled and precipitated in the freezer with 3 M NaOAc, pH 5.2 and 85% ethanol. Pellets were isolated by centrifugation, redissolved in water (50 mL), treated with 5 M NaCl (5 mL) and desalted by size exclusion chromatography.

The MMT group was removed with 25% acetic acid in water. Once the reaction was complete the solution was neutralized and the samples were desalted by size exclusion chromatography.

Single strands were analyzed by analytical LC-MS on a 2.1×50 mm XBridge C18 column (Waters) on a Dionex Ultimate 3000 (Thermo Fisher Scientific) HPLC system combined either with a LCQ Deca XP-plus Q-ESI-TOF mass spectrometer (Thermo Finnigan) or with a Compact ESI-Qq-TOF mass spectrometer (Bruker Daltonics). Buffer A was 16.3 mM triethylamine, 100 mM 1,1,1,3,3,3-hexafluoro-2-propanol (HFIP) and 1% MeOH in H₂O and buffer B contained buffer A in 95% MeOH. A flow rate of 250 μL/min and a temperature of 60° C. were employed. UV traces at 260 and 280 nm were recorded. A gradient of 1-40% B within 0.5 min followed by 40 to 100% B within 13 min was employed. Methanol (LC-MS grade), water (LC-MS grade), 1,1,1,3,3,3-hexafluoro-2-propanol (puriss. p.a.) and triethylamine (puriss. p.a.) were purchased from Sigma-Aldrich.

x) TriGalNAc Tether 2 (GalNAc-T2) Conjugation at 5′-End or 3′-End 5′-GalNAc-T2 Conjugates

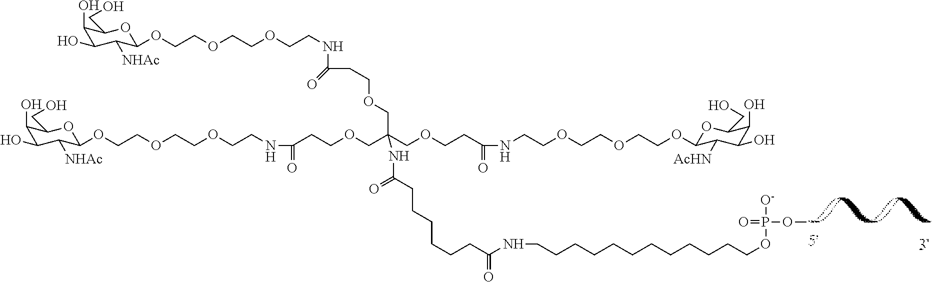 [SMILES: COP(=O)([O-])OCCCCCCCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

3′-GalNAc-T2 Conjugates

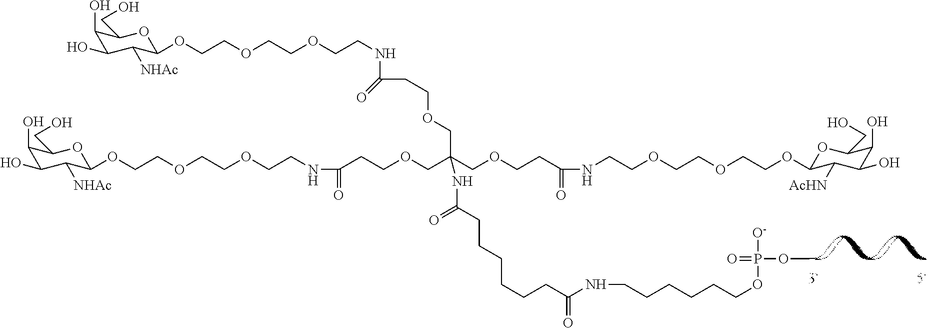 [SMILES: COP(=O)([O-])OCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

Preparation of TriGalNAc tether 2 NHS ester: To a solution of carboxylic acid tether 2 (compound 15, 227 mg, 121 μmol) in DMF (2.1 mL), N-hydroxysuccinimide (NHS) (15.3 mg, 133 μmol) and N,N′-diisopropylcarbodiimide (DIC) (19.7 μL, 127 μmol) were added. The solution was stirred at room temperature for 18 h and used without purification for the subsequent conjugation reactions.

General procedure for triGalNAc tether 2 conjugation: Amine-modified single strand was dissolved at 700 OD/mL in 50 mM carbonate/bicarbonate buffer pH 9.6/DMSO 4:6 (v/v) and to this solution was added one molar equivalent of Tether 2 NHS ester (57 mM) solution in DMF. The reaction was carried out at room temperature and after 1 h another molar equivalent of the NHS ester solution was added. The reaction was allowed to proceed for one more hour and reaction progress was monitored by LCMS. At least two molar equivalent excess of the NHS ester reagent relative to the amino modified oligonucleotide were needed to achieve quantitative consumption of the starting material. The reaction mixture was diluted 15-fold with water, filtered once through 1.2 μm filter from Sartorius and then purified by reserve phase (RP HPLC) on an Äkta Pure (GE Healthcare) instrument.

The purification was performed using a XBridge C18 Prep 19×50 mm column from Waters. Buffer A was 100 mM TEEAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleotides were pooled together, precipitated in the freezer with 3 M NaOAc, pH 5.2 and 85% ethanol and then dissolved at 1000 OD/mL in water. The O-acetates were removed with 20% ammonium hydroxide in water until completion (monitored by LC-MS).

The conjugates were desalted by size exclusion chromatography using Sephadex G25 Fine resin (GE Healthcare) on an Äkta Pure (GE Healthcare) instrument to yield the conjugated oligonucleotides in an isolated yield of 60-80%.

TABLE 20

| | | Purity |
|---|---|---|
| Sense | | by RP |
| strand | | HPLC |
| ID | Sense strand sequence 5′-3′ | (%) |
| X91409 | (GalNAc-T2)(NH2C12)gacuuuCfaUfCfCf | 85.0 |
| | uggaaauasusa(invabasic)(invabasic) | |
| X91410 | (GalNAc-T2)(NH2C12)aaGfcAfaGfaUfAf | 92.3 |
| | UfuUfuuAfuAfasusa(invabasic) | |
| | (invabasic) | |
| X91411 | (GalNAc-T2)(NH2C12)ugggauUfuCfAfUf | 92.7 |
| | guaaccaasgsa(invabasic)(invabasic) | |
| X91433 | (invabasic)(invabasic)gsascuuuCfaU | 85.3 |
| | fCfCfuggaaauasusa(NHC6)(GaINAc-T2) | |
| X91434 | (invabasic)(invabasic)asasGfcAfaGf | 85.8 |
| | aUfAfUfuUfuuAfuAfaua(NHC6) | |
| | (GalNAc-T2) | |
| X91435 | (invabasic)(invabasic)usgsggauUfuC | 84.0 |
| | fAfUfguaaccaaga(NHC6)(GalNAc-T2) | |

The conjugates were characterized by HPLC-MS analysis with a 2.1×50 mm XBridge C18 column (Waters) on a Dionex Ultimate 3000 (Thermo Fisher Scientific) HPLC system equipped with a Compact ESI-Qq-TOF mass spectrometer (Bruker Daltonics). Buffer A was 16.3 mM triethylamine, 100 mM HFIP in 1% MeOH in H2O and buffer B contained 95% MeOH in buffer A. A flow rate of 250 μL/min and a temperature of 60° C. were employed. UV traces at 260 and 280 nm were recorded. A gradient of 1-100% B within 31 min was employed.

xi) Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

GalNAc conjugates prepared are compiled in the table below. These were directed against 3 different target genes. siRNA coding along with the corresponding single strands, sequence information as well as purity for the duplexes is captured.

TABLE 21

| | | | | Duplex |
|---|---|---|---|---|
| | | | | Purity |
| | | | | by |
| | Duplex | SSRN | | HPLC |
| Target | ID | ID | ssRNA-Sequence 5′-3′ | (%) |
| GO | ETX002 | X91409 | (GalNAc-T2)(NH2C12)gacuuuCfaUfCfCfug | 94.1 |
| | | | gaaauasusa(invabasic)(invabasic) | |
| | | X38483 | usAfsuauUfuCfCfaggaUfgAfaagucscsa | |
| | ETX006 | X91433 | (invabasic)(invabasic)gsascuuuCfaUfC | 94.1 |
| | | | fCfuggaaauasusa(NHC6)(GalNAc-T2) | |
| | | X38483 | usAfsuauUfuCfCfaggaUfgAfaagucscsa | |
| C5 | ETX011 | X91410 | (GalNAc-T2)(NH2C12)aaGfcAfaGfaUfAfUf | 93.5 |
| | | | uUfuuAfuAfasusa(invabasic)(invabasic) | |
| | | X91381 | usAfsUfuAfuaAfaAfauaUfcUfuGfcuususud | |
| | | | TdT | |
| | ETX015 | X91434 | (invabasic)(invabasic)asasGfcAfaGfaU | 95.3 |
| | | | fAfUfuUfuuAfuAfaua(NHC6)(GalNAc-T2) | |
| | | X91381 | usAfsUfuAfuaAfaAfauaUfcUfuGfcuususud | |
| | | | TdT | |
| TTR | ETX020 | X91411 | (GalNAc-T2)(NH2C12)ugggauUfuCfAfUfgu | 97.5 |
| | | | aaccaasgsa(invabasic)(invabasic) | |
| | | X38104 | usCfsuugGfuuAfcaugAfaAfucccasusc | |
| | ETX024 | X91435 | (invabasic)(invabasic)usgsggauUfuCfA | 95.3 |
| | | | fUfguaaccaaga(NHC6)(GalNAc-T2) | |
| | | X38104 | usCfsuugGfuuAfcaugAfaAfucccasusc | |

The following schemes further set out the routes of synthesis:

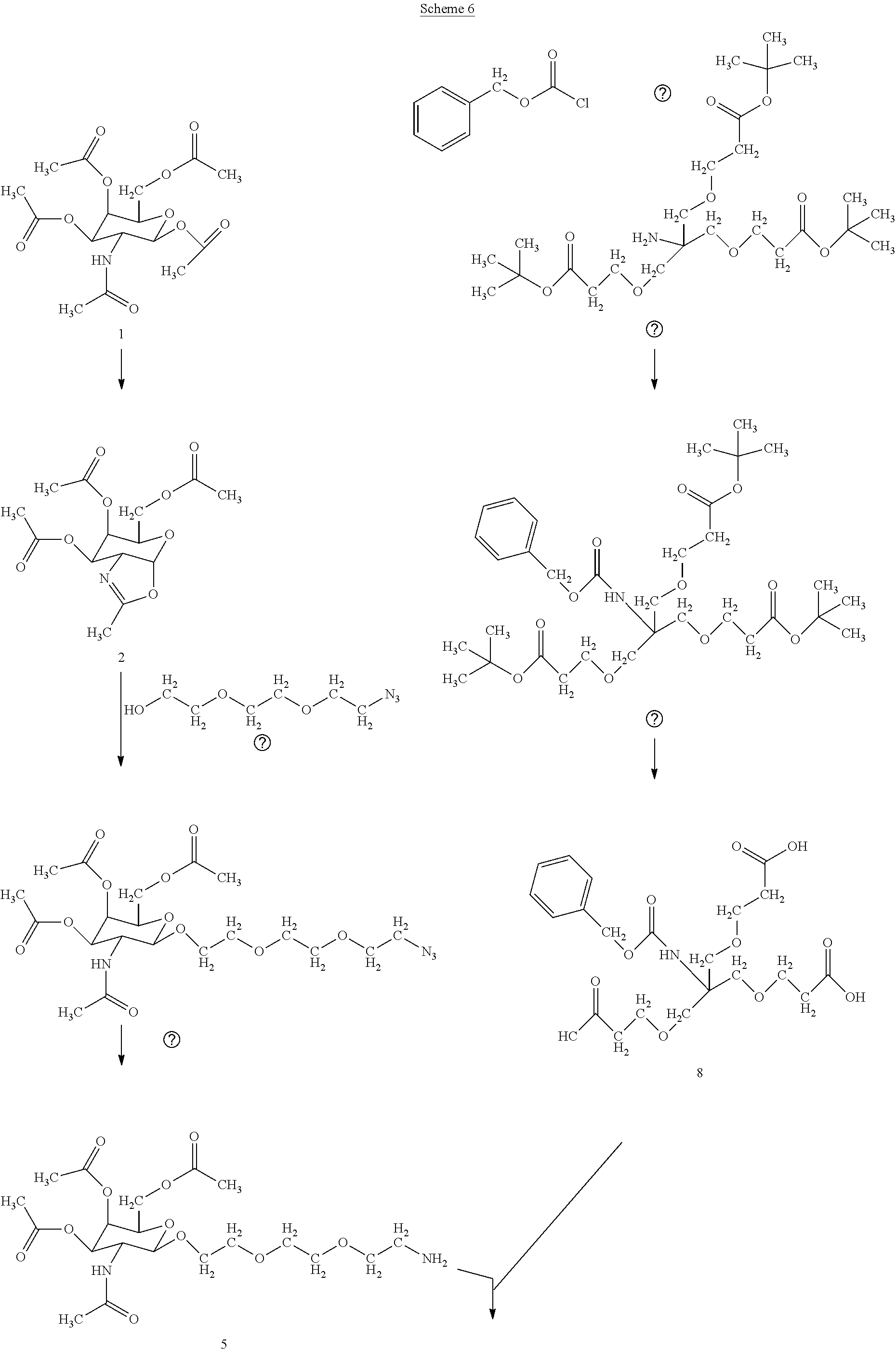

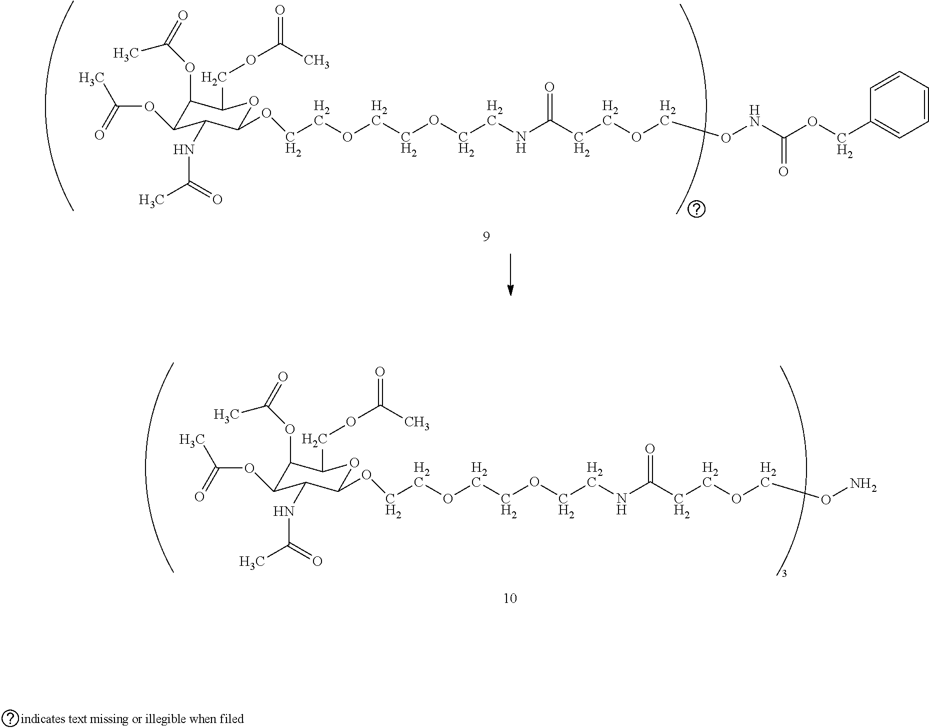

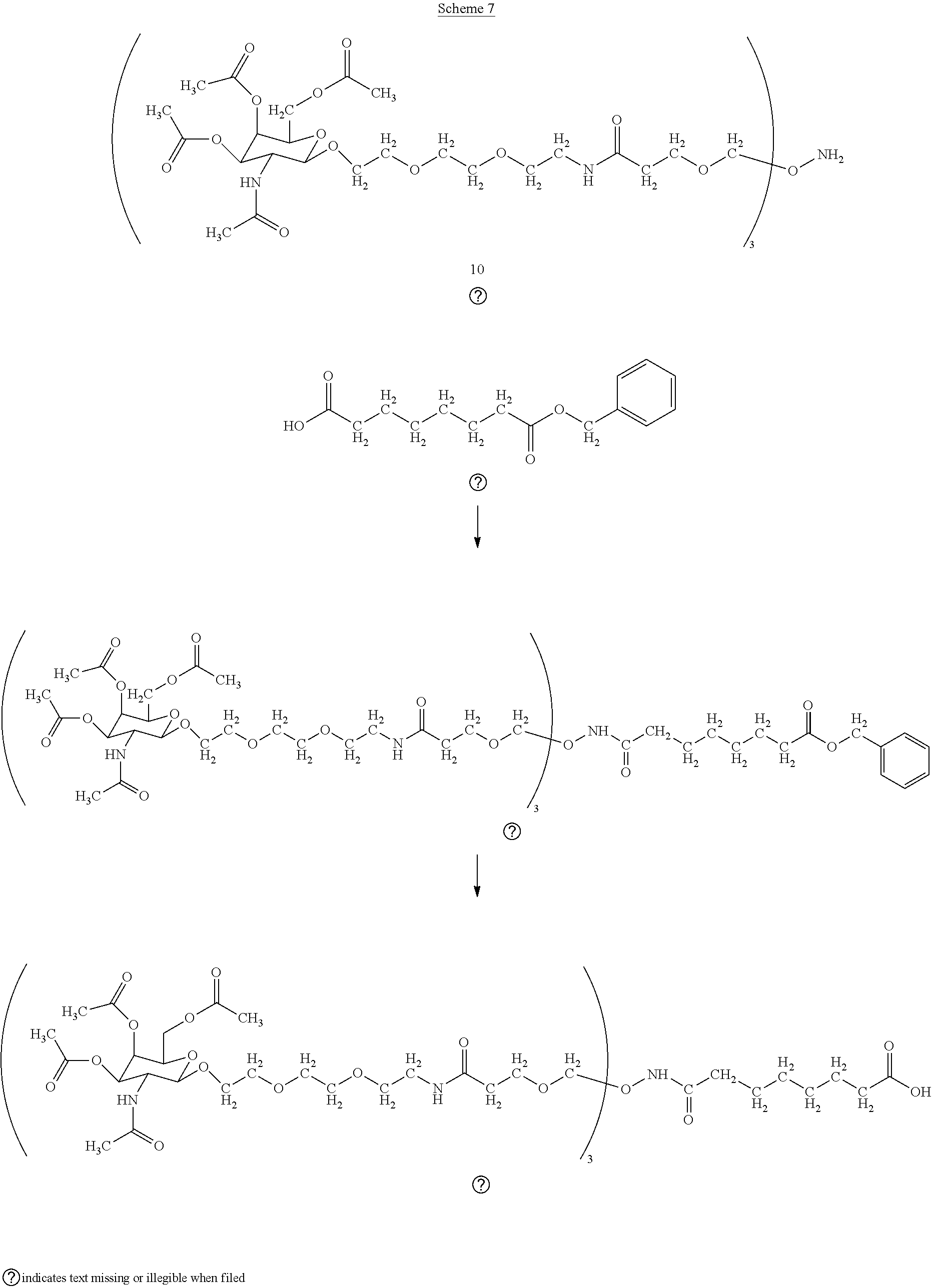

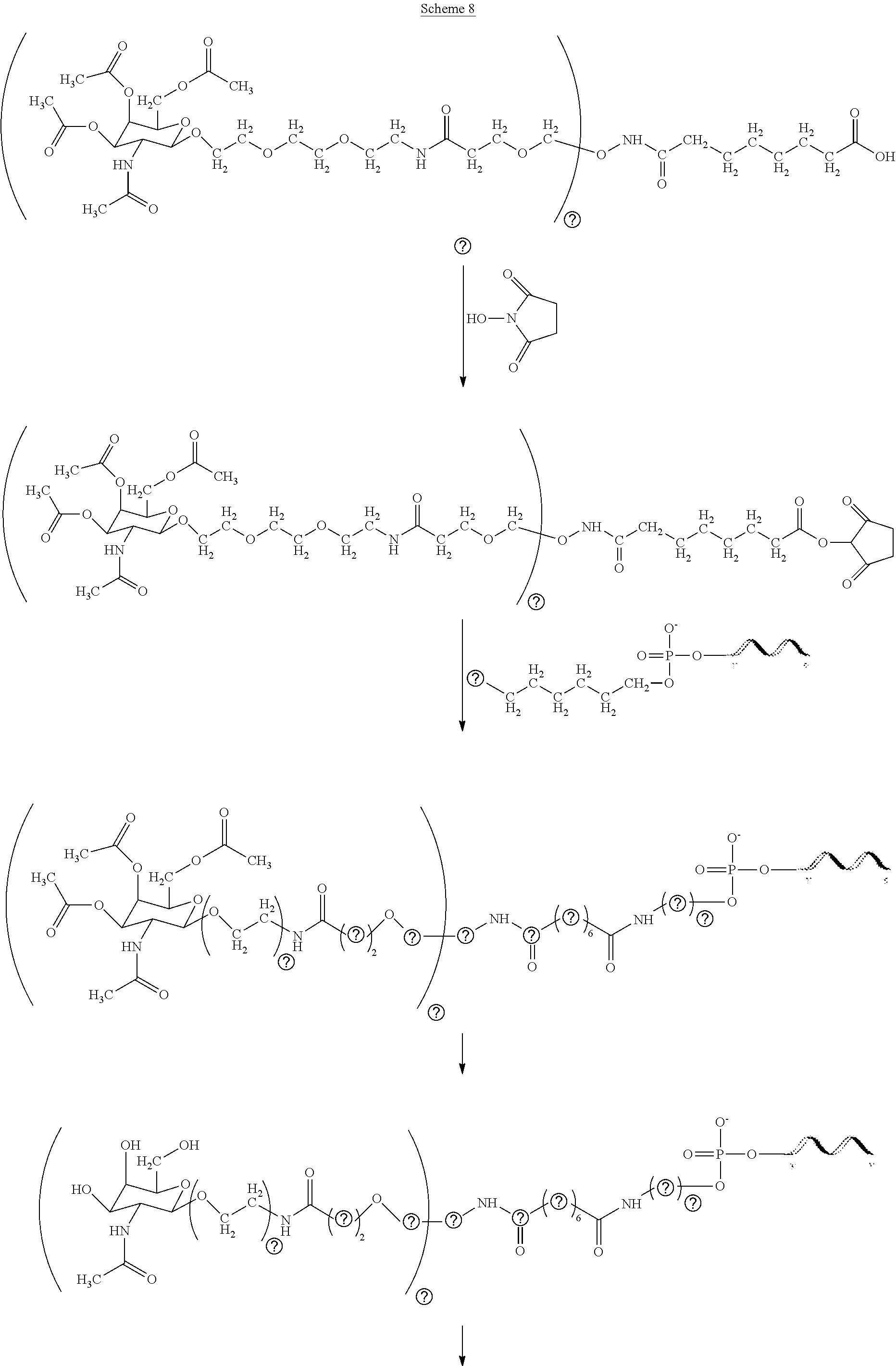

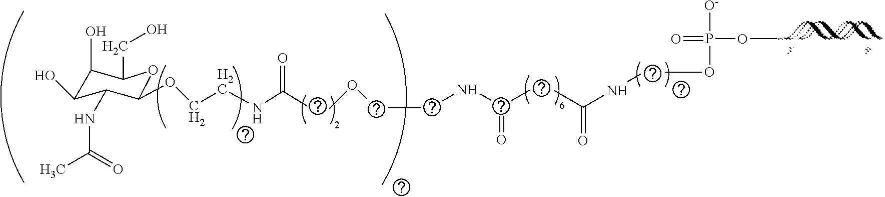

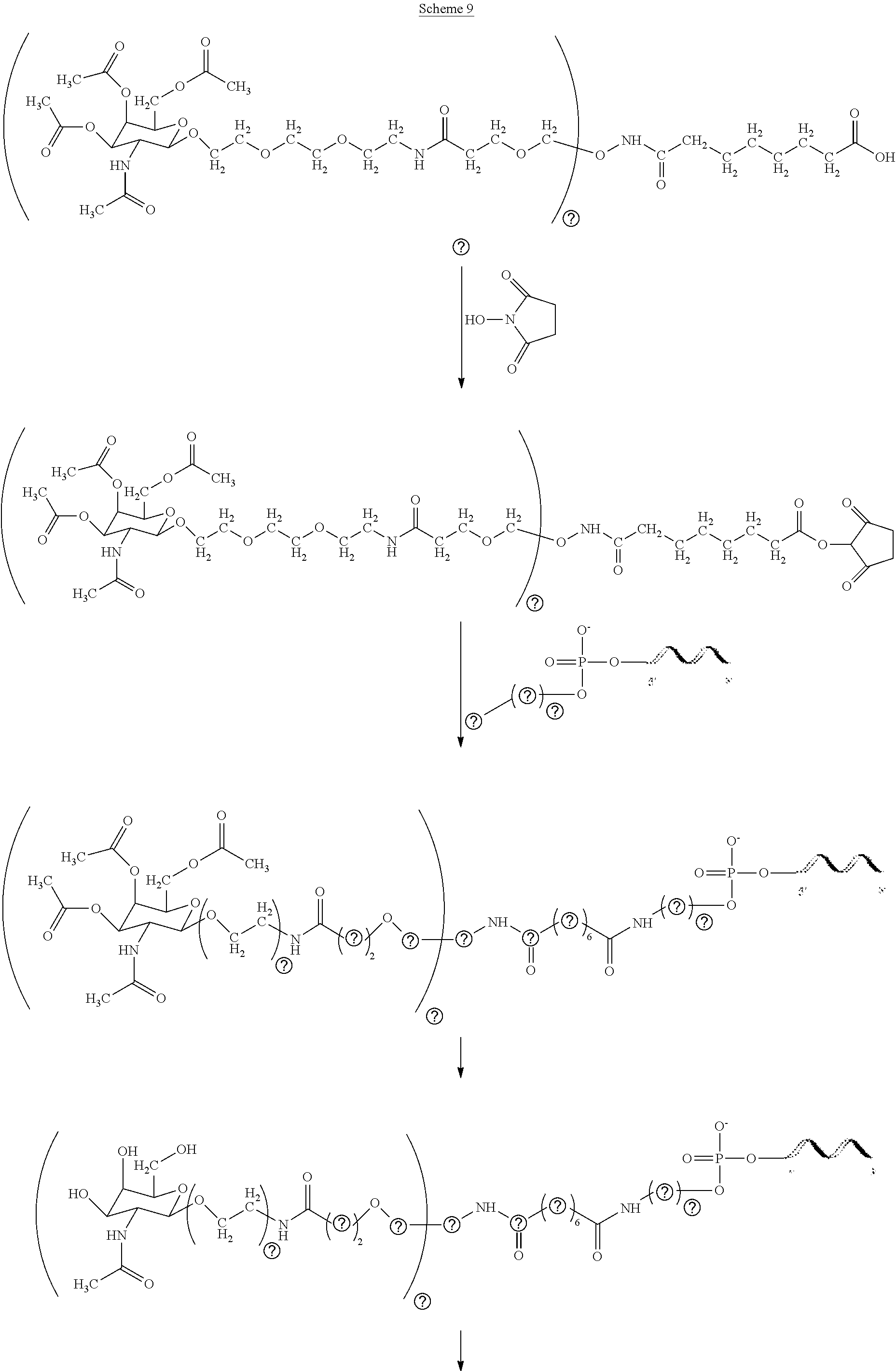

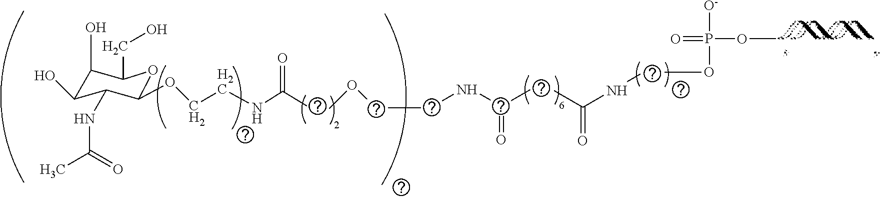

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Example 5

### Mouse Data for GalNAc-siRNA Constructs ETX005, ETX006, ETX014 and ETX015

ETX005 (Targeting HAO1 mRNA) T1a Inverted Abasic

An in vivo mouse pharmacology study was performed showing knockdown of HAO1 mRNA in liver tissue with an associated increase in serum glycolate level following a single subcutaneous dose of up to 3 mg/kg GalNAc conjugated modified siRNA ETX005.

Male C57BL/6 mice with an age of about 8 weeks were randomly assigned into groups of 21 mice. On day 0 of the study, the animals received a single subcutaneous dose of 0.3 or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was taken from a group of 5 untreated mice at day 0 to provide a baseline measurement of glycolate concentration.

Serum was stored at −80° C. until further analysis. Liver sample (approximately 50 mg) were treated with RNAlater and stored overnight at 4° C., before being stored at −80° C.

Liver samples were analysed using quantitative real-time PCR for HAO1 mRNA (Thermo assay ID Mm00439249_m1) and the housekeeping gene GAPDH mRNA (Thermo assay ID Mm99999915_g1). The delta delta Ct method was used to calculated changes in HAO1 expression normalised to GAPDH and relative to the saline control group.

A single 3 mg/kg dose of ETX005 inhibited HAO1 mRNA expression by greater than 80% after 7 days (FIG. 16). The suppression of HAO1 expression was durable, with a single 3 mg/kg dose of ETX005 maintaining greater than 60% inhibition of HAO1 mRNA at the end of the study on day 28. A single dose of 0.3 mg/kg ETX005 also inhibited HAO1 expression when compared with the saline control group, with HAO1 expression levels reaching normal levels only at day 28 of the study.

Suppression of HAO1 mRNA expression is expected to cause an increase in serum glycolate levels. Serum glycolate concentration was measured using LC-MS/MS (FIG. 17). A single 3 mg/kg dose of ETX005 caused a significant increase in serum glycolate concentration, reaching peak levels 14 days after dosing and remaining higher than baseline level (day 0) and the saline control group until the end of the study at day 28. A single 0.3 mg/kg dose of ETX005 showed a smaller and more transient increase in serum glycolate concentration above the level seen in a baseline and saline control group, demonstrating that a very small dose can suppress HAO1 mRNA at a magnitude sufficient to affect the concentration of a metabolic biomarker in serum.

FIG. 16. Single dose mouse pharmacology of ETX005. HAO1 mRNA expression is shown relative to the saline control group. Each point represents the mean and standard deviation of 3 mice.

FIG. 17. Single dose mouse pharmacology of ETX005. Serum glycolate concentration is shown. Each point represents the mean and standard deviation of 3 mice, except for baseline glycolate concentration (day 0) which was derived from a group of 5 mice.

ETX006 (Targeting HAO1 mRNA) T2a Inverted Abasic

An in vivo mouse pharmacology study was performed showing knockdown of HAO1 mRNA in liver tissue and a concomitant increase in serum glycolate levels following a single subcutaneous dose of up to 3 mg/kg GalNAc conjugated modified siRNA ETX006.

Male C57BL/6 mice with an age of about 8 weeks were randomly assigned into groups of 21 mice. On day 0 of the study, the animals received a single subcutaneous dose of 0.3 or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was taken from a group of 5 untreated mice at day 0 to provide a baseline measurement of glycolate concentration.

Serum was stored at −80° C. until further analysis. Liver sample (approximately 50 mg) were treated with RNAlater and stored overnight at 4° C., before being stored at −80° C.

Liver samples were analysed using quantitative real-time PCR for HAO1 mRNA (Thermo assay ID Mm00439249_m1) and the housekeeping gene GAPDH mRNA (Thermo assay ID Mm99999915_g1). The delta delta Ct method was used to calculated changes in HAO1 expression normalised to GAPDH and relative to the saline control group.

A single 3 mg/kg dose of ETX006 inhibited HAO1 mRNA expression by than 80% after 7 days (FIG. 18). The suppression of HAO1 expression was durable and continued until the end of the study, with ETX006 maintaining greater than 60% inhibition of HAO1 mRNA at day 28. A single dose of 0.3 mg/kg also inhibited HAO1 expression when compared with the saline control group, with HAO1 expression levels reaching normal levels only at day 28 of the study.

Suppression of HAO1 mRNA expression is expected to cause an increase in serum glycolate levels. Serum glycolate concentration was measured using LC-MS/MS (FIG. 19). A single 3 mg/kg dose of ETX006 caused a significant increase in serum glycolate concentration, reaching peak levels 14 days after dosing and remaining higher than baseline levels (day 0) and the saline control group until the end of the study at day 28. A single 0.3 mg/kg dose of ETX006 showed a smaller and more transient increase in serum glycolate concentration above the level seen in a baseline and saline control groups, demonstrating that a very small dose can also affect the concentration of a metabolic biomarker in serum.

FIG. 18. Single dose mouse pharmacology of ETX006. HAO1 mRNA expression is shown relative to the saline control group. Each point represents the mean and standard deviation of 3 mice.

FIG. 19. Single dose mouse pharmacology of ETX006. Serum glycolate concentration is shown. Each point represents the mean and standard deviation of 3 mice, except for baseline glycolate concentration (day 0) which was derived from a group of 5 mice.

ETX014 (Targeting C5 mRNA) T1a Inverted Abasic

An in vivo mouse pharmacology study was performed showing knockdown of C5 mRNA in liver tissue and the resulting decrease in serum C5 protein concentration following a single subcutaneous dose of up to 3 mg/kg GalNAc conjugated modified siRNA ETX014.

Male C57BL/6 mice with an age of about 8 weeks were randomly assigned into groups of 21 mice. On day 0 of the study, the animals received a single subcutaneous dose of 0.3, 1, or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was stored at −80° C. until further analysis. Liver sample (approximately 50 mg) were treated with RNAlater and stored overnight at 4° C., before being stored at −80° C.

Liver samples were analysed using quantitative real-time PCR for C5 mRNA (Thermo assay ID Mm00439275_m1) and the housekeeping gene GAPDH mRNA (Thermo assay ID Mm99999915_g1). The delta delta Ct method was used to calculated changes in C5 expression normalised to GAPDH and relative to the saline control group.

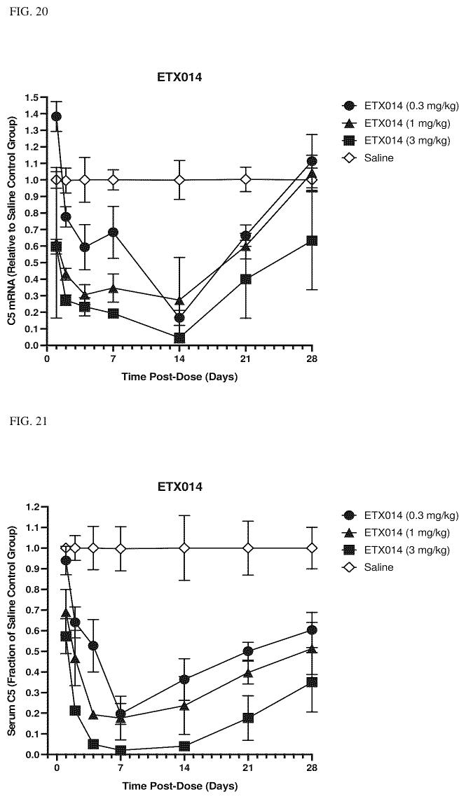

ETX014 inhibited C5 mRNA expression in a dose-dependent manner (FIG. 20) with the 3 mg/kg dose achieving greater than 90% reduction in C5 mRNA at day 14. The suppression of C5 expression by ETX014 was durable, with the 3 mg/kg dose of each molecule showing clear knockdown of C5 mRNA until the end of the study at day 28.

For C5 protein level analysis, serum samples were measured using a commercially available C5 ELISA kit (Abcam ab264609). Serum C5 levels were calculated relative to the saline group means at matching timepoints.

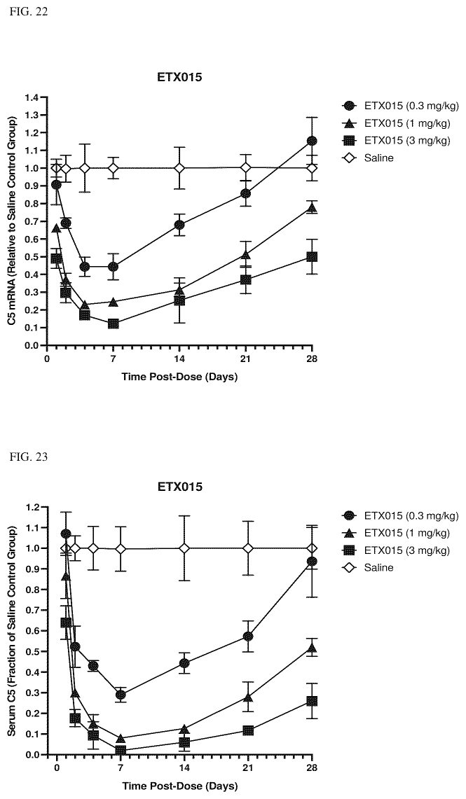

Serum protein data support the mRNA analysis (FIG. 21). Treatment with ETX014 caused a dose-dependent decrease in serum C5 protein concentration. All doses of ETX014 reduced C5 protein levels by greater than 70%, with the 3 mg/kg dose reducing C5 levels to almost undetectable levels at day 7 of the study. Reduction of serum C5 was sustained by all doses until study termination, with even the lowest dose of 0.3 mg/kg still showing inhibition of approximately 40% at day 28.

FIG. 20. Single dose mouse pharmacology of ETX014. C5 mRNA expression is shown relative to the saline control group. Each point represents the mean and standard deviation of 3 mice.

FIG. 21. Single dose mouse pharmacology of ETX0014. Serum C5 concentration is shown relative to the saline control group. Each point represents the mean and standard deviation of 3 mice.

ETX015 (Targeting C5 mRNA) T2a Inverted Abasic

An in vivo mouse pharmacology study was performed showing knockdown of C5 mRNA in liver tissue and the resulting decrease in serum C5 protein concentration following a single subcutaneous dose of up to 3 mg/kg GalNAc conjugated modified siRNA ETX015.

Male C57BL/6 mice with an age of about 8 weeks were randomly assigned into groups of 21 mice. On day 0 of the study, the animals received a single subcutaneous dose of 0.3, 1, or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was stored at −80° C. until further analysis. Liver sample (approximately 50 mg) were treated with RNAlater and stored overnight at 4° C., before being stored at −80° C.

Liver samples were analysed using quantitative real-time PCR for C5 mRNA (Thermo assay ID Mm00439275_m1) and the housekeeping gene GAPDH mRNA (Thermo assay ID Mm99999915_g1). The delta delta Ct method was used to calculated changes in C5 expression normalised to GAPDH and relative to the saline control group.

ETX015 inhibited C5 mRNA expression in a dose-dependent manner (FIG. 22) with the 3 mg/kg dose achieving greater than 85% reduction of C5 mRNA. The suppression of C5 expression was durable, with the 3 mg/kg dose of each molecule showing clear knockdown of C5 mRNA until the end of the study at day 28. Mice dosed with 3 mg/kg ETX015 still exhibited less than 50% of normal liver C5 mRNA levels 28 days after dosing.

For C5 protein level analysis, serum samples were measured using a commercially available C5 ELISA kit (Abcam ab264609). Serum C5 levels were calculated relative to the saline group means at matching timepoints.

Serum protein data support the mRNA analysis (FIG. 23). ETX015 caused a dose-dependent decrease in serum C5 protein concentration. The 3 mg/kg and 1 mg/kg doses of ETX015 achieved greater than 90% reduction of serum C5 protein levels. The highest dose exhibited durable suppression of C5 protein expression, with a greater than 70% reduction of C5 at day 28 of the study compared to saline control.

FIG. 22. Single dose mouse pharmacology of ETX015. C5 mRNA expression is shown relative to the saline control group. Each point represents the mean and standard deviation of 3 mice.

FIG. 23. Single dose mouse pharmacology of ETX0014. Serum C5 concentration is shown relative to the saline control group. Each point represents the mean and standard deviation of 3 mice.

### Example 6

### NHP Data for GalNAc-siRNA Constructs ETX023 and ETX024

ETX023 (Targeting TTR mRNA) T1a Inverted Abasic

ETX023 pharmacology was evaluated in non-human primate (NHP) by quantifying serum transthyretin (TTR) protein levels. A single subcutaneous dose of 1 mg/kg GalNAc conjugated modified siRNA ETX023 demonstrated durable suppression of TTR protein expression.

Male cynomolgus monkeys (3-5 years old, 2-3 kg) were assigned into groups of 3 animals. Animals were acclimatised for 2 weeks, and blood taken 14 days prior to dosing to provide baseline TTR concentration. A liver biopsy was performed 18 or 38 days prior to dosing to provide baseline mRNA levels. On day 0 of the study, the animals received a single subcutaneous dose of 1 mg/kg GalNAc-siRNA ETX023 dissolved in saline (sterile 0.9% sodium chloride). At day 3, day 14, day 28, day 42, day 56, day 70 and day 84 of the study, a liver biopsy was taken and RNA extracted for measurement of TTR mRNA. At day 1, day 3, day 7, day 14, day 28, day 42, day 56, day 70 and day 84 of the study, a blood sample was taken for measurement of serum TTR concentration and clinical blood chemistry analysis.

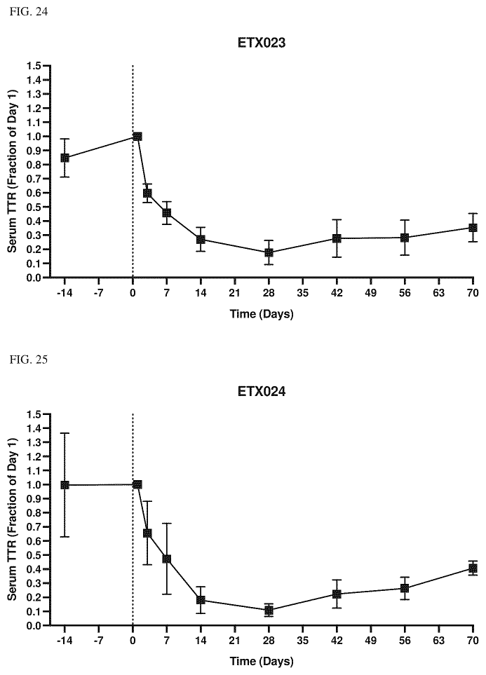

Suppression of TTR mRNA expression is expected to cause a decrease in serum TTR protein levels. Serum TTR protein concentration was measured by a commercially available ELISA kit (Abcam ab231920). TTR concentration as a fraction of day 1 was calculated for each individual animal and this was plotted as mean and standard deviation for the group of 3 animals (FIG. 24).

A single 1 mg/kg dose of ETX023 caused a rapid and significant reduction in serum TTR concentration, reaching nadir 28 days after dosing and remaining suppressed until day 70.

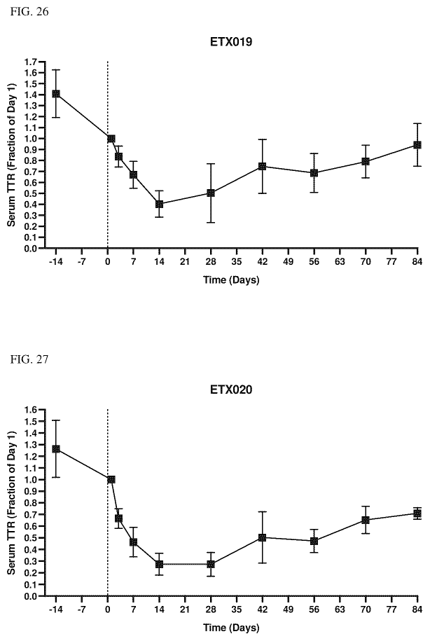

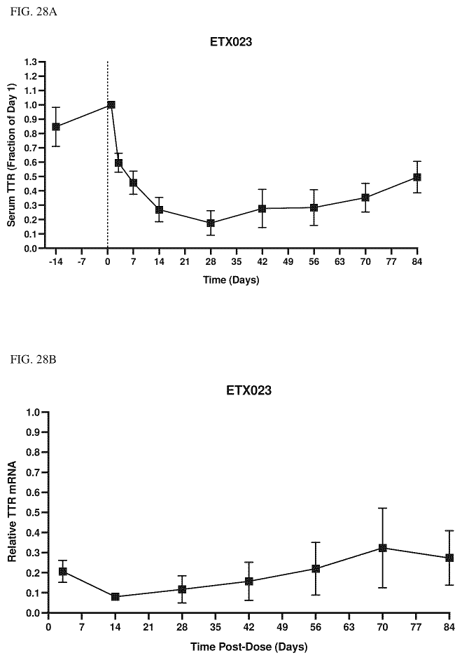

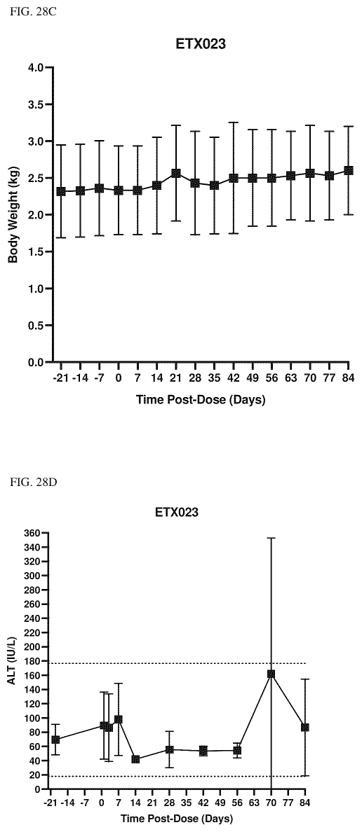

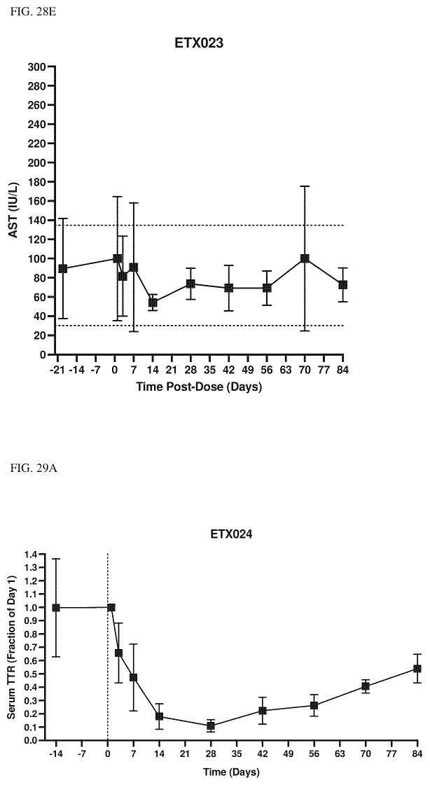

Data was further obtained until day 84. Identical experiments were carried out using ETX019. Data is provided for 84 days in FIG. 26 for ETX0019 and FIG. 28aA for ETX0023.

TTR mRNA was measured by real-time quantitative PCR using a TaqMan Gene expression kit TTR (Thermo, assay ID Mf02799963_m1). GAPDH expression was also measured (Thermo, assay ID Mf04392546_g1) to provide a reference. Relative TTR expression for each animal was calculated normalised to GAPDH and relative to pre-dose levels by the ΔΔCt method. A single 1 mg/kg dose of ETX023 also caused a rapid and significant reduction in liver TTR mRNA, reaching nadir 14 days after dosing and remaining suppressed until day 84 (FIG. 28bB).

Animal body weight was measured once a week during the study. No fluctuations or decrease in body weight was associated with dosing ETX023 and animals continued to gain weight throughout the study (FIG. 28cC).

Serum was analysed within 2 hours using an automatic biochemical analyser. A significant increase in ALT (alanine transaminase) and AST (aspartate transaminase) are commonly used to demonstrate liver toxicity. No increase in ALT (FIG. 28dD) or ALT (FIG. 28eE) was associated with dosing of ETX023.

ETX024 (Targeting TTR mRNA) T2a Inverted Abasic

ETX024 pharmacology was evaluated in non-human primate (NHP) by quantifying serum transthyretin (TTR) protein levels. A single subcutaneous dose of 1 mg/kg GalNAc conjugated modified siRNA ETX024 demonstrated durable suppression of TTR protein expression.

Male cynomolgus monkeys (3-5 years old, 2-3 kg) were assigned into groups of 3 animals. Animals were acclimatised for 2 weeks, and blood taken 14 days prior to dosing to provide baseline TTR concentration. A liver biopsy was performed 18 or 38 days prior to dosing to provide baseline mRNA levels. On day 0 of the study, the animals received a single subcutaneous dose of 1 mg/kg GalNAc-siRNA ETX024 dissolved in saline (sterile 0.9% sodium chloride). At day 3, day 14, day 28, day 42, day 56, day 70 and day 84 of the study, a liver biopsy was taken and RNA extracted for measurement of TTR mRNA. At day 1, day 3, day 7, day 14, day 28, day 42, day 56, 70 and day 84 of the study, a blood sample was taken for measurement of serum TTR concentration and clinical blood chemistry analysis.

Suppression of TTR mRNA expression is expected to cause a decrease in serum TTR protein levels. Serum TTR protein concentration was measured by a commercially available ELISA kit (Abcam ab231920). TTR concentration as a fraction of day 1 was calculated for each individual animal and this was plotted as mean and standard deviation for the group of 3 animals (FIG. 25).

A single 1 mg/kg dose of ETX024 caused a rapid and significant reduction in serum TTR concentration, reaching nadir 28 days after dosing and remaining suppressed until day 70.

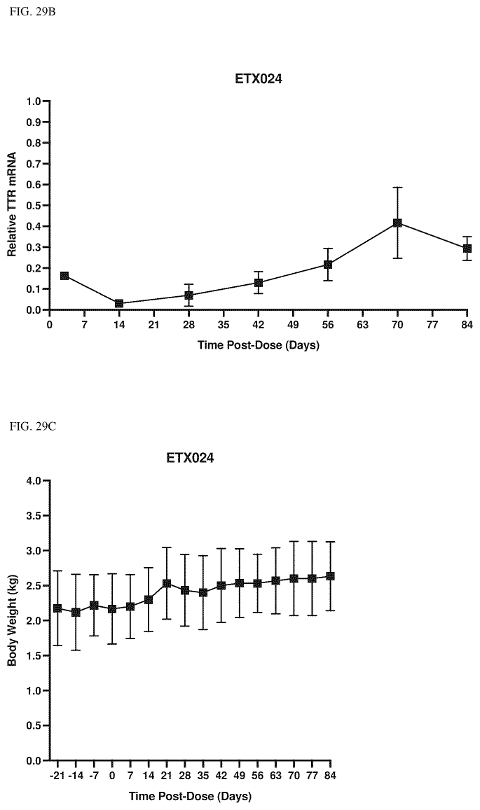

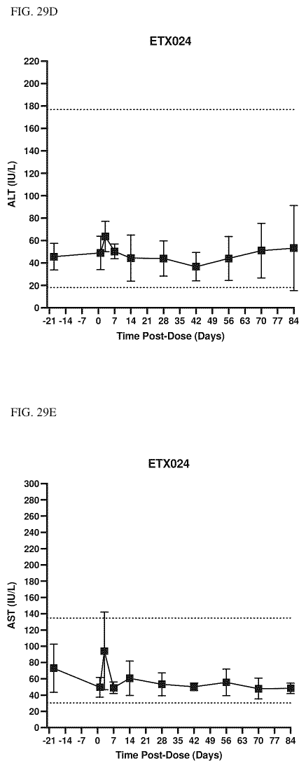

Data was further obtained with ETX024 until day 84. Identical experiments were carried out using ETX020. Data is provided for 84 days in FIG. 27 for ETX0020 and FIG. 29aA for ETX0024.

TTR mRNA was measured by real-time quantitative PCR using a TaqMan Gene expression kit TTR (Thermo, assay ID Mf02799963_m1). GAPDH expression was also measured (Thermo, assay ID Mf04392546_g1) to provide a reference. Relative TTR expression for each animal was calculated normalised to GAPDH and relative to pre-dose levels by the ΔΔCt method. A single 1 mg/kg dose of ETX024 caused a rapid and significant reduction in liver TTR mRNA, reaching nadir 14 days after dosing and remaining suppressed until day 84 (FIG. 29bB).

Animal body weight was measured once a week during the study. No fluctuations or decrease in body weight was associated with dosing ETX024 and animals continued to gain weight throughout the study (FIG. 29cC).

Serum was analysed within 2 hours using an automatic biochemical analyser. A significant increase in ALT (alanine transaminase) and AST (aspartate transaminase) are commonly used to demonstrate liver toxicity. No increase in ALT (FIG. 29dD) or ALT (FIG. 29eE) was associated with dosing of ETX024.

In preferred aspects, compounds of the invention are able to depress serum protein level of a target protein to a value below the initial (starting) concentration at day 0, over a period of up to at least about 14 days after day 0, up to at least about 21 days after day 0, up to at least about 28 days after day 0, up to at least about 35 days after day 0, up to at least about 42 days after day 0, up to at least about 49 days after day 0, up to at least about 56 days after day 0, up to at least about 63 days after day 0, up to at least about 70 days after day 0, up to at least about 77 days after day 0, or up to at least about 84 days after day 0, hereinafter referred to as the “dose duration”. “Day 0” as referred to herein is the day when dosing of a compound of the invention to a patient is initiated, in other words the start of the dose duration or the time post dose.

In preferred aspects, compounds of the invention are able to depress serum protein level of a target protein to a value of at least about 90% or below of the initial (starting) concentration at day 0, such as at least about 85% or below, at least about 80% or below, at least about 75% or below, at least about 70% or below, at least about 65% or below, at least about 60% or below, at least about 55% or below, at least about 50% or below, at least about 45% or below, at least about 40% or below, at least about 35% or below, at least about 30% or below, at least about 25% or below, at least about 20% or below, at least about 15% or below, at least about 10% or below, at least about 5% or below, of the initial (starting) concentration at day 0. Typically such depression of serum protein can be maintained over a period of up to at least about 14 days after day 0, up to at least about 21 days after day 0, up to at least about 28 days after day 0, up to at least about 35 days after day 0, up to at least about 42 days after day 0, up to at least about 49 days after day 0, up to at least about 56 days after day 0, up to at least about 63 days after day 0, up to at least about 70 days after day 0, up to at least about 77 days after day 0, or up to at least about 84 days after day 0. More preferably, at a period of up to at least about 84 days after day 0, the serum protein can be depressed to a value of at least about 90% or below of the initial (starting) concentration at day 0, such as at least about 85% or below, at least about 80% or below, at least about 75% or below, at least about 70% or below, at least about 65% or below, at least about 60% or below, at least about 55% or below, at least about 50% or below, at least about 45% or below, at least about 40% or below, of the initial (starting) concentration at day 0.

In preferred aspects, compounds of the invention are able to achieve a maximum depression of serum protein level of a target protein to a value of at least about 50% or below of the initial (starting) concentration at day 0, such as at least about 45% or below, at least about 40% or below, at least about 35% or below, at least about 30% or below, at least about 25% or below, at least about 20% or below, at least about 15% or below, at least about 10% or below, at least about 5% or below, of the initial (starting) concentration at day 0. Typically such maximum depression of serum protein occurs at about day 14 after day 0, at about day 21 after day 0, at about day 28 after day 0, at about day 35 after day 0, or at about day 42 after day 0. More typically, such maximum depression of serum protein occurs at about day 14 after day 0, at about day 21 after day 0, or at about day 28 after day 0.

Specific compounds of the invention can typically achieve a maximum % depression of serum protein level of a target protein and/or a % depression over a period of up to at least about 84 days as follows:

ETX019 can typically achieve at least 50% depression of serum protein level of a target protein, typically TTR, typically at about 7 to 21 days after day 0, in particular at about 14 days after day 0, and/or can typically maintain at least 90% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);

ETX020 can typically achieve at least 30% depression of serum protein level of a target protein, typically TTR, typically at about 7 to 21 days after day 0, in particular at about 14 days after day 0, and/or can typically maintain at least 80% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);

ETX023 can typically achieve at least 20% depression of serum protein level of a target protein, typically TTR, typically at about 7 to 21 days after day 0, in particular at about 14 days after day 0, and/or can typically maintain at least 50% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);

ETX024 can typically achieve at least 20% depression of serum protein level of a target protein, typically TTR, typically at about 7 to 21 days after day 0, in particular at about 14 days after day 0, and/or can typically maintain at least 60% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose).

Suitably the depression of serum level is determined in non-human primates by delivering a single subcutaneous dose of 1 mg/kg of the relevant active agent, eg ETX0023 or ETX0024, dissolved in saline (sterile 0.9% sodium chloride). Suitable methods are described herein. It will be appreciated that this is not limiting and other suitable methods with appropriate controls may be used.

### Example 7 ETX023 (Targeting TTR mRNA) T1a Inverted Abasic

Total bilirubin levels remained stable throughout the study (FIG. 34)

Kidney health was monitored by assessment of urea (blood urea nitrogen, BUN) and creatinine concentration throughout the study. Both blood urea concertation (BUN) and creatinine levels remained stable and within the expected range after a single 1 mg/kg dose of ETX023 (FIGS. 35 and 36).

### Example 8 ETX024 (Targeting TTR mRNA) T2a Inverted Abasic

Total bilirubin levels remained stable throughout the study (FIG. 37)

Kidney health was monitored by assessment of urea (blood urea nitrogen, BUN) and creatinine concentration throughout the study. Both blood urea concertation (BUN) and creatinine levels remained stable and within the expected range after a single 1 mg/kg dose of ETX024 (FIGS. 38 and 39).

A further aspect of the invention is described below, with non-limiting examples described in the following FIGS. 49-42FIGS. 40A-42D and Examples 9-18. The compounds described below are suitable for use in any of the aspects and embodiments disclosed above, for example in respect of the uses, nucleic acid lengths, definitions, pharmaceutically acceptable compositions, dosing, methods for inhibiting gene expression, and methods of treating or preventing diseases associated with gene expression, unless otherwise immediately apparent from the disclosure.

FIG. 40A depicts a tri-antennary GalNAc (N-acetylgalactosamine) unit.

FIG. 40B depicts an alternative tri-antennary GalNAc according to one embodiment of the invention, showing variance in linking groups.

FIG. 41A depicts tri-antennary GalNAc-conjugated siRNA according to the invention, showing variance in the linking groups.

FIG. 41B depicts a genera of tri-antennary GalNAc-conjugated siRNAs according to one embodiment of the invention.

FIG. 41C depicts a genera of bi-antennary GalNAc-conjugated siRNAs according to one embodiment of the invention, showing variance in the linking groups.

FIG. 41D depicts a genera of bi-antennary GalNAc-conjugated siRNAs according to another embodiment of the invention, showing variance in the linking groups.

FIG. 42A depicts another embodiment of the tri-antennary GalNAc-conjugated siRNA according to one embodiment of the invention.

FIG. 42B depicts a variant shown in FIG. 27A, having an alternative branching GalNAc conjugate.

FIG. 42C depicts a genera of tri-antennary GalNAc-conjugated siRNAs according to one embodiment of the invention, showing variance in the linking groups.

FIG. 42D depicts a genera of bi-antennary GalNAc-conjugated siRNAs according to one embodiment the invention, showing variance in the linking groups.

The further aspect discloses forms of ASGP-R ligand-conjugated, chemically modified RNAi agents, and methods of making and uses of such conjugated molecules.

In certain embodiments, the ASGP-R ligand comprises N-acetylgalactosamine (GalNAc). In certain embodiments, the invention provides an siRNA conjugated to tri-antennary or biantennary units of GalNAc of the following formula (I):

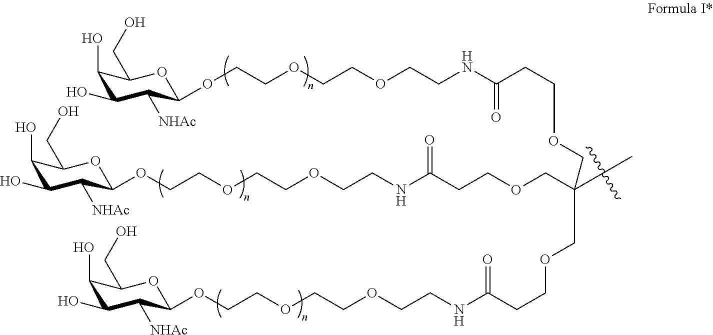

In Formula I*, n is 0, 1, 2, 3, or 4. In some embodiments, the number of the ethylene-glycol units may vary independently from each other in the different branches. For example, the middle branch may have n=4, while the side branches may have n=3, etc. Other embodiments my contain only two branches, as depicted in Formulae (II-a)

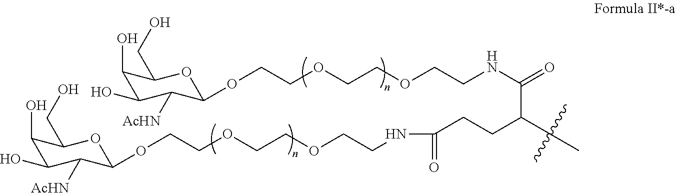

In Formulae II* and II*-a, n is chosen from 0, 1, 2, 3, or 4. In some embodiments, the number of the ethylene-glycol units may vary independently from each other in the different branches. For example, the one branch may have n=4 or 3, while the other branche(s) may have n=3 or 2, etc.

Additional GalNAc branches can also be added. for example, 4-, 5-, 6-, 7-, 8-, 9-branched GalNAc units may be used.

In related embodiments, the branched GalNAc can be chemically modified by the addition of another targeting moiety, e.g., a lipids, cholesterol, a steroid, a bile acid, targeting (poly)peptide, including polypeptides and proteins. (e.g., RGD peptide, transferrin, polyglutamate. polyaspartate, glycosylated peptide, biotin, asialoglycoprotein insulin and EGF.

Option 1. In further embodiments, the GalNAc units may be attached to the RNAi agent via a tether, such as the one shown in Formula (III*):

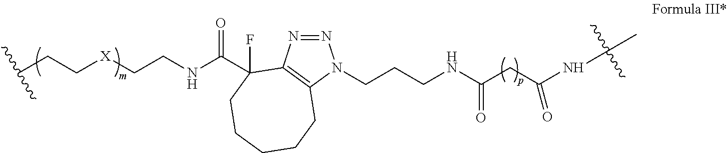

In Formula III*, m is chosen from 0, 1, 2, 3, 4, or 5, and p is chosen from 0, 1, 2, 3, 4, 5, 6, 7, 8, 9, 10, 11, 12, 13, or 14, independently of m, and X is either CH₂ or O.

In yet further embodiments, the tether can attach to the oligo via phosphate (Z═O) or a phosphorothioate group (Z═S), as shown in formula (IV*):

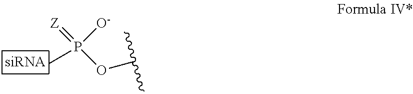

Such an attachment of the GalNAc branched units via the specified tethers is preferably at a 3′ or a 5′ end of the sense strand of the RNAi agent. In one embodiment, the attachment to the 3′ of RNAi agent is through C6 amino linker as shown in Formula (V*):

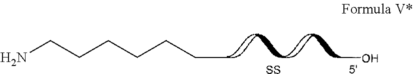

This linker is the starting point of the synthesis as shown in Example 15.

The same linkers and tethers as described above can be used with alternative branched GalNAc structures as shown in Formulas VI* and VII*:

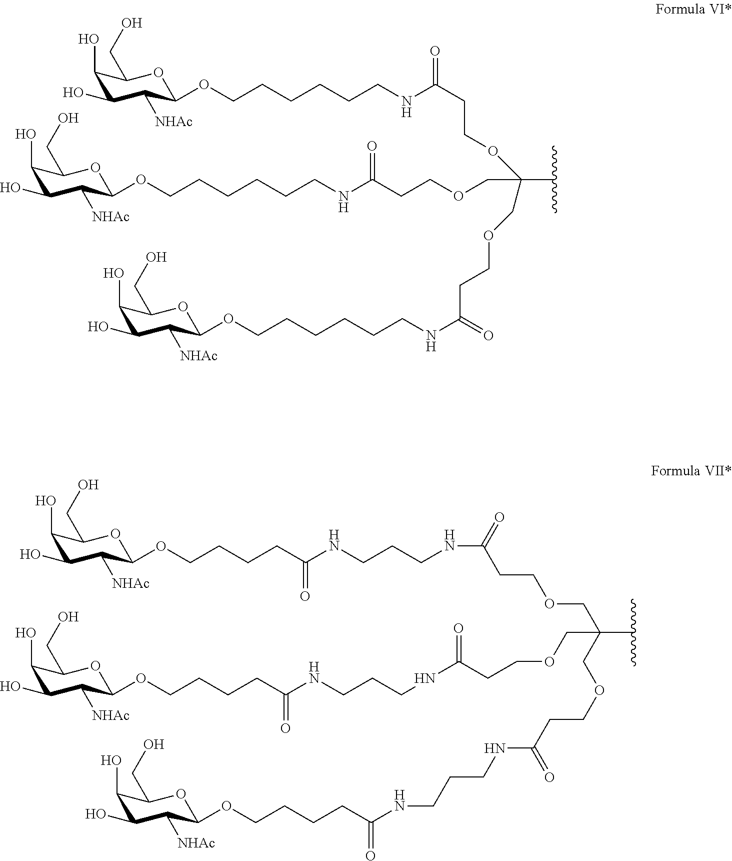

Similarly to Formula II*-a, a bi-antennary form of ligand based on Formulae VI* and VII* can be used in the compositions of the invention.

Option 2. In further embodiments. the GalNAc units may be attached to the RNAi agent via a tether, such as the one shown in Formula (III*-2):

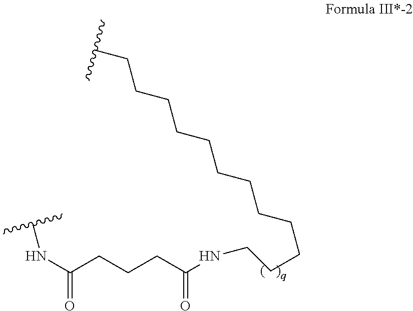

In Formula II*-2, q is chosen from 1, 2, 3, 4, 5, 6, 7 or 8.

In yet further embodiments, the tether can attach to the oligo via phosphate (Z═O) or a phosphorothioate group (Z═S), as shown in formula (IV*):

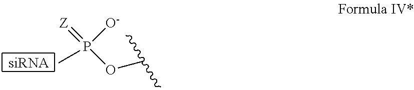

Such an attachment of the GalNAc branched units via the specified tethers preferably at a 3′ or a 5′ end of the sense strand of the double stranded RNAi agent. In one embodiment, the attachment to the 3′ of RNAi agent is as shown in Example 17. In one embodiment when the GalNAc tether is at attached to the 3′ site, the transitional linker between the tether and the 3′ end of the oligo comprises the structure of the formula (V*-a; see also FIG. 42C) or another suitable linker may be used, for example, C6 amino linker shown in Formula (V*-b):

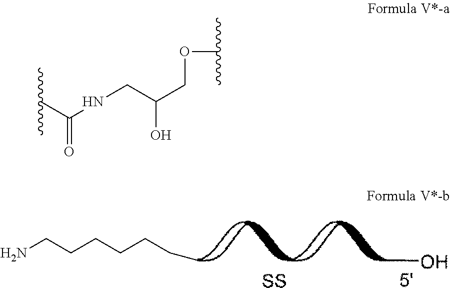

Additional and/or alternative conjugation sites may include any non-terminal nucleotide, including sugar residues, phosphate groups, or nucleic acid bases.

The same linkers and tether can be used with alternative branched GalNAc structures as shown in Formulas VI*-2 and VII*-2:

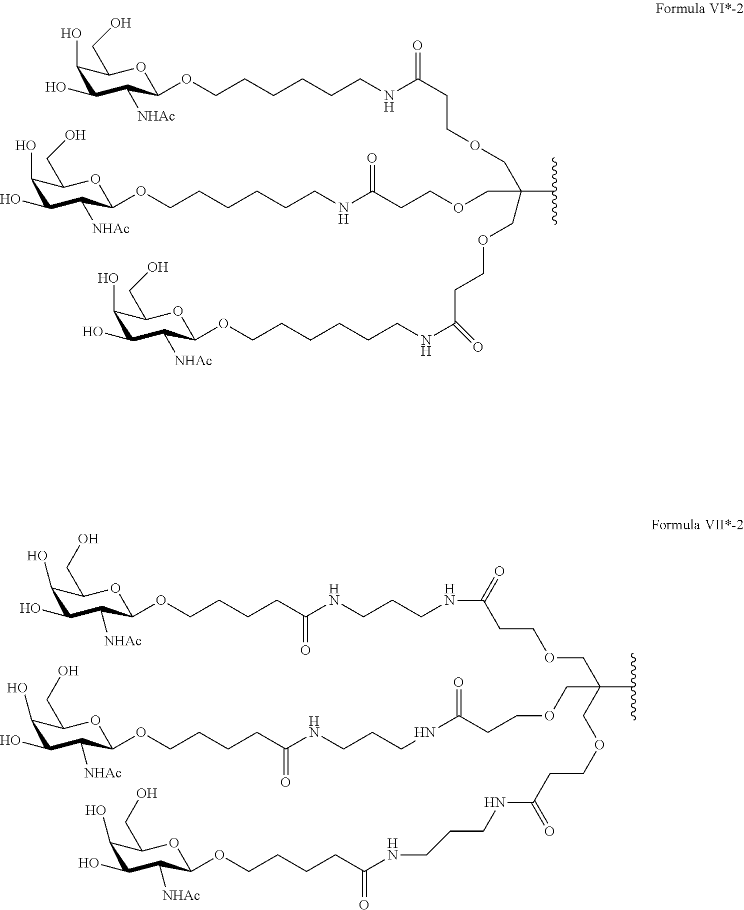

Characteristics of RNAi Agents of the Invention and their Chemical Modifications

In certain embodiments, the conjugated oligomeric compound (referred herein as RNA interference compound (RNAi compound)) comprises two strands, each having sequence of from 8 to 55 linked nucleotide monomer subunits (including inverted abasic (ia) nucleotide(s)) in either the antisense strand or in the sense strand. In certain embodiments, the conjugated oligomeric compound strands comprise, for example, a sequence of 16 to 55, 53, 49, 40, 25, 24, 23, 21, 20, 19, 18, 17, or up to (about) 18-25, 18-23, 21-23 linked nucleotide monomer subunits. In certain embodiments, RNAi agent of the invention may have a hairpin structure, having a single strand of the combined lengths of both strands as described above. (The term “nucleotide” as used throughout, may also refer to nucleosides (i.e., nucleotides without phosphate/phosphonothioate groups) where context so requires.)

In certain embodiments, the double stranded RNAi agent is blunt-ended or has an overhang at one or both ends. In some embodiments, the overhang is 1-6, 1-5, 1-4, 1-3, 2-4, 4, 3, 2 or 1 nucleotide(s) (at 3′ end or at 5′ end) of the antisense strand as well as 2-4, 3, or 2 or 1 nucleotide(s) (at 3′ end or at 5′ end) of the sense strand. In certain exemplary embodiments, see Ex. 9, constructs 9.1, 9.2, and 9.3, the RNAi agent comprises 2 nucleotide overhang at the 3′ end of the antisense strand and 2 nucleotide overhang at 3′ end of the sense strand. In certain other exemplary embodiments, see Ex. 10, constructs 10.1 and 10.3, Ex. 11, constructs 11.1 and 11.3; and Ex. 12, constructs 12.1 and 12.3, the RNAi agents comprise 2 nucleotide overhang at the 3′ end of the antisense strand and are blunt-ended on the other end. In certain other exemplary embodiment, see Ex. 10, construct 10.3, the construct is blunt-ended on both ends. In another exemplary embodiment, see Ex. 12, construct 12.2, the RNAi agent comprises 4 nucleotide overhang in the 3′ end of the antisense strand and blunt-ended on the other end.

In certain embodiments, the constructs are modified with a degradation protective moiety that prevents or inhibits nuclease cleavage by using a terminal cap, one or more inverted abasic nucleotides, one or more phosphorothioate linkages, one of more deoxynucleotides (e.g., D-ribonucleotide, D-2′-deoxyribonucleotide or another modified nucleotide), or a combination thereof. Such degradation protective moieties may be present at any one or all ends that are not conjugated to the ASGP-R ligand. In certain embodiments, the degradation protective moiety is chosen alone or as any combination from a group consisting of 1-4, 1-3, 1-2, or 1 phosphorothioate linkages, 1-4 1-3, 1-2, or 1 deoxynucleotides, and 1-4, 1-3, 1-2, or 1 inverted abasic nucleotides. In certain exemplary embodiments, the degradation protective moieties are configured as in one of the constructs 9.1, 9.2, 9.3, 10.1, 10.2, 10.3, 11.1, 11.2, 11.3, 12.1, 12.2, and 12.3, as shown in the Examples 9-18. Such exemplary protective moieties' configurations can be used in conjunction with any RNAi agents of the invention.

In certain embodiments, all or some riboses of the nucleotides in the sense and/or antisense strand (s) are modified. In certain embodiments, at least 50%, 60%, 70%, 80%, 90% or more (e.g., 100%) of riboses in the RNAi agent are modified. In certain embodiments, 1, 2, 3, 4, 5, 6, 7, 8, 9, 10 or more riboses are not modified.

In preferred embodiments, ribose modifications include 2′ substituent groups such as 2′-O-alkyl modifications, including 2′-O-methyl, and 2′-deoxyfluoro. Additional modifications are known in the art, including 2′-deoxy, LNA (e.g., 2′-O, 4′-C methylene bridge or 2′-O, 4′-C ethylene bridge), 2′-methoxythoxy (MOE), 2′-O—(CH2)OCH3, etc.

In certain embodiments, a number of modifications provide a distinct pattern of modifications, for example, as shown in constructs in the Examples 9-18, or as described in U.S. Pat. Nos. 7,452,987; 7,528,188; 8,273,866; 9,150,606; and 10,266,825; all of which are incorporated by reference herein.

In some embodiments, the siRNA comprises one or more thermally destabilizing nucleotides, e.g., GNA, ENA, etc., for example, at positions 11 (preferred), 12, 13 of the antisense strand and/or positions 9 and 10 (preferred) of the sense strand.

Additionally, nucleic acid bases could be modified, for example, at the C4 position as described in U.S. Pat. No. 10,119,136.

In general, the RNAi agents of the invention are directed against therapeutic targets, inhibition of which will result in prevention, alleviation, or treatment of a disease, including undesirable or pathological conditions. A great number of such targets is known in the art. Non-limiting examples of such targets include: ApoC, ApoB, ALAS1, TTR, GO, C5 (see Examples), etc. Generally, due to the abundant expression of ASGP-R on the surface of hepatocytes, such targets are preferably expressed in the liver, however, they could also be expressed in other tissues or organs. In preferred embodiments, targets are human, while the RNAi agent comprise an antisense strand fully or partially complementary to such a target. In certain embodiments, the RNAi agents may comprise two or more chemically linked RNAi agents directed against the same or different targets.

### EXAMPLES

### Example 9: Inverted Abasic Chemistry with 5′-GalNAc

In all RNAi agents depicted in the Examples, the following conventions are used:
ia=inverted abasic nucleotide;
m=2′-O-methyl nucleotide;
f=2′-deoxy-2′-fluoro nucleotide;
s=phosphorothioate internucleotide linkage;
Xd=2′-deoxy-nucleotide;
˜=tether.

Using standard synthesis techniques, the following constructs are synthesized in various versions, with tethers 1 and 2, and with various tri-antennary GalNAc units according to the invention, as described above or depicted in FIGS. 41A-42B.

```
9.1.
(Top strand (sense (ss)): SEQ ID NO: 1; bottom strand (antisense) SEQ ID NO: 2)
                  1 2 3 4 5 6 7 8 9 10 11 12 13 14 15 16 17 18 19 20 21 22 23
5′ GalNAc~Gm-Am-Cm-Um-Um-Um-Cf-Am-Uf-Cf-Cf-Um-Gm-Gm-Am-Am-Am-Um-AmsUmsAm-ia-ia 3′
3′ AmsCms-Cm-Um-Gm-Am-Am-Af-Gm-Uf-Am-Gm-Gm-Am-Cf-Cf-Um-Uf-Um-Am-UmsAfsUm 5′
      23 22 21 20 19 18 17 16 15 14 13 12 11 10 9 8 7 6 5 4 3 2 1
9.2.
(Top strand (sense (ss)): SEQ ID NO: 3; bottom strand (antisense) SEQ ID NO: 4)
                  1 2 3 4 5 6 7 8 9 10 11 12 13 14 15 16 17 18 19 20 21 22 23
5′ GalNAc~Am-Am-Gf-Cm-Af-Am-Gf-Am-Uf-Af-Uf-Um-Uf-Um-Um-Af-Um-Af-AmsUmsAm-ia-ia 3′
3′ Td-Td-UmsUmsUm-Um-Cm-Gf-Um-Uf-Cm-Uf-Am-Um-Am-Af-Am-Af-Am-Um-Af-Um-UfsAfsUm 5′
      25 25 23 22 21 20 19 18 17 16 15 14 13 12 11 10 9 8 7 6 5 4 3 2 1
9.3.
(Top strand (sense (ss)): SEQ ID NO: 5; bottom strand (antisense) SEQ ID NO: 6)
                  1 2 3 4 5 6 7 8 9 10 11 12 13 14 15 16 17 18 19 20 21 22 23
5′ GalNAc~Um-Gm-Gm-Gm-Am-Um-Uf-Um-Cf-Af-Uf-Gm-Um-Am-Am-Cm-Cm-Am-AmsGmsAm-ia-ia 3′
3′ CmsUmsAm-Cm-Cm-Cm-Um-Af-Am-Af-Gm-Um-Am-Cm-Af-Um-Um-Gf-Gm-Um-UmsCfsUm 5′
      23 22 21 20 19 18 17 16 15 14 13 12 11 10 9 8 7 6 5 4 3 2 1
```

### Example 10: Inverted Abasic Chemistry with 3′-GalNAc

Using standard synthesis techniques, the following constructs are synthesized in various versions, with tethers 1 and 2 according to the invention, and with various tri-antennary GalNAc units according to the invention, as described above or depicted in FIGS. 41A-42B. Same sequences as in Example 9 are shown for consistency.

```
10.1
(Top strand (sense (ss)): SEQ ID NO: 7; bottom strand (antisense) SEQ ID NO: 8)
      1 2 3 4 5 6 7 8 9 10 11 12 13 14 15 16 17 18 19 20 21 22 23
5′ ia-ia-GmsAmsCm-Um-Um-Um-Cf-Am-Uf-Cf-Cf-Um-Gm-Gm-Am-Am-Am-Um-Am-Um-Am~GalNAc 3′
3′ AmsCmsCm-Um-Gm-Am-Am-Af-Gm-Uf-Am-Gm-Gm-Am-Cf-Cf-Um-Uf-Um-Am-UmsAfsUm 5′
      23 22 21 20 19 18 17 16 15 14 13 12 11 10 9 8 7 6 5 4 3 2 1
10.2
(Top strand (sense (ss)): SEQ ID NO: 9; bottom strand (antisense) SEQ ID NO: 10)
      1 2 3 4 5 6 7 8 9 10 11 12 13 14 15 16 17 18 19 20 21 22 23
5′ ia-ia-AmsAmsGf-Cm-Af-Am-Gf-Am-Uf-Af-Uf-Um-Uf-Um-Um-Af-Um-Af-Am-Um-Am~GalNAc 3′
3′Td-Td-UmsUmsUm-Um-Cm-Gf-Um-Uf-Cm-Uf-Am-Um-Am-Af-Am-Af-Am-Um-Af-Um-UfsAfsUm 5′
    25 24 23 22 21 20 19 18 17 16 15 14 13 12 11 10 9 8 7 6 5 4 3 2 1
10.3.
(Top strand (sense (ss)): SEQ ID NO: 11; bottom strand (antisense) SEQ ID NO: 12)
                1 2 3 4 5 6 7 8 9 10 11 12 13 14 15 16 17 18 19 20 21 22 23
5′ ia-ia-UmsGmsGm-Gm-Am-Um-Uf-Um-Cf-Af-Uf-Gm-Um-Am-Am-Cm-Cm-Am-Am-Gm-Am~GalNAc 3′
3′ CmsUmsAm-Cm-Cm-Cm-Um-Af-Am-Af-Gm-Um-Am-Cm-Af-Um-Um-Gf-Gm-Um-UmsCfsUm 5′
      23 22 21 20 19 18 17 16 15 14 13 12 11 10 9 8 7 6 5 4 3 2 1
```

### Example 11: Inverted Abasic Chemistry with 5′-GalNAc with Alternative Modification Patterns

Using standard synthesis techniques, the following constructs are synthesized in various versions, with tethers 1 and 2 according to the invention, and with various tri-antennary GalNAc units according to the invention, as described above or depicted in FIGS. 41A-42B. Same sequences as in Example 9 are shown here for consistency.

```
11.1.
(Top strand (sense (ss)): SEQ ID NO: 13; bottom strand (antisense) SEQ ID NO: 14)
                  1 2 3 4 5 6 7 8 9 10 11 12 13 14 15 16 17 18 19 20 21
5′ GalNAc~Gf-Am-Cf-Um-Uf-Um-Cf-Am-Uf-Cm-Cf-Um-Gf-Gm-Af-Am-Af-Um-AfsUmsAf 3′
3′ AmsCfsCm-Uf-Gm-Af-Am-Af-Gm-Uf-Am-Gf-Gm-Af-Cm-Cf-Um-Uf-Um-Af-UmsAfsUm 5′
      23 22 21 20 19 18 17 16 15 14 13 12 11 10 9 8 7 6 5 4 3 2 1
11.2
(Top strand (sense (ss)): SEQ ID NO: 15; bottom strand (antisense) SEQ ID NO: 16)
                1 2 3 4 5 6 7 8 9 10 11 12 13 14 15 16 17 18 19 20 21
5′ GalNAc~Af-Am-Gf-Cm-Af-Am-Gf-Am-Uf-Am-Uf-Um-Uf-Um-Uf-Am-Uf-Am-AfsUmsAf 3′
3′ Td-Td-UmsUfsUm-Uf-Cm-Gf-Um-Uf-Cm-Uf-Am-Uf-Am-Af-Am-Af-Am-Uf-Am-Uf-UmsAfsUm 5′
      25 24 23 22 21 20 19 18 17 16 15 14 13 12 11 10 9 8 7 6 5 4 3 2 1
11.3.
(Top strand (sense (ss)): SEQ ID NO: 17; bottom strand (antisense) SEQ ID NO: 18)
                  1 2 3 4 5 6 7 8 9 10 11 12 13 14 15 16 17 18 19 20 21
5′ GalNAc~Uf-Gm-Gf-Gm-Af-Um-Uf-Um-Cf-Am-Uf-Gm-Uf-Am-Af-Cm-Cf-Am-AfsGmsAf 3′
3′ CmsUfsAm-Cf-Cm-Cf-Um-Af-Am-Af-Gm-Uf-Am-Cf-Am-Uf-Um-Gf-Gm-Uf-UmsCfsUm 5′
      23 22 21 20 19 18 17 16 15 14 13 12 11 10 9 8 7 6 5 4 3 2 1
```

### Example 12: Inverted Abasic Chemistry with 5′-GalNAc with Alternative Modification Patterns

Using standard synthesis techniques, the following constructs are synthesized in various versions, with tethers 1 and 2 according to the invention, and with various tri-antennary GalNAc units according to the invention, as described above or depicted in FIGS. 41A-42B. Same sequences as in Example 9 are shown for consistency. 12.1.

```
(Top strand (sense (ss)): SEQ ID NO: 19; bottom strand (antisense) SEQ ID NO: 20)
    1 2 3 4 5 6 7 8 9 10 11 12 13 14 15 16 17 18 19 20 21
5′ GfsAmsCf-Um-Uf-Um-Cf-Am-Uf-Cm-Cf-Um-Gf-Gm-Af-Am-Af-Um-Af-Um-Af~GalNAc 3′
3′ AmsCfsCm-Uf-Gm-Af-Am-Af-Gm-Uf-Am-Gf-Gm-Af-Cm-Cf-Um-Uf-Um-Af-UmsAfsUm 5′
      23 22 21 20 19 18 17 16 15 14 13 12 11 10 9 8 7 6 5 4 3 2 1
12.2
(Top strand (sense (ss)): SEQ ID NO: 21; bottom strand (antisense) SEQ ID NO: 22)
          1 2 3 4 5 6 7 8 9 10 11 12 13 14 15 16 17 18 19 20 21
5′ AfsAmsGf-Cm-Af-Am-Gf-Am-Uf-Am-Uf-Um-Uf-Um-Uf-Am-Uf-Am-Af-Um-Af~GalNAc 3′
3′ Td-Td-UmsUfsUm-Uf-Cm-Gf-Um-Uf-Cm-Uf-Am-Uf-Am-Af-Am-Af-Am-Uf-Am-Uf-UmsAfsUm 5′
      25 24 23 22 21 20 19 18 17 16 15 14 13 12 11 10 9 8 7 6 5 4 3 2 1
12.3
(Top strand (sense (ss)): SEQ ID NO: 23; bottom strand (antisense) SEQ ID NO: 24)
      1 2 3 4 5 6 7 8 9 10 11 12 13 14 15 16 17 18 19 20 21
5′ UfsGmsGf-Gm-Af-Um-Uf-Um-Cf-Am-Uf-Gm-Uf-Am-Af-Cm-Cf-Am-Af-Gm-Af~GalNAc 3′
3′ CmsUfsAm-Cf-Cm-Cf-Um-Af-Am-Af-Gm-Uf-Am-Cf-Am-Uf-Um-Gf-Gm-Uf-UmsCfsUm 5′
      23 22 21 20 19 18 17 16 15 14 13 12 11 10 9 8 7 6 5 4 3 2 1
```

### Example 13: Benchmarking of siRNA-GalNAc Conjugates

The constructs used in Examples 9-18 are referred to by their numbers and are listed in Table 22. Tether 1 and Tether 2 are shown in FIGS. 41 and 42 respectively.

TABLE 22

| Construct | Target A (GO) | Target B (C5) | Target C (TTR) |
|---|---|---|---|
| Tether 1 | 6.1 | 6.2 | 6.3 |
| Tether 2 | 6.1 | 6.2 | 6.3 |
| Tether 1 | 8.1 | 8.2 | 8.3 |
| Tether 2 | 8.1 | 8.2 | 8.3 |
| Tether 1 | 7.1 | 7.2 | 7.3 |
| Tether 2 | 7.1 | 7.2 | 7.3 |
| Tether 1 | 9.1 | 9.2 | 9.3 |
| Tether 2 | 9.1 | 9.2 | 9.3 |
| 3′-GalNAc control | | | |
| (eg see FIG. 25A) | | | |

The following Table 23 reflects benchmarking to be performed with various select constructs of the invention.

TABLE 23
In vitro Experiments for Benchmarking siRNA-GalNAc

| Target | | A | | B | | C | |
|---|---|---|---|---|---|---|---|
| In | | Human | ASPGR | Human | ASPGR | Human | ASPGR |
| Vitro | | Primary | human | Primary | human | Primary | human |
| siRNA | | hepatocyte | hepatocyte | hepatocyte | hepatocyte | hepatocyte | hepatocyte |
| Group | Benchmark RNAI agents | uptake | binding | uptake | binding | uptake | binding |
| 1 | tether option 1 at 5′-end of sense | 0, 4 | Determine | 0, 4 | Determine | 0, 4 (update), | Determine |
| | strand | (update), | KD for | (update), | KD for | and 24 hr | KD for |
| | Inverted abasic chemistry | and 24 hr | ASPGR | and 24 hr | ASPGR | (silencing) | ASPGR |
| 2 | tether option 2 at 5′-end of sense | (silencing) | binding | (silencing) | binding | incubations, | binding with |
| | strand | incubations, | with | incubations, | with | or Hep3B | siRNA- |
| | Inverted abasic chemistry | or Hep3B | siRNA- | or Hep3B | siRNA- | Transfection | GaINAc |
| 3 | tether option 1 at 5′ end of sense | Transfection | GaINAc | Transfection | GaINAc | w/RNAiMax | |
| | strand | w/RNAiMax | | w/RNAiMax | | for 24 h | |
| | Alternating chemistry | for 24 h | | for 24 h | | | |
| 4 | tether option 2 at 5′ -end of sense | | | | | | |
| | strand | | | | | | |
| | Alternating Chemistry | | | | | | |
| 5 | tether option 1 at 3′ -end of sense | | | | | | |
| | strand | | | | | | |
| | Inverted abasic chemistry | | | | | | |
| 6 | tether option 2 at 3′ -end of sense | | | | | | |
| | strand | | | | | | |
| | Inverted abasic chemistry | | | | | | |
| 7 | tether option 1 at 3′ -end of sense | | | | | | |
| | strand | | | | | | |
| | Alternating chemistry | | | | | | |
| 8 | tether option 2 at 3′ end of sense | | | | | | |
| | strand | | | | | | |
| | Alternating chemistry | | | | | | |
| 9 | 3′-GalNAc control (eg see FIG. | | | | | | |
| | 25A) | | | | | | |

### In Vitro Pharmacodynamic Characterization

The in vitro pharmacodynamics activity, binding affinity, and liver uptake for 8 constructs, listed in Table 1 (GO1 siRNA-GalNAc, C5 siRNA-GalNAc, and TTR siRNA-GalNAc analogues) are benchmarked against the clinically validated versions of these molecules.

Human Liver Cell Line (HepG2 or Hep3B) Transfection Assay—Each GO1 siRNA-GalNAc, C5 siRNA-GalNAc, and TTR siRNA-GalNAc analogue molecule is incubated at 37° C. for 0 and 24 hours at 10 different concentrations in human liver cell line in the presence of transfection reagent (e.g RNAiMAX). All incubations at each concentration are run in quadruplicate. Following incubations, each sample is lysed and analyzed for HAO1 C5, TTR and housekeeping gene (such as GAPDH) mRNA concentrations by bDNA or RT-qPCR assay. mRNA concentrations data obtained is used for analysis to determine the silencing activity and IC₅₀ for each of the GO1 siRNA-GalNAc, C5 siRNA-GalNAc, and TTR siRNA-GalNAc molecules.

Primary Human Hepatocytes Uptake Assay—The liver uptake and silencing activity for each of the GO1 siRNA-GalNAc, C5 siRNA-GalNAc, and TTR siRNA-GalNAc molecules are evaluated in primary human hepatocytes. Each GO1 siRNA-GalNAc, C5 siRNA-GalNAc, and TTR siRNA-GalNAc analogues molecule is incubated at 37° C. for 0, 4, and 72 hours at 10 different concentrations in primary human hepatocytes. All incubations at each concentration are run in quadruplicate. Following incubations, each sample is lysed and analyzed for HAO1, C5, TTR and housekeeping gene(s) (such as GAPDH) mRNA concentrations by bDNA or RT-qPCR assay. mRNA concentrations data obtained are used for analysis to determine the silencing activity, uptake and IC₅₀ for each of the GO1 siRNA-GalNAc, C5 siRNA-GalNAc, and TTR siRNA-GalNAc molecules.

### In Vivo Pharmacodynamic Characterization

The in vivo pharmacodynamics activity for 8 constructs each of GO1 siRNA-GalNAc, C5 siRNA-GalNAc, and TTR siRNA-GalNAc analogues is compared to the in vivo pharmacodynamic activity of clinically validated of each GO1siRNA-GalNAc, C5 siRNA-GalNAc, and TTR siRNA-GalNAc molecules following a single subcutaneous administration to male mice or cynomolgus monkeys.

For the in vivo mice pharmacology of GO1 siRNA-GalNAc of each of analogues is evaluated following a single subcutaneous dose at 0.3 or 3 mg/kg as provided in Table 24 below. There are 2 dose groups in which each of the GO1 siRNA-GalNAc analogues is administered subcutaneously to C57BL/6 male mice (n=3/timepoint/group) at 0.3 or 1 mg/kg. Blood samples to obtain serum samples and liver biopsy samples are obtained at various time points to determine the concentration of serum glycolate by LCMS and to determine the concentration of HAO1 mRNA by RT-qPCR or bDNA assay. The animals from each group at each specified time point are sacrificed and blood (approximately 0.5 mL/animal) and liver (approximately 100 mg) are collected. For Groups 1 through 9, blood (approximately 0.5 mL/animal) and liver (approximately 100 mg) are collected from 3 animals/time point/group at 24, 48, 96, 168, 336, 504, and 672 hours post-dosing. Group 10 (n=3) is a control group that is not dosed to provide baseline values for serum glycolate and mRNA HAO1 concentrations. The pharmacodynamic effect of the increase of serum glycolate and the silencing of HAO1 mRNA in the liver at various time points post-dosing is compared to the Group 10 control serum and liver samples.

TABLE 24
GO1 siRNA-GalNAc Analogues Mice Pharmacology Study Design

| | Number of | | Number | Target Dose | Target Dose | Target Dose |
|---|---|---|---|---|---|---|
| | Animals | Dose | of Doses/ | Level | Concentration | Volume |
| Group | Male | Route | Animal | (mg/kg) | (mg/mL) | (mL/kg) |
| 1 | 21 | SC | 1 | 0.3 or 3 | 3 | 1.5 |
| 2 | 21 | SC | 1 | 0.3 or 3 | 3 | 1.5 |
| 3 | 21 | SC | 1 | 0.3 or 3 | 3 | 1.5 |
| 4 | 21 | SC | 1 | 0.3 or 3 | 3 | 1.5 |
| 5 | 21 | SC | 1 | 0.3 or 3 | 3 | 1.5 |
| 6 | 21 | SC | 1 | 0.3 or 3 | 3 | 1.5 |
| 7 | 21 | SC | 1 | 0.3 or 3 | 3 | 1.5 |
| 8 | 21 | SC | 1 | 0.3 or 3 | 3 | 1.5 |
| 9 | 21 | SC | 1 | 0.3 or 3 | 3 | 1.5 |
| 10^{a} | 5 | NA | NA | NA | NA | NA |

Table Legend:
SC Subcutaneous
NA Not Applicable
^{a}Group 10 animals are control animals and are not be dosed
c Animals in Groups 1 to 9 are dosed on Day 1

### Example 14: 5′ Conjugation Using Click-Chemistry (Option 1)

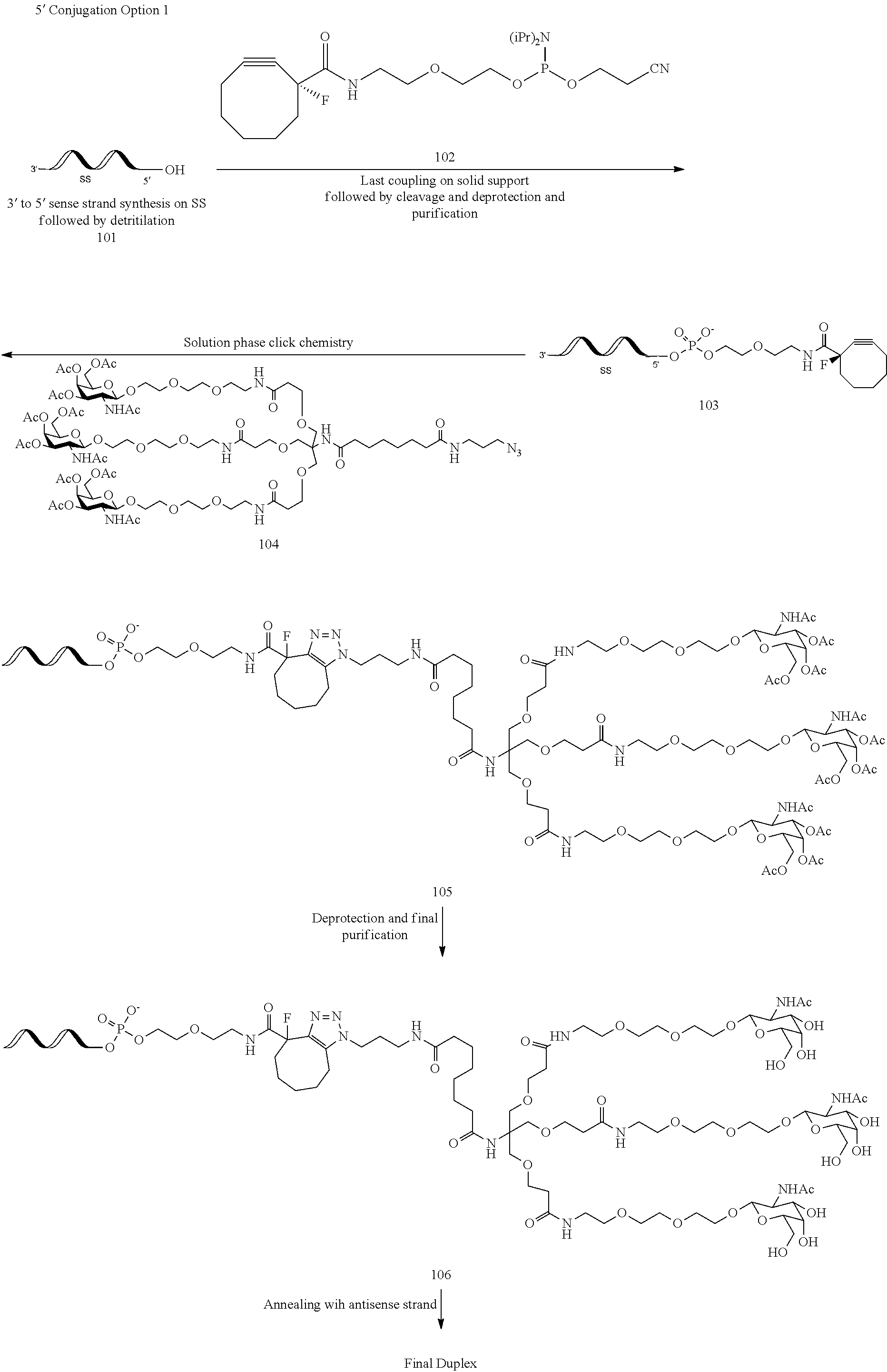

In this embodiment, the sense strand of the oligonucleotide 101 is synthesized on solid support and coupled with the commercially available octyne amidite 102 to give the required oligonucleotide with the click chemistry precursor on the solid support. This after standard cleavage and deprotection provides the pure oligo nucleotide 103. The azide 104 is dissolved in DMSO (150 μL/mg) and this solution is added to 10 OD of oligo 103 in 100 μL of water. The reaction mixture is then incubated at room temperature overnight. The conjugated oligo 105 is desalted on a Glen Gel-Pak™ to remove organics and the acetoxy protecting groups were removed by treating with methylamine followed by prep HPLC to give pure Oligo 106 which is annealed with an equimolar amount of sense strand to give the final duplex.

### Example 15: 5′ Conjugation (Option 2)

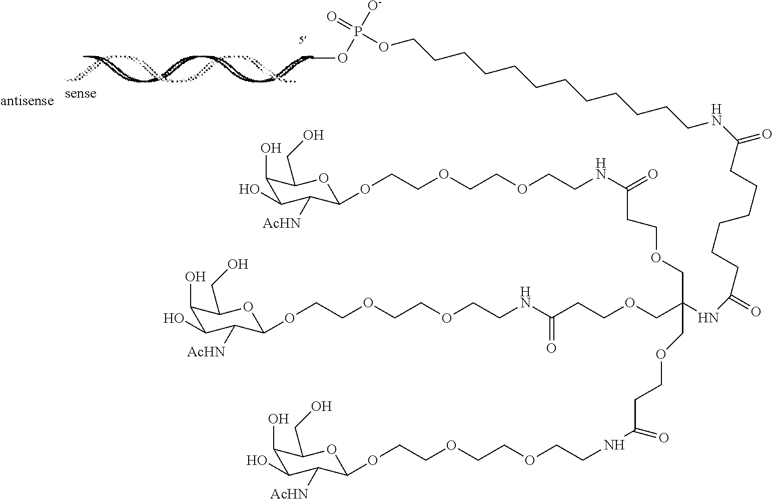 [SMILES: COP(=O)([O-])OCCCCCCCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

In this embodiment, the sense strand of the oligonucleotide 101 is synthesized on solid support and coupled with the commercially available amidite 108 to give the required oligonucleotide on the solid support. This after standard cleavage and deprotection provides the pure oligo nucleotide 109. The amine 109 is dissolved in water (15 μL/OD) and this solution is added to a solution of the acid 110 in DMSO (100 mL/mg) followed by 10 molar equivalents of EDC and 10 equivalents of HOBT and the reaction mixture is incubated at room temperature overnight. The conjugated oligo 111 is then desalted on a Glen Gel-Pak™ to remove organics and the acetoxy protecting groups were removed by treating with methylamine followed by prep HPLC to give pure Oligo 112 which is annealed with an equimolar amount of sense strand to give the final duplex.

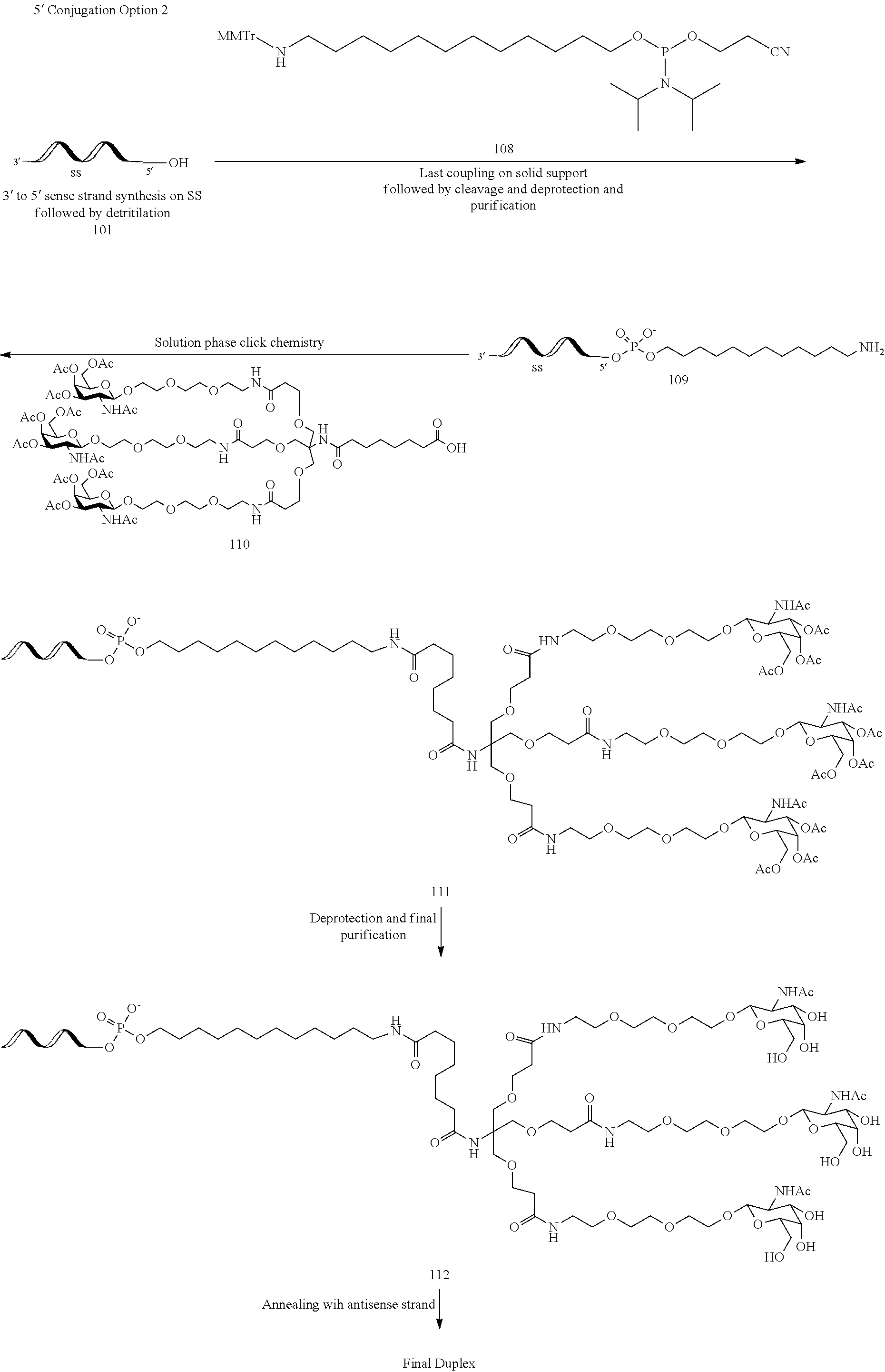

### Example 16: 5′ Conjugation Using Click-Chemistry (Option 1)

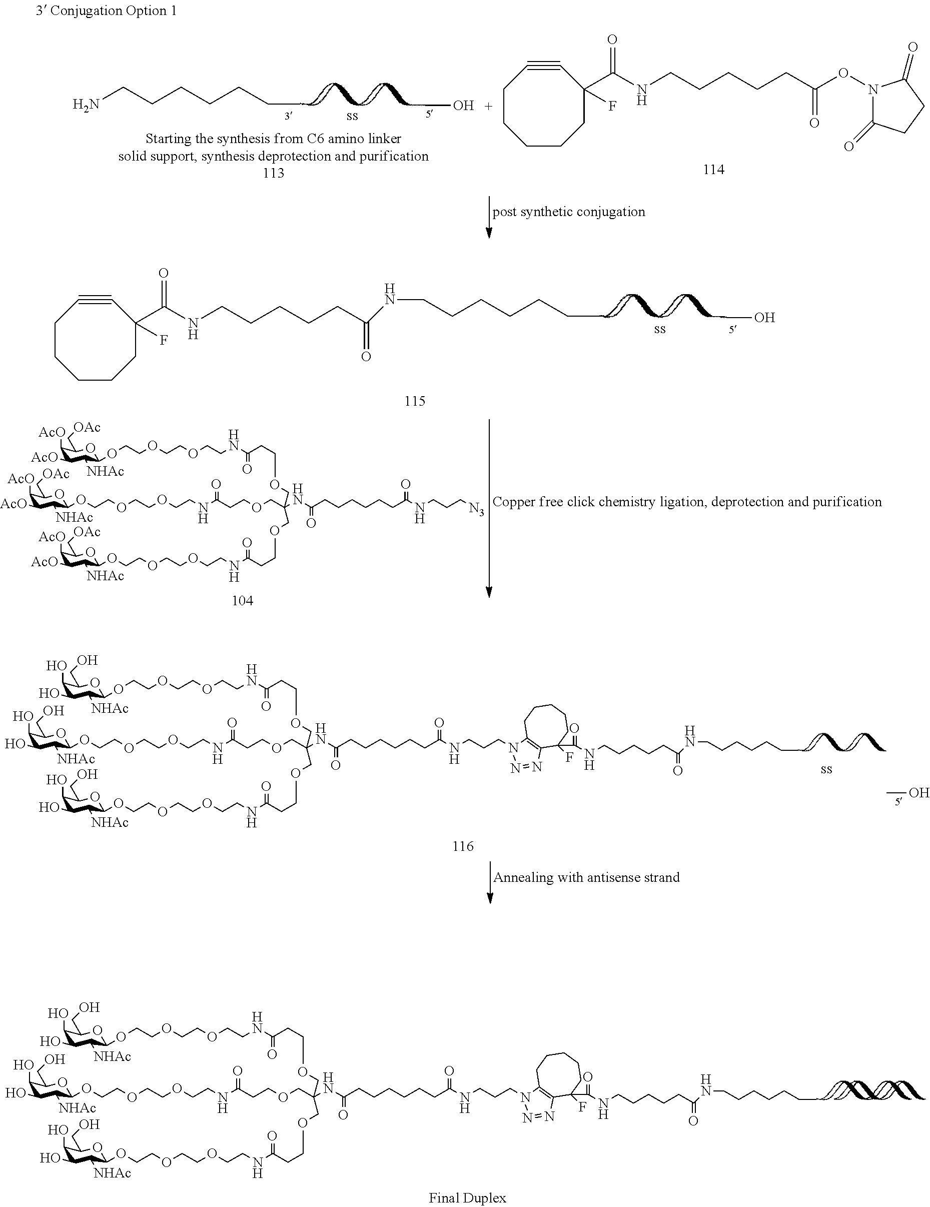

For the synthesis of oligo construct 119 a similar approach is adapted where the triantennary GalNAc conjugate is loaded on to the solid support 118 (CPG) and the oligo synthesis is performed. After cleavage and deprotection and purification provides the pure oligo 119 which is annealed with antisense strand to give the required final duplex in a pure form. In another approach the 3′ conjugate is also synthesized analogous to the synthesis of 116 starting from amino linked oligo 113 and post synthetically conjugating the GalNAc carboxylic acid to give the conjugated oligo 119.

### Example 17: 3′ Conjugation (Option 2)

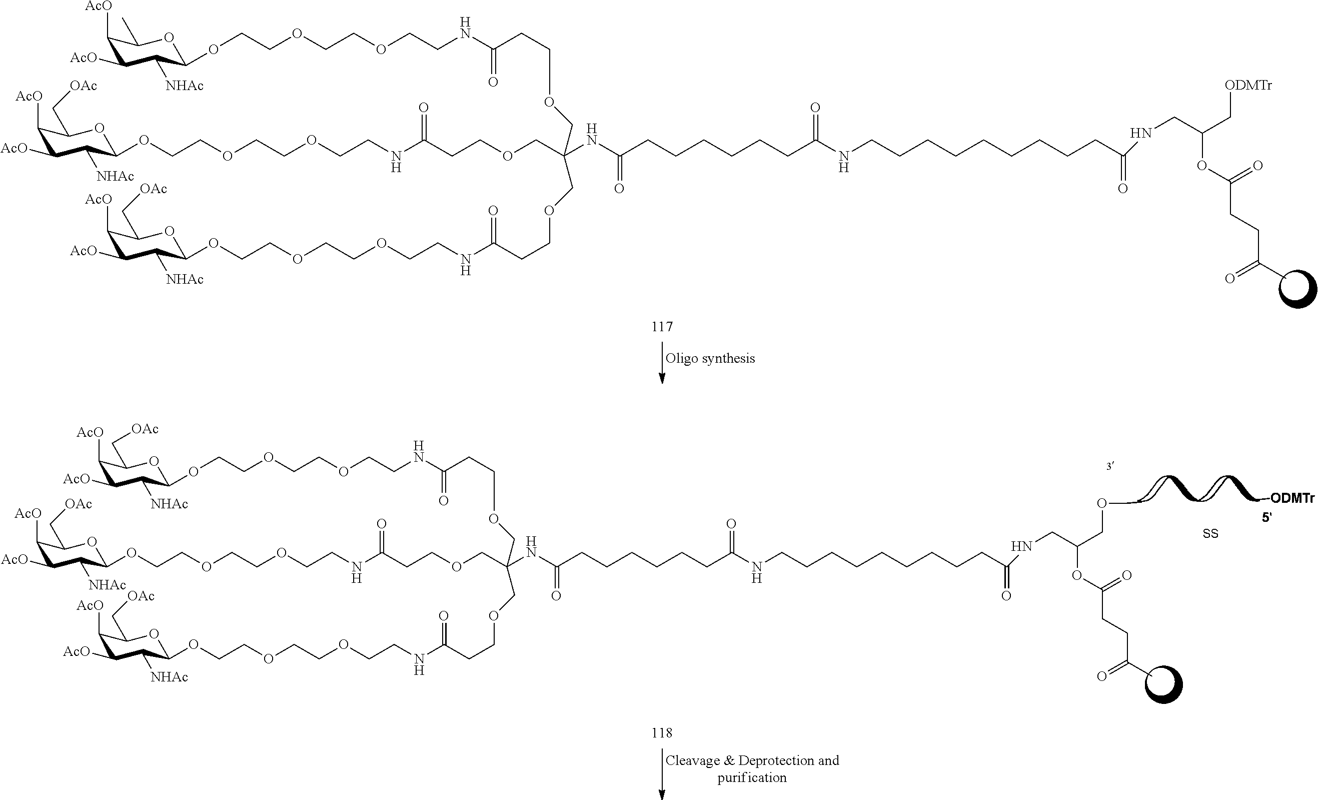

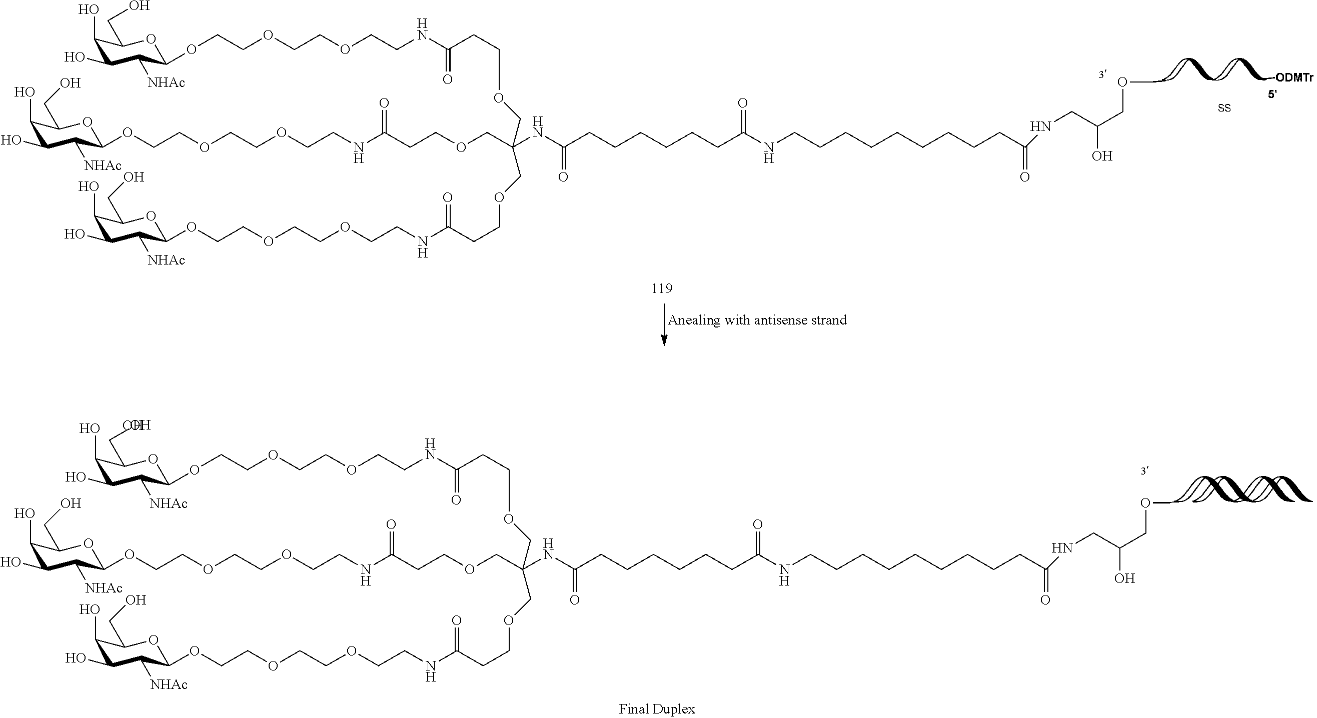

For the synthesis of oligo construct 119 a similar approach is adapted where the tri-antennary GalNAc conjugate is loaded on to the solid support 118 (CPG) and the oligo synthesis is performed. After cleavage and deprotection and purification provided the pure oligo 119 which is annealed with antisense strand to give the required final duplex in a pure form. In another approach, the 3′ conjugate is also synthesized analogous to the synthesis of 116 starting from amino linked oligo 113 and post synthetically conjugating the GalNAc carboxylic acid to give the conjugated oligo 119.

### Example 18: Post-Synthetic Conjugation Approach

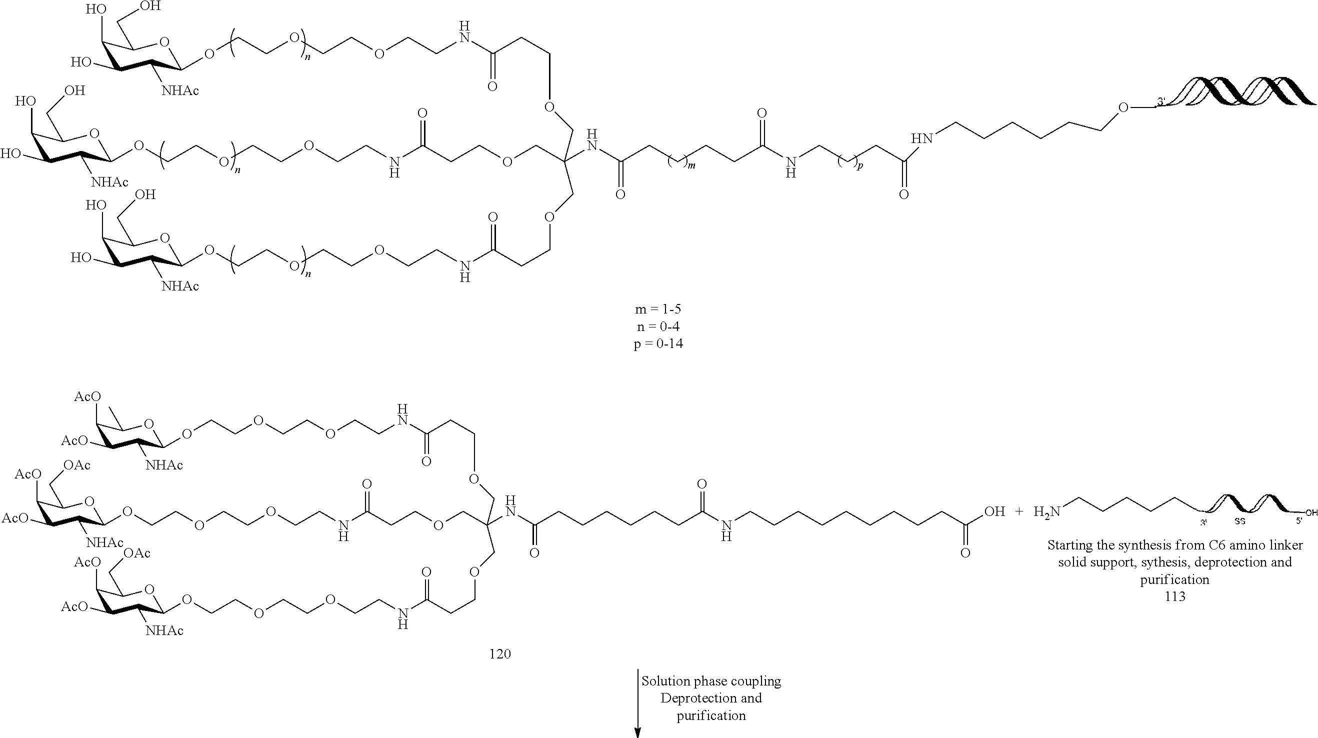

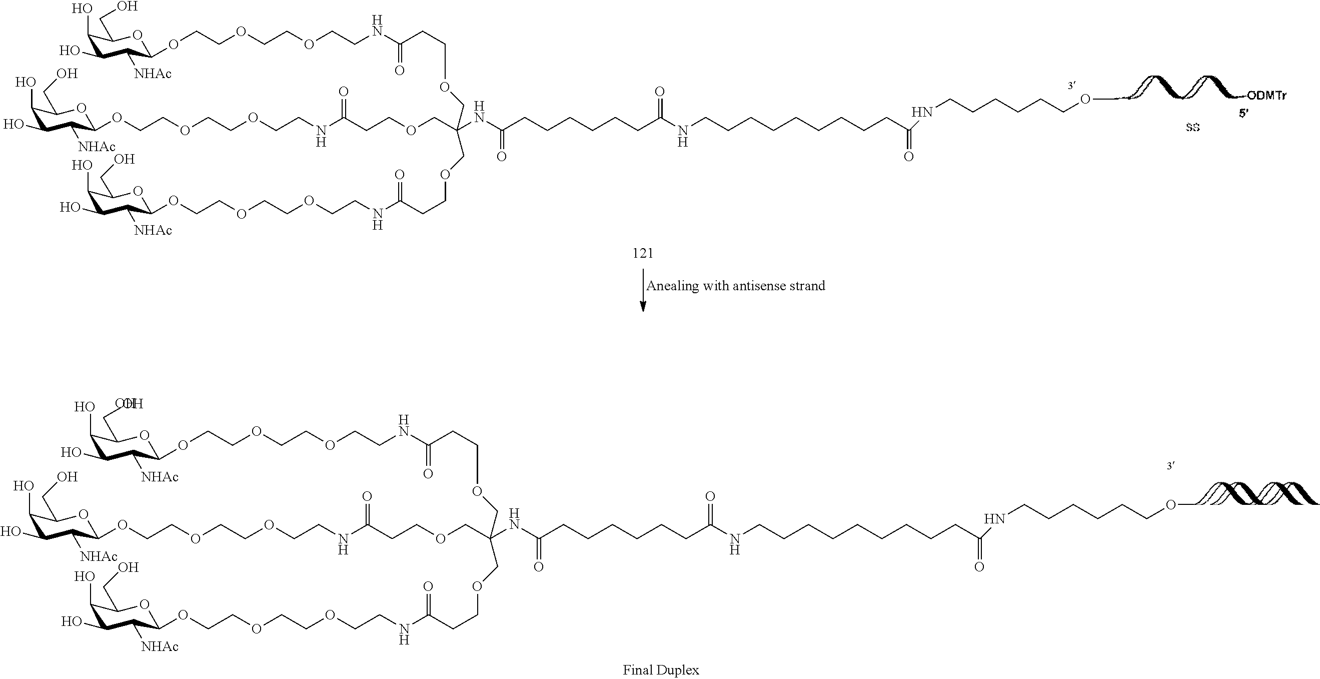

In this approach, the 3′ conjugate is also synthesized analogous to the synthesis of 116 starting from amino linked oligo 113 and post synthetically conjugating the GalNAc carboxylic acid to give the conjugated oligo 121 which is annealed with antisense strand to give the required final duplex in a pure form.

The preceding Examples are not intended to be limiting. Those of skill in the art will, in light of the present disclosure, appreciate that many changes can be made in the specific materials and which are disclosed and still obtain a like or similar result without departing from the spirit and scope of the invention.

### STATEMENTS

1. A modified RNAi agent comprising an RNA interference compound (RNAi compound) conjugated via a tether to an ASGP-R ligand, wherein the tether comprises:

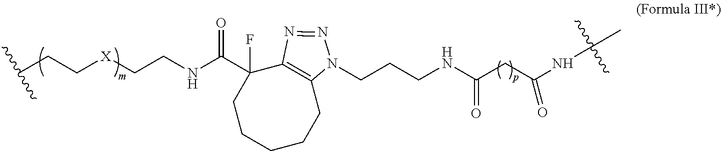

wherein m is chosen from 0, 1, 2, 3, 4, or 5, and p is chosen from 0, 1, 2, 3, 4, 5, 6, 7, 8, 9, 10, 11, 12, 13, or 14, independently of m: and X is chosen from O and CH₂.
2. The modified RNAi agent of statement 1, wherein m=1.
3. The modified RNAi agent of statement 1, wherein x is O.
4. The modified RNAi agent of statement 1, wherein p=6.
5. The modified RNAi agent of statement 1, wherein m=1, p=6, and x is O.
6. The modified RNAi agent of statement 1, wherein the ASGP-R ligand comprises a branched GalNAc.
7. The modified RNAi agent of statement 6, wherein the branched GalNAc is selected from the group consisting of:

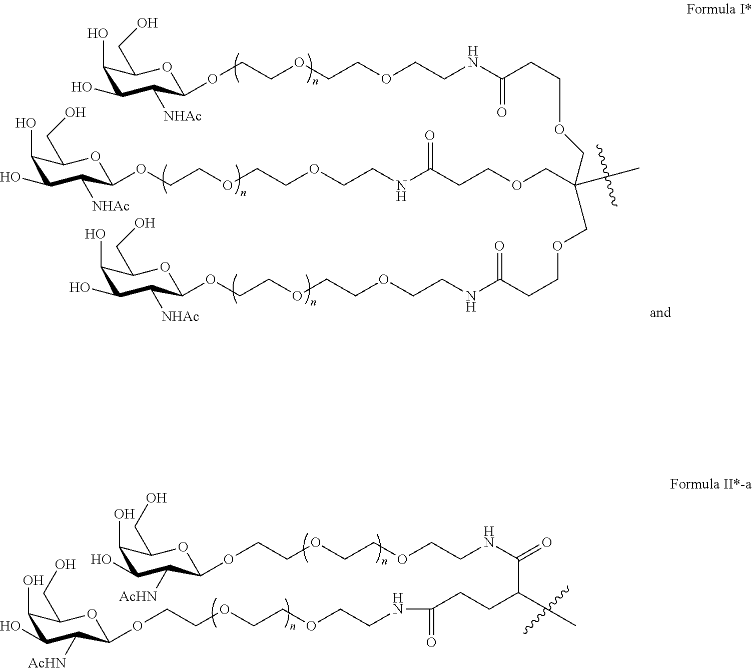

wherein n is 0, 1, 2, 3, or 4.
8. The modified RNAi agent of statement 7, wherein n=1.
9. The modified RNAi agent of statement 6, wherein the branched GalNAc comprises

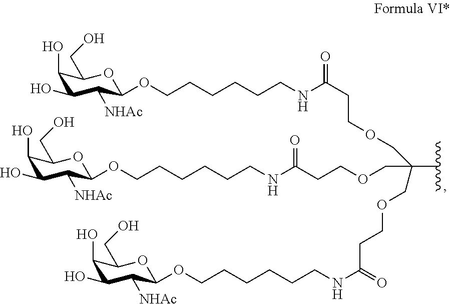

or a bi-antennary form thereof.
10. The modified RNAi agent of statement 6, wherein the branched GalNAc comprises or a bi-antennary form thereof.

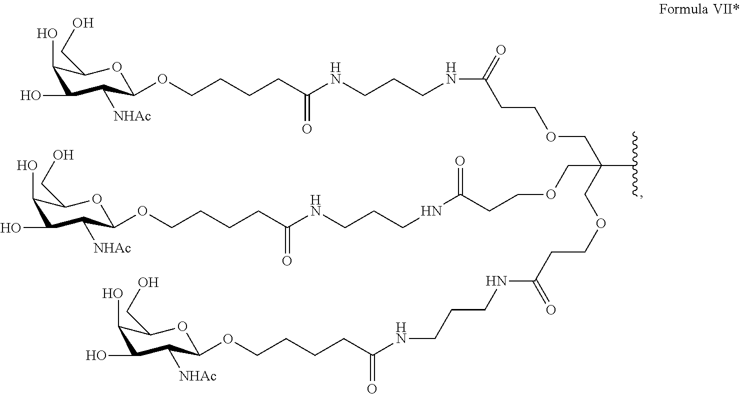

or a bi-antennary form thereof.
11. The modified RNAi agent of statement 1, wherein the tether is attached to the 5′ end of the sense strand.
12. The modified RNAi agent of statement 11, wherein the tether is attached as shown in Formula IV*.

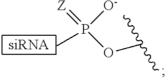

wherein Z is P or S.
13. The modified RNAi agent of statement 11, wherein the tether is attached to the 3′ end of the sense strand.
14. The modified RNAi agent of statement 13, wherein the tether is attached as shown in Formulae V*-a or V*-b:

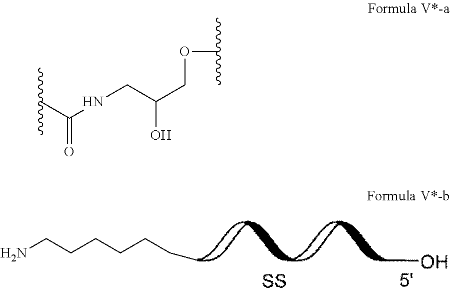

15. The modified RNAi agent of statement 1, as shown in FIG. 41A, 41B, 41C, or 41D.
16. The modified RNAi agent of statement 15, wherein the RNAi compound comprises modified riboses that are modified at the 2′ position.
17. The modified RNAi agent of statement 16, wherein the modifications are chosen from 2′-O-methyl, 2′-deoxy-fluoro, and 2′-deoxy.
18. The modified RNAi agent of statement 1, wherein RNAI compound contains one or more degradation protective moieties at any or all ends that are not conjugated to the ASGP-R ligand.
19. The modified RNAi agent of statement 18, wherein the degradation protective moiety is chosen alone or as any combination from a group consisting of 1-4 phosphorothioate linkages, 1-4 deoxynucleotides, and 1-4 inverted abasic nucleotides.
20. The modified RNAi agent of statement 19, wherein the degradation protective moieties are chosen from the configuration present in one of the following constructs 9.1, 9.2, 9.3, 10, 1, 10.2, 10.3, 11.1, 11.2, 11.3, 12.1, 12.2, and 12.3.
21. A modified RNAi agent comprising an RNA interference compound (RNAi compound) conjugated via a tether to an ASGP-R ligand, wherein the tether comprises:

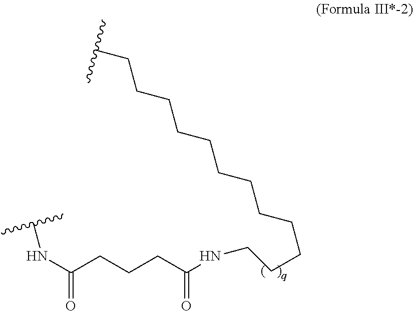

wherein q is chosen from 1, 2, 3, 4, 5, 6, 7, or 8.
22. The modified RNAi agent of statement 21, wherein q=1.
23. The modified RNAi agent of statement 21, wherein the ASGP-R ligand comprises a branched GalNAc.
24. The modified RNAi agent of statement 23, wherein the branched GalNAc is selected from the group consisting of:

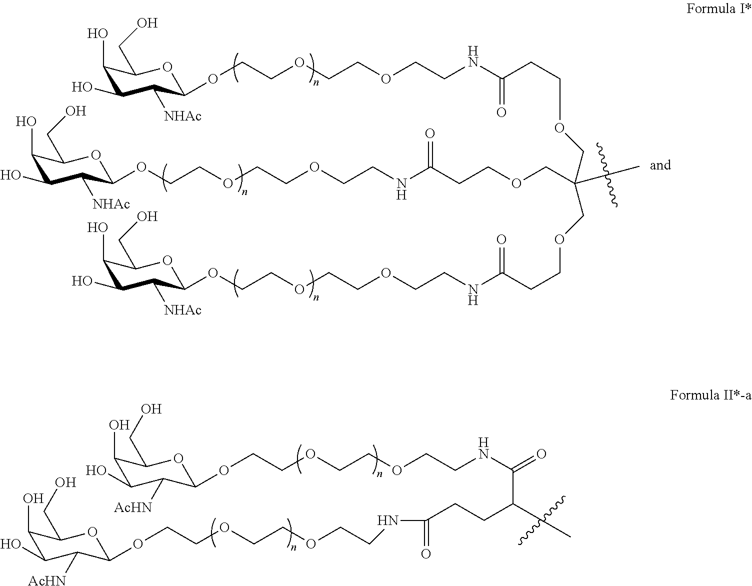

wherein n is 0, 1, 2, 3, or 4.
25. The modified RNAi agent of statement 24, wherein n=1.
26. The modified RNAi agent of statement 23, wherein the branched GalNAc comprises

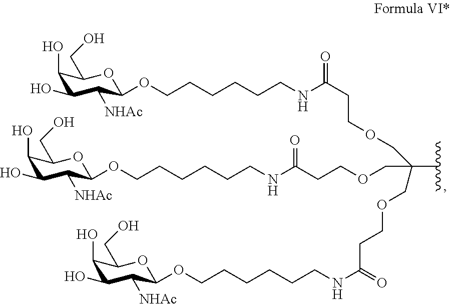

or a bi-antennary form thereof.
27. The modified RNAi agent of statement 23, wherein the branched GalNAc comprises or a bi-antennary form thereof.

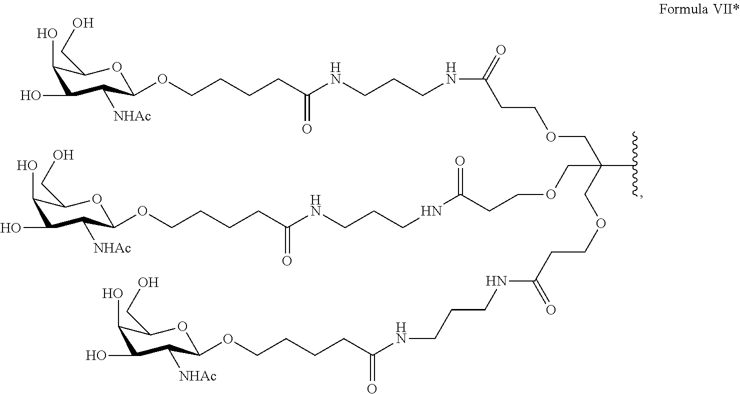

or a bi-antennary form thereof.
28. The modified RNAi agent of statement 21, wherein the tether is attached to the 5′ end of the sense strand.
29. The modified RNAi agent of statement 28, wherein the tether is attached as shown in Formula IV*.

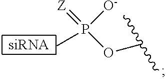

wherein Z is P or S.
30. The modified RNAi agent of statement 28, wherein the tether is attached to the 3′ end of the sense strand.
31. The modified RNAi agent of statement 30, wherein the tether is attached as shown in Formulae V*-a or V*-b:

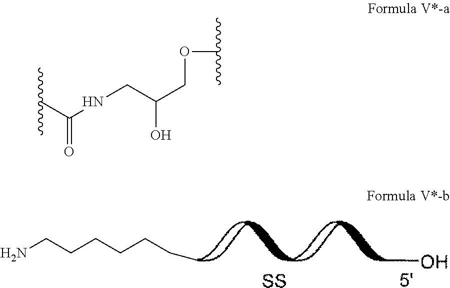

32. The modified RNAi agent of statement 31, as shown in FIG. 42A, 42B, 42C, or 42D.
33. The modified RNAi agent of statement 21, wherein the RNAi compound comprises modified riboses that are modified at the 2′ position.
34. The modified RNAi agent of statement 33, wherein the modifications are chosen from 2′-O-methyl, 2′deoxy-fluoro, and 2′-deoxy.
35. The modified RNAi agent of statement 21, wherein siRNA contains one or more degradation protective moieties at any or all ends that are not conjugated to the ASGP-R ligand.
36. The modified RNAi agent of statement 35, wherein the degradation protective moiety is chosen alone or as any combination from a group consisting of 1-4 phosphorothioate linkages, 1-4 deoxynucleotides, and 1-4 inverted abasic nucleotides.
37. The modified RNAi agent of statement 36, wherein the degradation protective moieties are chosen from the configuration present in one of the following constructs 9.1, 9.2, 9.3, 10.1, 10.2, 10.3, 11.1, 11.2, 11.3, 12.1, 12.2, and 12.3.
38. A method of making the RNAi agent of statements 1 or 2, said method being shown in Examples 11-15.
39. A method of preventing, alleviating, or treating a disease in a subject, the method comprising administering, to the subject, the RNAi agent of statements 1 or 2 in a therapeutically amount effective to prevent, alleviate or treat the disease, thereby preventing, alleviating, or treating a disease.
40. The method of statement 40, wherein the subject is human.

TABLE A
Summary of Sequences

| Seq | Duplex | SSRN | | | |
|---|---|---|---|---|---|
| ID | ID | ID | Sense Sequence | Antisense Sequence | Clean Sequence |
| 1. | | | 5′ GalNAc~Gm-Am-Cm-Um-Um-Um-Cf-Am-Uf- | | gacuuucauccuggaaauaua |
| | | | Cf-Cf-Um-Gm-Gm-Am-Am-Am-Um- | | |
| | | | AmsUmsAm-ia-ia 3′ | | |
| 2. | | | | 3′ AmsCms-Cm-Um-Gm- | accugaaaguaggaccuuuauau |
| | | | | Am-Am-Af-Gm-Uf-Am-Gm- | |
| | | | | Gm-Am-Cf-Cf-Um-Uf-Um- | |
| | | | | Am-UmsAfsUm 5′ | |
| 3. | | | 5′ GalNAc~Am-Am-Gf-Cm-Af-Am-Gf-Am- | | aagcaagauauuuuuauaaua |
| | | | Uf-Af-Uf-Um-Uf-Um-Um-Af-Um-Af- | | |
| | | | AmsUmsAm-ia-ia 3′ | | |
| 4. | | | | 3′ Td-Td-UmsUmsUm-Um- | uuuucguucuauaaaaauauuau |
| | | | | Cm-Gf-Um-Uf-Cm-Uf-Am- | |
| | | | | Um-Am-Af-Am-Af-Am-Um- | |
| | | | | Af-Um-UfsAfsUm 5′ | |
| 5. | | | 5′GalNAc~Um-Gm-Gm-Gm-Am-Um-Uf-Um-Cf- | | ugggauuucauguaaccaaga |
| | | | Af-Uf-Gm-Um-Am-Am-Cm-Cm-Am- | | |
| | | | AmsGmsAm-ia-ia 3′ | | |
| 6. | | | | 3′ CmsUmsAm-Cm-Cm-Cm- | cuacccuaaaguacauugguucu |
| | | | | Um-Af-Am-Af-Gm-Um-Am- | |
| | | | | Cm-Af-Um-Um-Gf-Gm-Um- | |
| | | | | UmsCfsUm 5′ | |
| 7. | | | 5′ ia-ia-GmsAmsCm-Um-Um-Um-Cf-Am-Uf-Cf- | | gacuuucauccuggaaauaua |
| | | | Cf-Um-Gm-Gm-Am-Am-Am-Um-Am-Um- | | |
| | | | Am~GalNAc 3′ | | |
| 8. | | | | 3′ AmsCmsCm-Um-Gm- | accugaaaguaggaccuuuauau |
| | | | | Am-Am-Af-Gm-Uf-Am-Gm- | |
| | | | | Gm-Am-Cf-Cf-Um-Uf-Um- | |
| | | | | Am-UmsAfsUm 5′ | |
| 9. | | | 5′ ia-ia-AmsAmsGf-Cm-Af-Am-Gf-Am-Uf-Af- | | aagcaagauauuuuuauaaua |
| | | | Uf-Um-Uf-Um-Um-Af-Um-Af-Am-Um- | | |
| | | | Am~GalNAc 3′ | | |
| 10. | | | | 3′Td-Td-UmsUmsUm-Um- | uuuucguucuauaaaaauauuau |
| | | | | Cm-Gf-Um-Uf-Cm-Uf-Am- | |
| | | | | Um-Am-Af-Am-Af-Am-Um- | |
| | | | | Af-Um-UfsAfsUm 5′ | |
| 11. | | | 5′ ia-ia-UmsGmsGm-Gm-Am-Um-Uf-Um-Cf-Af- | | ugggauuucauguaaccaaga |
| | | | Uf-Gm-Um-Am-Am-Cm-Cm-Am-Am-Gm- | | |
| | | | Am~GalNAc 3′ | | |
| 12. | | | | 3′ CmsUmsAm-Cm-Cm-Cm- | cuacccuaaaguacauugguucu |
| | | | | Um-Af-Am-Af-Gm-Um-Am- | |
| | | | | Cm-Af-Um-Um-Gf-Gm-Um- | |
| | | | | UmsCfsUm 5′ | |
| 13. | | | 5′GalNAc~Gf-Am-Cf-Um-Uf-Um-Cf-Am-Uf-Cm- | | gacuuucauccuggaaauaua |
| | | | Cf-Um-Gf-Gm-Af-Am-Af-Um-AfsUmsAf 3′ | | |
| 14. | | | | 3′ AmsCfsCm-Uf-Gm-Af- | accugaaaguaggaccuuuauau |
| | | | | Am-Af-Gm-Uf-Am-Gf-Gm- | |
| | | | | Af-Cm-Cf-Um-Uf-Um-Af- | |
| | | | | UmsAfsUm 5′ | |
| 15. | | | 5′ GalNAc~Af-Am-Gf-Cm-Af-Am-Gf-Am-Uf- | | aagcaagauauuuuuauaaua |
| | | | Am-Uf-Um-Uf-Um-Uf-Am-Uf-Am-AfsUmsAf 3′ | | |
| 16. | | | | 3′ Td-Td-UmsUfsUm-Uf- | uuuucguucuauaaaaauauuau |
| | | | | Cm-Gf-Um-Uf-Cm-Uf-Am- | |
| | | | | Uf-Am-Af-Am-Af-Am-Uf- | |
| | | | | Am-Uf-UmsAfsUm 5′ | |
| 17. | | | 5′ GalNAc~Uf-Gm-Gf-Gm-Af-Um-Uf-Um-Cf- | | ugggauuucauguaaccaaga |
| | | | Am-Uf-Gm-Uf-Am-Af-Cm-Cf-Am-AfsGmsAf 3′ | | |
| 18. | | | | 3′ CmsUfsAm-Cf-Cm-Cf- | cuacccuaaaguacauugguucu |
| | | | | Um-Af-Am-Af-Gm-Uf-Am- | |
| | | | | Cf-Am-Uf-Um-Gf-Gm-Uf- | |
| | | | | UmsCfsUm 5′ | |
| 19. | | | 5′ GfsAmsCf-Um-Uf-Um-Cf-Am-Uf-Cm-Cf- | | gacuuucauccuggaaauaua |
| | | | Um-Gf-Gm-Af-Am-Af-Um-Af-Um-Af~GalNAc | | |
| | | | 3 | | |
| 20. | | | | 3′ AmsCfsCm-Uf-Gm-Af- | accugaaaguaggaccuuuauau |
| | | | | Am-Af-Gm-Uf-Am-Gf-Gm- | |
| | | | | Af-Cm-Cf-Um-Uf-Um-Af- | |
| | | | | UmsAfsUm 5′ | |
| 21. | | | 5′ AfsAmsGf-Cm-Af-Am-Gf-Am-Uf-Am- | | aagcaagauauuuuuauaaua |
| | | | Uf-Um-Uf-Um-Uf-Am-Uf-Am-Af-Um- | | |
| | | | Af~GalNAc 3′ | | |
| 22. | | | | 3′ Td-Td-UmsUfsUm-Uf- | uuuucguucuauaaaaauauuau |
| | | | | Cm-Gf-Um-Uf-Cm-Uf-Am- | |
| | | | | Uf-Am-Af-Am-Af-Am-Uf- | |
| | | | | Am-Uf-UmsAfsUm 5′ | |
| 23. | | | 5′ UfsGmsGf-Gm-Af-Um-Uf-Um-Cf-Am-Uf- | | ugggauuucauguaaccaaga |
| | | | Gm-Uf-Am-Af-Cm-Cf-Am-Af-Gm-Af~GalNAc 3′ | | |
| 24. | | | | 3′ CmsUfsAm-Cf-Cm-Cf- | cuacccuaaaguacauugguucu |
| | | | | Um-Af-Am-Af-Gm-Uf-Am- | |
| | | | | Cf-Am-Uf-Um-Gf-Gm-Uf- | |
| | | | | UmsCfsUm 5′ | |
| Seq | Duplex | SSRN | | | |
| ID | ID | ID | Sense Sequence 5′ → 3′ | Antisense Sequence 5′ → 3′ | Clean Sequence |
| 25. | ETX005 | | (invabasic)(invabasic)gsascu | | gacuuucauccuggaaauaua |
| | | | uuCfaUfCfCfuggaaauasusa | | |
| | | | (NHC6)(MFCO)(ET- | | |
| | | | GalNAc-T1N3) | | |
| 26. | ETX005 | | | usAfsuauUfuCfCfaggaUfgAfaagucscsa | uauauuuccaggaugaaagucca |
| 27. | ETX001 | | (ET-GalNAc- | | gacuuucauccuggaaauaua |
| | | | TIN3)(MFCO)(NH- | | |
| | | | DEG)gacuuuCfaUfCfCfugg | | |
| | | | aaauasusa(invabasic) | | |
| | | | (invabasic) | | |
| 28. | ETX001 | | | usAfsuauUfuCfCfaggaUfgAfaagucscsa | uauauuuccaggaugaaagucca |
| 29. | ETX014 | | (invabasic)(invabasic)asasGf | | aagcaagauauuuuuauaaua |
| | | | cAfaGfaUfAfUfuUfuuAfuA | | |
| | | | faua(NHC6)(MFCO)(ET- | | |
| | | | GalNAc-T1N3) | | |
| 30. | ETX014 | | | usAfsUfuAfuaAfaAfauaUfcUfuGfcuus | uauuauaaaaauaucuugcuuuu |
| | | | | usudTdT | |
| 31. | ETX010 | | (ET-GalNAc- | | aagcaagauauuuuuauaaua |
| | | | T1N3)(MFCO)(NH- | | |
| | | | DEG)aaGfcAfaGfaUfAfUfu | | |
| | | | UfuuAfuAfasusa(invabasic) | | |
| | | | (invabasic) | | |
| 32. | ETX010 | | | usAfsUfuAfuaAfaAfauaUfcUfuGfcuus | uauuauaaaaauaucuugcuuuu |
| | | | | usudTdT | |
| 33. | ETX019 | | (ET-GalNAc- | | ugggauuucauguaaccaaga |
| | | | T1N3)(MFCO)(NH- | | |
| | | | DEG)ugggauUfuCfAfUfgua | | |
| | | | accaasgsa(invabasic) | | |
| | | | (invabasic) | | |
| 34. | ETX019 | | | usCfsuugGfuuAfcaugAfaAfucccasusc | ucuugguuacaugaaaucccauc |
| 35. | ETX023 | | (invabasic)(invabasic)usgsg | | ugggauuucauguaaccaaga |
| | | | gauUfuCfAfUfguaaccaaga | | |
| | | | (NHC6)(MFCO)(ET- | | |
| | | | GalNAc-T1N3) | | |
| 36. | ETX023 | | | usCfsuugGfuuAfcaugAfaAfucccasusc | ucuugguuacaugaaaucccauc |
| 37. | | XD- | cuuAcGcuGAGuAcuucGAd | | cuuacgcugaguacuucga |
| | | 00914 | TsdT | | |
| 38. | | XD- | | UCGAAGuACUcAGCGuAAGdTsdT | ucgaaguacucagcguaag |
| | | 00914 | | | |
| 39. | | XD- | AGAuAuGcAcAcAcAcGG | | agauaugcacacacacgga |
| | | 03999 | AdTsdT | | |
| 40. | | XD- | | UCCGUGUGUGUGcAuAUCUdTsdT | uccgugugugugcauaucu |
| | | 03999 | | | |
| 41. | | XD- | uscsUfcGfuGfgCfcUfuAfaU | | ucucguggccuuaaugaaa |
| | | 15421 | fgAfaAf(invdT) | | |
| 42. | | XD- | | UfsUfsuCfaUfuAfaGfgCfcAfcGfaGfas | uuucauuaaggccacgagauu |
| | | 15421 | | usu | |
| 43. | | X91382 | (NH2- | | gacuuucauccuggaaauaua |
| | | | DEG)gacuuuCfaUfCfCfugg | | |
| | | | aaauasusa(invabasic) | | |
| | | | (invabasic) | | |
| 44. | | X91383 | (NH2- | | aagcaagauauuuuuauaaua |
| | | | DEG)aaGfcAfaGfaUfAfUfu | | |
| | | | UfuuAfuAfasusa(invabasic) | | |
| | | | (invabasic) | | |
| 45. | | X91384 | (NH2- | | ugggauuucauguaaccaaga |
| | | | DEG)ugggauUfuCfAfUfgua | | |
| | | | accaasgsa(invabasic) | | |
| | | | (invabasic) | | |
| 46. | | X91403 | (NH2C12)gacuuuCfaUfCfC | | gacuuucauccuggaaauaua |
| | | | fuggaaauasusa(invabasic) | | |
| | | | (invabasic) | | |
| 47. | | X91404 | (NH2C12)aaGfcAfaGfaUfA | | aagcaagauauuuuuauaaua |
| | | | fUfuUfuuAfuAfasusa | | |
| | | | (invabasic)(invabasic) | | |
| 48. | | X91405 | (NH2C12)ugggauUfuCfAfU | | ugggauuucauguaaccaaga |
| | | | fguaaccaasgsa(invabasic) | | |
| | | | (invabasic) | | |
| 49. | | X91415 | (invabasic)(invabasic)gsascu | | gacuuucauccuggaaauaua |
| | | | uuCfaUfCfCfuggaaauasusa | | |
| | | | (NH2C6) | | |
| 50. | | X91416 | (invabasic)(invabasic)asasGf | | aagcaagauauuuuuauaaua |
| | | | cAfaGfaUfAfUfuUfuuAfuA | | |
| | | | faua(NH2C6) | | |
| 51. | | X91417 | (invabasic)(invabasic)usgsg | | ugggauuucauguaaccaaga |
| | | | gauUfuCfAfUfguaaccaaga | | |
| | | | (NH2C6) | | |
| 52. | | X91379 | gsascuuuCfaUfCfCfuggaaau | | gacuuucauccuggaaauaua |
| | | | aua(GalNAc) | | |
| 53. | | X91380 | asasGfcAfaGfaUfAfUfuUfu | | aagcaagauauuuuuauaaua |
| | | | uAfuAfaua(GalNAc) | | |
| 54. | | X91446 | usgsggauUfuCfAfUfguaacca | | ugggauuucauguaaccaaga |
| | | | aga(GalNAc) | | |
| 55. | | X38483 | usAfsuau UfuCfCfaggaUfgA | | uauauuuccaggaugaaagucca |
| | | | faagucscsa | | |
| 56. | | X91381 | usAfsUfuAfuaAfaAfauaUfc | | uauuauaaaaauaucuugcuuuu |
| | | | UfuGfcuususudTdT | | |
| 57. | | X38104 | usCfsuugGfuuAfcaugAfaAf | | ucuugguuacaugaaaucccauc |
| | | | ucccasusc | | |
| 58. | | X91388 | (MFCO)(NH- | | gacuuucauccuggaaauaua |
| | | | DEG)gacuuuCfaUfCfCfugg | | |
| | | | aaauasusa(invabasic) | | |
| | | | (invabasic) | | |
| 59. | | X91389 | (MFCO)(NH- | | aagcaagauauuuuuauaaua |
| | | | DEG)aaGfcAfaGfaUfAfUfu | | |
| | | | UfuuAfuAfasusa(invabasic) | | |
| | | | (invabasic) | | |
| 60. | | X91390 | (MFCO)(NH- | | ugggauuucauguaaccaaga |
| | | | DEG)ugggauUfuCfAfUfgua | | |
| | | | accaasgsa(invabasic) | | |
| | | | (invabasic) | | |
| 61. | | X91421 | (invabasic)(invabasic)gsascu | | gacuuucauccuggaaauaua |
| | | | uuCfaUfCfCfuggaaauasusa | | |
| | | | (NHC6)(MFCO) | | |
| 62. | | X91422 | (invabasic)(invabasic)asasGf | | aagcaagauauuuuuauaaua |
| | | | cAfaGfaUfAfUfuUfuuAfuA | | |
| | | | faua(NHC6)(MFCO) | | |
| 63. | | X91423 | (invabasic)(invabasic)usgsg | | ugggauuucauguaaccaaga |
| | | | gauUfuCfAfUfguaaccaaga | | |
| | | | (NHC6)(MFCO) | | |
| 64. | | X91394 | (GalNAc-T1)(MFCO)(NH- | | gacuuucauccuggaaauaua |
| | | | DEG)gacuuuCfaUfCfCfugg | | |
| | | | aaauasusa(invabasic) | | |
| | | | (invabasic) | | |
| 65. | | X91395 | (GalNAc-T1)(MFCO)(NH- | | aagcaagauauuuuuauaaua |
| | | | DEG)aaGfcAfaGfaUfAfUfu | | |
| | | | UfuuAfuAfasusa(invabasic) | | |
| | | | (invabasic) | | |
| 66. | | X91396 | (GalNAc-T1)(MFCO)(NH- | | ugggauuucauguaaccaaga |
| | | | DEG)ugggauUfuCfAfUfgua | | |
| | | | accaasgsa(invabasic) | | |
| | | | (invabasic) | | |
| 67. | | X91427 | (invabasic)(invabasic)gsascu | | gacuuucauccuggaaauaua |
| | | | uuCfaUfCfCfuggaaauasusa | | |
| | | | (NHC6)(MFCO)(GalNAc- | | |
| | | | T1) | | |
| 68. | | X91428 | (invabasic)(invabasic)asasGf | | aagcaagauauuuuuauaaua |
| | | | cAfaGfaUfAfUfuUfuuAfuA | | |
| | | | faua(NHC6)(MFCO) | | |
| | | | (GalNAc-T1) | | |
| 69. | | X91429 | (invabasic)(invabasic)usgsg | | ugggauuucauguaaccaaga |
| | | | gauUfuCfAfUfguaaccaaga | | |
| | | | (NHC6)(MFCO)(GalNAc- | | |
| | | | T1) | | |
| 70. | ETX001 | X91394 | (GalNAc- | | gacuuucauccuggaaauaua |
| | | | T1)(MFCO)(NHDEG)gacuu | | |
| | | | uCfaUfCfCfuggaaauasusa | | |
| | | | (invabasic)(invabasic) | | |
| 71. | | X38483 | usAfsuauUfuCfCfaggaUfgA | | uauauuuccaggaugaaagucca |
| | | | faagucscsa | | |
| 72. | ETX005 | X91427 | (invabasic)(invabasic)gsascu | | gacuuucauccuggaaauaua |
| | | | uuCfaUfCfCfuggaaauasusa | | |
| | | | (NHC6)(MFCO)(GalNAc- | | |
| | | | T1) | | |
| 73. | | X38483 | usAfsuauUfuCfCfaggaUfgA | | uauauuuccaggaugaaagucca |
| | | | faagucscsa | | |
| 74. | ETX010 | X91395 | (GalNAc-T1)(MFCO)(NH- | | aagcaagauauuuuuauaaua |
| | | | DEG)aaGfcAfaGfaUfAfUfu | | |
| | | | UfuuAfuAfasusa(invabasic) | | |
| | | | (invabasic) | | |
| 75. | | X91381 | usAfsUfuAfuaAfaAfauaUfc | | uauuauaaaaauaucuugcuuuu |
| | | | UfuGfcuususudTdT | | |
| 76. | ETX014 | X91428 | (invabasic)(invabasic)asasGf | | aagcaagauauuuuuauaaua |
| | | | cAfaGfaUfAfUfuUfuuAfuA | | |
| | | | faua(NHC6)(MFCO) | | |
| | | | (GalNAc-T1) | | |
| 77. | | X91381 | usAfsUfuAfuaAfaAfauaUfc | | uauuauaaaaauaucuugcuuuu |
| | | | UfuGfcuususudTdT | | |
| 78. | ETX019 | X91396 | (GalNAc-T1)(MFCO)(NH- | | ugggauuucauguaaccaaga |
| | | | DEG)ugggauUfuCfAfUfgua | | |
| | | | accaasgsa(invabasic) | | |
| | | | (invabasic) | | |
| 79. | | X38104 | usCfsuugGfuuAfcaugAfaAf | | ucuugguuacaugaaaucccauc |
| | | | ucccasusc | | |
| 80. | ETX023 | X91429 | (invabasic)(invabasic)usgsg | | ugggauuucauguaaccaaga |
| | | | gauUfuCfAfUfguaaccaaga | | |
| | | | (NHC6)(MFCO)(GalNAc- | | |
| | | | T1) | | |
| 81. | | X38104 | usCfsuugGfuuAfcaugAfaAf | | ucuugguuacaugaaaucccauc |
| | | | ucccasusc | | |
| 82. | ETX006 | | (invabasic)(invabasic)gsascu | | gacuuucauccuggaaauaua |
| | | | uuCfaUfCfCfuggaaauasusa | | |
| | | | (NHC6)(ET-GalNAc-T2CO) | | |
| 83. | ETX006 | | | usAfsuauUfuCfCfaggaUfgAfaagucscsa | uauauuuccaggaugaaagucca |
| 84. | ETX002 | | (ET-GalNAc- | | gacuuucauccuggaaauaua |
| | | | T2CO)(NH2C12)gacuuuCfa | | |
| | | | UfCfCfuggaaauasusa | | |
| | | | (invabasic)(invabasic) | | |
| 85. | ETX002 | | | usAfsuauUfuCfCfaggaUfgAfaagucscsa | uauauuuccaggaugaaagucca |
| 86. | ETX004 | | (ET-GalNAc- | | gacuuucauccuggaaauaua |
| | | | T2CO)(NH2C12)GfaCfuUf | | |
| | | | uCfaUfcCfuGfgAfaAfuAfsu | | |
| | | | sAf | | |
| 87. | ETX004 | | | usAfsuAfuUfuCfcAfgGfaUfgAfaAfgUf | uauauuuccaggaugaaagucca |
| | | | | csCfsa | |
| 88. | ETX008 | | GfsasCfuUfuCfaUfcCfuGfg | | gacuuucauccuggaaauaua |
| | | | AfaAfuAfuAf(NHC6)(ET- | | |
| | | | GalNAc-T2CO) | | |
| 89. | ETX008 | | | usAfsuAfuUfuCfcAfgGfaUfgAfaAfgUf | uauauuuccaggaugaaagucca |
| | | | | csCfsa | |
| 90. | ECX008 | | GfsasCfuUfuCfaUfcCfuGfg | | gacuuucauccuggaaauaua |
| | (lower | | AfaAfuAfuAf(NHC6)(ET- | | |
| | purity) | | GalNAc-T2CO) | | |
| 91. | ECX008 | | | usAfsuAfuUfuCfcAfgGfaUfgAfaAfgUf | uauauuuccaggaugaaagucca |
| | (lower | | | csCfsa | |
| | purity) | | | | |
| 92. | ETX011 | | (ET-GalNAc- | | aagcaagauauuuuuauaaua |
| | | | T2CO)(NH2C12)aaGfcAfa | | |
| | | | GfaUfAfUfuUfuuAfuAfasus | | |
| | | | a(invabasic)(invabasic) | | |
| 93. | ETX011 | | | usAfsUfuAfuaAfaAfauaUfcUfuGfcuus | uauuauaaaaauaucuugcuuuu |
| | | | | usudTdT | |
| 94. | ETX015 | | (invabasic)(invabasic)asasGf | | aagcaagauauuuuuauaaua |
| | | | cAfaGfaUfAfUfuUfuuAfuA | | |
| | | | faua(NHC6)(ET-GalNAc- | | |
| | | | T2CO) | | |
| 95. | ETX015 | | | usAfs UfuAfuaAfaAfauaUfcUfuGfcuus | uauuauaaaaauaucuugcuuuu |
| | | | | usudTdT | |
| 96. | ETX013 | | (ET-GalNAc- | | aagcaagauauuuuuauaaua |
| | | | T2CO)(NH2C12)AfaGfcAfa | | |
| | | | GfaUfaUfuUfuUfaUfaAfsus | | |
| | | | Af | | |
| 97. | ETX013 | | | usAfsuUfaUfaAfaAfaUfaUfcUfuGfcUf | uauuauaaaaauaucuugcuuuu |
| | | | | usUfsudTdT | |
| 98. | ETX017 | | AfsasGfcAfaGfaUfaUfuUfu | | aagcaagauauuuuuauaaua |
| | | | UfaUfaAfuAf(NHC6)(ET- | | |
| | | | GalNAc-T2CO) | | |
| 99. | ETX017 | | | usAfsuUfaUfaAfaAfaUfaUfcUfuGfcUf | uauuauaaaaauaucuugcuuuu |
| | | | | usUfsudTdT | |
| 100. | ETX020 | | (ET-GalNAc- | | ugggauuucauguaaccaaga |
| | | | T2CO)(NH2C12)ugggauUfu | | |
| | | | CfAfUfguaaccaasgsa | | |
| | | | (invabasic)(invabasic) | | |
| 101. | ETX020 | | | usCfsuugGfuuAfcaugAfaAfucccasusc | ucuugguuacaugaaaucccauc |
| 102. | ETX022 | | (ET-GalNAc- | | ugggauuucauguaaccaaga |
| | | | T2CO)(NH2C12)UfgGfgAf | | |
| | | | uUfuCfaUfgUfaAfcCfaAfsg | | |
| | | | sAf | | |
| 103. | ETX022 | | | usCfsuUfgGfuUfaCfaUfgAfaAfuCfcCf | ucuugguuacaugaaaucccauc |
| | | | | asUfsc | |
| 104. | ETX024 | | (invabasic)(invabasic)usgsg | | ugggauuucauguaaccaaga |
| | | | gauUfuCfAfUfguaaccaaga | | |
| | | | (NHC6)(ET-GalNAc-T2CO) | | |
| 105. | ETX024 | | | usCfsuugGfuuAfcaugAfaAfucccasusc | ucuugguuacaugaaaucccauc |
| 106. | ETX026 | | UfsgsGfgAfuUfuCfaUfgUfa | | ugggauuucauguaaccaaga |
| | | | AfcCfaAfgAf(NHC6)(ET- | | |
| | | | GalNAc-T2CO) | | |
| 107. | ETX026 | | | usCfsuUfgGfuUfaCfaUfgAfaAfuCfcCf | ucuugguuacaugaaaucccauc |
| | | | | asUfsc | |
| 108. | XD- | | cuuAcGcuGAGuAcuucGAd | | cuuacgcugaguacuucga |
| | 00914 | | TsdT | | |
| 109. | XD- | | | UCGAAGuACUcAGCGuAAGdTsdT | ucgaaguacucagcguaag |
| | 00914 | | | | |
| 110. | XD- | | AGAuAuGcAcAcAcAcGG | | agauaugcacacacacgga |
| | 03999 | | AdTsdT | | |
| 111. | XD- | | | UCCGUGUGUGUGcAuAUCUdTsdT | uccgugugugugcauaucu |
| | 03999 | | | | |
| 112. | XD- | | uscsUfcGfuGfgCfcUfuAfaU | | ucucguggccuuaaugaaa |
| | 15421 | | fgAfaAf(invdT) | | |
| 113. | XD- | | | UfsUfsuCfaUfuAfaGfgCfcAfcGfaGfas | uuucauuaaggccacgagauu |
| | 15421 | | | usu | |
| 114. | | X91382 | (NH2- | | gacuuucauccuggaaauaua |
| | | | DEG)gacuuuCfaUfCfCfugg | | |
| | | | aaauasusa(invabasic) | | |
| | | | (invabasic) | | |
| 115. | | X91383 | (NH2- | | aagcaagauauuuuuauaaua |
| | | | DEG)aaGfcAfaGfaUfAfUfu | | |
| | | | UfuuAfuAfasusa(invabasic) | | |
| | | | (invabasic) | | |
| 116. | | X91384 | (NH2- | | ugggauuucauguaaccaaga |
| | | | DEG)ugggauUfuCfAfUfgua | | |
| | | | accaasgsa(invabasic) | | |
| | | | (invabasic) | | |
| 117. | | X91385 | (NH2- | | gacuuucauccuggaaauaua |
| | | | DEG)GfaCfuUfuCfaUfcCfu | | |
| | | | GfgAfaAfuAfsusAf | | |
| 118. | | X91386 | (NH2- | | aagcaagauauuuuuauaaua |
| | | | DEG)AfaGfcAfaGfaUfaUfu | | |
| | | | UfuUfaUfaAfsusAf | | |
| 119. | | X91387 | (NH2- | | ugggauuucauguaaccaaga |
| | | | DEG)UfgGfgAfuUfuCfaUfg | | |
| | | | UfaAfcCfaAfsgsAf | | |
| 120. | | X91403 | (NH2C12)gacuuuCfaUfCfC | | gacuuucauccuggaaauaua |
| | | | fuggaaauasusa(invabasic) | | |
| | | | (invabasic) | | |
| 121. | | X91404 | (NH2C12)aaGfc AfaGfaUfA | | aagcaagauauuuuuauaaua |
| | | | fUfuUfuuAfuAfasusa | | |
| | | | (invabasic)(invabasic) | | |
| 122. | | X91405 | (NH2C12)ugggauUfuCfAfU | | ugggauuucauguaaccaaga |
| | | | fguaaccaasgsa(invabasic) | | |
| | | | (invabasic) | | |
| 123. | | X91406 | (NH2C12)GfaCfuUfuCfaUf | | gacuuucauccuggaaauaua |
| | | | cCfuGfgAfaAfuAfsusAf | | |
| 124. | | X91407 | (NH2C12)AfaGfcAfaGfaUf | | aagcaagauauuuuuauaaua |
| | | | aUfuUfuUfaUfaAfsusAf | | |
| 125 | | X91408 | (NH2C12)UfgGfgAfuUfuCf | | ugggauuucauguaaccaaga |
| | | | aUfgUfaAfcCfaAfsgsAf | | |
| 126. | | X91415 | (invabasic)(invabasic)gsascu | | gacuuucauccuggaaauaua |
| | | | uuCfaUfCfCfuggaaauasusa | | |
| | | | (NH2C6) | | |
| 127. | | X91416 | (invabasic)(invabasic)asasGf | | aagcaagauauuuuuauaaua |
| | | | cAfaGfaUfAfUfuUfuuAfuA | | |
| | | | faua(NH2C6) | | |
| 128. | | X91417 | (invabasic)(invabasic)usgsg | | ugggauuucauguaaccaaga |
| | | | gauUfuCfAfUfguaaccaaga | | |
| | | | (NH2C6) | | |
| 129. | | X91418 | GfsasCfuUfuCfaUfcCfuGfg | | gacuuucauccuggaaauaua |
| | | | AfaAfuAfuAf(NH2C6) | | |
| 130. | | X91419 | AfsasGfcAfaGfaUfaUfuUfu | | aagcaagauauuuuuauaaua |
| | | | UfaUfaAfuAf(NH2C6) | | |
| 131. | | X91420 | UfsgsGfgAfuUfuCfaUfgUfa | | ugggauuucauguaaccaaga |
| | | | AfcCfaAfgAf(NH2C6) | | |
| 132. | | X91379 | gsascuuuCfaUfCfCfuggaaau | | gacuuucauccuggaaauaua |
| | | | aua(GalNAc) | | |
| 133. | | X91380 | asasGfcAfaGfaUfAfUfuUfu | | aagcaagauauuuuuauaaua |
| | | | uAfuAfaua(GalNAc) | | |
| 134. | | X91446 | usgsggauUfuCfAfUfguaacca | | ugggauuucauguaaccaaga |
| | | | aga(GalNAc) | | |
| 135. | | X38483 | usAfsuauUfuCfCfaggaUfgA | | uauauuuccaggaugaaagucca |
| | | | faagucscsa | | |
| 136. | | X91381 | usAfsUfuAfuaAfaAfauaUfc | | uauuauaaaaauaucuugcuuuu |
| | | | UfuGfcuususudTdT | | |
| 137. | | X38104 | usCfsuugGfuuAfcaugAfaAf | | ucuugguuacaugaaaucccauc |
| | | | ucccasusc | | |
| 138. | | X91398 | usAfsuAfuUfuCfcAfgGfaUf | | uauauuuccaggaugaaagucca |
| | | | gAfaAfgUfcsCfsa | | |
| 139. | | X91400 | usAfsuUfaUfaAfaAfaUfaUf | | uauuauaaaaauaucuugcuuuu |
| | | | cUfuGfcUfusUfsudTdT | | |
| 140. | | X91402 | usCfsuUfgGfuUfaCfaUfgAf | | ucuugguuacaugaaaucccauc |
| | | | aAfuCfcCfasUfsc | | |
| 141. | | X91409 | (GalNAc- | | gacuuucauccuggaaauaua |
| | | | T2)(NH2C12)gacuuuCfaUf | | |
| | | | CfCfuggaaauasusa(invabasic) | | |
| | | | (invabasic) | | |
| 142. | | X91410 | (GalNAc- | | aagcaagauauuuuuauaaua |
| | | | T2)(NH2C12)aaGfcAfaGfa | | |
| | | | UfAfUfuUfuuAfuAfasusa | | |
| | | | (invabasic)(invabasic) | | |
| 143. | | X91411 | (GalNAc- | | ugggauuucauguaaccaaga |
| | | | T2)(NH2C12)ugggauUfuCf | | |
| | | | AfUfguaaccaasgsa(invabasic) | | |
| | | | (invabasic) | | |
| 144. | | X91412 | (GalNAc- | | gacuuucauccuggaaauaua |
| | | | T2)(NH2C12)GfaCfuUfuCf | | |
| | | | aUfcCfuGfgAfaAfuAfsusAf | | |
| 145. | | X91413 | (GalNAc- | | aagcaagauauuuuuauaaua |
| | | | T2)(NH2C12)AfaGfcAfaGf | | |
| | | | aUfaUfuUfuUfaUfaAfsusAf | | |
| 146 | | X91414 | (GalNAc- | | ugggauuucauguaaccaaga |
| | | | T2)(NH2C12)UfgGfgAfuUf | | |
| | | | uCfaUfgUfaAfcCfaAfsgsAf | | |
| 147. | | X91433 | (invabasic)(invabasic)gsascu | | gacuuucauccuggaaauaua |
| | | | uuCfaUfCfCfuggaaauasusa | | |
| | | | (NHC6)(GalNAc-T2) | | |
| 148. | | X91434 | (invabasic)(invabasic)asasGf | | aagcaagauauuuuuauaaua |
| | | | cAfaGfaUfAfUfuUfuuAfuA | | |
| | | | faua(NHC6)(GalNAc-T2) | | |
| 149. | | X91435 | (invabasic)(invabasic)usgsg | | ugggauuucauguaaccaaga |
| | | | gauUfuCfAfUfguaaccaaga | | |
| | | | (NHC6)(GalNAc-T2) | | |
| 150. | | X91436 | GfsasCfuUfuCfaUfcCfuGfg | | gacuuucauccuggaaauaua |
| | | | AfaAfuAfuAf(NHC6) | | |
| | | | (GalNAc-T2) | | |
| 151. | | X91437 | AfsasGfcAfaGfaUfaUfuUfu | | aagcaagauauuuuuauaaua |
| | | | UfaUfaAfuAf(NHC6) | | |
| | | | (GalNAc-T2) | | |
| 152. | | X91438 | UfsgsGfgAfuUfuCfaUfgUfa | | ugggauuucauguaaccaaga |
| | | | AfcCfaAfgAf(NHC6) | | |
| | | | (GalNAc-T2) | | |
| 153. | ETX002 | X91409 | (GalNAc- | | gacuuucauccuggaaauaua |
| | | | T2)(NH2C12)gacuuuCfaUf | | |
| | | | CfCfuggaaauasusa(invabasic) | | |
| | | | (invabasic) | | |
| 154. | | X38483 | usAfsuauUfuCfCfaggaUfgA | | uauauuuccaggaugaaagucca |
| | | | faagucscsa | | |
| 155. | ETX004 | X91412 | (ET-GalNAc- | | gacuuucauccuggaaauaua |
| | | | T2CO)(NH2C12)GfaCfuUf | | |
| | | | uCfaUfcCfuGfgAfaAfuAfsu | | |
| | | | sAf | | |
| 156. | | X91398 | usAfsuAfuUfuCfcAfgGfaUf | | uauauuuccaggaugaaagucca |
| | | | gAfaAfgUfcsCfsa | | |
| 157. | ETX006 | X91433 | (invabasic)(invabasic)gsascu | | gacuuucauccuggaaauaua |
| | | | uuCfaUfCfCfuggaaauasusa | | |
| | | | (NHC6)(GalNAc-T2) | | |
| 158. | | X38483 | usAfsuauUfuCfCfaggaUfgA | | uauauuuccaggaugaaagucca |
| | | | faagucscsa | | |
| 159. | ETX008 | X91436 | GfsasCfuUfuCfaUfcCfuGfg | | gacuuucauccuggaaauaua |
| | | | AfaAfuAfuAf(NHC6) | | |
| | | | (GalNAc-T2) | | |
| 160. | | X91398 | usAfsuAfuUfuCfcAfgGfaUf | | uauauuuccaggaugaaagucca |
| | | | gAfaAfgUfcsCfsa | | |
| 161. | ETX011 | X91410 | (GalNAc- | | aagcaagauauuuuuauaaua |
| | | | T2)(NH2C12)aaGfcAfaGfa | | |
| | | | UfAfUfuUfuuAfuAfasusa | | |
| | | | (invabasic)(invabasic) | | |
| 162. | | X91381 | usAfsUfuAfuaAfaAfauaUfc | | uauuauaaaaauaucuugcuuuu |
| | | | UfuGfcuususudTdT | | |
| 163. | ETX013 | X91413 | (GalNAc- | | aagcaagauauuuuuauaaua |
| | | | T2)(NH2C12)AfaGfcAfaGf | | |
| | | | aUfaUfuUfuUfaUfaAfsusAf | | |
| 164. | | X91400 | usAfsuUfaUfaAfaAfaUfaUf | | uauuauaaaaauaucuugcuuuu |
| | | | cUfuGfcUfusUfsudTdT | | |
| 165. | ETX015 | X91434 | (invabasic)(invabasic)asasGf | | aagcaagauauuuuuauaaua |
| | | | cAfaGfaUfAfUfuUfuuAfuA | | |
| | | | faua(NHC6)(GalNAc-T2) | | |
| 166. | | X91381 | usAfsUfuAfuaAfaAfauaUfc | | uauuauaaaaauaucuugcuuuu |
| | | | UfuGfcuususudTdT | | |
| 167. | ETX017 | X91437 | AfsasGfcAfaGfaUfaUfuUfu | | aagcaagauauuuuuauaaua |
| | | | UfaUfaAfuAf(NHC6) | | |
| | | | (GalNAc-T2) | | |
| 168. | | X91400 | usAfsuUfaUfaAfaAfaUfaUf | | uauuauaaaaauaucuugcuuuu |
| | | | cUfuGfcUfusUfsudTdT | | |
| 169. | ETX020 | X91411 | (GalNAc- | | ugggauuucauguaaccaaga |
| | | | T2)(NH2C12)ugggauUfuCf | | |
| | | | AfUfguaaccaasgsa(invabasic) | | |
| | | | (invabasic) | | |
| 170. | | X38104 | usCfsuugGfuuAfcaugAfaAf | | ucuugguuacaugaaaucccauc |
| | | | ucccasusc | | |
| 171. | ETX022 | X91414 | (GalNAc- | | ugggauuucauguaaccaaga |
| | | | T2)(NH2C12)UfgGfgAfuUf | | |
| | | | uCfaUfgUfaAfcCfaAfsgsAf | | |
| 172. | | X91402 | usCfsuUfgGfuUfaCfaUfgAf | | ucuugguuacaugaaaucccauc |
| | | | aAfuCfcCfasUfsc | | |
| 173. | ETX024 | X91435 | (invabasic)(invabasic)usgsg | | ugggauuucauguaaccaaga |
| | | | gauUfuCfAfUfguaaccaaga | | |
| | | | (NHC6)(GalNAc-T2) | | |
| 174. | | X38104 | usCfsuugGfuuAfcaugAfaAf | | ucuugguuacaugaaaucccauc |
| | | | ucccasusc | | |
| 175. | ETX026 | X91438 | UfsgsGfgAfuUfuCfaUfgUfa | | ugggauuucauguaaccaaga |
| | | | AfcCfaAfgAf(NHC6) | | |
| | | | (GalNAc-T2) | | |
| 176. | | X91402 | usCfsuUfgGfuUfaCfaUfgAf | | ucuugguuacaugaaaucccauc |
| | | | aAfuCfcCfasUfsc | | |

### Key:

### Key for SEQ ID NOs: 1-24

ia inverted abasic nucleotide (1,2-dideoxyribose)
m 2′-O-methyl nucleotide
f 2′-deoxy-2′-fluoro nucleotide
s phosphorothioate internucleotide linkage (Phosphorothioate backbone modification)
˜ tether
Td Deoxythymidine
Key for SEQ ID NOs: 25-176
dG, dC, dA, dT DNA residues (
GalNAc N-Acetylgalactosamine
G, C, A, U RNA residues
g, c, a, u 2′-O-Methyl modified residues
Gf, Cf, Af, Uf 2′-Fluoro modified residues
s Phosphorothioate backbone modification
siRNA small interfering RNA
MFCO Monofluoro cyclooctyne
invabasic 1,2-dideoxyribose
(invabasic)(invabasic)Nucleotides in an overall polynucleotide which are the terminal 2 nucleotides which have sugar moieties that are (i) abasic, and (ii) in an inverted configuration, whereby the bond between the penultimate nucleotide and the antepenultimate nucleotide has a
reversed linkage, namely either a 5-5 or a 3-3 linkage
NH2-DEG/NHDEG Aminoethoxyethyl linker
NH2C12 Aminododecyl linker
NH2C6/NHC6 Aminohexyl linker
ET (E-therapeutics—company reference)
(ET-GalNAc-T1N3)(MFCO)(NH-DEG) tether T1b
(ET-GalNAc-T2CO)(NH2C12) tether T2b
(NHC6)(MFCO)(ET-GalNAc-T1N3)tether Tia
(NHC6)(ET-GalNAc-T2CO) tether T2a
T1N3 T1 tether
T2Co T2 tether
(GalNAc-)T1 GalNac T1 tether
(GalNAc-)T2 GalNac T2 tether
Note: the key refers to the sense and antisense sequence, whereas the clean sequence contains the underlying RNA nucleotide only.

1: A nucleic acid, comprising at least one duplex region that comprises at least a portion of a first strand and at least a portion of a second strand that is at least partially complementary to the first strand, wherein the first strand is at least partially complementary to at least a portion of RNA transcribed from the target gene to be inhibited,
wherein the second strand comprises two consecutive abasic nucleotides in the 5′ terminal region of the second strand, wherein one such abasic nucleotide is a terminal nucleotide at the 5′ terminal region of the second strand and the other abasic nucleotide is a penultimate nucleotide at the 5′ terminal region of the second strand, wherein
a) the penultimate nucleotide of the second strand is connected to an adjacent nucleotide which is not the terminal nucleotide (called the antepenultimate nucleotide herein) through a reversed internucleotide linkage,
b) the reversed internucleotide linkage is a 5-5′ reversed linkage, and
c) the linkages between the terminal and penultimate abasic nucleotides is 3′-5′ when reading towards the terminus comprising the terminal and penultimate abasic nucleotides.

2: The nucleic acid according to claim 1,
wherein the abasic nucleotides are present in an overhang.

3-7. (canceled)

8: The nucleic acid according to claim 1, wherein one or more nucleotides on the first strand and the second strand are modified, to form modified nucleotides.

9: The nucleic acid according to claim 8, wherein the modification is a modification at the 2′—OH group of the ribose sugar selected from 2′-Me or 2′-F modifications.

10-11. (canceled)

12: The nucleic acid according to claim 9, wherein the first and second strand each comprise 2′-Me and 2′-F modifications.

13: The nucleic acid according to claim 1, wherein the nucleic acid comprises at least one thermally destabilizing modification, suitably at one or more of positions 1 to 9 of the first strand, or at one or more of positions on the second strand aligned with positions 1 to 9 of the first strand, wherein the destabilizing modification is selected from a modified unlocked nucleic acid (IMUNA) and a glycol nucleic acid (GNA)).

14: The nucleic acid according to claim 13, wherein the nucleic acid is a double stranded molecule, which has a melting temperature in the range of about 40 to 80° C.

15-41. (canceled)

42: The nucleic acid according to claim 1, wherein the nucleic acid further comprises phosphorothioate internucleotide linkages respectively between at least three consecutive positions in a 5′ or 3′ near terminal region of the second strand.

43: The nucleic acid according to claim 1, wherein the nucleic acid further comprises phosphorothioate internucleotide linkages respectively between at least three consecutive positions in a 5′ terminal region of the first strand.

44-47. (canceled)

48: A pharmaceutical composition comprising the nucleic acid according to claim 1 and a physiologically acceptable excipient.

49: The nucleic acid according to claim 13, wherein the destabilizing modification is a GNA.

50: The nucleic acid according to claim 14, wherein the nucleic acid is double stranded RNA.

51: The nucleic acid according to claim 42, wherein the near terminal region is adjacent to the terminal region wherein the abasic nucleotides of the second strand are located.


SEQUENCE LISTING (176 sequences)

SEQ ID NO: 1 - RNA, 21 nt, synthetic construct
  misc_feature 1..21: note = modified siRNA
  source 1..21: mol_type = other RNA
gactttcatcctggaaatata

SEQ ID NO: 2 - RNA, 23 nt, synthetic construct
  misc_feature 1..23: note = modified siRNA
  source 1..23: mol_type = other RNA
acctgaaagtaggacctttatat

SEQ ID NO: 3 - RNA, 21 nt, synthetic construct
  misc_feature 1..21: note = modified siRNA
  source 1..21: mol_type = other RNA
aagcaagatatttttataata

SEQ ID NO: 4 - RNA, 23 nt, synthetic construct
  misc_feature 1..23: note = modified siRNA
  source 1..23: mol_type = other RNA
ttttcgttctataaaaatattat

SEQ ID NO: 5 - RNA, 21 nt, synthetic construct
  misc_feature 1..21: note = modified siRNA
  source 1..21: mol_type = other RNA
tgggatttcatgtaaccaaga

SEQ ID NO: 6 - RNA, 23 nt, synthetic construct
  misc_feature 1..23: note = modified siRNA
  source 1..23: mol_type = other RNA
ctaccctaaagtacattggttct

SEQ ID NO: 7 - RNA, 21 nt, synthetic construct
  misc_feature 1..21: note = modified siRNA
  source 1..21: mol_type = other RNA
gactttcatcctggaaatata

SEQ ID NO: 8 - RNA, 23 nt, synthetic construct
  misc_feature 1..23: note = modified siRNA
  source 1..23: mol_type = other RNA
acctgaaagtaggacctttatat

SEQ ID NO: 9 - RNA, 21 nt, synthetic construct
  misc_feature 1..21: note = modified siRNA
  source 1..21: mol_type = other RNA
aagcaagatatttttataata

SEQ ID NO: 10 - RNA, 23 nt, synthetic construct
  misc_feature 1..23: note = modified siRNA
  source 1..23: mol_type = other RNA
ttttcgttctataaaaatattat

SEQ ID NO: 11 - RNA, 21 nt, synthetic construct
  misc_feature 1..21: note = modified siRNA
  source 1..21: mol_type = other RNA
tgggatttcatgtaaccaaga

SEQ ID NO: 12 - RNA, 23 nt, synthetic construct
  misc_feature 1..23: note = modified siRNA
  source 1..23: mol_type = other RNA
ctaccctaaagtacattggttct

SEQ ID NO: 13 - RNA, 21 nt, synthetic construct
  misc_feature 1..21: note = modified siRNA
  source 1..21: mol_type = other RNA
gactttcatcctggaaatata

SEQ ID NO: 14 - RNA, 23 nt, synthetic construct
  misc_feature 1..23: note = modified siRNA
  source 1..23: mol_type = other RNA
acctgaaagtaggacctttatat

SEQ ID NO: 15 - RNA, 21 nt, synthetic construct
  misc_feature 1..21: note = modified siRNA
  source 1..21: mol_type = other RNA
aagcaagatatttttataata

SEQ ID NO: 16 - RNA, 23 nt, synthetic construct
  misc_feature 1..23: note = modified siRNA
  source 1..23: mol_type = other RNA
ttttcgttctataaaaatattat

SEQ ID NO: 17 - RNA, 21 nt, synthetic construct
  misc_feature 1..21: note = modified siRNA
  source 1..21: mol_type = other RNA
tgggatttcatgtaaccaaga

SEQ ID NO: 18 - RNA, 23 nt, synthetic construct
  misc_feature 1..23: note = modified siRNA
  source 1..23: mol_type = other RNA
ctaccctaaagtacattggttct

SEQ ID NO: 19 - RNA, 21 nt, synthetic construct
  misc_feature 1..21: note = modified siRNA
  source 1..21: mol_type = other RNA
gactttcatcctggaaatata

SEQ ID NO: 20 - RNA, 23 nt, synthetic construct
  misc_feature 1..23: note = modified siRNA
  source 1..23: mol_type = other RNA
acctgaaagtaggacctttatat

SEQ ID NO: 21 - RNA, 21 nt, synthetic construct
  misc_feature 1..21: note = modified siRNA
  source 1..21: mol_type = other RNA
aagcaagatatttttataata

SEQ ID NO: 22 - RNA, 23 nt, synthetic construct
  misc_feature 1..23: note = modified siRNA
  source 1..23: mol_type = other RNA
ttttcgttctataaaaatattat

SEQ ID NO: 23 - RNA, 21 nt, synthetic construct
  misc_feature 1..21: note = modified siRNA
  source 1..21: mol_type = other RNA
tgggatttcatgtaaccaaga

SEQ ID NO: 24 - RNA, 23 nt, synthetic construct
  misc_feature 1..23: note = modified siRNA
  source 1..23: mol_type = other RNA
ctaccctaaagtacattggttct

SEQ ID NO: 25 - RNA, 21 nt, synthetic construct
  misc_feature 1..21: note = modified siRNA
  source 1..21: mol_type = other RNA
gactttcatcctggaaatata

SEQ ID NO: 26 - RNA, 23 nt, synthetic construct
  misc_feature 1..23: note = modified siRNA
  source 1..23: mol_type = other RNA
tatatttccaggatgaaagtcca

SEQ ID NO: 27 - RNA, 21 nt, synthetic construct
  misc_feature 1..21: note = modified siRNA
  source 1..21: mol_type = other RNA
gactttcatcctggaaatata

SEQ ID NO: 28 - RNA, 23 nt, synthetic construct
  misc_feature 1..23: note = modified siRNA
  source 1..23: mol_type = other RNA
tatatttccaggatgaaagtcca

SEQ ID NO: 29 - RNA, 21 nt, synthetic construct
  misc_feature 1..21: note = modified siRNA
  source 1..21: mol_type = other RNA
aagcaagatatttttataata

SEQ ID NO: 30 - RNA, 23 nt, synthetic construct
  misc_feature 1..23: note = modified siRNA
  source 1..23: mol_type = other RNA
tattataaaaatatcttgctttt

SEQ ID NO: 31 - RNA, 21 nt, synthetic construct
  misc_feature 1..21: note = modified siRNA
  source 1..21: mol_type = other RNA
aagcaagatatttttataata

SEQ ID NO: 32 - RNA, 23 nt, synthetic construct
  misc_feature 1..23: note = modified siRNA
  source 1..23: mol_type = other RNA
tattataaaaatatcttgctttt

SEQ ID NO: 33 - RNA, 21 nt, synthetic construct
  misc_feature 1..21: note = modified siRNA
  source 1..21: mol_type = other RNA
tgggatttcatgtaaccaaga

SEQ ID NO: 34 - RNA, 23 nt, synthetic construct
  misc_feature 1..23: note = modified siRNA
  source 1..23: mol_type = other RNA
tcttggttacatgaaatcccatc

SEQ ID NO: 35 - RNA, 21 nt, synthetic construct
  misc_feature 1..21: note = modified siRNA
  source 1..21: mol_type = other RNA
tgggatttcatgtaaccaaga

SEQ ID NO: 36 - RNA, 23 nt, synthetic construct
  misc_feature 1..23: note = modified siRNA
  source 1..23: mol_type = other RNA
tcttggttacatgaaatcccatc

SEQ ID NO: 37 - RNA, 19 nt, synthetic construct
  misc_feature 1..19: note = modified siRNA
  source 1..19: mol_type = other RNA
cttacgctgagtacttcga

SEQ ID NO: 38 - RNA, 19 nt, synthetic construct
  misc_feature 1..19: note = modified siRNA
  source 1..19: mol_type = other RNA
tcgaagtactcagcgtaag

SEQ ID NO: 39 - RNA, 19 nt, synthetic construct
  misc_feature 1..19: note = modified siRNA
  source 1..19: mol_type = other RNA
agatatgcacacacacgga

SEQ ID NO: 40 - RNA, 19 nt, synthetic construct
  misc_feature 1..19: note = modified siRNA
  source 1..19: mol_type = other RNA
tccgtgtgtgtgcatatct

SEQ ID NO: 41 - RNA, 19 nt, synthetic construct
  misc_feature 1..19: note = modified siRNA
  source 1..19: mol_type = other RNA
tctcgtggccttaatgaaa

SEQ ID NO: 42 - RNA, 21 nt, synthetic construct
  misc_feature 1..21: note = modified siRNA
  source 1..21: mol_type = other RNA
tttcattaaggccacgagatt

SEQ ID NO: 43 - RNA, 21 nt, synthetic construct
  misc_feature 1..21: note = modified siRNA
  source 1..21: mol_type = other RNA
gactttcatcctggaaatata

SEQ ID NO: 44 - RNA, 21 nt, synthetic construct
  misc_feature 1..21: note = modified siRNA
  source 1..21: mol_type = other RNA
aagcaagatatttttataata

SEQ ID NO: 45 - RNA, 21 nt, synthetic construct
  misc_feature 1..21: note = modified siRNA
  source 1..21: mol_type = other RNA
tgggatttcatgtaaccaaga

SEQ ID NO: 46 - RNA, 21 nt, synthetic construct
  misc_feature 1..21: note = modified siRNA
  source 1..21: mol_type = other RNA
gactttcatcctggaaatata

SEQ ID NO: 47 - RNA, 21 nt, synthetic construct
  misc_feature 1..21: note = modified siRNA
  source 1..21: mol_type = other RNA
aagcaagatatttttataata

SEQ ID NO: 48 - RNA, 21 nt, synthetic construct
  misc_feature 1..21: note = modified siRNA
  source 1..21: mol_type = other RNA
tgggatttcatgtaaccaaga

SEQ ID NO: 49 - RNA, 21 nt, synthetic construct
  misc_feature 1..21: note = modified siRNA
  source 1..21: mol_type = other RNA
gactttcatcctggaaatata

SEQ ID NO: 50 - RNA, 21 nt, synthetic construct
  misc_feature 1..21: note = modified siRNA
  source 1..21: mol_type = other RNA
aagcaagatatttttataata

SEQ ID NO: 51 - RNA, 21 nt, synthetic construct
  misc_feature 1..21: note = modified siRNA
  source 1..21: mol_type = other RNA
tgggatttcatgtaaccaaga

SEQ ID NO: 52 - RNA, 21 nt, synthetic construct
  misc_feature 1..21: note = modified siRNA
  source 1..21: mol_type = other RNA
gactttcatcctggaaatata

SEQ ID NO: 53 - RNA, 21 nt, synthetic construct
  misc_feature 1..21: note = modified siRNA
  source 1..21: mol_type = other RNA
aagcaagatatttttataata

SEQ ID NO: 54 - RNA, 21 nt, synthetic construct
  misc_feature 1..21: note = modified siRNA
  source 1..21: mol_type = other RNA
tgggatttcatgtaaccaaga

SEQ ID NO: 55 - RNA, 23 nt, synthetic construct
  misc_feature 1..23: note = modified siRNA
  source 1..23: mol_type = other RNA
tatatttccaggatgaaagtcca

SEQ ID NO: 56 - RNA, 23 nt, synthetic construct
  misc_feature 1..23: note = modified siRNA
  source 1..23: mol_type = other RNA
tattataaaaatatcttgctttt

SEQ ID NO: 57 - RNA, 23 nt, synthetic construct
  misc_feature 1..23: note = modified siRNA
  source 1..23: mol_type = other RNA
tcttggttacatgaaatcccatc

SEQ ID NO: 58 - RNA, 21 nt, synthetic construct
  misc_feature 1..21: note = modified siRNA
  source 1..21: mol_type = other RNA
gactttcatcctggaaatata

SEQ ID NO: 59 - RNA, 21 nt, synthetic construct
  misc_feature 1..21: note = modified siRNA
  source 1..21: mol_type = other RNA
aagcaagatatttttataata

SEQ ID NO: 60 - RNA, 21 nt, synthetic construct
  misc_feature 1..21: note = modified siRNA
  source 1..21: mol_type = other RNA
tgggatttcatgtaaccaaga

SEQ ID NO: 61 - RNA, 21 nt, synthetic construct
  misc_feature 1..21: note = modified siRNA
  source 1..21: mol_type = other RNA
gactttcatcctggaaatata

SEQ ID NO: 62 - RNA, 21 nt, synthetic construct
  misc_feature 1..21: note = modified siRNA
  source 1..21: mol_type = other RNA
aagcaagatatttttataata

SEQ ID NO: 63 - RNA, 21 nt, synthetic construct
  misc_feature 1..21: note = modified siRNA
  source 1..21: mol_type = other RNA
tgggatttcatgtaaccaaga

SEQ ID NO: 64 - RNA, 21 nt, synthetic construct
  misc_feature 1..21: note = modified siRNA
  source 1..21: mol_type = other RNA
gactttcatcctggaaatata

SEQ ID NO: 65 - RNA, 21 nt, synthetic construct
  misc_feature 1..21: note = modified siRNA
  source 1..21: mol_type = other RNA
aagcaagatatttttataata

SEQ ID NO: 66 - RNA, 21 nt, synthetic construct
  misc_feature 1..21: note = modified siRNA
  source 1..21: mol_type = other RNA
tgggatttcatgtaaccaaga

SEQ ID NO: 67 - RNA, 21 nt, synthetic construct
  misc_feature 1..21: note = modified siRNA
  source 1..21: mol_type = other RNA
gactttcatcctggaaatata

SEQ ID NO: 68 - RNA, 21 nt, synthetic construct
  misc_feature 1..21: note = modified siRNA
  source 1..21: mol_type = other RNA
aagcaagatatttttataata

SEQ ID NO: 69 - RNA, 21 nt, synthetic construct
  misc_feature 1..21: note = modified siRNA
  source 1..21: mol_type = other RNA
tgggatttcatgtaaccaaga

SEQ ID NO: 70 - RNA, 21 nt, synthetic construct
  misc_feature 1..21: note = modified siRNA
  source 1..21: mol_type = other RNA
gactttcatcctggaaatata

SEQ ID NO: 71 - RNA, 23 nt, synthetic construct
  misc_feature 1..23: note = modified siRNA
  source 1..23: mol_type = other RNA
tatatttccaggatgaaagtcca

SEQ ID NO: 72 - RNA, 21 nt, synthetic construct
  misc_feature 1..21: note = modified siRNA
  source 1..21: mol_type = other RNA
gactttcatcctggaaatata

SEQ ID NO: 73 - RNA, 23 nt, synthetic construct
  misc_feature 1..23: note = modified siRNA
  source 1..23: mol_type = other RNA
tatatttccaggatgaaagtcca

SEQ ID NO: 74 - RNA, 21 nt, synthetic construct
  misc_feature 1..21: note = modified siRNA
  source 1..21: mol_type = other RNA
aagcaagatatttttataata

SEQ ID NO: 75 - RNA, 23 nt, synthetic construct
  misc_feature 1..23: note = modified siRNA
  source 1..23: mol_type = other RNA
tattataaaaatatcttgctttt

SEQ ID NO: 76 - RNA, 21 nt, synthetic construct
  misc_feature 1..21: note = modified siRNA
  source 1..21: mol_type = other RNA
aagcaagatatttttataata

SEQ ID NO: 77 - RNA, 23 nt, synthetic construct
  misc_feature 1..23: note = modified siRNA
  source 1..23: mol_type = other RNA
tattataaaaatatcttgctttt

SEQ ID NO: 78 - RNA, 21 nt, synthetic construct
  misc_feature 1..21: note = modified siRNA
  source 1..21: mol_type = other RNA
tgggatttcatgtaaccaaga

SEQ ID NO: 79 - RNA, 23 nt, synthetic construct
  misc_feature 1..23: note = modified siRNA
  source 1..23: mol_type = other RNA
tcttggttacatgaaatcccatc

SEQ ID NO: 80 - RNA, 21 nt, synthetic construct
  misc_feature 1..21: note = modified siRNA
  source 1..21: mol_type = other RNA
tgggatttcatgtaaccaaga

SEQ ID NO: 81 - RNA, 23 nt, synthetic construct
  misc_feature 1..23: note = modified siRNA
  source 1..23: mol_type = other RNA
tcttggttacatgaaatcccatc

SEQ ID NO: 82 - RNA, 21 nt, synthetic construct
  misc_feature 1..21: note = modified siRNA
  source 1..21: mol_type = other RNA
gactttcatcctggaaatata

SEQ ID NO: 83 - RNA, 23 nt, synthetic construct
  misc_feature 1..23: note = modified siRNA
  source 1..23: mol_type = other RNA
tatatttccaggatgaaagtcca

SEQ ID NO: 84 - RNA, 21 nt, synthetic construct
  misc_feature 1..21: note = modified siRNA
  source 1..21: mol_type = other RNA
gactttcatcctggaaatata

SEQ ID NO: 85 - RNA, 23 nt, synthetic construct
  misc_feature 1..23: note = modified siRNA
  source 1..23: mol_type = other RNA
tatatttccaggatgaaagtcca

SEQ ID NO: 86 - RNA, 21 nt, synthetic construct
  misc_feature 1..21: note = modified siRNA
  source 1..21: mol_type = other RNA
gactttcatcctggaaatata

SEQ ID NO: 87 - RNA, 23 nt, synthetic construct
  misc_feature 1..23: note = modified siRNA
  source 1..23: mol_type = other RNA
tatatttccaggatgaaagtcca

SEQ ID NO: 88 - RNA, 21 nt, synthetic construct
  misc_feature 1..21: note = modified siRNA
  source 1..21: mol_type = other RNA
gactttcatcctggaaatata

SEQ ID NO: 89 - RNA, 23 nt, synthetic construct
  misc_feature 1..23: note = modified siRNA
  source 1..23: mol_type = other RNA
tatatttccaggatgaaagtcca

SEQ ID NO: 90 - RNA, 21 nt, synthetic construct
  misc_feature 1..21: note = modified siRNA
  source 1..21: mol_type = other RNA
gactttcatcctggaaatata

SEQ ID NO: 91 - RNA, 23 nt, synthetic construct
  misc_feature 1..23: note = modified siRNA
  source 1..23: mol_type = other RNA
tatatttccaggatgaaagtcca

SEQ ID NO: 92 - RNA, 21 nt, synthetic construct
  misc_feature 1..21: note = modified siRNA
  source 1..21: mol_type = other RNA
aagcaagatatttttataata

SEQ ID NO: 93 - RNA, 23 nt, synthetic construct
  misc_feature 1..23: note = modified siRNA
  source 1..23: mol_type = other RNA
tattataaaaatatcttgctttt

SEQ ID NO: 94 - RNA, 21 nt, synthetic construct
  misc_feature 1..21: note = modified siRNA
  source 1..21: mol_type = other RNA
aagcaagatatttttataata

SEQ ID NO: 95 - RNA, 23 nt, synthetic construct
  misc_feature 1..23: note = modified siRNA
  source 1..23: mol_type = other RNA
tattataaaaatatcttgctttt

SEQ ID NO: 96 - RNA, 21 nt, synthetic construct
  misc_feature 1..21: note = modified siRNA
  source 1..21: mol_type = other RNA
aagcaagatatttttataata

SEQ ID NO: 97 - RNA, 23 nt, synthetic construct
  misc_feature 1..23: note = modified siRNA
  source 1..23: mol_type = other RNA
tattataaaaatatcttgctttt

SEQ ID NO: 98 - RNA, 21 nt, synthetic construct
  misc_feature 1..21: note = modified siRNA
  source 1..21: mol_type = other RNA
aagcaagatatttttataata

SEQ ID NO: 99 - RNA, 23 nt, synthetic construct
  misc_feature 1..23: note = modified siRNA
  source 1..23: mol_type = other RNA
tattataaaaatatcttgctttt

SEQ ID NO: 100 - RNA, 21 nt, synthetic construct
  misc_feature 1..21: note = modified siRNA
  source 1..21: mol_type = other RNA
tgggatttcatgtaaccaaga

SEQ ID NO: 101 - RNA, 23 nt, synthetic construct
  misc_feature 1..23: note = modified siRNA
  source 1..23: mol_type = other RNA
tcttggttacatgaaatcccatc

SEQ ID NO: 102 - RNA, 21 nt, synthetic construct
  misc_feature 1..21: note = modified siRNA
  source 1..21: mol_type = other RNA
tgggatttcatgtaaccaaga

SEQ ID NO: 103 - RNA, 23 nt, synthetic construct
  misc_feature 1..23: note = modified siRNA
  source 1..23: mol_type = other RNA
tcttggttacatgaaatcccatc

SEQ ID NO: 104 - RNA, 21 nt, synthetic construct
  misc_feature 1..21: note = modified siRNA
  source 1..21: mol_type = other RNA
tgggatttcatgtaaccaaga

SEQ ID NO: 105 - RNA, 23 nt, synthetic construct
  misc_feature 1..23: note = modified siRNA
  source 1..23: mol_type = other RNA
tcttggttacatgaaatcccatc

SEQ ID NO: 106 - RNA, 21 nt, synthetic construct
  misc_feature 1..21: note = modified siRNA
  source 1..21: mol_type = other RNA
tgggatttcatgtaaccaaga

SEQ ID NO: 107 - RNA, 23 nt, synthetic construct
  misc_feature 1..23: note = modified siRNA
  source 1..23: mol_type = other RNA
tcttggttacatgaaatcccatc

SEQ ID NO: 108 - RNA, 19 nt, synthetic construct
  misc_feature 1..19: note = modified siRNA
  source 1..19: mol_type = other RNA
cttacgctgagtacttcga

SEQ ID NO: 109 - RNA, 19 nt, synthetic construct
  misc_feature 1..19: note = modified siRNA
  source 1..19: mol_type = other RNA
tcgaagtactcagcgtaag

SEQ ID NO: 110 - RNA, 19 nt, synthetic construct
  misc_feature 1..19: note = modified siRNA
  source 1..19: mol_type = other RNA
agatatgcacacacacgga

SEQ ID NO: 111 - RNA, 19 nt, synthetic construct
  misc_feature 1..19: note = modified siRNA
  source 1..19: mol_type = other RNA
tccgtgtgtgtgcatatct

SEQ ID NO: 112 - RNA, 19 nt, synthetic construct
  misc_feature 1..19: note = modified siRNA
  source 1..19: mol_type = other RNA
tctcgtggccttaatgaaa

SEQ ID NO: 113 - RNA, 21 nt, synthetic construct
  misc_feature 1..21: note = modified siRNA
  source 1..21: mol_type = other RNA
tttcattaaggccacgagatt

SEQ ID NO: 114 - RNA, 21 nt, synthetic construct
  misc_feature 1..21: note = modified siRNA
  source 1..21: mol_type = other RNA
gactttcatcctggaaatata

SEQ ID NO: 115 - RNA, 21 nt, synthetic construct
  misc_feature 1..21: note = modified siRNA
  source 1..21: mol_type = other RNA
aagcaagatatttttataata

SEQ ID NO: 116 - RNA, 21 nt, synthetic construct
  misc_feature 1..21: note = modified siRNA
  source 1..21: mol_type = other RNA
tgggatttcatgtaaccaaga

SEQ ID NO: 117 - RNA, 21 nt, synthetic construct
  misc_feature 1..21: note = modified siRNA
  source 1..21: mol_type = other RNA
gactttcatcctggaaatata

SEQ ID NO: 118 - RNA, 21 nt, synthetic construct
  misc_feature 1..21: note = modified siRNA
  source 1..21: mol_type = other RNA
aagcaagatatttttataata

SEQ ID NO: 119 - RNA, 21 nt, synthetic construct
  misc_feature 1..21: note = modified siRNA
  source 1..21: mol_type = other RNA
tgggatttcatgtaaccaaga

SEQ ID NO: 120 - RNA, 21 nt, synthetic construct
  misc_feature 1..21: note = modified siRNA
  source 1..21: mol_type = other RNA
gactttcatcctggaaatata

SEQ ID NO: 121 - RNA, 21 nt, synthetic construct
  misc_feature 1..21: note = modified siRNA
  source 1..21: mol_type = other RNA
aagcaagatatttttataata

SEQ ID NO: 122 - RNA, 21 nt, synthetic construct
  misc_feature 1..21: note = modified siRNA
  source 1..21: mol_type = other RNA
tgggatttcatgtaaccaaga

SEQ ID NO: 123 - RNA, 21 nt, synthetic construct
  misc_feature 1..21: note = modified siRNA
  source 1..21: mol_type = other RNA
gactttcatcctggaaatata

SEQ ID NO: 124 - RNA, 21 nt, synthetic construct
  misc_feature 1..21: note = modified siRNA
  source 1..21: mol_type = other RNA
aagcaagatatttttataata

SEQ ID NO: 125 - RNA, 21 nt, synthetic construct
  misc_feature 1..21: note = modified siRNA
  source 1..21: mol_type = other RNA
tgggatttcatgtaaccaaga

SEQ ID NO: 126 - RNA, 21 nt, synthetic construct
  misc_feature 1..21: note = modified siRNA
  source 1..21: mol_type = other RNA
gactttcatcctggaaatata

SEQ ID NO: 127 - RNA, 21 nt, synthetic construct
  misc_feature 1..21: note = modified siRNA
  source 1..21: mol_type = other RNA
aagcaagatatttttataata

SEQ ID NO: 128 - RNA, 21 nt, synthetic construct
  misc_feature 1..21: note = modified siRNA
  source 1..21: mol_type = other RNA
tgggatttcatgtaaccaaga

SEQ ID NO: 129 - RNA, 21 nt, synthetic construct
  misc_feature 1..21: note = modified siRNA
  source 1..21: mol_type = other RNA
gactttcatcctggaaatata

SEQ ID NO: 130 - RNA, 21 nt, synthetic construct
  misc_feature 1..21: note = modified siRNA
  source 1..21: mol_type = other RNA
aagcaagatatttttataata

SEQ ID NO: 131 - RNA, 21 nt, synthetic construct
  misc_feature 1..21: note = modified siRNA
  source 1..21: mol_type = other RNA
tgggatttcatgtaaccaaga

SEQ ID NO: 132 - RNA, 21 nt, synthetic construct
  misc_feature 1..21: note = modified siRNA
  source 1..21: mol_type = other RNA
gactttcatcctggaaatata

SEQ ID NO: 133 - RNA, 21 nt, synthetic construct
  misc_feature 1..21: note = modified siRNA
  source 1..21: mol_type = other RNA
aagcaagatatttttataata

SEQ ID NO: 134 - RNA, 21 nt, synthetic construct
  misc_feature 1..21: note = modified siRNA
  source 1..21: mol_type = other RNA
tgggatttcatgtaaccaaga

SEQ ID NO: 135 - RNA, 23 nt, synthetic construct
  misc_feature 1..23: note = modified siRNA
  source 1..23: mol_type = other RNA
tatatttccaggatgaaagtcca

SEQ ID NO: 136 - RNA, 23 nt, synthetic construct
  misc_feature 1..23: note = modified siRNA
  source 1..23: mol_type = other RNA
tattataaaaatatcttgctttt

SEQ ID NO: 137 - RNA, 23 nt, synthetic construct
  misc_feature 1..23: note = modified siRNA
  source 1..23: mol_type = other RNA
tcttggttacatgaaatcccatc

SEQ ID NO: 138 - RNA, 23 nt, synthetic construct
  misc_feature 1..23: note = modified siRNA
  source 1..23: mol_type = other RNA
tatatttccaggatgaaagtcca

SEQ ID NO: 139 - RNA, 23 nt, synthetic construct
  misc_feature 1..23: note = modified siRNA
  source 1..23: mol_type = other RNA
tattataaaaatatcttgctttt

SEQ ID NO: 140 - RNA, 23 nt, synthetic construct
  misc_feature 1..23: note = modified siRNA
  source 1..23: mol_type = other RNA
tcttggttacatgaaatcccatc

SEQ ID NO: 141 - RNA, 21 nt, synthetic construct
  misc_feature 1..21: note = modified siRNA
  source 1..21: mol_type = other RNA
gactttcatcctggaaatata

SEQ ID NO: 142 - RNA, 21 nt, synthetic construct
  misc_feature 1..21: note = modified siRNA
  source 1..21: mol_type = other RNA
aagcaagatatttttataata

SEQ ID NO: 143 - RNA, 21 nt, synthetic construct
  misc_feature 1..21: note = modified siRNA
  source 1..21: mol_type = other RNA
tgggatttcatgtaaccaaga

SEQ ID NO: 144 - RNA, 21 nt, synthetic construct
  misc_feature 1..21: note = modified siRNA
  source 1..21: mol_type = other RNA
gactttcatcctggaaatata

SEQ ID NO: 145 - RNA, 21 nt, synthetic construct
  misc_feature 1..21: note = modified siRNA
  source 1..21: mol_type = other RNA
aagcaagatatttttataata

SEQ ID NO: 146 - RNA, 21 nt, synthetic construct
  misc_feature 1..21: note = modified siRNA
  source 1..21: mol_type = other RNA
tgggatttcatgtaaccaaga

SEQ ID NO: 147 - RNA, 21 nt, synthetic construct
  misc_feature 1..21: note = modified siRNA
  source 1..21: mol_type = other RNA
gactttcatcctggaaatata

SEQ ID NO: 148 - RNA, 21 nt, synthetic construct
  misc_feature 1..21: note = modified siRNA
  source 1..21: mol_type = other RNA
aagcaagatatttttataata

SEQ ID NO: 149 - RNA, 21 nt, synthetic construct
  misc_feature 1..21: note = modified siRNA
  source 1..21: mol_type = other RNA
tgggatttcatgtaaccaaga

SEQ ID NO: 150 - RNA, 21 nt, synthetic construct
  misc_feature 1..21: note = modified siRNA
  source 1..21: mol_type = other RNA
gactttcatcctggaaatata

SEQ ID NO: 151 - RNA, 21 nt, synthetic construct
  misc_feature 1..21: note = modified siRNA
  source 1..21: mol_type = other RNA
aagcaagatatttttataata

SEQ ID NO: 152 - RNA, 21 nt, synthetic construct
  misc_feature 1..21: note = modified siRNA
  source 1..21: mol_type = other RNA
tgggatttcatgtaaccaaga

SEQ ID NO: 153 - RNA, 21 nt, synthetic construct
  misc_feature 1..21: note = modified siRNA
  source 1..21: mol_type = other RNA
gactttcatcctggaaatata

SEQ ID NO: 154 - RNA, 23 nt, synthetic construct
  misc_feature 1..23: note = modified siRNA
  source 1..23: mol_type = other RNA
tatatttccaggatgaaagtcca

SEQ ID NO: 155 - RNA, 21 nt, synthetic construct
  misc_feature 1..21: note = modified siRNA
  source 1..21: mol_type = other RNA
gactttcatcctggaaatata

SEQ ID NO: 156 - RNA, 23 nt, synthetic construct
  misc_feature 1..23: note = modified siRNA
  source 1..23: mol_type = other RNA
tatatttccaggatgaaagtcca

SEQ ID NO: 157 - RNA, 21 nt, synthetic construct
  misc_feature 1..21: note = modified siRNA
  source 1..21: mol_type = other RNA
gactttcatcctggaaatata

SEQ ID NO: 158 - RNA, 23 nt, synthetic construct
  misc_feature 1..23: note = modified siRNA
  source 1..23: mol_type = other RNA
tatatttccaggatgaaagtcca

SEQ ID NO: 159 - RNA, 21 nt, synthetic construct
  misc_feature 1..21: note = modified siRNA
  source 1..21: mol_type = other RNA
gactttcatcctggaaatata

SEQ ID NO: 160 - RNA, 23 nt, synthetic construct
  misc_feature 1..23: note = modified siRNA
  source 1..23: mol_type = other RNA
tatatttccaggatgaaagtcca

SEQ ID NO: 161 - RNA, 21 nt, synthetic construct
  misc_feature 1..21: note = modified siRNA
  source 1..21: mol_type = other RNA
aagcaagatatttttataata

SEQ ID NO: 162 - RNA, 23 nt, synthetic construct
  misc_feature 1..23: note = modified siRNA
  source 1..23: mol_type = other RNA
tattataaaaatatcttgctttt

SEQ ID NO: 163 - RNA, 21 nt, synthetic construct
  misc_feature 1..21: note = modified siRNA
  source 1..21: mol_type = other RNA
aagcaagatatttttataata

SEQ ID NO: 164 - RNA, 23 nt, synthetic construct
  misc_feature 1..23: note = modified siRNA
  source 1..23: mol_type = other RNA
tattataaaaatatcttgctttt

SEQ ID NO: 165 - RNA, 21 nt, synthetic construct
  misc_feature 1..21: note = modified siRNA
  source 1..21: mol_type = other RNA
aagcaagatatttttataata

SEQ ID NO: 166 - RNA, 23 nt, synthetic construct
  misc_feature 1..23: note = modified siRNA
  source 1..23: mol_type = other RNA
tattataaaaatatcttgctttt

SEQ ID NO: 167 - RNA, 21 nt, synthetic construct
  misc_feature 1..21: note = modified siRNA
  source 1..21: mol_type = other RNA
aagcaagatatttttataata

SEQ ID NO: 168 - RNA, 23 nt, synthetic construct
  misc_feature 1..23: note = modified siRNA
  source 1..23: mol_type = other RNA
tattataaaaatatcttgctttt

SEQ ID NO: 169 - RNA, 21 nt, synthetic construct
  misc_feature 1..21: note = modified siRNA
  source 1..21: mol_type = other RNA
tgggatttcatgtaaccaaga

SEQ ID NO: 170 - RNA, 23 nt, synthetic construct
  misc_feature 1..23: note = modified siRNA
  source 1..23: mol_type = other RNA
tcttggttacatgaaatcccatc

SEQ ID NO: 171 - RNA, 21 nt, synthetic construct
  misc_feature 1..21: note = modified siRNA
  source 1..21: mol_type = other RNA
tgggatttcatgtaaccaaga

SEQ ID NO: 172 - RNA, 23 nt, synthetic construct
  misc_feature 1..23: note = modified siRNA
  source 1..23: mol_type = other RNA
tcttggttacatgaaatcccatc

SEQ ID NO: 173 - RNA, 21 nt, synthetic construct
  misc_feature 1..21: note = modified siRNA
  source 1..21: mol_type = other RNA
tgggatttcatgtaaccaaga

SEQ ID NO: 174 - RNA, 23 nt, synthetic construct
  misc_feature 1..23: note = modified siRNA
  source 1..23: mol_type = other RNA
tcttggttacatgaaatcccatc

SEQ ID NO: 175 - RNA, 21 nt, synthetic construct
  misc_feature 1..21: note = modified siRNA
  source 1..21: mol_type = other RNA
tgggatttcatgtaaccaaga

SEQ ID NO: 176 - RNA, 23 nt, synthetic construct
  misc_feature 1..23: note = modified siRNA
  source 1..23: mol_type = other RNA
tcttggttacatgaaatcccatc